(19) United States
(12) Patent Application Publication
(10) Pub. No.: US 2025/0282776 A1
(43) Pub. Date: Sep. 11, 2025

(54) Bicyclic TLR7 Agonists and Uses Thereof

(71) Applicant: Bristol-Myers Squibb Company, Princeton, NJ (US)

(72) Inventors: Yam Bahadur Poudel, Fremont, CA (US); Julian Castro Lo, Burlingame, CA (US); Matthew Cox, San Francisco, CA (US); Liqi He, San Jose, CA (US); Dahlia Ruth Weiss, San Mateo, CA (US); Qian Zhang, Danville, CA (US); Murugaiah Andappan Murugaiah Subbaiah, Tamil Nadu (IN)

(73) Assignee: Bristol-Myers Squibb Company, Princeton, NJ (US)

(21) Appl. No.: 19/069,357

(22) Filed: Mar. 4, 2025

Related U.S. Application Data
(60) Provisional application No. 63/561,583, filed on Mar. 5, 2024.

(51) Int. Cl. C07D 471/04; A61K 31/519; A61K 47/68; C07D 519/00

(52) U.S. Cl. CPC C07D 471/04; A61K 31/519; A61K 47/6803; A61K 47/6849; A61K 47/6851; A61K 47/6869; C07D 519/00

(57) ABSTRACT

Compounds according to Formula (I) are useful as agonists of Toll-like receptor 7 (TLR7).

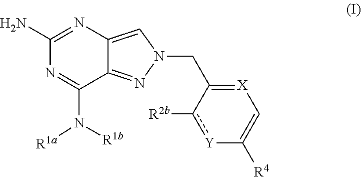

Such compounds can be used alone or as part of an antibody-drug conjugate in cancer treatment, for example in combination with an anti-cancer immunotherapy agent. Such compounds may also be used as vaccine adjuvants.

### CROSS-REFERENCE TO RELATED APPLICATION

This application claims the benefit of U.S. Provisional Application No. 63/561,583, filed Mar. 5, 2024, which is incorporated by reference herein in its entirety for any purpose.

### FIELD

This disclosure relates to Toll-like receptor 7 (“TLR7”) agonists and conjugates thereof, and methods for the preparation and use of such agonists and their conjugates.

### BACKGROUND

Toll-like receptors (“TLRs”) are receptors that recognize pathogen-associated molecular patterns (“PAMPs”), which are small molecular motifs conserved in certain classes of pathogens. TLRs can be located either on a cell's surface or intracellularly. Activation of a TLR by the binding of its cognate PAMP signals the presence of the associated pathogen inside the host—i.e., an infection—and stimulates the host's immune system to fight the infection. Humans have 10 TLRs, named TLR1, TLR2, TLR3, and so on.

The activation of a TLR—with TLR7 being the most studied—by an agonist can have a positive effect on the action of vaccines and immunotherapy agents in treating a variety of conditions other than actual pathogen infection, by stimulating the immune response overall. Thus, there is considerable interest in the use of TLR7 agonists as vaccine adjuvants or as enhancers in cancer immunotherapy. See, for example, Vasilakos and Tomai 2013, Sato-Kaneko et al. 2017, Smits et al. 2008, and Ota et al. 2019.

TLR7, an intracellular receptor located on the membrane of endosomes, recognizes PAMPs associated with single-stranded RNA viruses. Its activation induces secretion of Type I interferons such as IFNα and IFNβ (Lund et al. 2004). TLR7 has two binding sites, one for single stranded RNA ligands (Berghofer et al. 2007) and one for small molecules such as guanosine (Zhang et al. 2016).

TLR7 can bind to, and be activated by, guanosine-like synthetic agonists such as imiquimod, resiquimod, and gardiquimod, which are based on a 1H-imidazo[4,5-c]quinoline scaffold. For a review of small-molecule TLR7 agonists, see Cortez and Va 2018.

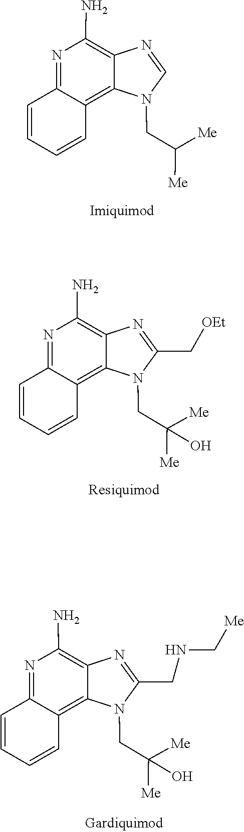 [SMILES: CC(C)Cn1cnc2c(N)nc3ccccc3c21 ; CCOCc1nc2c(N)nc3ccccc3c2n1CC(C)(C)O ; CCNCc1nc2c(N)nc3ccccc3c2n1CC(C)(C)O]

Synthetic TLR7 agonists based on a pteridinone molecular scaffold are also known, as exemplified by vesatolimod (Desai et al. 2015).

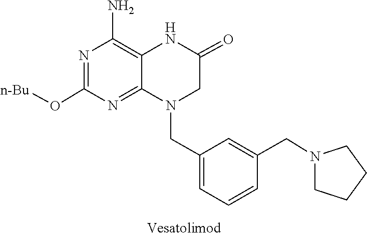

Other synthetic TLR7 agonists based on a purine-like scaffold have been disclosed, frequently according to the general formula (A):

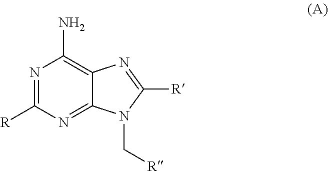

where R, R′, and R″ are structural variables, with R″ typically containing an unsubstituted or substituted aromatic or heteroaromatic ring.

Disclosures of bioactive molecules having a purine-like scaffold and their uses in treating conditions such as fibrosis, inflammatory disorders, cancer, or pathogenic infections include: Akinbobuyi et al. 2015 and 2016; Barberis et al. 2012; Carson et al. 2014; Ding et al. 2016, 2017a, and 2017b; Graupe et al. 2015; Hashimoto et al. 2009; He et al. 2019a and 2019b; Holldack et al. 2012; Isobe et al. 2009a and 2012; Poudel et al. 2019a and 2019b; Pryde 2010; and Young et al. 2019.

The group R″ can be pyridyl: Bonfanti et al. 2015a and 2015b; Halcomb et al. 2015; Hirota et al. 2000; Isobe et al. 2002, 2004, 2006, 2009a, 2009b, 2011, and 2012; Kasibhatla et al. 2007; Koga-Yamakawa et al. 2013; Musmuca et al. 2009; Nakamura 2012; Ogita et al. 2007; and Yu et al. 2013.

There are disclosures of related molecules in which the 6,5-fused ring system of formula (A)—a pyrimidine six member ring fused to an imidazole five member ring—is modified. (a) Dellaria et al. 2007, Jones et al. 2010 and 2012, and Pilatte et al. 2017 disclose compounds in which the pyrimidine ring is replaced by a pyridine ring. (b) Chen et al. 2011, Coe et al. 2017, and Zhang et al. 2018 disclose compounds in which the imidazole ring is replaced by a pyrazole ring. (c) Cortez et al. 2017 and 2018; Li et al. 2018; and McGowan et al. 2016a, 2016b, and 2017 disclose compounds in which the imidazole ring is replaced by a pyrrole ring.

Bonfanti et al. 2015b and 2016 and Purandare et al. 2019 disclose TLR7 modulators in which the two rings of a purine moiety are spanned by a macrocycle:

A TLR7 agonist can be conjugated to a partner molecule, which can be, for example, a phospholipid, a poly(ethylene glycol) (“PEG”), an antibody, or another TLR (commonly TLR2). Exemplary disclosures include: Carson et al. 2013, 2015, and 2016, Chan et al. 2009 and 2011, Cortez et al. 2017, Gadd et al. 2015, Lioux et al. 2016, Maj et al. 2015, Vernejoul et al. 2014, and Zurawski et al. 2012. A frequent conjugation site is at the R″ group of formula (A).

Jensen et al. 2015 discloses the use of cationic lipid vehicles for the delivery of TLR7 agonists.

Some TLR7 agonists, including resiquimod are dual TLR7/TLR8 agonists. See, for example, Beesu et al. 2017, Embrechts et al. 2018, Lioux et al. 2016, and Vernejoul et al. 2014.

Full citations for the documents cited herein by first author or inventor and year are listed at the end of this specification.

### BRIEF SUMMARY OF THE DISCLOSURE

This specification relates to compounds having a 1H-pyrazolo[4,3-d]pyrimidine aromatic system, having activity as TLR7 agonists.

In some embodiments, a TLR7 agonist provided herein is also an agonist of TLR8.

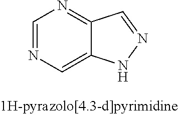

In one aspect, there is provided a compound with a structure according to Formula (I)

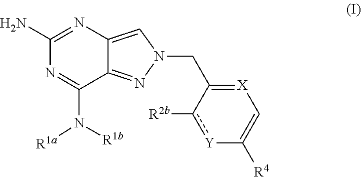

or a pharmaceutically acceptable salt thereof, wherein:
R^{1a} and R^{1b} are each independently C₁-C₆ alkyl, optionally substituted by halo, —OH or —O(C₁-C₃ alkyl);
X is N or CR^{2a};
R^{2a} and R^{2b} are each independently H, —O(C₁-C₃ alkyl), —(C₁-C₃ haloalkyl), halo, —CN, or —NH(C₁-C₃ alkyl);
Y is CR³, NR³, or N;
R³ is H or —O(C₁-C₃ alkyl);
or R^{2b} and R³ can be taken together to form 5- to 6-membered heteroaryl or 5- to 6-membered heterocyclyl, optionally substituted with C₁-C₃ alkyl, —C(O)CH₂N(C₁-C₃ alkyl)₂, or —C(O)(CH₂)NH(C₃-C₆ cycloalkyl), wherein the heteroaryl and heterocyclyl contain 1-3 heteroatoms selected from N, O, and S;
R⁴ is H, —CH₂OH, optionally substituted 3- to 6-membered heterocyclyl containing 1 heteroatom selected from N and O, optionally substituted C₃-C₆ cycloalkyl, or —CH₂NR^{5a}R^{5b}; and
R^{5a} is H and R^{5b} is optionally substituted 4- to 7-membered heterocyclyl, optionally substituted C₃-C₆ cycloalkyl, optionally substituted C₁-C₆ alkyl, wherein the heterocyclyl contains 1 heteroatom selected from N and O, or R^{5a} and R^{5b} are taken together to form optionally substituted 4- to 8-membered heterocyclyl containing 1-2 heteroatoms selected from N and O.

Compounds disclosed herein have activity as TLR7 agonists and some can be conjugated to an antibody for targeted delivery to a target tissue or organ of intended action. They can also be PEGylated, to modulate their pharmaceutical properties.

Compounds disclosed herein, or their conjugates or their PEGylated derivatives, can be used in the treatment of a subject suffering from a condition amenable to treatment by activation of the immune system, by administering to such subject a therapeutically effective amount of such a compound or a conjugate thereof or a PEGylated derivative thereof, especially in combination with a vaccine or a cancer immunotherapy agent.

### DETAILED DESCRIPTION

### Definitions

“Antibody” as used herein means whole antibodies and any antigen binding fragment (i.e., “antigen-binding portion”) or single chain variants thereof. A whole, or full length, antibody is a protein comprising at least two heavy (H) chains and two light (L) chains inter-connected by disulfide bonds. Each heavy chain comprises a heavy chain variable region (V_{H}) and a heavy chain constant region comprising three domains, C_{H1}, C_{H2} and C_{H3}. Each light chain comprises a light chain variable region (V_{L} or Vₖ) and a light chain constant region comprising one single domain, C_{L}. The V_{H} and V_{L} regions can be further subdivided into regions of hypervariability, termed complementarity determining regions (CDRs), interspersed with more conserved framework regions (FRs). Each V_{H} and V_{L} comprises three CDRs and four FRs, arranged from amino- to carboxy-terminus in the following order: FR1, CDR1, FR2, CDR2, FR3, CDR3, and FR4. The variable regions contain a binding domain that interacts with an antigen. The constant regions may mediate the binding of the antibody to host tissues or factors, including various cells of the immune system (e.g., effector cells) and the first component (Clq) of the classical complement system. An antibody is said to “specifically bind” to an antigen X if the antibody binds to antigen X with a K_{D} of 5×10⁻⁸ M or less, more preferably 1×10⁻⁸ M or less, more preferably 6×10⁻⁹ M or less, more preferably 3×10⁻⁹ M or less, even more preferably 2×10⁻⁹ M or less. The antibody can be chimeric, humanized, or, preferably, human. The heavy chain constant region can be engineered to affect glycosylation type or extent, to extend antibody half-life, to enhance or reduce interactions with effector cells or the complement system, or to modulate some other property. The engineering can be accomplished by replacement, addition, or deletion of one or more amino acids or by replacement of a domain with a domain from another immunoglobulin type, or a combination of the foregoing.

“Antigen binding fragment” and “antigen binding portion” of an antibody (or simply “antibody portion” or “antibody fragment”) mean one or more fragments of an antibody that retain the ability to specifically bind to an antigen. It has been shown that the antigen-binding function of an antibody can be performed by fragments of a full-length antibody, such as (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fab′ fragment, which is essentially an Fab with part of the hinge region (see, for example, Abbas et al., Cellular and Molecular Immunology, 6th Ed., Saunders Elsevier 2007); (iv) a Fd fragment consisting of the V_{H} and C_{H1} domains; (v) a Fv fragment consisting of the V_{L} and V_{H} domains of a single arm of an antibody, (vi) a dAb fragment (Ward et al., (1989) Nature 341:544-546), which consists of a V_{H} domain; (vii) an isolated complementarity determining region (CDR); and (viii) a nanobody, a heavy chain variable region containing a single variable domain and two constant domains. Preferred antigen binding fragments are Fab, F(ab′)₂, Fab′, Fv, and Fd fragments. Furthermore, although the two domains of the Fv fragment, V_{L} and V_{H}, are encoded by separate genes, they can be joined, using recombinant methods, by a synthetic linker that enables them to be made as a single protein chain in which the V_{L} and V_{H} regions pair to form monovalent molecules (known as single chain Fv, or scFv); see, e.g., Bird et al. (1988) Science 242:423-426; and Huston et al. (1988) Proc. Natl. Acad. Sci. USA 85:5879-5883). Such single chain antibodies are also encompassed within the term “antigen-binding portion” of an antibody.

Unless indicated otherwise—for example by reference to the linear numbering in a SEQ ID NO: listing—references to the numbering of amino acid positions in an antibody heavy or light chain variable region (V_{H} or V_{L}) are according to the Kabat system (Kabat et al., “Sequences of proteins of immunological interest, 5th ed., Pub. No. 91-3242, U.S. Dept. Health & Human Services, NIH, Bethesda, Md., 1991, hereinafter “Kabat”) and references to the numbering of amino acid positions in an antibody heavy or light chain constant region (C_{H1}, C_{H2}, C_{H3}, or C_{L}) are according to the EU index as set forth in Kabat. See Lazar et al., US 2008/0248028 A1, the disclosure of which is incorporated herein by reference, for examples of such usage. Further, the ImMunoGeneTics Information System (IMGT) provides at its website a table entitled “IMGT Scientific Chart: Correspondence between C Numberings” showing the correspondence between its numbering system, EU numbering, and Kabat numbering for the heavy chain constant region.

An “isolated antibody” means an antibody that is substantially free of other antibodies having different antigenic specificities (e.g., an isolated antibody that specifically binds antigen X is substantially free of antibodies that specifically bind antigens other than antigen X). An isolated antibody that specifically binds antigen X may, however, have cross-reactivity to other antigens, such as antigen X molecules from other species. In certain embodiments, an isolated antibody specifically binds to human antigen X and does not cross-react with other (non-human) antigen X antigens. Moreover, an isolated antibody may be substantially free of other cellular material and/or chemicals.

“Monoclonal antibody” or “monoclonal antibody composition” means a preparation of antibody molecules of single molecular composition, which displays a single binding specificity and affinity for a particular epitope.

“Human antibody” means an antibody having variable regions in which both the framework and CDR regions (and the constant region, if present) are derived from human germline immunoglobulin sequences. Human antibodies may include later modifications, including natural or synthetic modifications. Human antibodies may include amino acid residues not encoded by human germline immunoglobulin sequences (e.g., mutations introduced by random or site-specific mutagenesis in vitro or by somatic mutation in vivo). However, “human antibody” does not include antibodies in which CDR sequences derived from the germline of another mammalian species, such as a mouse, have been grafted onto human framework sequences.

“Human monoclonal antibody” means an antibody displaying a single binding specificity, which has variable regions in which both the framework and CDR regions are derived from human germline immunoglobulin sequences. In one embodiment, human monoclonal antibodies are produced by a hybridoma that includes a B cell obtained from a transgenic nonhuman animal, e.g., a transgenic mouse, having a genome comprising a human heavy chain transgene and a light chain transgene fused to an immortalized cell.

An “alkyl” group is a saturated, partially saturated, or unsaturated straight chain or branched non-cyclic hydrocarbon having from 1 to 10 carbon atoms (C₁-C₁₀ alkyl), typically from 1 to 8 carbons (C₁-C₈ alkyl) or, in some embodiments, from 1 to 6 (C₁-C₆ alkyl), 1 to 4 (C₁-C₄ alkyl), 1 to 3 (C₁-C₃ alkyl), or 2 to 6 (C₂-C₆ alkyl) carbon atoms. In some embodiments, the alkyl group is a saturated alkyl group. Representative saturated alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like. In some embodiments, an alkyl group is an unsaturated alkyl group, also termed an alkenyl or alkynyl group. An “alkenyl” group is an alkyl group that contains one or more carbon-carbon double bonds. An “alkynyl” group is an alkyl group that contains one or more carbon-carbon triple bonds. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₂CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen; hydroxy; alkoxy; cycloalkyloxy, aryloxy, heterocyclyloxy, heteroaryloxy, heterocycloalkyloxy, cycloalkylalkyloxy, aralkyloxy, heterocyclylalkyloxy, heteroarylalkyloxy, heterocycloalkylalkyloxy; oxo (═O); amino, alkylamino, cycloalkylamino, arylamino, heterocyclylamino, heteroarylamino, heterocycloalkylamino, cycloalkylalkylamino, aralkylamino, heterocyclylalkylamino, heteroaralkylamino, heterocycloalkylalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

An “alkylene” group refers to the same residues as alkyl, but having bivalency. Particular alkylene groups are those having from 1 to 10 carbon atoms (C₁-C₁₀ alkylene), typically from 1 to 8 carbons (C₁-C₈ alkylene) or, in some embodiments, from 1 to 6 (C₁-C₆ alkylene) or 1 to 3 (C₁-C₃ alkylene) carbon atoms. Examples of alkylene include, but are not limited to, groups such as methylene (—CH₂—), ethylene (—CH₂CH₂—), propylene (—CH₂CH₂CH₂—), isopropylene (—CH₂CH(CH₃)—), butylene (—CH₂(CH₂)₂CH₂—), isobutylene (—CH₂CH(CH₃)CH₂—), pentylene (—CH₂(CH₂)₃CH₂—), hexylene (—CH₂(CH₂)₄CH₂—), heptylene (—CH₂(CH₂)₅CH₂—), octylene (—CH₂(CH₂)₆CH₂—), and the like.

A “cycloalkyl” group is a saturated, or partially saturated cyclic alkyl group of from 3 to 10 carbon atoms (C₃-C₁₀ cycloalkyl) having a single cyclic ring or multiple condensed or bridged rings that can be optionally substituted. In some embodiments, the cycloalkyl group has 3 to 8 ring carbon atoms (C₃-C₈ cycloalkyl), whereas in other embodiments the number of ring carbon atoms ranges from 3 to 5 (C₃-C₅ cycloalkyl), 3 to 6 (C₃-C₆ cycloalkyl), or 3 to 7 (C₃-C₇ cycloalkyl). In some embodiments, the cycloalkyl groups are saturated cycloalkyl groups. Such saturated cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. In other embodiments, the cycloalkyl groups are unsaturated cycloalkyl groups. Examples of unsaturared cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

A “heterocyclyl” is a non-aromatic cycloalkyl in which one to four of the ring carbon atoms are independently replaced with a heteroatom selected from O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. Heterocyclyl groups encompass saturated and partially saturated ring systems. Further, the term heterocyclyl is intended to encompass any non-aromatic ring containing at least one heteroatom, which ring may be fused to an aryl or heteroaryl ring, regardless of the attachment to the remainder of the molecule. The phrase also includes bridged polycyclic ring systems containing a heteroatom. Representative examples of a heterocyclyl group include, but are not limited to, aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, or tetrahydropyrimidin-2(1H)-one. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “heterocyclylene” group refers to a divalent “heterocyclyl” group.

An “aryl” group is an aromatic carbocyclic group of from 6 to 14 carbon atoms (C₆-C₁₄ aryl) having a single ring (e.g., phenyl) or multiple condensed rings (e.g., naphthyl or anthryl). In some embodiments, aryl groups contain 6-14 carbons (C₆-C₁₄ aryl), and in others from 6 to 12 (C₆-C₁₂ aryl) or even 6 to 10 carbon atoms (C₆-C₁₀ aryl) in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heteroaryl” group is an aromatic ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1H-pyrrolo[2,3-b]pyridyl), indazolyl, benzimidazolyl (e.g., 1H-benzo[d]imidazolyl), imidazopyridyl (e.g., azabenzimidazolyl or 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4-dihydroisoquinolin-1(2H)-onyl), tetrahydroquinolinyl, quinoxalinyl, and quinazolinyl groups. A heteroaryl group can be substituted or unsubstituted.

A “halogen” or “halo” is fluorine, chlorine, bromine or iodine.

An “alkoxy” group is —O-(alkyl), wherein alkyl is defined above.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. In some embodiments, the haloalkyl group has one to six carbon atoms and is substituted by one or more halo radicals (C₁-C₆ haloalkyl), or the haloalkyl group has one to three carbon atoms and is substituted by one or more halo radicals (C₁-C₃ haloalkyl). The halo radicals may be all the same or the halo radicals may be different. Unless specifically stated otherwise, a haloalkyl group is optionally substituted.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

Embodiments of the disclosure are meant to encompass pharmaceutically acceptable salts, tautomers, isotopologues, and stereoisomers of the compounds provided herein, such as the compounds of Formula (I).

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds of Formula (I) include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, maleic, phosphoric, sulfuric, and methanesulfonic acids. Examples of specific salts thus include hydrochloride, formic, and mesylate salts. Others are well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereoisomerically pure” means one stereoisomer of a particular compound that is substantially free of other stereoisomers of that compound. For example, a stereoisomerically pure compound having one chiral center will be substantially free of the opposite enantiomer of the compound. A stereoisomerically pure compound having two chiral centers will be substantially free of other diastereomers of the compound. A typical stereoisomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds disclosed herein can have chiral centers and can occur as racemates, individual enantiomers or diastereomers, and mixtures thereof. All such isomeric forms are included within the embodiments disclosed herein, including mixtures thereof.

The use of stereoisomerically pure forms of the compounds disclosed herein, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); Wilen, S. H., Tables of Resolving Agents and Optical Resolutions p. 268 (E. L. Eliel, Ed., Univ. of Notre Dame Press, Notre Dame, IN, 1972); Todd, M., Separation Of Enantiomers: Synthetic Methods (Wiley-VCH Verlag GmbH & Co. KGaA, Weinheim, Germany, 2014); Toda, F., Enantiomer Separation: Fundamentals and Practical Methods (Springer Science & Business Media, 2007); Subramanian, G. Chiral Separation Techniques: A Practical Approach (John Wiley & Sons, 2008); Ahuj a, S., Chiral Separation Methods for Pharmaceutical and Biotechnological Products (John Wiley & Sons, 2011).

It should also be noted the compounds disclosed herein can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

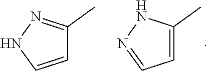

As readily understood by one skilled in the art, a wide variety of functional groups and other structures may exhibit tautomerism and all tautomers of compounds of Formula (I) are within the scope of the present disclosure.

It should also be noted the compounds disclosed herein can contain unnatural proportions of atomic isotopes at one or more of the atoms. For example, the compounds may be radiolabeled with radioactive isotopes, such as for example tritium (³H), iodine-125 (¹²⁵I) sulfur-35 (³⁵S), or carbon-14 (¹⁴C), or may be isotopically enriched, such as with deuterium (2H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds disclosed herein, for example, the isotopologues are deuterium, carbon-13, and/or nitrogen-15 enriched compounds. As used herein, “deuterated”, means a compound wherein at least one hydrogen (H) has been replaced by deuterium (indicated by D or ²H), that is, the compound is enriched in deuterium in at least one position.

It is understood that, independently of stereoisomerical or isotopic composition, each compound disclosed herein can be provided in the form of any of the pharmaceutically acceptable salts discussed herein. Equally, it is understood that the isotopic composition may vary independently from the stereoisomerical composition of each compound referred to herein. Further, the isotopic composition, while being restricted to those elements present in the respective compound or salt thereof disclosed herein, may otherwise vary independently from the selection of the pharmaceutically acceptable salt of the respective compound.

Where a range is stated, as in “C₁-C₅ alkyl” or “5 to 10%,” such range includes the end points of the range, as in C₁ and C₅ in the first instance and 5% and 10% in the second instance.

“Pharmaceutically acceptable salt” means a salt of a compound suitable for pharmaceutical formulation. Where a compound has one or more basic groups, the salt can be an acid addition salt, such as a sulfate, hydrobromide, tartrate, mesylate, maleate, citrate, phosphate, acetate, pamoate (embonate), hydroiodide, nitrate, hydrochloride, lactate, methylsulfate, fumarate, benzoate, succinate, mesylate, lactobionate, suberate, tosylate, and the like. Where a compound has one or more acidic groups, the salt can be a salt such as a calcium salt, potassium salt, magnesium salt, meglumine salt, ammonium salt, zinc salt, piperazine salt, tromethamine salt, lithium salt, choline salt, diethylamine salt, 4-phenylcyclohexylamine salt, benzathine salt, sodium salt, tetramethylammonium salt, and the like. Polymorphic crystalline forms and solvates are also encompassed within the scope of this invention.

“Subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human. In various embodiments, a subject or patient is a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. The “treatment of cancer”, refers to one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

In the formulae of this specification, a wavy line ( [symbol] ) transverse to a bond at the end of the bond or a wavy line ( [symbol] ) transverse to a bond at the middle of the bond denotes a covalent attachment site. For instance, a statement that R is

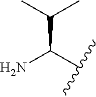 [SMILES: *[C@@H](N)C(C)C]

or that R is

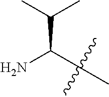 [SMILES: *[C@@H](N)C(C)C]

in the formula

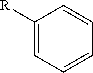

means

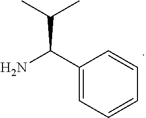

In the formulae of this specification, a bond traversing an aromatic ring between two carbons thereof means that the group attached to the bond may be located at any of the positions of the aromatic ring made available by removal of the hydrogen that is implicitly there. By way of illustration, the formula

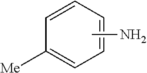

represents

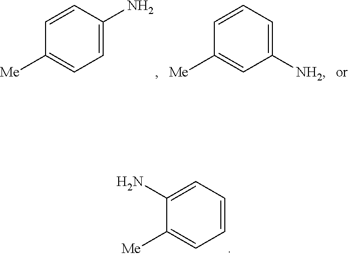 [SMILES: Cc1ccc(N)cc1 ; Cc1cccc(N)c1 ; Cc1ccccc1N]

In other illustrations,

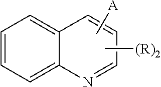

represents

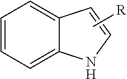

represents

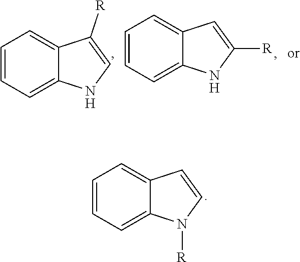

Those skilled in the art will appreciate that certain structures can be drawn in one tautomeric form or another—for example, keto versus enol—and that the two forms are equivalent.

### Compounds

In one aspect, provided herein is a compound of Formula (I):

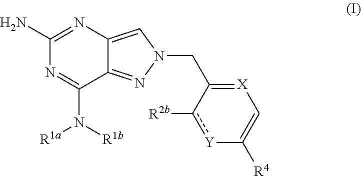

or a pharmaceutically acceptable salt thereof, wherein:
R^{1a} and R^{1b} are each independently C₁-C₆ alkyl, optionally substituted by halo, —OH or —O(C₁-C₃ alkyl);
X is N or CR^{2a};
R^{2a} and R^{2b} are each independently H, —O(C₁-C₃ alkyl), —(C₁-C₃ haloalkyl), halo, —CN, or —NH(C₁-C₃ alkyl);
Y is CR³, NR³, or N;
R³ is H or —O(C₁-C₃ alkyl);
or R^{2b} and R³ can be taken together to form 5- to 6-membered heteroaryl or 5- to 6-membered heterocyclyl, optionally substituted with C₁-C₃ alkyl, —C(O)CH₂N(C₁-C₃ alkyl)₂, or —C(O)(CH₂)NH(C₃-C₆ cycloalkyl), wherein the heteroaryl and heterocyclyl contain 1-3 heteroatoms selected from N, O, and S;
R⁴ is H, —CH₂OH, optionally substituted 3- to 6-membered heterocyclyl containing 1 heteroatom selected from N and O, optionally substituted C₃-C₆ cycloalkyl, or —CH₂NR^{5a}R^{5b}; and
R^{5a} is H and R^{5b} is optionally substituted 4- to 7-membered heterocyclyl, optionally substituted C₃-C₆ cycloalkyl, optionally substituted C₁-C₆ alkyl, wherein the heterocyclyl contains 1 heteroatom selected from N and O, or R^{5a} and R^{5b} are taken together to form optionally substituted 4- to 8-membered heterocyclyl containing 1-2 heteroatoms selected from N and O.

In some embodiments, R^{1a} and R^{1b} are each independently C₁-C₆ alkyl, optionally substituted by halo, —OH or —O(C₁-C₃ alkyl). In some embodiments, R^{1a} and R^{1b} are each independently C₁-C₅ alkyl, optionally substituted by F, Cl, Br, I, —OH or —O(C₁-C₃ alkyl). In some embodiments, R^{1a} and R^{1b} are each independently C₁-C₅ alkyl, optionally substituted by F, —OH, —OCH₃, —OCH₂CH₃, or —OCH₂CH₂CH₃. In some embodiments, R^{1a} and R^{1b} are each independently C₁-C₅ alkyl, optionally substituted by F, —OH or —OCH₃. In some embodiments, R^{1a} and R^{1b} are each independently methyl, ethyl, propyl, butyl, or pentyl, optionally substituted by F, —OH or —OCH₃.

In some embodiments, X is N or CR^{2a}. In some embodiments, X is N. In some embodiments, X is CR^{2a}.

In some embodiments, R^{2a} and R^{2b} are each independently H, —O(C₁-C₃ alkyl), —(C₁-C₃ haloalkyl), halo, —CN, or —NH(C₁-C₃ alkyl). In some embodiments, R^{2a} and R^{2b} are each independently H, —O(C₁-C₃ alkyl), —(C₁ haloalkyl), halo, —CN, or —NH(C₁-C₃ alkyl). In some embodiments, R^{2a} and R^{2b} are each independently H, —OCH₃, —OCH₂CH₃, —OCH₂CH₂CH₃, —CH₂F, —CHF₂, —CF₃, F, Cl, Br, I, —CN, —NHCH₃, —NHCH₂CH₃, or —NHCH₂CH₂CH₃. In some embodiments, R^{2a} and R^{2b} are each independently H, —OCH₃, —CHF₂, F, Cl, Br, I, —CN, or —NHCH₃.

In some embodiments, Y is CR³, NR³, or N. In some embodiments, Y is CR³. In some embodiments, Y is NR³. In some embodiments, Y is N.

In some embodiments, R³ is H or —O(C₁-C₃ alkyl). In some embodiments, R³ is H. In some embodiments, R³ is —O(C₁-C₃ alkyl). In some embodiments, R³ is —OCH₃, —OCH₂CH₃, or —OCH₂CH₂CH₃. In some embodiments, R³ is —OCH₃.

In some embodiments, R^{2b} and R³ can be taken together to form 5- to 6-membered heteroaryl or 5- to 6-membered heterocyclyl, optionally substituted with C₁-C₃ alkyl, —C(O)CH₂N(C₁-C₃ alkyl)₂, or —C(O)(CH₂)NH(C₃-C₆ cycloalkyl), wherein the heteroaryl and heterocyclyl contain 1-3 heteroatoms selected from N, O, and S. In some embodiments, R^{2b} and R³ are taken together to form 5- to 6-membered heteroaryl or 5- to 6-membered heterocyclyl, optionally substituted with methyl, ethyl, propyl, —C(O)CH₂N(CH₃)₂, —C(O)CH₂N(CH₂CH₃)₂, —C(O)CH₂N(CH₂CH₂CH₃)₂, —C(O)(CH₂)NH(cyclopropyl), —C(O)(CH₂)NH(cyclobutyl), —C(O)(CH₂)NH(cyclopentyl), or —C(O)(CH₂)NH(cyclohexyl), wherein the heteroaryl and heterocyclyl contain 1-3 heteroatoms selected from N, O, and S. In some embodiments, R^{2b} and R³ are taken together to form 5- to 6-membered heteroaryl or 5- to 6-membered heterocyclyl, optionally substituted with methyl, —C(O)CH₂N(CH₃)₂, or —C(O)(CH₂)NH(cyclobutyl), wherein the heteroaryl and heterocyclyl contain 1-3 heteroatoms selected from N, O, and S.

In some embodiments, R^{2b} and R³ can be taken together to form 5- to 6-membered heteroaryl, optionally substituted with C₁-C₃ alkyl, —C(O)CH₂N(C₁-C₃ alkyl)₂, or —C(O)(CH₂)NH(C₃-C₆ cycloalkyl), wherein the heteroaryl contains 1-3 heteroatoms selected from N, O, and S. In some embodiments, R^{2b} and R³ are taken together to form 5- to 6-membered heteroaryl, optionally substituted with methyl, ethyl, propyl, —C(O)CH₂N(CH₃)₂, —C(O)CH₂N(CH₂CH₃)₂, —C(O)CH₂N(CH₂CH₂CH₃)₂, —C(O)(CH₂)NH(cyclopropyl), —C(O)(CH₂)NH(cyclobutyl), —C(O)(CH₂)NH(cyclopentyl), or —C(O)(CH₂)NH(cyclohexyl), wherein the heteroaryl contains 1-3 heteroatoms selected from N, O, and S. In some embodiments, R^{2b} and R³ are taken together to form 5- to 6-membered heteroaryl, optionally substituted with methyl, —C(O)CH₂N(CH₃)₂, or —C(O)(CH₂)NH(cyclobutyl), wherein the heteroaryl contains 1-3 heteroatoms selected from N, O, and S.

In some embodiments, R^{2b} and R³ can be taken together to form 5- to 6-membered heterocyclyl, optionally substituted with C₁-C₃ alkyl, —C(O)CH₂N(C₁-C₃ alkyl)₂, or —C(O)(CH₂)NH(C₃-C₆ cycloalkyl), wherein the heterocyclyl contains 1-3 heteroatoms selected from N, O, and S. In some embodiments, R^{2b} and R³ are taken together to form 5- to 6-membered heterocyclyl, optionally substituted with methyl, ethyl, propyl, —C(O)CH₂N(CH₃)₂, —C(O)CH₂N(CH₂CH₃)₂, —C(O)CH₂N(CH₂CH₂CH₃)₂, —C(O)(CH₂)NH(cyclopropyl), —C(O)(CH₂)NH(cyclobutyl), —C(O)(CH₂)NH(cyclopentyl), or —C(O)(CH₂)NH(cyclohexyl), wherein the heterocyclyl contains 1-3 heteroatoms selected from N, O, and S. In some embodiments, R^{2b} and R³ are taken together to form 5- to 6-membered heterocyclyl, optionally substituted with methyl, —C(O)CH₂N(CH₃)₂, or —C(O)(CH₂)NH(cyclobutyl), wherein the heterocyclyl contains 1-3 heteroatoms selected from N, O, and S.

In some embodiments, R is

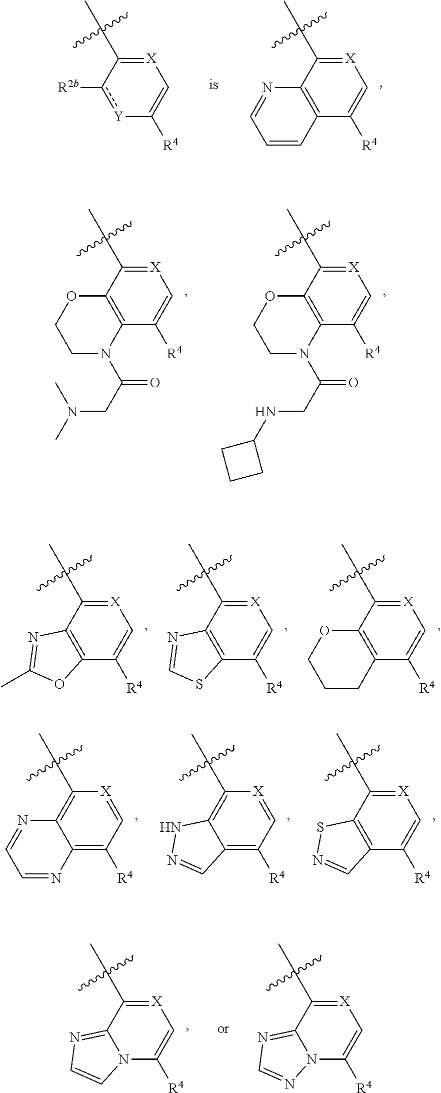

In some embodiments, R⁴ is H, —CH₂OH, optionally substituted 3- to 6-membered heterocyclyl containing 1 heteroatom selected from N and O, optionally substituted C₃-C₆ cycloalkyl, or —CH₂NR^{5a}R^{5b}. In some embodiments, R⁴ is H, —CH₂OH, optionally substituted 5- to 6-membered heterocyclyl containing 1 heteroatom selected from N and O, optionally substituted C₅-C₆ cycloalkyl, or —CH₂NR^{5a}R^{5b}. In some embodiments, R⁴ is H, —CH₂OH, optionally substituted 5- to 6-membered heterocyclyl containing 1 heteroatom selected from N and O, optionally substituted cyclopentyl, optionally substituted cyclohexyl, or —CH₂NR^{5a}R^{5b}. In some embodiments, R⁴ is H, —CH₂OH, optionally substituted 5- to 6-membered heterocyclyl containing 1 heteroatom selected from N and O, optionally substituted cyclohexyl, or —CH₂NR^{5a}R^{5b}. In some embodiments, R⁴ is H. In some embodiments, R⁴ is —CH₂OH. In some embodiments, R⁴ is optionally substituted 3- to 6-membered heterocyclyl containing 1 heteroatom selected from N and O. In some embodiments, R⁴ is optionally substituted 5- to 6-membered heterocyclyl containing 1 heteroatom selected from N and O. In some embodiments, R⁴ is optionally substituted C₃-C₆ cycloalkyl. In some embodiments, R⁴ is optionally substituted C₅-C₆ cycloalkyl. In some embodiments, R⁴ is optionally substituted cyclopentyl or optionally substituted cyclohexyl. In some embodiments, R⁴ is optionally substituted cyclohexyl. In some embodiments, R⁴ is —CH₂NR^{5a}R^{5b}.

In some embodiments, R^{5a} is H and R^{5b} is optionally substituted 4- to 7-membered heterocyclyl, optionally substituted C₃-C₆ cycloalkyl, or optionally substituted C₁-C₆ alkyl, wherein the heterocyclyl contains 1 heteroatom selected from N and O. In some embodiments, R^{5a} is H and R^{5b} is optionally substituted 4- to 7-membered heterocyclyl, optionally substituted C₃-C₆ cycloalkyl, or C₁-C₆ alkyl optionally substituted with —OH, —O(C₁-C₃ alkyl), or C₃-C₆ cycloalkyl, wherein the heterocyclyl contains 1 heteroatom selected from N and O. In some embodiments, R^{5a} is H and R^{5b} is optionally substituted 4- to 7-membered heterocyclyl, optionally substituted C₃-C₆ cycloalkyl, or methyl, ethyl, propyl, butyl, pentyl, or hexyl, each optionally substituted with —OH, —O(C₁-C₃ alkyl), or C₃-C₆ cycloalkyl, wherein the heterocyclyl contains 1 heteroatom selected from N and O. In some embodiments, R^{5a} is H and R^{5b} is optionally substituted 4- to 7-membered heterocyclyl, optionally substituted C₃-C₆ cycloalkyl, or methyl, ethyl, propyl, butyl, pentyl, or hexyl, each optionally substituted with —OH, —OCH₃, —OCH₂CH₃, —OCH₂CH₂CH₃, or C₃-C₆ cycloalkyl, wherein the heterocyclyl contains 1 heteroatom selected from N and O.

In some embodiments, R^{5a} is H and R^{5b} is optionally substituted 4- to 7-membered heterocyclyl, wherein the heterocyclyl contains 1 heteroatom selected from N and O. In some embodiments, R^{5a} is H and R^{5b} is optionally substituted C₃-C₆ cycloalkyl. In some embodiments, R^{5a} is H and R^{5b} is optionally substituted C₁-C₆ alkyl. In some embodiments, R^{5a} is H and R^{5b} is C₁-C₆ alkyl optionally substituted with —OH, —O(C₁-C₃ alkyl), or C₃-C₆ cycloalkyl. In some embodiments, R^{5a} is H and R^{5b} is methyl, ethyl, propyl, butyl, pentyl, or hexyl, each optionally substituted with —OH, —O(C₁-C₃ alkyl), or C₃-C₆ cycloalkyl. In some embodiments, R^{5a} is H and R^{5b} is methyl, ethyl, propyl, butyl, pentyl, or hexyl, each optionally substituted with —OH, —OCH₃, —OCH₂CH₃, —OCH₂CH₂CH₃, or C₃-C₆ cycloalkyl.

In some embodiments, R^{5a} and R^{5b} are taken together to form optionally substituted 4- to 8-membered heterocyclyl containing 1-2 heteroatoms selected from N and O.

In some embodiments, R⁴ is

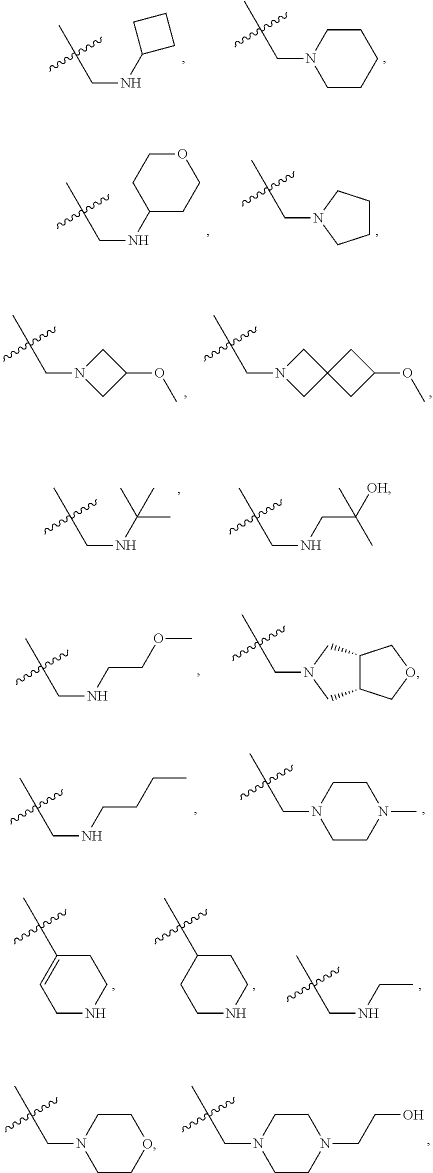

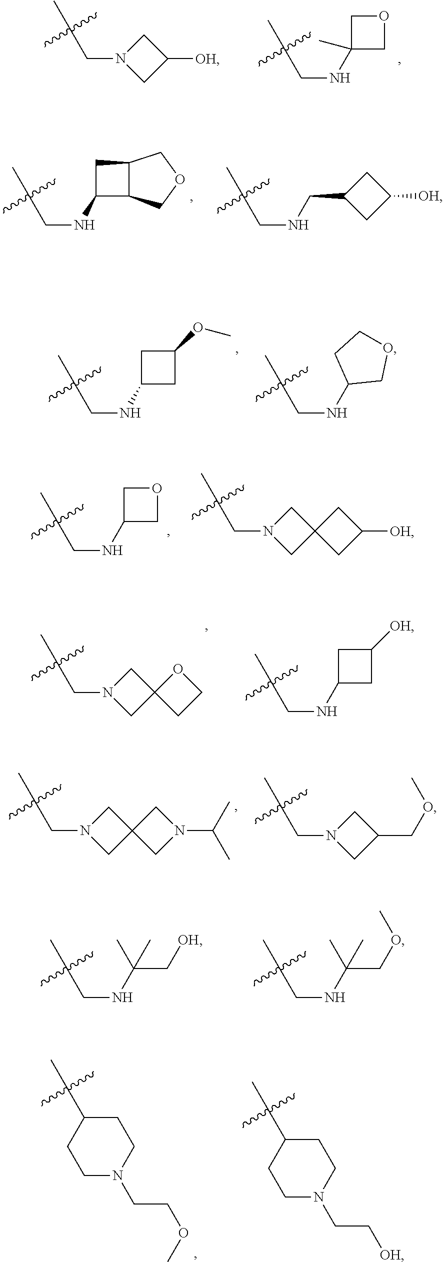

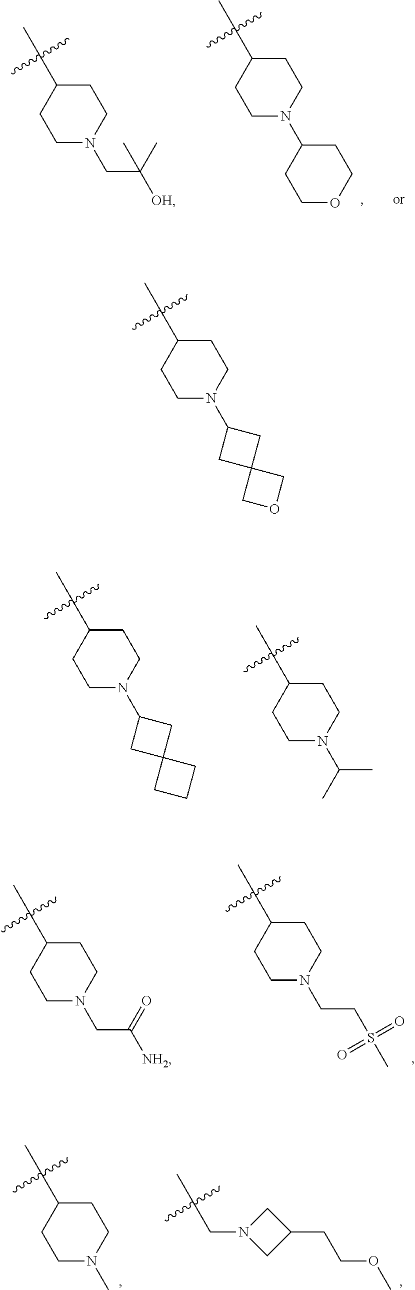

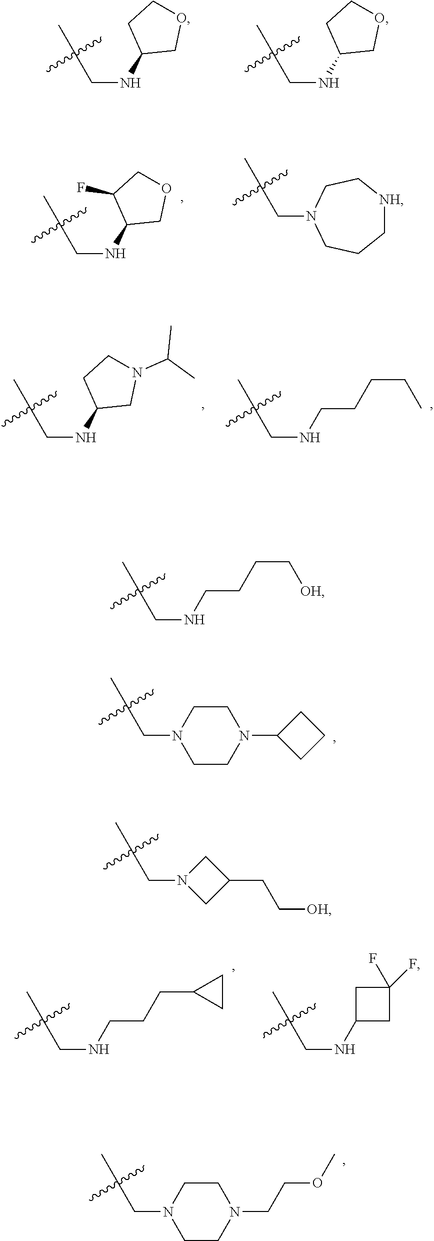

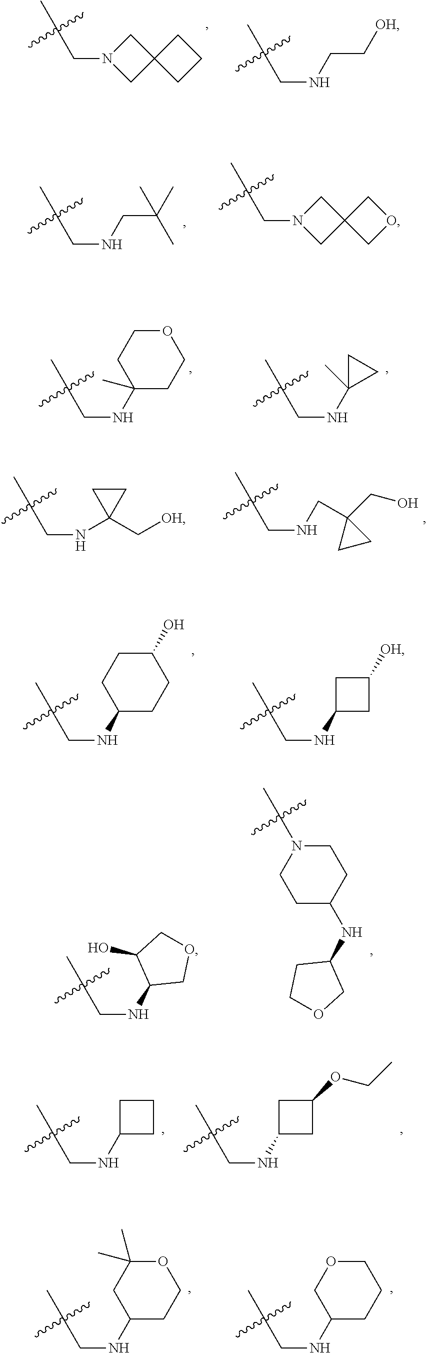

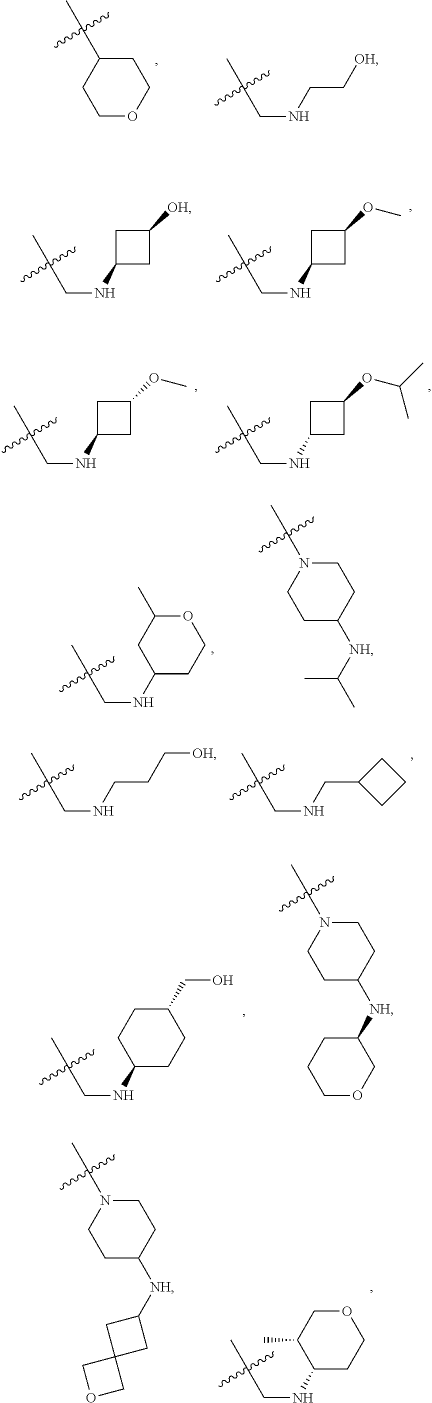

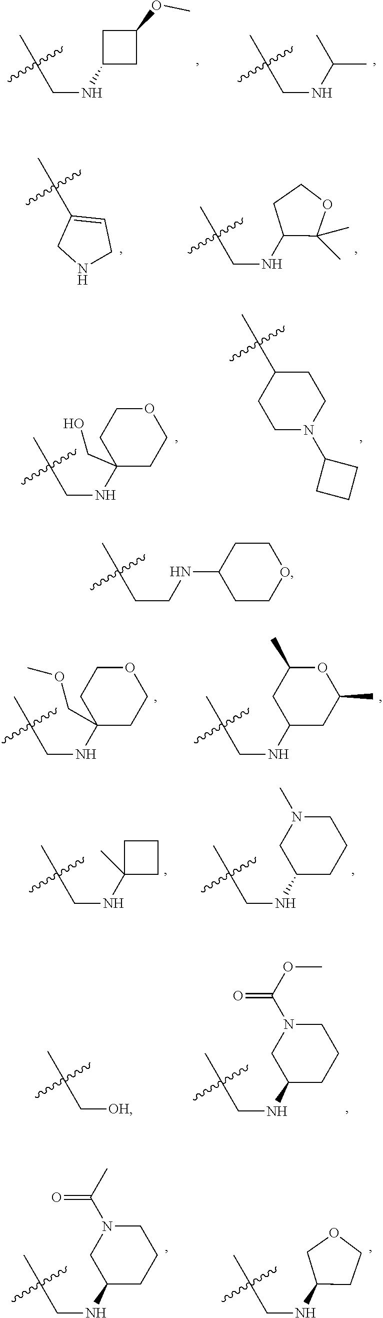

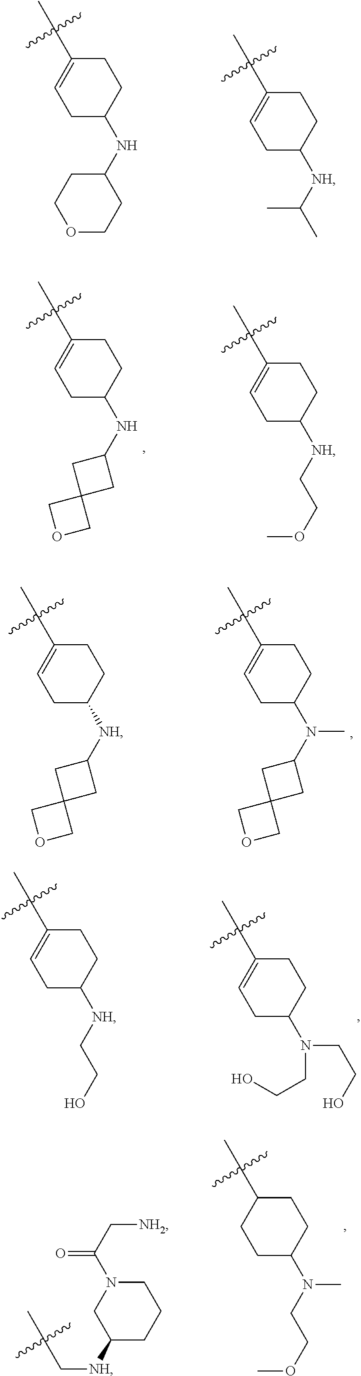

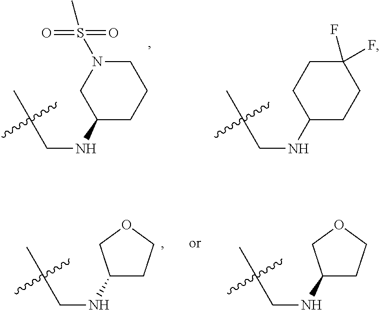 [SMILES: *CN[C@@H]1CCCN(S(C)(=O)=O)C1 ; *CNC1CCC(F)(F)CC1 ; *CN[C@H]1CCOC1 ; *CN[C@@H]1CCOC1]

In some embodiments, the compound of Formula (I) is a compound of Formula (II).

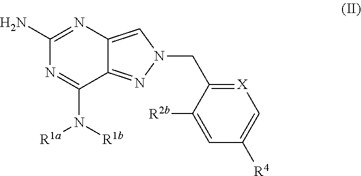

wherein R^{1a}, R^{1b}, R^{2a}, R^{2b}, and R⁴ are as described for Formula (I).

In some embodiments, the compound of Formula (I) is a compound of Formula (IIa), (IIb), (IIc), (IId), (IIe), (IIf), or (IIg).

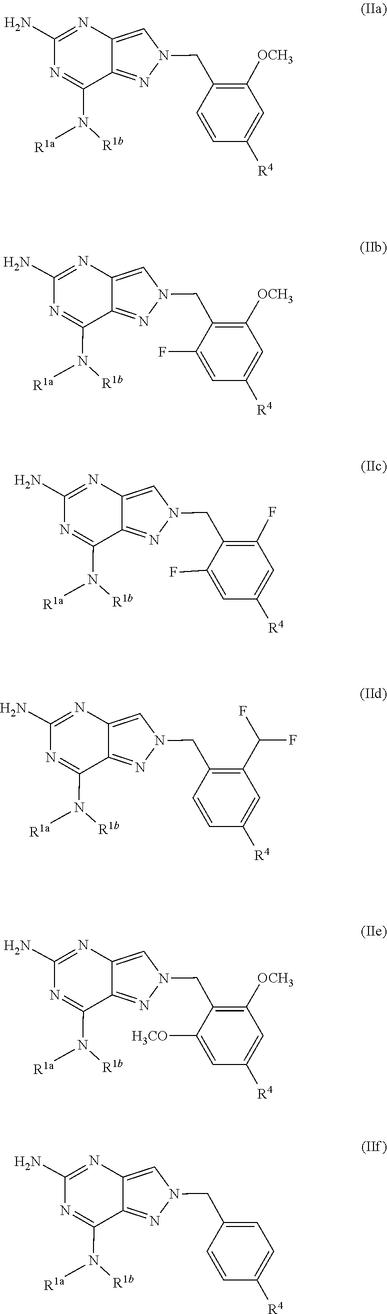

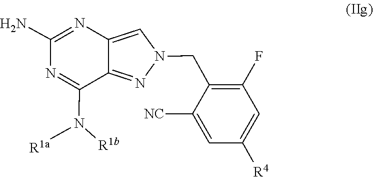

wherein R^{1a}, R^{1b}, and R⁴ are as described for Formula (I).

In some embodiments, the compound of Formula (I) is a compound of Formula (III):

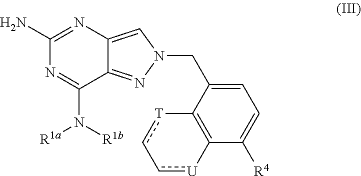

wherein T is N or O, U is CH, N, NC(O)CH₂N(CH₃)₂, or NC(O)(CH₂)NH(cyclobutyl), and Ria R^{1b}, and R⁴ are as described for Formula (I).

In some embodiments, the compound of Formula (I) is a compound of Formula (IVa) or (IVb):

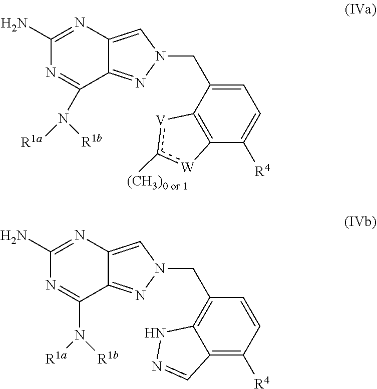

wherein V is N or S, W is N, O, or S, and R^{1a}, R^{1b}, and R⁴ are as described for Formula (I).

In some embodiments, the compound of Formula (I) is a compound of Formula (V):

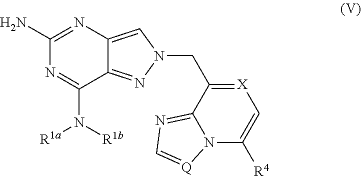

wherein Q is N or CH and R^{1a}, R^{1b}, and R⁴ are as described for Formula (I).

In the descriptions herein, it is understood that every description, variation, embodiment, or aspect of a moiety may be combined with every description, variation, embodiment, or aspect of other moieties, the same as if each and every combination of descriptions is specifically and individually listed. For example, every description, variation, embodiment, or aspect provided herein with respect to R^{1a} of Formula (I) may be combined with every description, variation, embodiment, or aspect of R^{1b}, X, R^{2a}, R^{2b}, Y, R³, R⁴, R^{1a}, and R^{1b} the same as if each and every combination were specifically and individually listed. It is also understood that all descriptions, variations, embodiments, or aspects of Formula (I), where applicable, apply equally to other formulae detailed herein, and are equally described, the same as if each and every description, variation, embodiment, or aspect were separately and individually listed for all formulae. For example, all descriptions, variations, embodiments, or aspects of Formula (I), where applicable, apply equally to any of the formulae as detailed herein, such as Formulae (II), (IIa), (IIb), (IIc), (IId), (IIe), (IIf), (IIg), (III), (IVa), (IVb), and (V), and are equally described, the same as if each and every description, variation, embodiment, or aspect were separately and individually listed for all formulae.

In some embodiments, provided is a compound selected from the compounds in Table 1A or Table 1B or a pharmaceutically acceptable salt thereof. Although certain compounds described in the present disclosure, including in Tables 1A and 1, are presented as specific stereoisomers and/or in a non-stereochemical form, it is understood that any or all stereochemical forms, including any enantiomeric or diastereomeric forms, and any tautomers or other forms of any of the compounds of the present disclosure, including in Table 1A and Table 1B, are herein described.

TABLE 1A

| Compound No. | Structure |
|---|---|
| 1 | 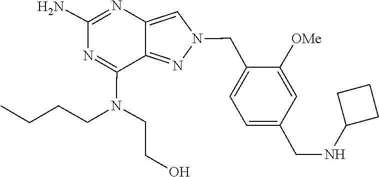 [SMILES: CCCCN(CCO)c1nc(N)nc2cn(Cc3ccc(CNC4CCC4)cc3OC)nc12] |
| 2 | 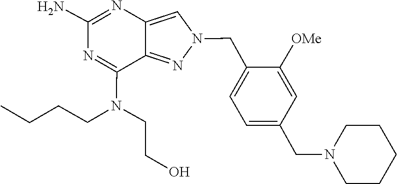 [SMILES: CCCCN(CCO)c1nc(N)nc2cn(Cc3ccc(CN4CCCCC4)cc3OC)nc12] |
| 3 | 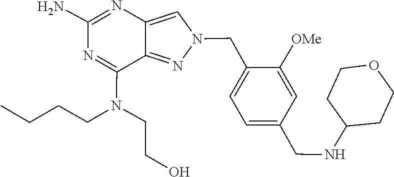 [SMILES: CCCCN(CCO)c1nc(N)nc2cn(Cc3ccc(CNC4CCOCC4)cc3OC)nc12] |
| 4 | 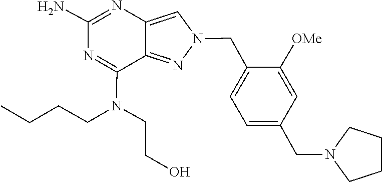 [SMILES: CCCCN(CCO)c1nc(N)nc2cn(Cc3ccc(CN4CCCC4)cc3OC)nc12] |
| 5 | 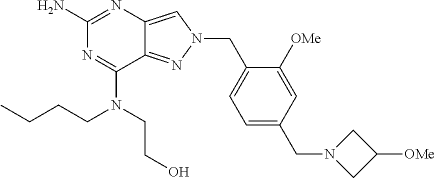 [SMILES: CCCCN(CCO)c1nc(N)nc2cn(Cc3ccc(CN4CC(OC)C4)cc3OC)nc12] |
| 6 | 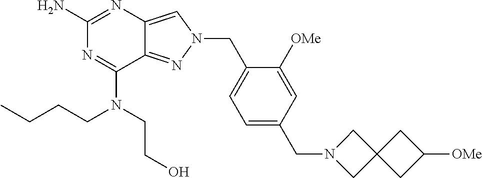 [SMILES: CCCCN(CCO)c1nc(N)nc2cn(Cc3ccc(CN4CC5(CC(OC)C5)C4)cc3OC)nc12] |
| 7 | 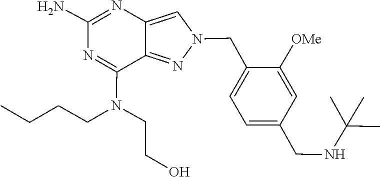 [SMILES: CCCCN(CCO)c1nc(N)nc2cn(Cc3ccc(CNC(C)(C)C)cc3OC)nc12] |
| 8 | 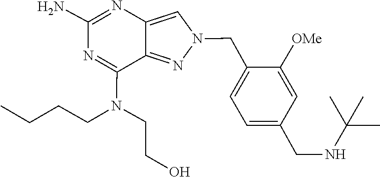 [SMILES: CCCCN(CCO)c1nc(N)nc2cn(Cc3ccc(CNC(C)(C)C)cc3OC)nc12] |
| 9 | 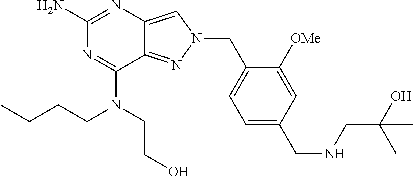 [SMILES: CCCCN(CCO)c1nc(N)nc2cn(Cc3ccc(CNCC(C)(C)O)cc3OC)nc12] |
| 10 | 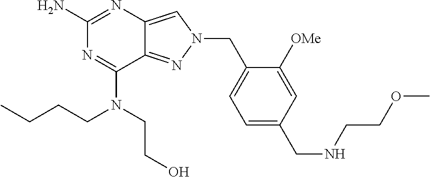 [SMILES: CCCCN(CCO)c1nc(N)nc2cn(Cc3ccc(CNCCOC)cc3OC)nc12] |
| 11 | 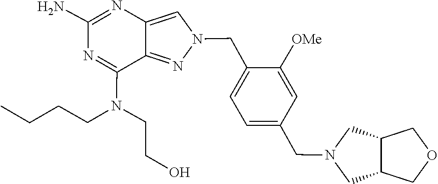 [SMILES: CCCCN(CCO)c1nc(N)nc2cn(Cc3ccc(CN4C[C@H]5COC[C@H]5C4)cc3OC)nc12] |
| 12 | 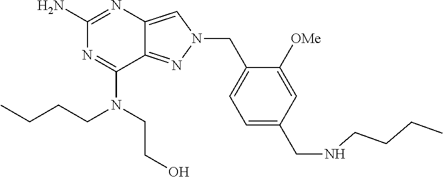 [SMILES: CCCCNCc1ccc(Cn2cc3nc(N)nc(N(CCO)CCCC)c3n2)c(OC)c1] |
| 13 | 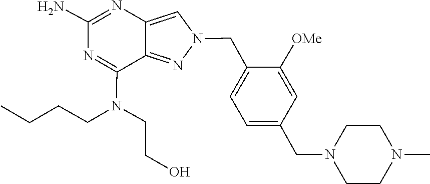 [SMILES: CCCCN(CCO)c1nc(N)nc2cn(Cc3ccc(CN4CCN(C)CC4)cc3OC)nc12] |
| 15 | 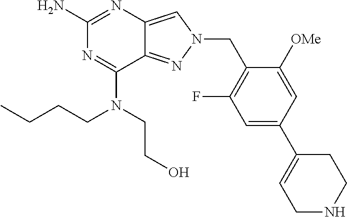 [SMILES: CCCCN(CCO)c1nc(N)nc2cn(Cc3c(F)cc(C4=CCNCC4)cc3OC)nc12] |
| 16 | 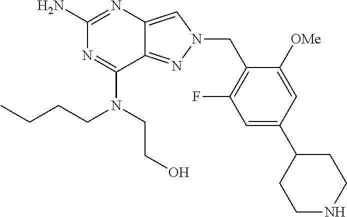 [SMILES: CCCCN(CCO)c1nc(N)nc2cn(Cc3c(F)cc(C4CCNCC4)cc3OC)nc12] |
| 17 | 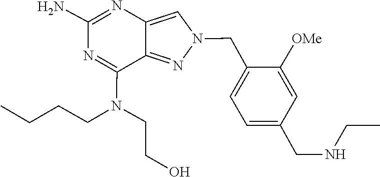 [SMILES: CCCCN(CCO)c1nc(N)nc2cn(Cc3ccc(CNCC)cc3OC)nc12] |
| 18 | 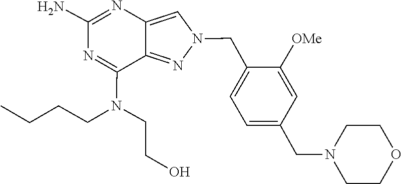 [SMILES: CCCCN(CCO)c1nc(N)nc2cn(Cc3ccc(CN4CCOCC4)cc3OC)nc12] |
| 19 | 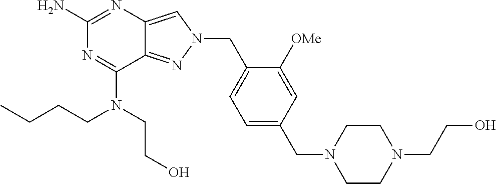 [SMILES: CCCCN(CCO)c1nc(N)nc2cn(Cc3ccc(CN4CCN(CCO)CC4)cc3OC)nc12] |
| 20 | 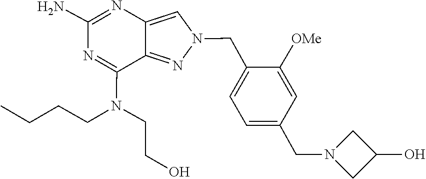 [SMILES: CCCCN(CCO)c1nc(N)nc2cn(Cc3ccc(CN4CC(O)C4)cc3OC)nc12] |
| 21 | 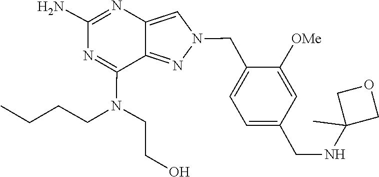 [SMILES: CCCCN(CCO)c1nc(N)nc2cn(Cc3ccc(CNC4(C)COC4)cc3OC)nc12] |
| 22 | 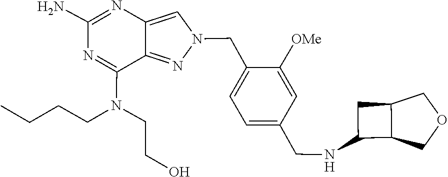 |
| 23 | 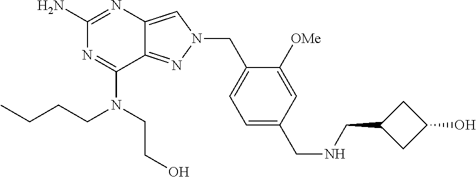 |
| 24 | 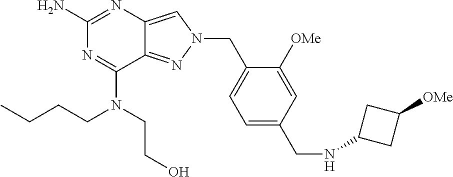 |
| 25 | 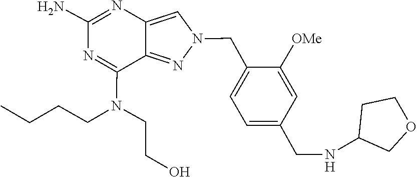 [SMILES: CCCCN(CCO)c1nc(N)nc2cn(Cc3ccc(CNC4CCOC4)cc3OC)nc12] |
| 26 | 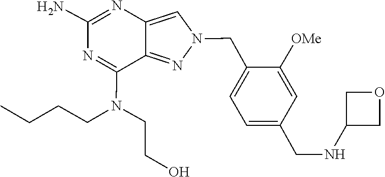 [SMILES: CCCCN(CCO)c1nc(N)nc2cn(Cc3ccc(CNC4COC4)cc3OC)nc12] |
| 27 | 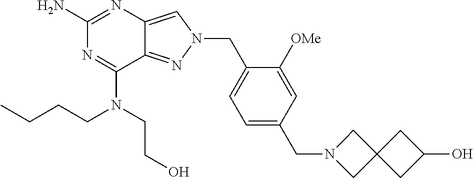 [SMILES: CCCCN(CCO)c1nc(N)nc2cn(Cc3ccc(CN4CC5(CC(O)C5)C4)cc3OC)nc12] |
| 28 | 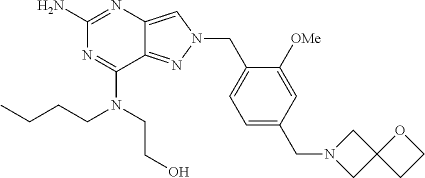 [SMILES: CCCCN(CCO)c1nc(N)nc2cn(Cc3ccc(CN4CC5(CCO5)C4)cc3OC)nc12] |
| 29 | 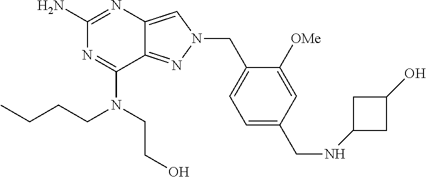 [SMILES: CCCCN(CCO)c1nc(N)nc2cn(Cc3ccc(CNC4CC(O)C4)cc3OC)nc12] |
| 30 | 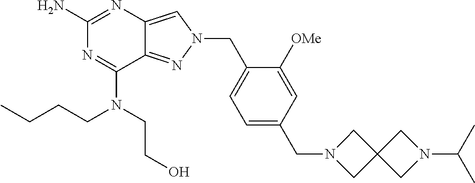 [SMILES: CCCCN(CCO)c1nc(N)nc2cn(Cc3ccc(CN4CC5(C4)CN(C(C)C)C5)cc3OC)nc12] |
| 31 | 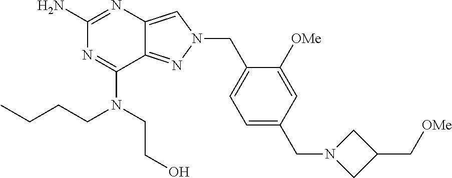 [SMILES: CCCCN(CCO)c1nc(N)nc2cn(Cc3ccc(CN4CC(COC)C4)cc3OC)nc12] |
| 32 | 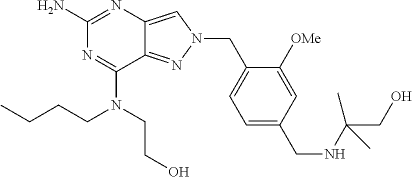 [SMILES: CCCCN(CCO)c1nc(N)nc2cn(Cc3ccc(CNC(C)(C)CO)cc3OC)nc12] |
| 33 | 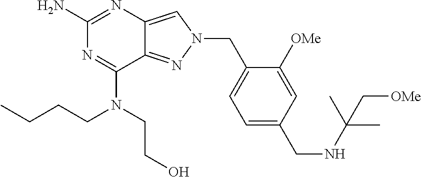 [SMILES: CCCCN(CCO)c1nc(N)nc2cn(Cc3ccc(CNC(C)(C)COC)cc3OC)nc12] |
| 34 | 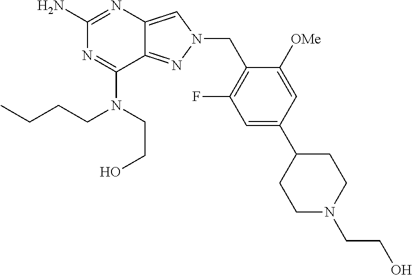 [SMILES: CCCCN(CCO)c1nc(N)nc2cn(Cc3c(F)cc(C4CCN(CCO)CC4)cc3OC)nc12] |
| 35 | 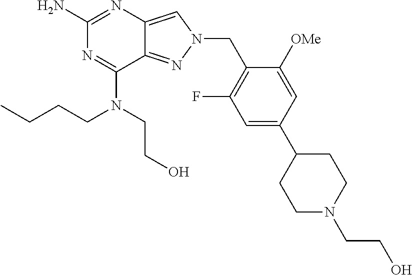 [SMILES: CCCCN(CCO)c1nc(N)nc2cn(Cc3c(F)cc(C4CCN(CCO)CC4)cc3OC)nc12] |
| 36 | 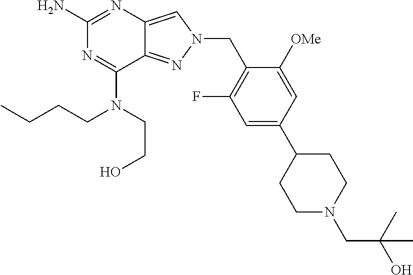 [SMILES: CCCCN(CCO)c1nc(N)nc2cn(Cc3c(F)cc(C4CCN(CC(C)(C)O)CC4)cc3OC)nc12] |
| 37 | 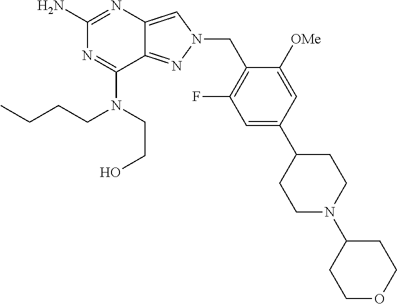 [SMILES: CCCCN(CCO)c1nc(N)nc2cn(Cc3c(F)cc(C4CCN(C5CCOCC5)CC4)cc3OC)nc12] |
| 38 | 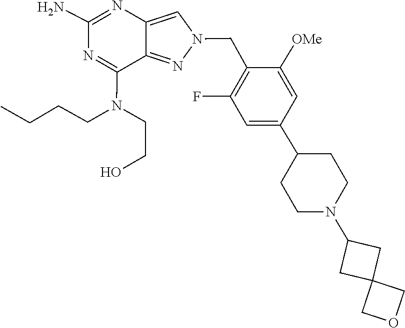 [SMILES: CCCCN(CCO)c1nc(N)nc2cn(Cc3c(F)cc(C4CCN(C5CC6(COC6)C5)CC4)cc3OC)nc12] |
| 39 | 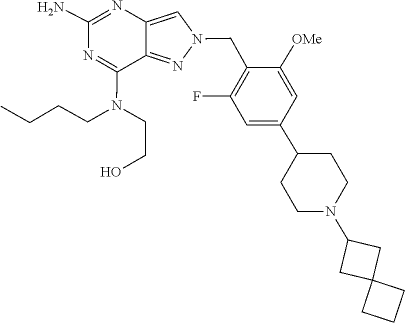 [SMILES: CCCCN(CCO)c1nc(N)nc2cn(Cc3c(F)cc(C4CCN(C5CC6(CCC6)C5)CC4)cc3OC)nc12] |
| 40 | 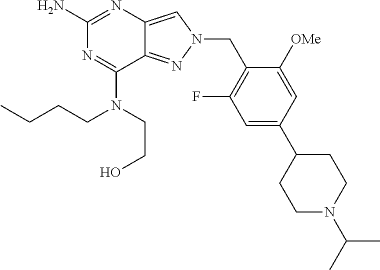 [SMILES: CCCCN(CCO)c1nc(N)nc2cn(Cc3c(F)cc(C4CCN(C(C)C)CC4)cc3OC)nc12] |
| 41 | 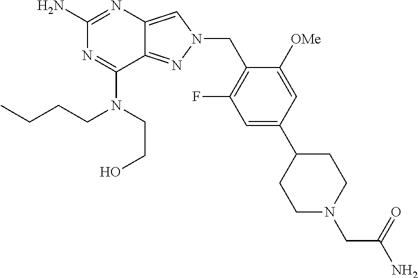 [SMILES: CCCCN(CCO)c1nc(N)nc2cn(Cc3c(F)cc(C4CCN(CC(N)=O)CC4)cc3OC)nc12] |
| 42 | 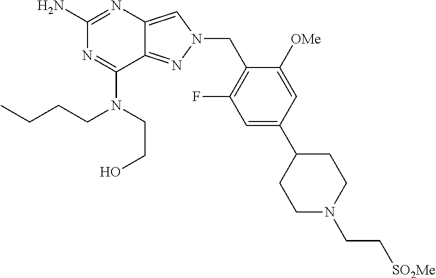 [SMILES: CCCCN(CCO)c1nc(N)nc2cn(Cc3c(F)cc(C4CCN(CCS(C)(=O)=O)CC4)cc3OC)nc12] |
| 43 | 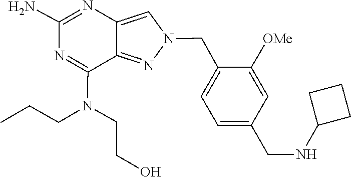 [SMILES: CCCN(CCO)c1nc(N)nc2cn(Cc3ccc(CNC4CCC4)cc3OC)nc12] |
| 44 | 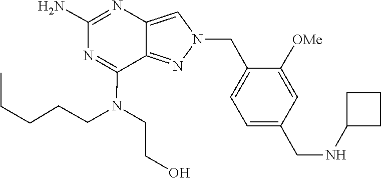 [SMILES: CCCCCN(CCO)c1nc(N)nc2cn(Cc3ccc(CNC4CCC4)cc3OC)nc12] |
| 45 | 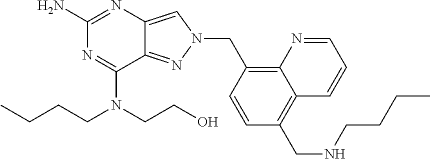 [SMILES: CCCCNCc1ccc(Cn2cc3nc(N)nc(N(CCO)CCCC)c3n2)c2ncccc12] |
| 46 | 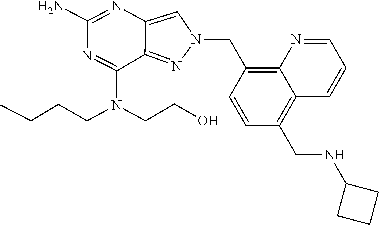 [SMILES: CCCCN(CCO)c1nc(N)nc2cn(Cc3ccc(CNC4CCC4)c4cccnc34)nc12] |
| 47 | 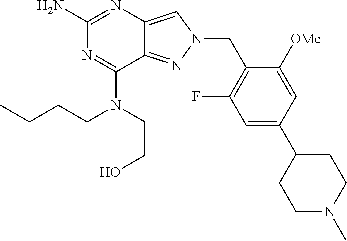 [SMILES: CCCCN(CCO)c1nc(N)nc2cn(Cc3c(F)cc(C4CCN(C)CC4)cc3OC)nc12] |
| 48 | 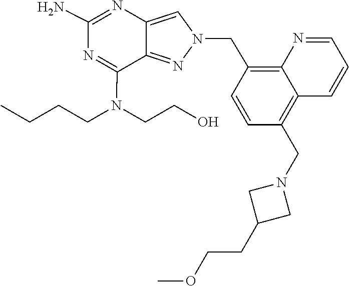 [SMILES: CCCCN(CCO)c1nc(N)nc2cn(Cc3ccc(CN4CC(CCOC)C4)c4cccnc34)nc12] |
| 49 | 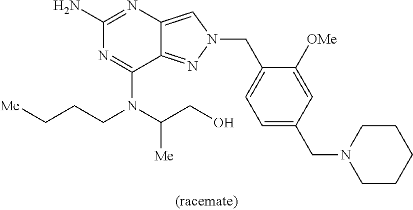 [SMILES: CCCCN(c1nc(N)nc2cn(Cc3ccc(CN4CCCCC4)cc3OC)nc12)C(C)CO] |
| 49-1 | 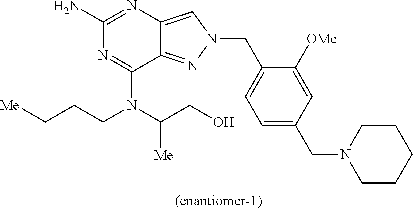 [SMILES: CCCCN(c1nc(N)nc2cn(Cc3ccc(CN4CCCCC4)cc3OC)nc12)C(C)CO] |
| 49-2 | 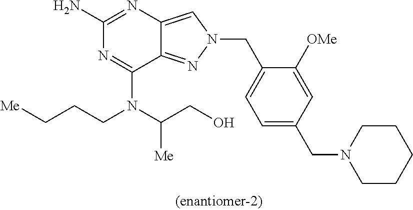 [SMILES: CCCCN(c1nc(N)nc2cn(Cc3ccc(CN4CCCCC4)cc3OC)nc12)C(C)CO] |
| 50 | 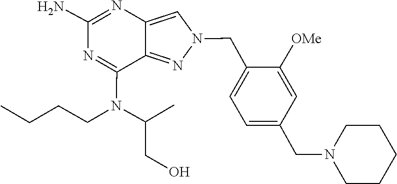 [SMILES: CCCCN(c1nc(N)nc2cn(Cc3ccc(CN4CCCCC4)cc3OC)nc12)C(C)CO] |
| 52 | 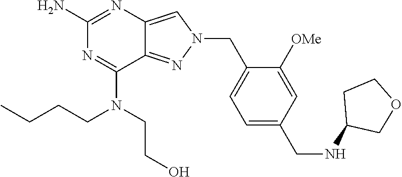 [SMILES: CCCCN(CCO)c1nc(N)nc2cn(Cc3ccc(CN[C@H]4CCOC4)cc3OC)nc12] |
| 53 | 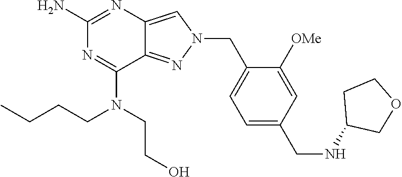 [SMILES: CCCCN(CCO)c1nc(N)nc2cn(Cc3ccc(CN[C@@H]4CCOC4)cc3OC)nc12] |
| 54 | 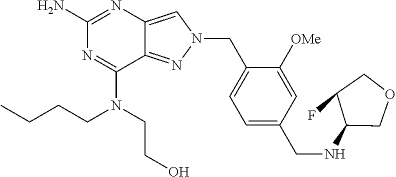 [SMILES: CCCCN(CCO)c1nc(N)nc2cn(Cc3ccc(CN[C@@H]4COC[C@@H]4F)cc3OC)nc12] |
| 55 | 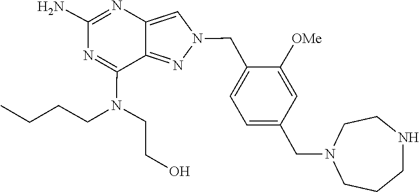 [SMILES: CCCCN(CCO)c1nc(N)nc2cn(Cc3ccc(CN4CCCNCC4)cc3OC)nc12] |
| 56 | 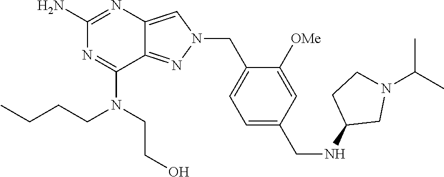 [SMILES: CCCCN(CCO)c1nc(N)nc2cn(Cc3ccc(CN[C@H]4CCN(C(C)C)C4)cc3OC)nc12] |
| 57 | 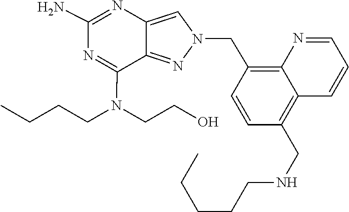 [SMILES: CCCCCNCc1ccc(Cn2cc3nc(N)nc(N(CCO)CCCC)c3n2)c2ncccc12] |
| 58 | 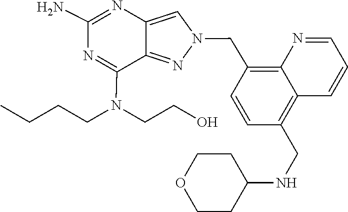 [SMILES: CCCCN(CCO)c1nc(N)nc2cn(Cc3ccc(CNC4CCOCC4)c4cccnc34)nc12] |
| 59 | 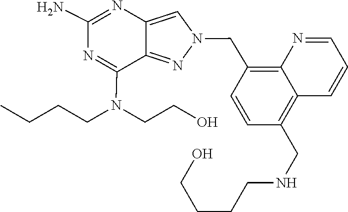 [SMILES: CCCCN(CCO)c1nc(N)nc2cn(Cc3ccc(CNCCCCO)c4cccnc34)nc12] |
| 60 | 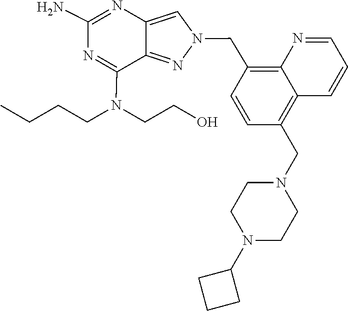 [SMILES: CCCCN(CCO)c1nc(N)nc2cn(Cc3ccc(CN4CCN(C5CCC5)CC4)c4cccnc34)nc12] |
| 61 | 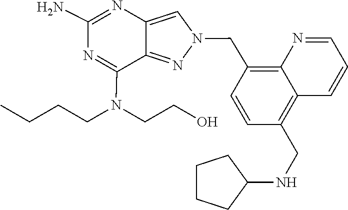 [SMILES: CCCCN(CCO)c1nc(N)nc2cn(Cc3ccc(CNC4CCCC4)c4cccnc34)nc12] |
| 62 | 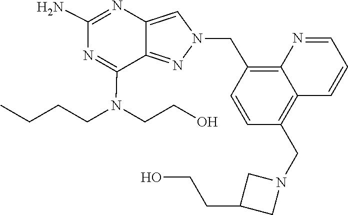 [SMILES: CCCCN(CCO)c1nc(N)nc2cn(Cc3ccc(CN4CC(CCO)C4)c4cccnc34)nc12] |
| 63 | 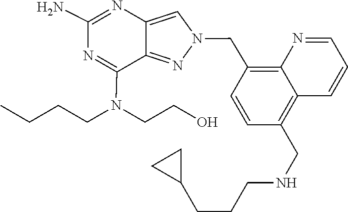 [SMILES: CCCCN(CCO)c1nc(N)nc2cn(Cc3ccc(CNCCCC4CC4)c4cccnc34)nc12] |
| 64 | 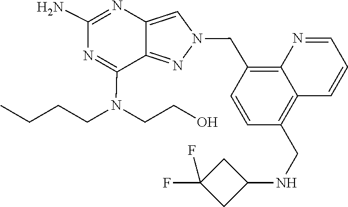 [SMILES: CCCCN(CCO)c1nc(N)nc2cn(Cc3ccc(CNC4CC(F)(F)C4)c4cccnc34)nc12] |
| 65 | 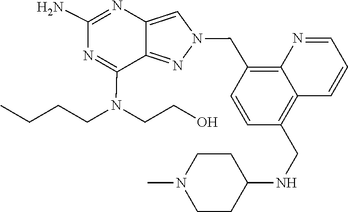 [SMILES: CCCCN(CCO)c1nc(N)nc2cn(Cc3ccc(CNC4CCN(C)CC4)c4cccnc34)nc12] |
| 66 | 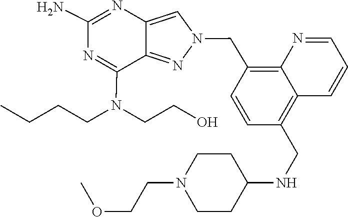 [SMILES: CCCCN(CCO)c1nc(N)nc2cn(Cc3ccc(CNC4CCN(CCOC)CC4)c4cccnc34)nc12] |
| 67 | 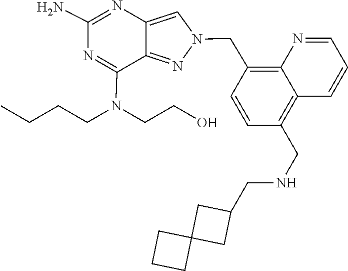 [SMILES: CCCCN(CCO)c1nc(N)nc2cn(Cc3ccc(CNCC4CC5(CCC5)C4)c4cccnc34)nc12] |
| 68 | 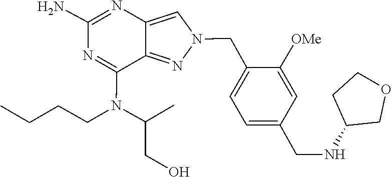 [SMILES: CCCCN(c1nc(N)nc2cn(Cc3ccc(CN[C@@H]4CCOC4)cc3OC)nc12)C(C)CO] |
| 70 | 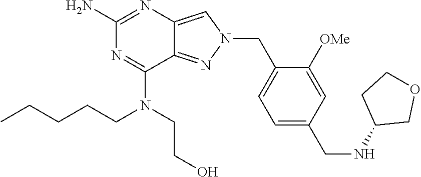 [SMILES: CCCCCN(CCO)c1nc(N)nc2cn(Cc3ccc(CN[C@@H]4CCOC4)cc3OC)nc12] |
| 71 | 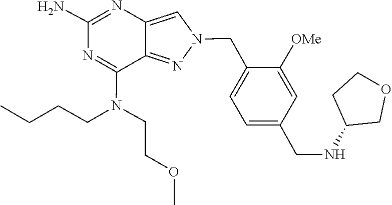 [SMILES: CCCCN(CCOC)c1nc(N)nc2cn(Cc3ccc(CN[C@@H]4CCOC4)cc3OC)nc12] |
| 72 | 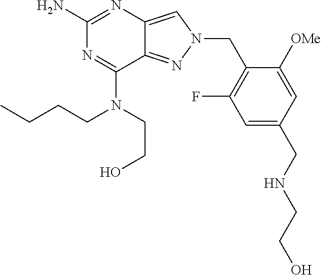 [SMILES: CCCCN(CCO)c1nc(N)nc2cn(Cc3c(F)cc(CNCCO)cc3OC)nc12] |
| 73 | 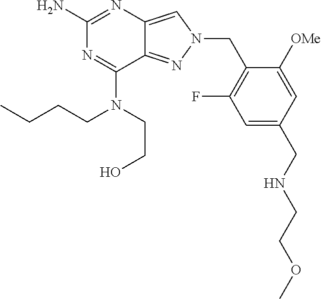 [SMILES: CCCCN(CCO)c1nc(N)nc2cn(Cc3c(F)cc(CNCCOC)cc3OC)nc12] |
| 74 | 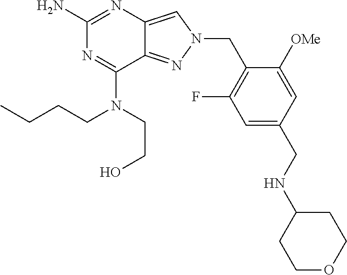 [SMILES: CCCCN(CCO)c1nc(N)nc2cn(Cc3c(F)cc(CNC4CCOCC4)cc3OC)nc12] |
| 75 | 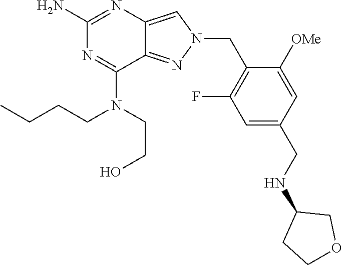 [SMILES: CCCCN(CCO)c1nc(N)nc2cn(Cc3c(F)cc(CN[C@@H]4CCOC4)cc3OC)nc12] |
| 80 | 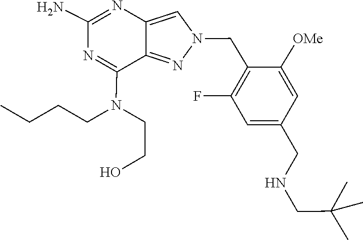 [SMILES: CCCCN(CCO)c1nc(N)nc2cn(Cc3c(F)cc(CNCC(C)(C)C)cc3OC)nc12] |
| 81 | 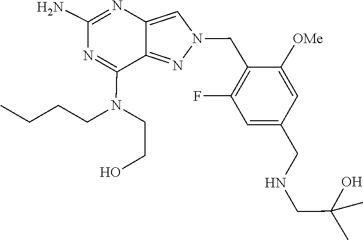 [SMILES: CCCCN(CCO)c1nc(N)nc2cn(Cc3c(F)cc(CNCC(C)(C)O)cc3OC)nc12] |
| 82 | 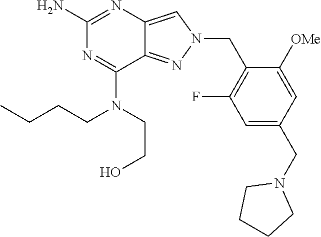 [SMILES: CCCCN(CCO)c1nc(N)nc2cn(Cc3c(F)cc(CN4CCCC4)cc3OC)nc12] |
| 83 | 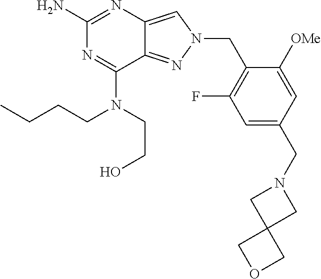 [SMILES: CCCCN(CCO)c1nc(N)nc2cn(Cc3c(F)cc(CN4CC5(COC5)C4)cc3OC)nc12] |
| 84 | 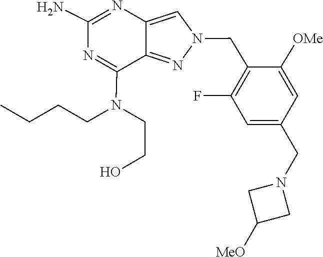 [SMILES: CCCCN(CCO)c1nc(N)nc2cn(Cc3c(F)cc(CN4CC(OC)C4)cc3OC)nc12] |
| 85 | 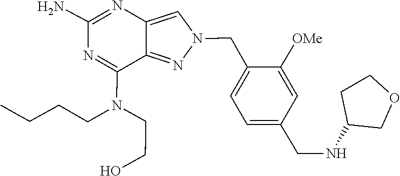 |
| 86 | 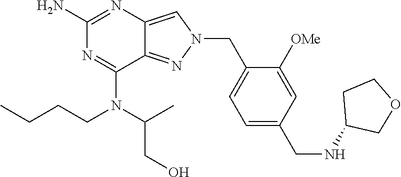 [SMILES: CCCCN(c1nc(N)nc2cn(Cc3ccc(CN[C@@H]4CCOC4)cc3OC)nc12)C(C)CO] |
| 87 | 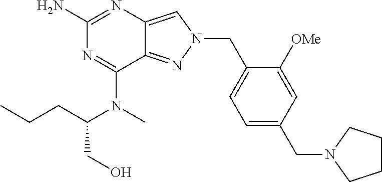 [SMILES: CCC[C@@H](CO)N(C)c1nc(N)nc2cn(Cc3ccc(CN4CCCC4)cc3OC)nc12] |
| 88 | 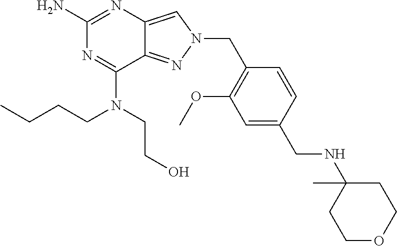 [SMILES: CCCCN(CCO)c1nc(N)nc2cn(Cc3ccc(CNC4(C)CCOCC4)cc3OC)nc12] |
| 89 | 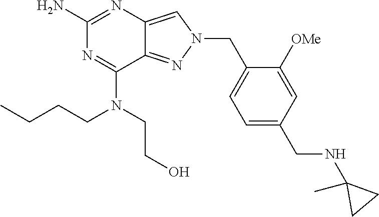 [SMILES: CCCCN(CCO)c1nc(N)nc2cn(Cc3ccc(CNC4(C)CC4)cc3OC)nc12] |
| 90 | 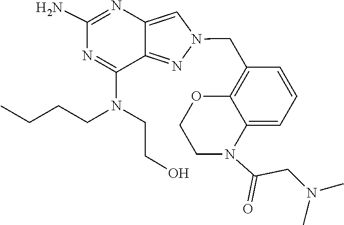 [SMILES: CCCCN(CCO)c1nc(N)nc2cn(Cc3cccc4c3OCCN4C(=O)CN(C)C)nc12] |
| 91 | 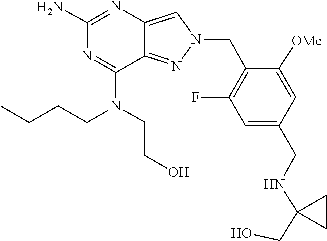 [SMILES: CCCCN(CCO)c1nc(N)nc2cn(Cc3c(F)cc(CNC4(CO)CC4)cc3OC)nc12] |
| 92 | 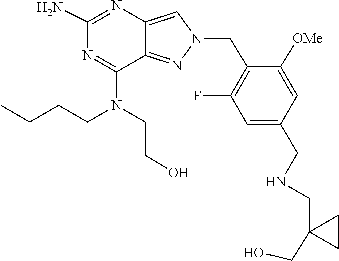 [SMILES: CCCCN(CCO)c1nc(N)nc2cn(Cc3c(F)cc(CNCC4(CO)CC4)cc3OC)nc12] |
| 93 | 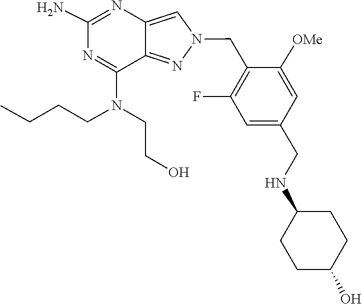 [SMILES: CCCCN(CCO)c1nc(N)nc2cn(Cc3c(F)cc(CN[C@H]4CC[C@H](O)CC4)cc3OC)nc12] |
| 94 | 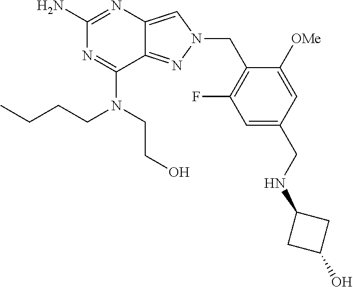 [SMILES: CCCCN(CCO)c1nc(N)nc2cn(Cc3c(F)cc(CN[C@H]4C[C@H](O)C4)cc3OC)nc12] |
| 95 | 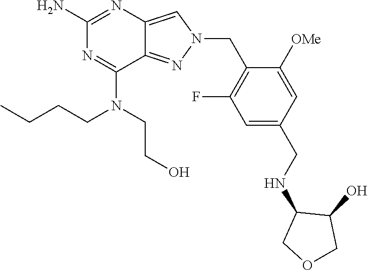 [SMILES: CCCCN(CCO)c1nc(N)nc2cn(Cc3c(F)cc(CN[C@@H]4COC[C@@H]4O)cc3OC)nc12] |
| 96 | 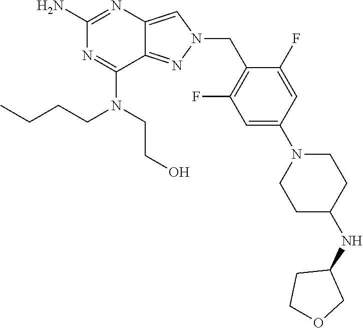 [SMILES: CCCCN(CCO)c1nc(N)nc2cn(Cc3c(F)cc(N4CCC(N[C@@H]5CCOC5)CC4)cc3F)nc12] |
| 97 | 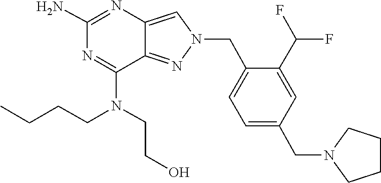 [SMILES: CCCCN(CCO)c1nc(N)nc2cn(Cc3ccc(CN4CCCC4)cc3C(F)F)nc12] |
| 98 | 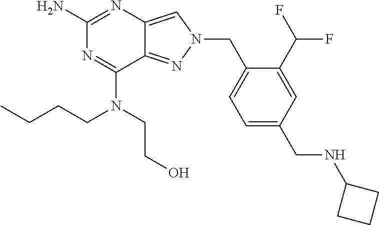 [SMILES: CCCCN(CCO)c1nc(N)nc2cn(Cc3ccc(CNC4CCC4)cc3C(F)F)nc12] |
| 99 | 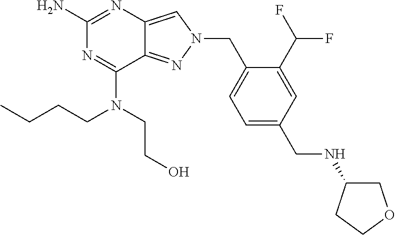 [SMILES: CCCCN(CCO)c1nc(N)nc2cn(Cc3ccc(CN[C@H]4CCOC4)cc3C(F)F)nc12] |
| 100 | 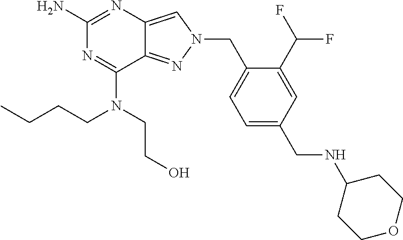 [SMILES: CCCCN(CCO)c1nc(N)nc2cn(Cc3ccc(CNC4CCOCC4)cc3C(F)F)nc12] |
| 101 | 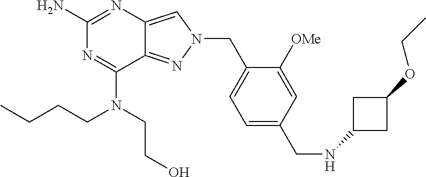 |
| 102 | 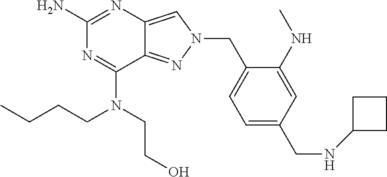 [SMILES: CCCCN(CCO)c1nc(N)nc2cn(Cc3ccc(CNC4CCC4)cc3NC)nc12] |
| 103 | 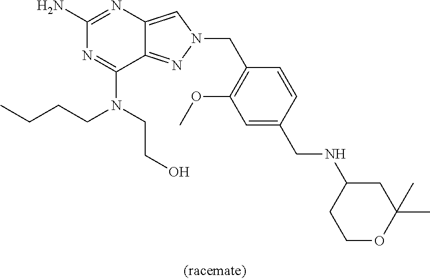 [SMILES: CCCCN(CCO)c1nc(N)nc2cn(Cc3ccc(CNC4CCOC(C)(C)C4)cc3OC)nc12] |
| 104 | 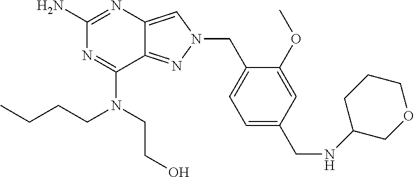 [SMILES: CCCCN(CCO)c1nc(N)nc2cn(Cc3ccc(CNC4CCCOC4)cc3OC)nc12] |
| 105 | 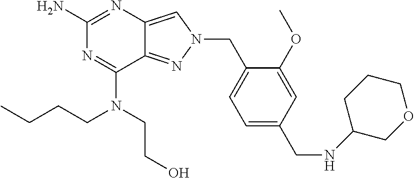 [SMILES: CCCCN(CCO)c1nc(N)nc2cn(Cc3ccc(CNC4CCCOC4)cc3OC)nc12] |
| 106 | 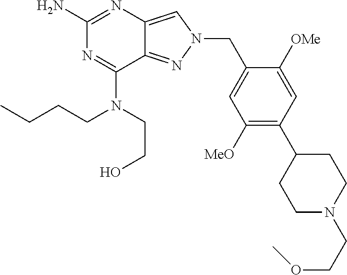 [SMILES: CCCCN(CCO)c1nc(N)nc2cn(Cc3cc(OC)c(C4CCN(CCOC)CC4)cc3OC)nc12] |
| 107 | 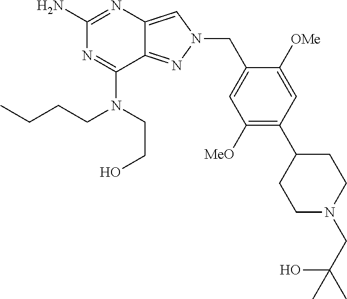 [SMILES: CCCCN(CCO)c1nc(N)nc2cn(Cc3cc(OC)c(C4CCN(CC(C)(C)O)CC4)cc3OC)nc12] |
| 108 | 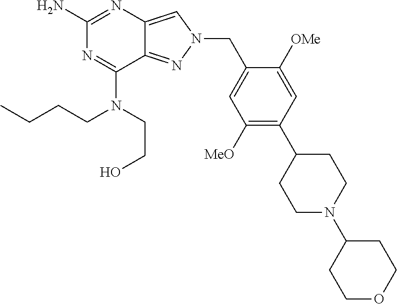 [SMILES: CCCCN(CCO)c1nc(N)nc2cn(Cc3cc(OC)c(C4CCN(C5CCOCC5)CC4)cc3OC)nc12] |
| 109 | 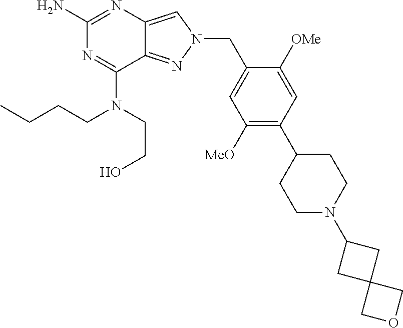 [SMILES: CCCCN(CCO)c1nc(N)nc2cn(Cc3cc(OC)c(C4CCN(C5CC6(COC6)C5)CC4)cc3OC)nc12] |
| 110 | 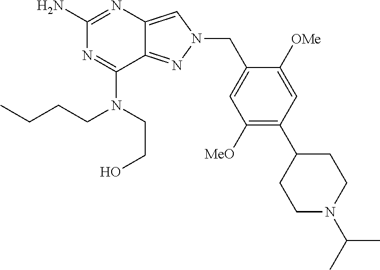 [SMILES: CCCCN(CCO)c1nc(N)nc2cn(Cc3cc(OC)c(C4CCN(C(C)C)CC4)cc3OC)nc12] |
| 111 | 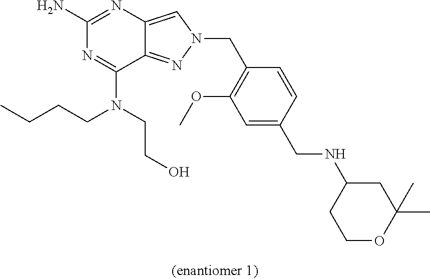 [SMILES: CCCCN(CCO)c1nc(N)nc2cn(Cc3ccc(CNC4CCOC(C)(C)C4)cc3OC)nc12] |
| 112 | 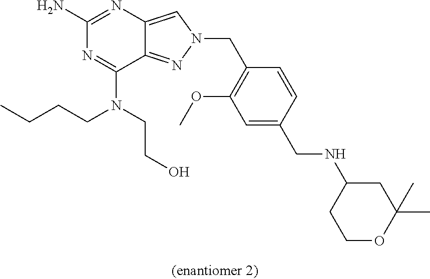 [SMILES: CCCCN(CCO)c1nc(N)nc2cn(Cc3ccc(CNC4CCOC(C)(C)C4)cc3OC)nc12] |
| 113 | 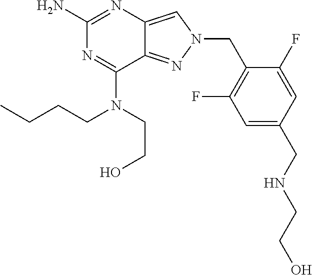 [SMILES: CCCCN(CCO)c1nc(N)nc2cn(Cc3c(F)cc(CNCCO)cc3F)nc12] |
| 114 | 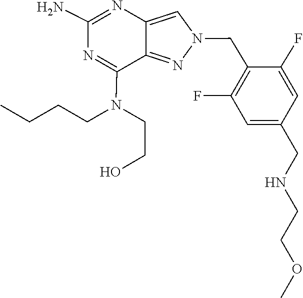 [SMILES: CCCCN(CCO)c1nc(N)nc2cn(Cc3c(F)cc(CNCCOC)cc3F)nc12] |
| 115 | 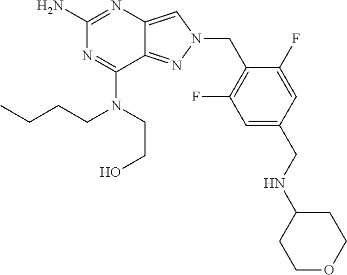 [SMILES: CCCCN(CCO)c1nc(N)nc2cn(Cc3c(F)cc(CNC4CCOCC4)cc3F)nc12] |
| 116 | 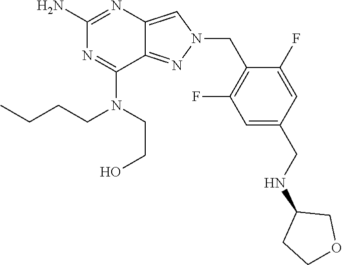 [SMILES: CCCCN(CCO)c1nc(N)nc2cn(Cc3c(F)cc(CN[C@@H]4CCOC4)cc3F)nc12] |
| 117 | 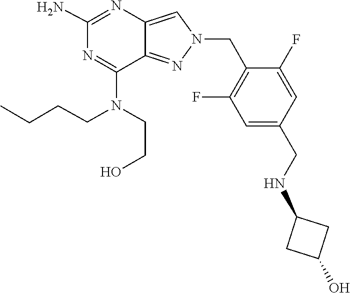 [SMILES: CCCCN(CCO)c1nc(N)nc2cn(Cc3c(F)cc(CN[C@H]4C[C@H](O)C4)cc3F)nc12] |
| 118 | 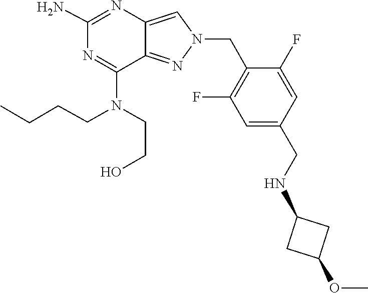 [SMILES: CCCCN(CCO)c1nc(N)nc2cn(Cc3c(F)cc(CN[C@H]4C[C@@H](OC)C4)cc3F)nc12] |
| 119 | 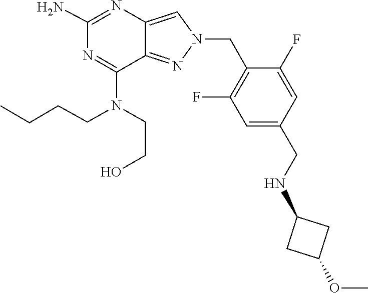 [SMILES: CCCCN(CCO)c1nc(N)nc2cn(Cc3c(F)cc(CN[C@H]4C[C@H](OC)C4)cc3F)nc12] |
| 120 | 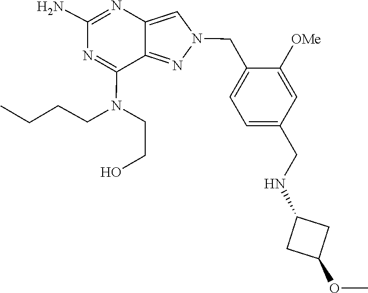 [SMILES: CCCCN(CCO)c1nc(N)nc2cn(Cc3ccc(CN[C@H]4C[C@H](OC)C4)cc3OC)nc12] |
| 121 (stereoisomer 1) | 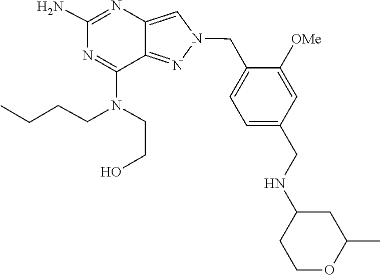 [SMILES: CCCCN(CCO)c1nc(N)nc2cn(Cc3ccc(CNC4CCOC(C)C4)cc3OC)nc12] |
| 122 (stereoisomer 2) | 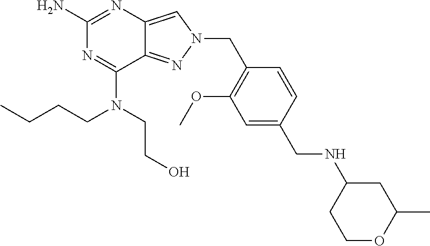 [SMILES: CCCCN(CCO)c1nc(N)nc2cn(Cc3ccc(CNC4CCOC(C)C4)cc3OC)nc12] |
| 123 | 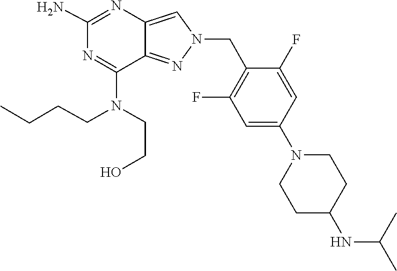 [SMILES: CCCCN(CCO)c1nc(N)nc2cn(Cc3c(F)cc(N4CCC(NC(C)C)CC4)cc3F)nc12] |
| 124 | 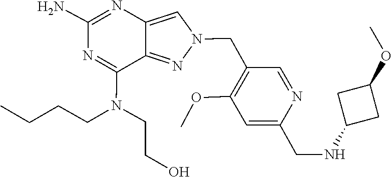 [SMILES: CCCCN(CCO)c1nc(N)nc2cn(Cc3cnc(CN[C@H]4C[C@H](OC)C4)cc3OC)nc12] |
| 125 | 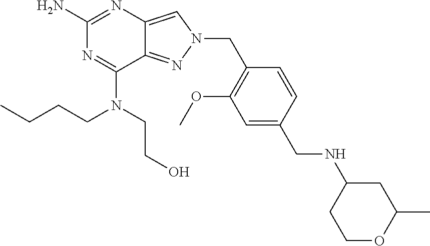 [SMILES: CCCCN(CCO)c1nc(N)nc2cn(Cc3ccc(CNC4CCOC(C)C4)cc3OC)nc12] |
| 126 | 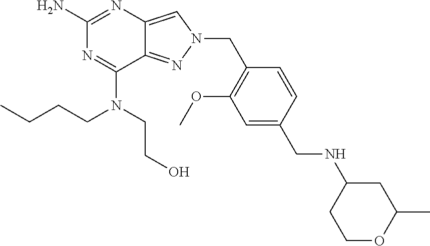 [SMILES: CCCCN(CCO)c1nc(N)nc2cn(Cc3ccc(CNC4CCOC(C)C4)cc3OC)nc12] |
| 127 | 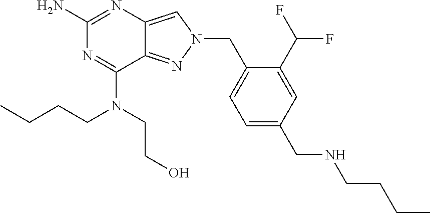 [SMILES: CCCCNCc1ccc(Cn2cc3nc(N)nc(N(CCO)CCCC)c3n2)c(C(F)F)c1] |
| 128 | 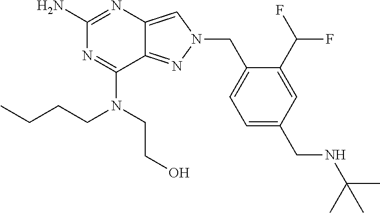 [SMILES: CCCCN(CCO)c1nc(N)nc2cn(Cc3ccc(CNC(C)(C)C)cc3C(F)F)nc12] |
| 129 | 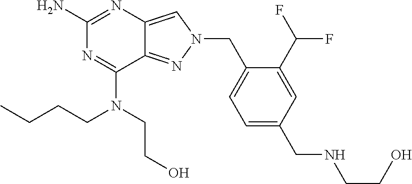 [SMILES: CCCCN(CCO)c1nc(N)nc2cn(Cc3ccc(CNCCO)cc3C(F)F)nc12] |
| 130 | 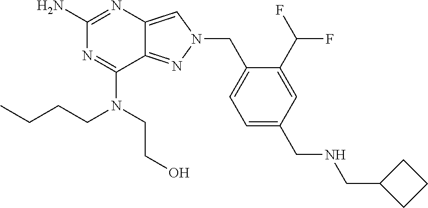 [SMILES: CCCCN(CCO)c1nc(N)nc2cn(Cc3ccc(CNCC4CCC4)cc3C(F)F)nc12] |
| 131 | 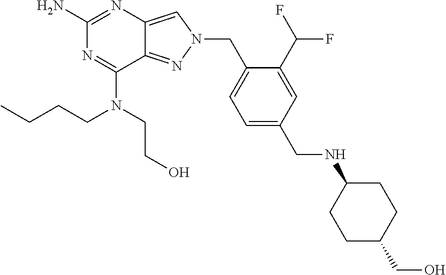 [SMILES: CCCCN(CCO)c1nc(N)nc2cn(Cc3ccc(CN[C@H]4CC[C@H](CO)CC4)cc3C(F)F)nc12] |
| 132 | 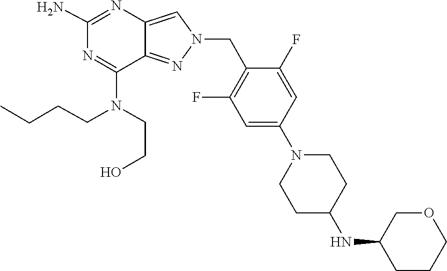 [SMILES: CCCCN(CCO)c1nc(N)nc2cn(Cc3c(F)cc(N4CCC(N[C@@H]5CCCOC5)CC4)cc3F)nc12] |
| 133 | 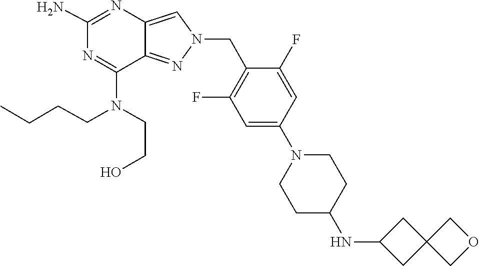 [SMILES: CCCCN(CCO)c1nc(N)nc2cn(Cc3c(F)cc(N4CCC(NC5CC6(COC6)C5)CC4)cc3F)nc12] |
| 134 | 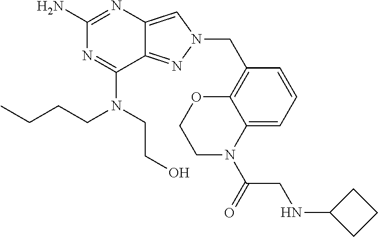 [SMILES: CCCCN(CCO)c1nc(N)nc2cn(Cc3cccc4c3OCCN4C(=O)CNC3CCC3)nc12] |
| 135 | 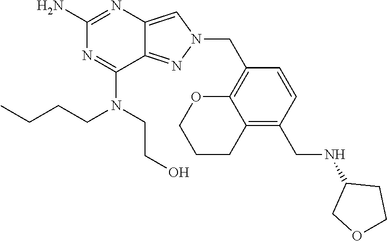 |
| 136 | 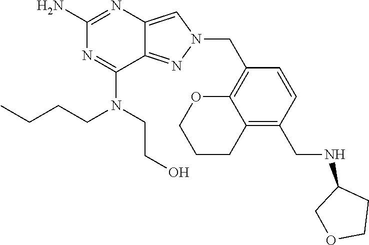 [SMILES: CCCCN(CCO)c1nc(N)nc2cn(Cc3ccc(CN[C@H]4CCOC4)c4c3OCCC4)nc12] |
| 137 | 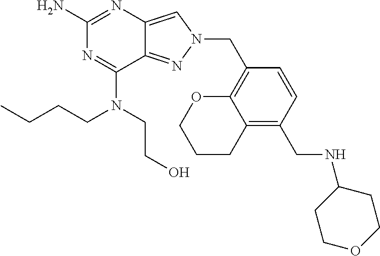 [SMILES: CCCCN(CCO)c1nc(N)nc2cn(Cc3ccc(CNC4CCOCC4)c4c3OCCC4)nc12] |
| 138 | 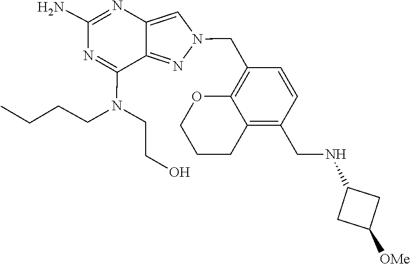 |
| 139 | 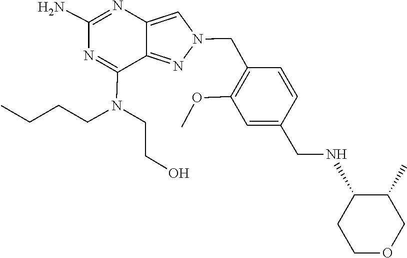 [SMILES: CCCCN(CCO)c1nc(N)nc2cn(Cc3ccc(CN[C@H]4CCOC[C@H]4C)cc3OC)nc12] |
| 140 | 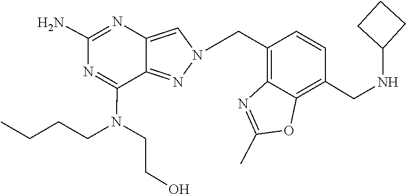 [SMILES: CCCCN(CCO)c1nc(N)nc2cn(Cc3ccc(CNC4CCC4)c4oc(C)nc34)nc12] |
| 141 | 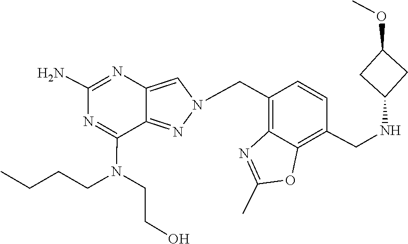 [SMILES: CCCCN(CCO)c1nc(N)nc2cn(Cc3ccc(CN[C@H]4C[C@H](OC)C4)c4oc(C)nc34)nc12] |
| 142 | 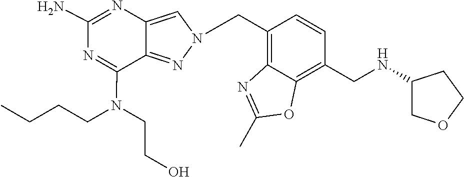 |
| 143 | 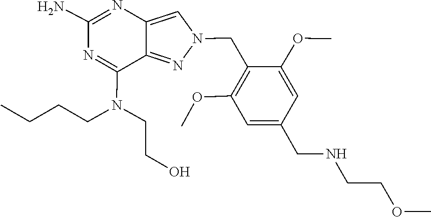 [SMILES: CCCCN(CCO)c1nc(N)nc2cn(Cc3c(OC)cc(CNCCOC)cc3OC)nc12] |
| 144 | 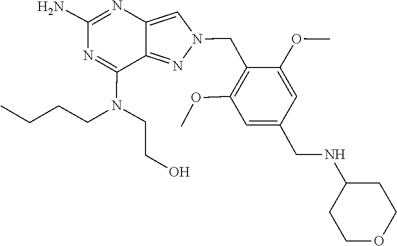 [SMILES: CCCCN(CCO)c1nc(N)nc2cn(Cc3c(OC)cc(CNC4CCOCC4)cc3OC)nc12] |
| 145 | 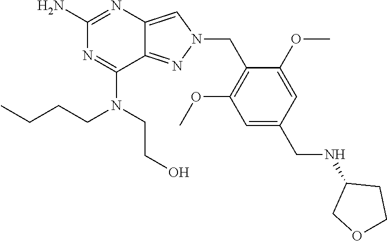 [SMILES: CCCCN(CCO)c1nc(N)nc2cn(Cc3c(OC)cc(CN[C@@H]4CCOC4)cc3OC)nc12] |
| 146 | 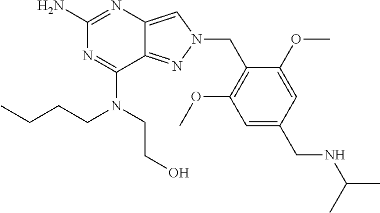 [SMILES: CCCCN(CCO)c1nc(N)nc2cn(Cc3c(OC)cc(CNC(C)C)cc3OC)nc12] |
| 147 | 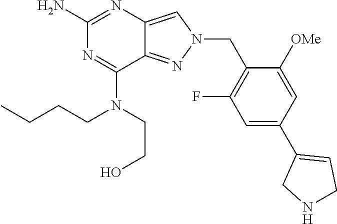 [SMILES: CCCCN(CCO)c1nc(N)nc2cn(Cc3c(F)cc(C4=CCNC4)cc3OC)nc12] |
| 148 | 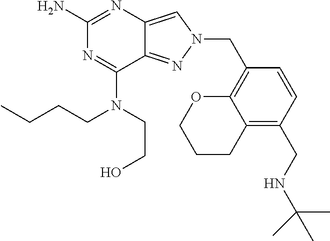 [SMILES: CCCCN(CCO)c1nc(N)nc2cn(Cc3ccc(CNC(C)(C)C)c4c3OCCC4)nc12] |
| 149 | 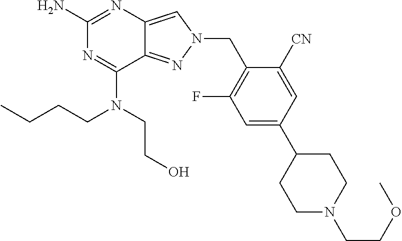 [SMILES: CCCCN(CCO)c1nc(N)nc2cn(Cc3c(F)cc(C4CCN(CCOC)CC4)cc3C#N)nc12] |
| 150 | 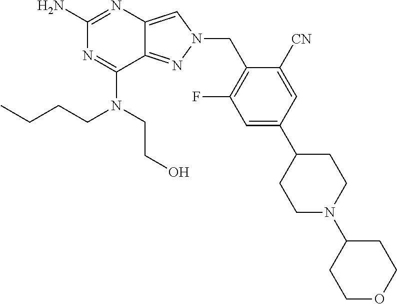 [SMILES: CCCCN(CCO)c1nc(N)nc2cn(Cc3c(F)cc(C4CCN(C5CCOCC5)CC4)cc3C#N)nc12] |
| 151 | 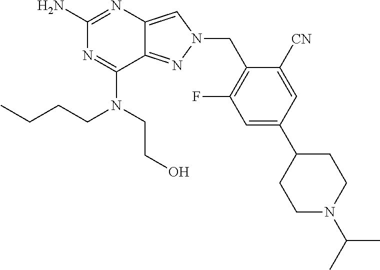 [SMILES: CCCCN(CCO)c1nc(N)nc2cn(Cc3c(F)cc(C4CCN(C(C)C)CC4)cc3C#N)nc12] |
| 152 | 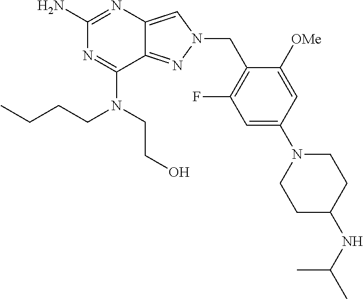 [SMILES: CCCCN(CCO)c1nc(N)nc2cn(Cc3c(F)cc(N4CCC(NC(C)C)CC4)cc3OC)nc12] |
| 153 | 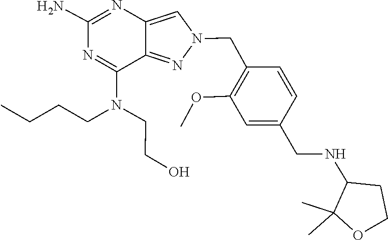 [SMILES: CCCCN(CCO)c1nc(N)nc2cn(Cc3ccc(CNC4CCOC4(C)C)cc3OC)nc12] |
| 154 | 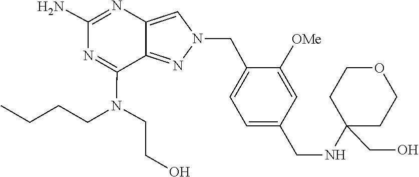 [SMILES: CCCCN(CCO)c1nc(N)nc2cn(Cc3ccc(CNC4(CO)CCOCC4)cc3OC)nc12] |
| 155 | 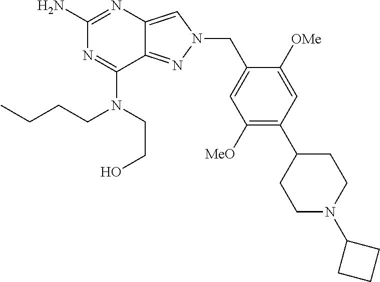 [SMILES: CCCCN(CCO)c1nc(N)nc2cn(Cc3cc(OC)c(C4CCN(C5CCC5)CC4)cc3OC)nc12] |
| 156 | 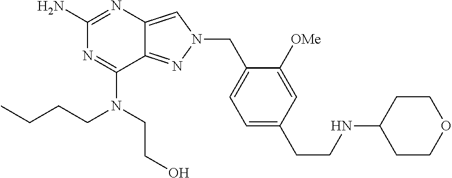 [SMILES: CCCCN(CCO)c1nc(N)nc2cn(Cc3ccc(CCNC4CCOCC4)cc3OC)nc12] |
| 157 | 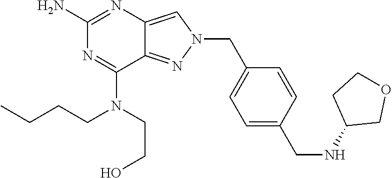 [SMILES: CCCCN(CCO)c1nc(N)nc2cn(Cc3ccc(CN[C@@H]4CCOC4)cc3)nc12] |
| 158 | 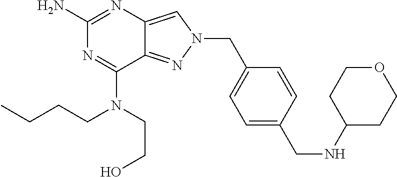 [SMILES: CCCCN(CCO)c1nc(N)nc2cn(Cc3ccc(CNC4CCOCC4)cc3)nc12] |
| 159 | 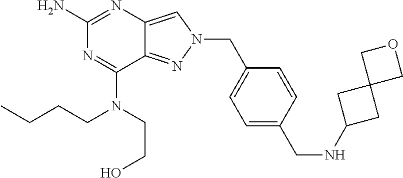 [SMILES: CCCCN(CCO)c1nc(N)nc2cn(Cc3ccc(CNC4CC5(COC5)C4)cc3)nc12] |
| 160 | 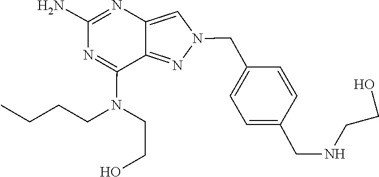 [SMILES: CCCCN(CCO)c1nc(N)nc2cn(Cc3ccc(CNCCO)cc3)nc12] |
| 161 | 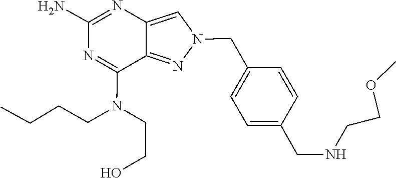 [SMILES: CCCCN(CCO)c1nc(N)nc2cn(Cc3ccc(CNCCOC)cc3)nc12] |
| 162 | 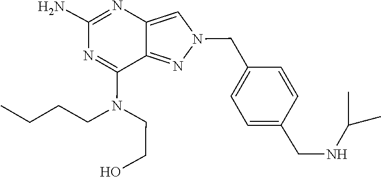 [SMILES: CCCCN(CCO)c1nc(N)nc2cn(Cc3ccc(CNC(C)C)cc3)nc12] |
| 163 | 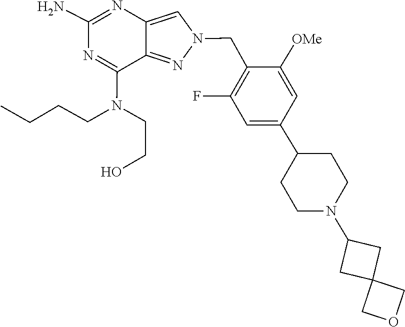 [SMILES: CCCCN(CCO)c1nc(N)nc2cn(Cc3c(F)cc(C4CCN(C5CC6(COC6)C5)CC4)cc3OC)nc12] |
| 164 | 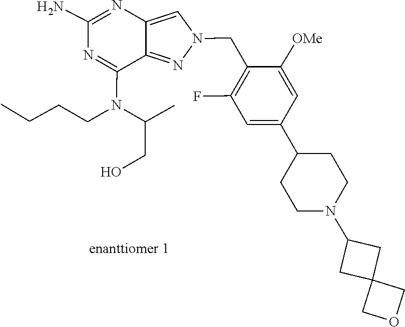 [SMILES: CCCCN(c1nc(N)nc2cn(Cc3c(F)cc(C4CCN(C5CC6(COC6)C5)CC4)cc3OC)nc12)C(C)CO] |
| 165 | 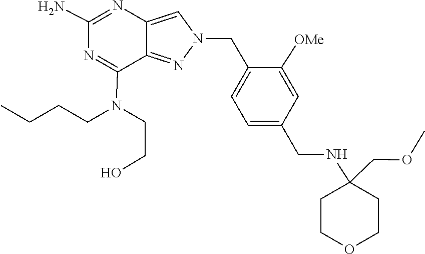 [SMILES: CCCCN(CCO)c1nc(N)nc2cn(Cc3ccc(CNC4(COC)CCOCC4)cc3OC)nc12] |
| 166 | 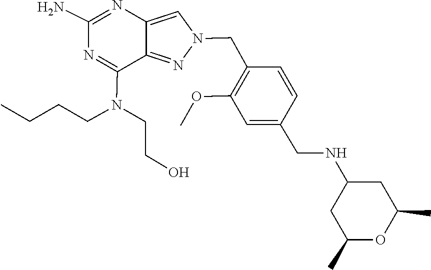 [SMILES: CCCCN(CCO)c1nc(N)nc2cn(Cc3ccc(CNC4C[C@@H](C)O[C@@H](C)C4)cc3OC)nc12] |
| 167 | 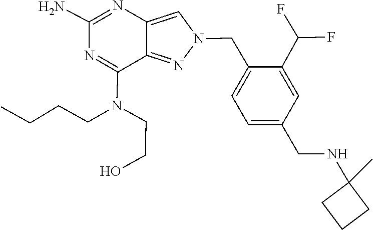 [SMILES: CCCCN(CCO)c1nc(N)nc2cn(Cc3ccc(CNC4(C)CCC4)cc3C(F)F)nc12] |
| 168 | 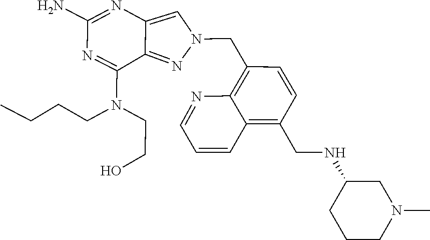 [SMILES: CCCCN(CCO)c1nc(N)nc2cn(Cc3ccc(CN[C@H]4CCCN(C)C4)c4cccnc34)nc12] |
| 169 | 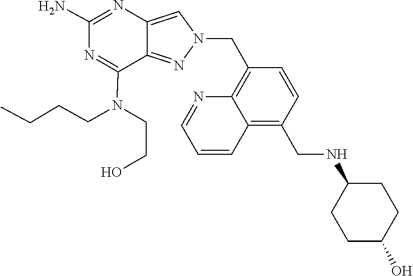 [SMILES: CCCCN(CCO)c1nc(N)nc2cn(Cc3ccc(CN[C@H]4CC[C@H](O)CC4)c4cccnc34)nc12] |
| 170 | 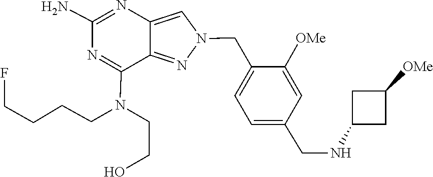 [SMILES: COc1cc(CN[C@H]2C[C@H](OC)C2)ccc1Cn1cc2nc(N)nc(N(CCO)CCCCF)c2n1] |
| 171 | 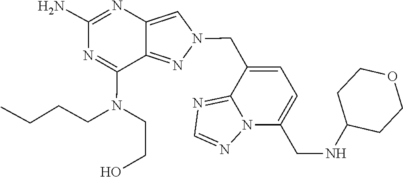 [SMILES: CCCCN(CCO)c1nc(N)nc2cn(Cc3ccc(CNC4CCOCC4)n4ncnc34)nc12] |
| 172 | 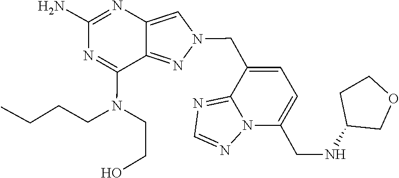 [SMILES: CCCCN(CCO)c1nc(N)nc2cn(Cc3ccc(CN[C@@H]4CCOC4)n4ncnc34)nc12] |
| 173 | 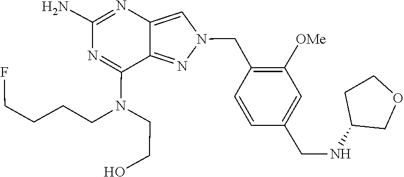 [SMILES: COc1cc(CN[C@@H]2CCOC2)ccc1Cn1cc2nc(N)nc(N(CCO)CCCCF)c2n1] |
| 174 | 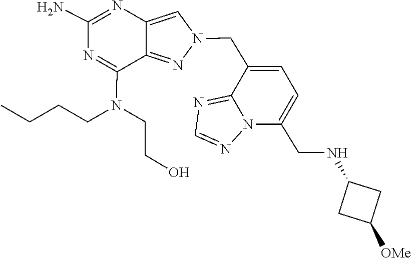 [SMILES: CCCCN(CCO)c1nc(N)nc2cn(Cc3ccc(CN[C@H]4C[C@H](OC)C4)n4ncnc34)nc12] |
| 175 | 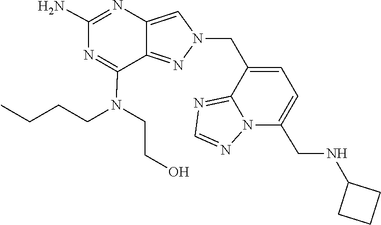 [SMILES: CCCCN(CCO)c1nc(N)nc2cn(Cc3ccc(CNC4CCC4)n4ncnc34)nc12] |
| 176 | 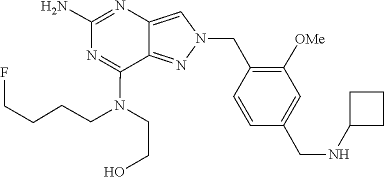 [SMILES: COc1cc(CNC2CCC2)ccc1Cn1cc2nc(N)nc(N(CCO)CCCCF)c2n1] |
| 177 | 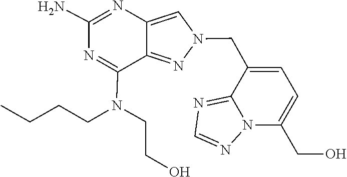 [SMILES: CCCCN(CCO)c1nc(N)nc2cn(Cc3ccc(CO)n4ncnc34)nc12] |
| 178 | 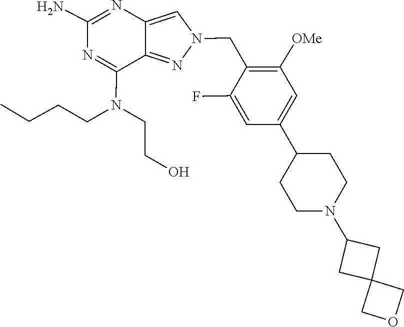 [SMILES: CCCCN(CCO)c1nc(N)nc2cn(Cc3c(F)cc(C4CCN(C5CC6(COC6)C5)CC4)cc3OC)nc12] |
| 179 | 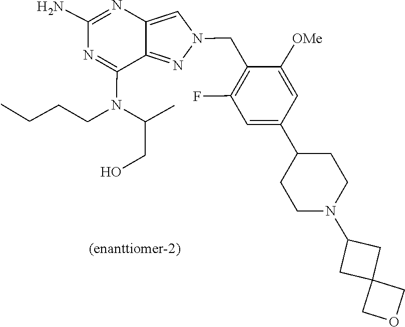 [SMILES: CCCCN(c1nc(N)nc2cn(Cc3c(F)cc(C4CCN(C5CC6(COC6)C5)CC4)cc3OC)nc12)C(C)CO] |
| 180 | 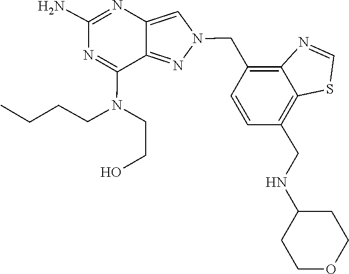 [SMILES: CCCCN(CCO)c1nc(N)nc2cn(Cc3ccc(CNC4CCOCC4)c4scnc34)nc12] |
| 182 | 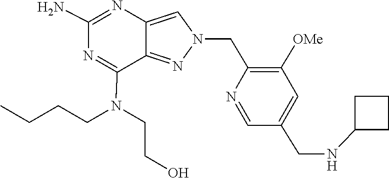 [SMILES: CCCCN(CCO)c1nc(N)nc2cn(Cc3ncc(CNC4CCC4)cc3OC)nc12] |
| 183 | 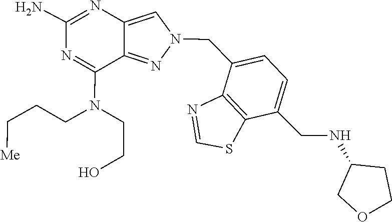 [SMILES: CCCCN(CCO)c1nc(N)nc2cn(Cc3ccc(CN[C@@H]4CCOC4)c4scnc34)nc12] |
| 184 | 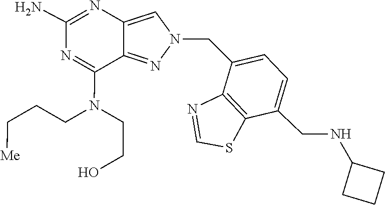 [SMILES: CCCCN(CCO)c1nc(N)nc2cn(Cc3ccc(CNC4CCC4)c4scnc34)nc12] |
| 185 | 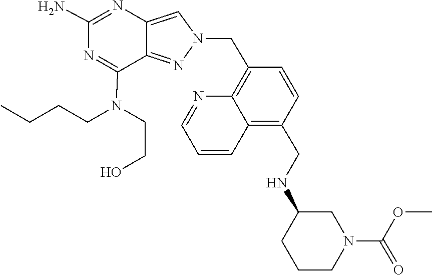 [SMILES: CCCCN(CCO)c1nc(N)nc2cn(Cc3ccc(CN[C@@H]4CCCN(C(=O)OC)C4)c4cccnc34)nc12] |
| 186 | 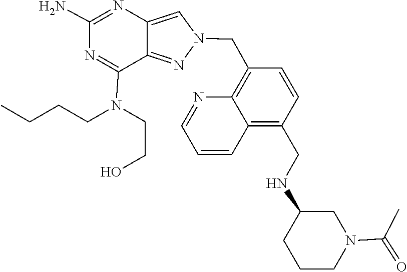 [SMILES: CCCCN(CCO)c1nc(N)nc2cn(Cc3ccc(CN[C@@H]4CCCN(C(C)=O)C4)c4cccnc34)nc12] |
| 187 | 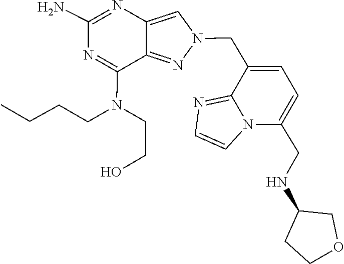 [SMILES: CCCCN(CCO)c1nc(N)nc2cn(Cc3ccc(CN[C@@H]4CCOC4)n4ccnc34)nc12] |
| 188 | 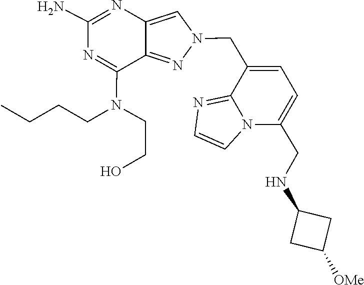 [SMILES: CCCCN(CCO)c1nc(N)nc2cn(Cc3ccc(CN[C@H]4C[C@H](OC)C4)n4ccnc34)nc12] |
| 189 | 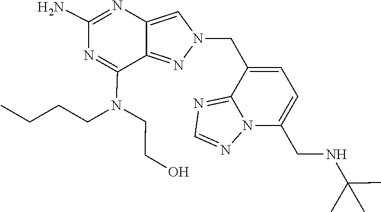 [SMILES: CCCCN(CCO)c1nc(N)nc2cn(Cc3ccc(CNC(C)(C)C)n4ncnc34)nc12] |
| 190 | 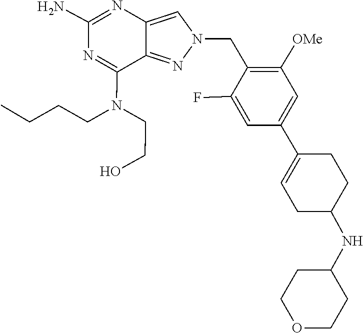 [SMILES: CCCCN(CCO)c1nc(N)nc2cn(Cc3c(F)cc(C4=CCC(NC5CCOCC5)CC4)cc3OC)nc12] |
| 191 | 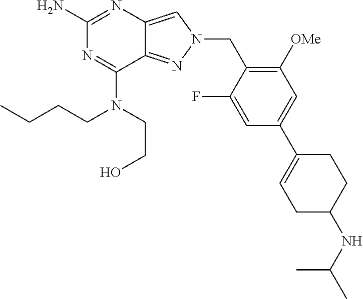 [SMILES: CCCCN(CCO)c1nc(N)nc2cn(Cc3c(F)cc(C4=CCC(NC(C)C)CC4)cc3OC)nc12] |
| 192 | 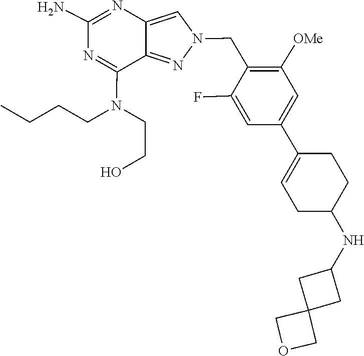 [SMILES: CCCCN(CCO)c1nc(N)nc2cn(Cc3c(F)cc(C4=CCC(NC5CC6(COC6)C5)CC4)cc3OC)nc12] |
| 193 | 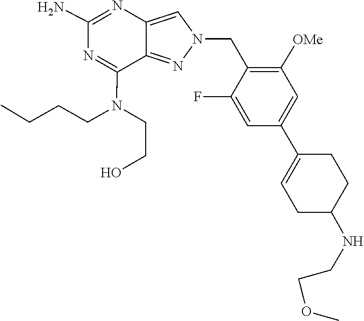 [SMILES: CCCCN(CCO)c1nc(N)nc2cn(Cc3c(F)cc(C4=CCC(NCCOC)CC4)cc3OC)nc12] |
| 194 | 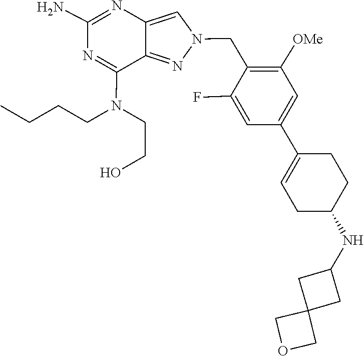 [SMILES: CCCCN(CCO)c1nc(N)nc2cn(Cc3c(F)cc(C4=CC[C@@H](NC5CC6(COC6)C5)CC4)cc3OC)nc12] |
| 195 | 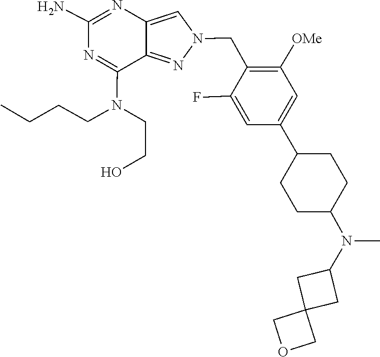 [SMILES: CCCCN(CCO)c1nc(N)nc2cn(Cc3c(F)cc(C4CCC(N(C)C5CC6(COC6)C5)CC4)cc3OC)nc12] |
| 196 | 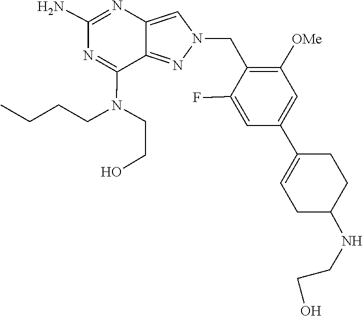 [SMILES: CCCCN(CCO)c1nc(N)nc2cn(Cc3c(F)cc(C4=CCC(NCCO)CC4)cc3OC)nc12] |
| 197 | 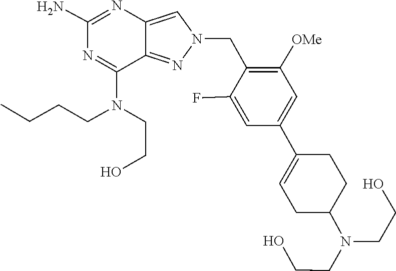 [SMILES: CCCCN(CCO)c1nc(N)nc2cn(Cc3c(F)cc(C4=CCC(N(CCO)CCO)CC4)cc3OC)nc12] |
| 198 | 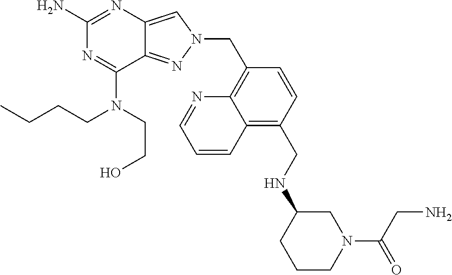 [SMILES: CCCCN(CCO)c1nc(N)nc2cn(Cc3ccc(CN[C@@H]4CCCN(C(=O)CN)C4)c4cccnc34)nc12] |
| 199 | 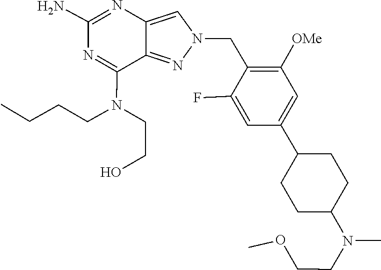 [SMILES: CCCCN(CCO)c1nc(N)nc2cn(Cc3c(F)cc(C4CCC(N(C)CCOC)CC4)cc3OC)nc12] |
| 200 | 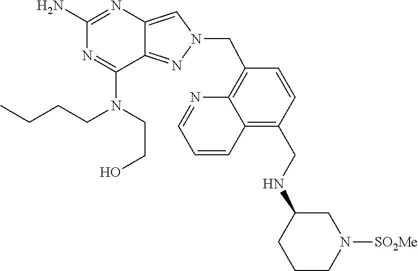 [SMILES: CCCCN(CCO)c1nc(N)nc2cn(Cc3ccc(CN[C@@H]4CCCN(S(C)(=O)=O)C4)c4cccnc34)nc12] |
| 201 | 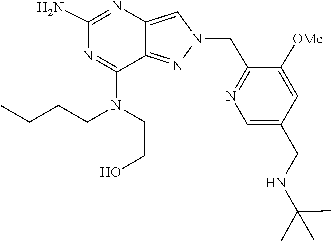 [SMILES: CCCCN(CCO)c1nc(N)nc2cn(Cc3ncc(CNC(C)(C)C)cc3OC)nc12] |
| 202 | 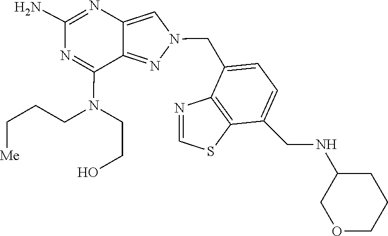 [SMILES: CCCCN(CCO)c1nc(N)nc2cn(Cc3ccc(CNC4CCCOC4)c4scnc34)nc12] |
| 203 | 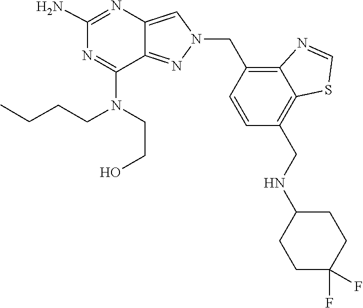 [SMILES: CCCCN(CCO)c1nc(N)nc2cn(Cc3ccc(CNC4CCC(F)(F)CC4)c4scnc34)nc12] |
| 204 | 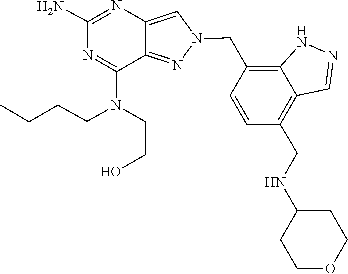 [SMILES: CCCCN(CCO)c1nc(N)nc2cn(Cc3ccc(CNC4CCOCC4)c4cn[nH]c34)nc12] |
| 205 | 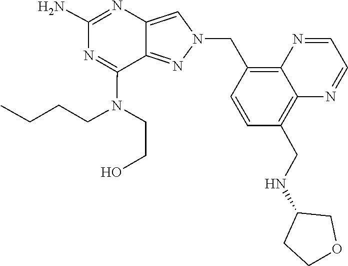 [SMILES: CCCCN(CCO)c1nc(N)nc2cn(Cc3ccc(CN[C@H]4CCOC4)c4nccnc34)nc12] |
| 206 | 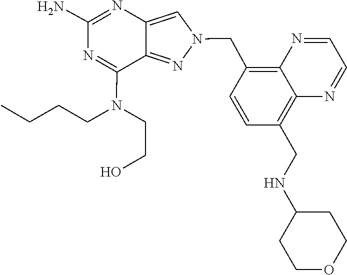 [SMILES: CCCCN(CCO)c1nc(N)nc2cn(Cc3ccc(CNC4CCOCC4)c4nccnc34)nc12] |
| 207 | 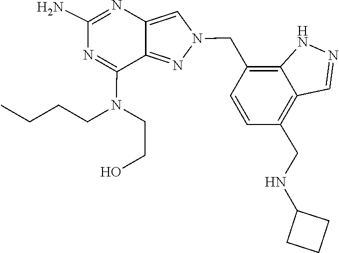 [SMILES: CCCCN(CCO)c1nc(N)nc2cn(Cc3ccc(CNC4CCC4)c4cn[nH]c34)nc12] |
| 208 | 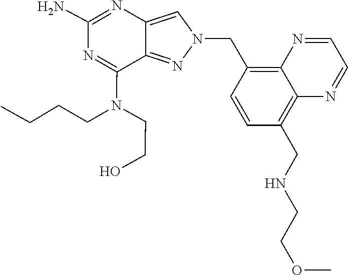 [SMILES: CCCCN(CCO)c1nc(N)nc2cn(Cc3ccc(CNCCOC)c4nccnc34)nc12] |
| 209 | 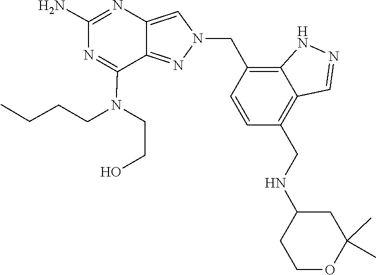 [SMILES: CCCCN(CCO)c1nc(N)nc2cn(Cc3ccc(CNC4CCOC(C)(C)C4)c4cn[nH]c34)nc12] |
| 210 | 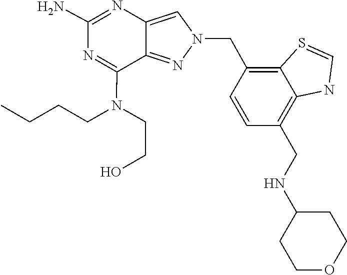 |
| 211 | 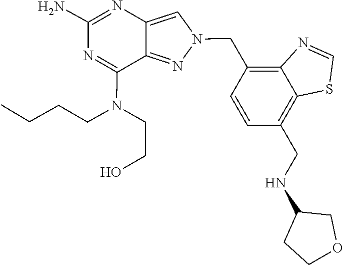 |
| 212 | 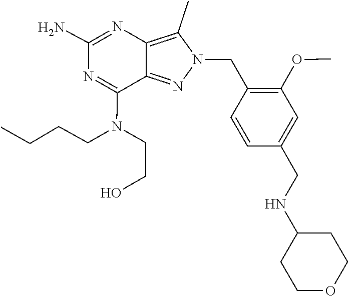 [SMILES: CCCCN(CCO)c1nc(N)nc2c(C)n(Cc3ccc(CNC4CCOCC4)cc3OC)nc12] |
| 213 | 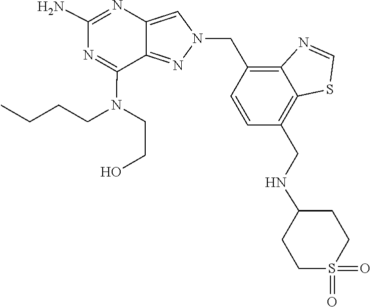 [SMILES: CCCCN(CCO)c1nc(N)nc2cn(Cc3ccc(CNC4CCS(=O)(=O)CC4)c4scnc34)nc12] |

TABLE 1B

| Compound | |
|---|---|
| No. | Structure |
| 34 | 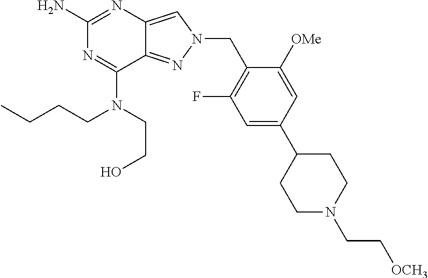 [SMILES: CCCCN(CCO)c1nc(N)nc2cn(Cc3c(F)cc(C4CCN(CCOC)CC4)cc3OC)nc12] |
| 42 | 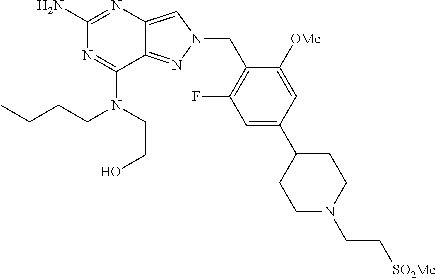 [SMILES: CCCCN(CCO)c1nc(N)nc2cn(Cc3c(F)cc(C4CCN(CCS(C)(=O)=O)CC4)cc3OC)nc12] |
| 45 | 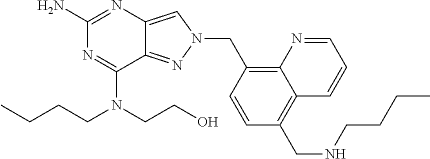 [SMILES: CCCCNCc1ccc(Cn2cc3nc(N)nc(N(CCO)CCCC)c3n2)c2ncccc12] |
| 86 | 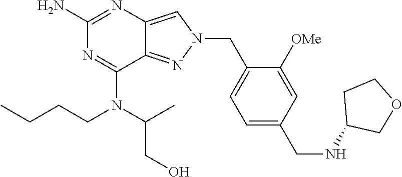 [SMILES: CCCCN(c1nc(N)nc2cn(Cc3ccc(CN[C@@H]4CCOC4)cc3OC)nc12)C(C)CO] |
| 110 | 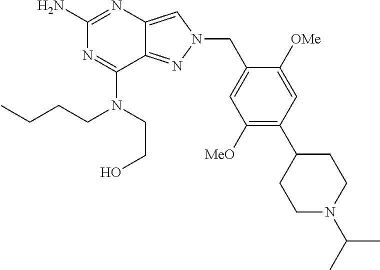 [SMILES: CCCCN(CCO)c1nc(N)nc2cn(Cc3cc(OC)c(C4CCN(C(C)C)CC4)cc3OC)nc12] |
| 136 | 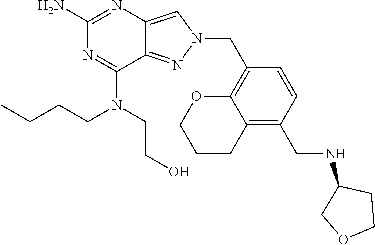 [SMILES: CCCCN(CCO)c1nc(N)nc2cn(Cc3ccc(CN[C@H]4CCOC4)c4c3OCCC4)nc12] |
| 141 | 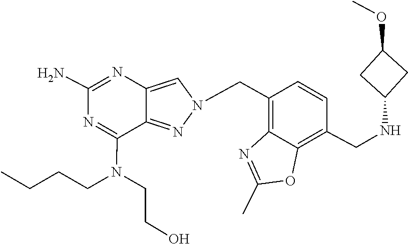 [SMILES: CCCCN(CCO)c1nc(N)nc2cn(Cc3ccc(CN[C@H]4C[C@H](OC)C4)c4oc(C)nc34)nc12] |
| 175 | 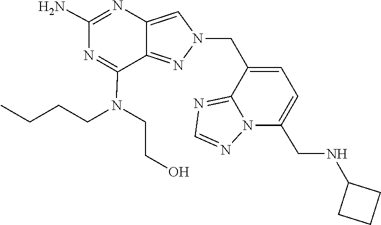 [SMILES: CCCCN(CCO)c1nc(N)nc2cn(Cc3ccc(CNC4CCC4)n4ncnc34)nc12] |
| 183 | 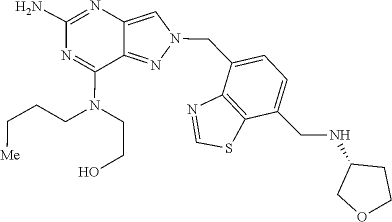 [SMILES: CCCCN(CCO)c1nc(N)nc2cn(Cc3ccc(CN[C@@H]4CCOC4)c4scnc34)nc12] |
| 212 | 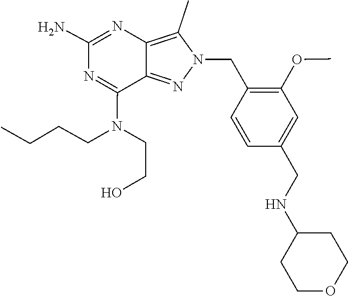 [SMILES: CCCCN(CCO)c1nc(N)nc2c(C)n(Cc3ccc(CNC4CCOCC4)cc3OC)nc12] |

It is understood that in the present description, combinations of substituents and/or variables of the depicted formulae are permissible only if such contributions result in stable compounds.

Furthermore, all compounds of Formula (I) that exist in free base or acid form can be converted to their pharmaceutically acceptable salts by treatment with the appropriate inorganic or organic base or acid by methods known to one skilled in the art. Salts of the compounds of Formula (I) can be converted to their free base or acid form by standard techniques.

### Conjugates

### General

TLR7 agonists disclosed herein can be delivered to the site of intended action by localized administration or by targeted delivery in a conjugate with a targeting moiety. Preferably, the targeting moiety is an antibody or antigen binding portion thereof and its antigen is found at the locality of intended action, for example a tumor associated antigen if the intended site of action is at a tumor (cancer). Preferably, the tumor associated antigen is uniquely expressed or overexpressed by the cancer cell, compared to a normal cell. The tumor associated antigen can be located on the surface of the cancer cell or secreted by the cancer cell into its environs.

In one aspect, there is provided a conjugate comprising a compound provided herein and a targeting agent, represented by Formula (VI)

[D(X^{D})ₐ(C)_{c}(X^{Z})_{b}]ₘZ (VI)

where Z is a targeting moiety, D is a compound provided herein, and —(X^{D})ₐ(C)_{c}(X^{Z})_{b}— are collectively referred to as a “linker moiety” or “linker” because they link Z and D. Within the linker, C is a cleavable group designed to be cleaved at or near the site of intended biological action of D; X^{D} and X^{Z} are spacer moieties (or “spacers”) that space apart D and C and C and Z, respectively; subscripts a, b, and c are independently 0 or 1 (that is, the presence of X^{D}, X^{Z} and C are optional). Subscript m is 1, 2, 3, 4, 5, 6, 7, 8, 9, or 10 (preferably 1, 2, 3, or 4). D, X^{D}, C, X^{Z} and Z are more fully described hereinbelow.

By binding to a target tissue or cell where its antigen or receptor is located, Z directs the conjugate there. Cleavage of group C at the target tissue or cell releases D to exert its effect locally. In this manner, precise delivery of D is achieved at the site of intended action, reducing the dosage needed. Also, D is normally biologically inactive (or significantly less active) in its conjugated state, thereby reducing off-target effects.

As reflected by the subscript m, each Z can conjugate with more than one D, depending on the number of sites Z has available for conjugation and the experimental conditions employed. Those skilled in the art will appreciate that, while each individual Z is conjugated to an integer number of Ds, a preparation of the conjugate may analyze for a non-integer ratio of D to Z, reflecting a statistical average. This ratio is referred to as the substitution ratio (“SR”) or the drug-antibody ratio (“DAR”).

In some embodiments, Z is conjugated to D (i.e., a compound of Formula I, II, III, IVA, IVB, or V) through a linker attached to an —OH substituent at R^{1a} or R^{1b} of the compound of Formula I, II, III, IVA, IVB, or V. In some embodiments, Z is conjugated to D (i.e., a compound of Formula I, II, III, IVA, IVB, or V) through a linker attached to an amine substituent of R⁴ of the compound of Formula I, II, III, IVA, IVB, or V. In some embodiments, Z is conjugated to D (i.e., a compound of Formula I, II, III, IVA, IVB, or V) through a linker, where the point of attachment of the linker to D is a heteroatom on D, where the linker replaces a hydrogen on the heteroatom (e.g., the linker is attached to D by replacing a hydrogen on a hydroxyl group or a primary or secondary amine).

### Targeting Moiety Z

Preferably, targeting moiety Z is an antibody. For convenience and brevity and not by way of limitation, the detailed discussion in this specification about Z and its conjugates is written in the context of its being an antibody, but those skilled in the art will understand that other types of Z can be conjugated, mutatis mutandis. For example, conjugates with folic acid as the targeting moiety can target cells having the folate receptor on their surfaces (Leamon et al., Cancer Res. 2008, 68 (23), 9839). For the same reasons, the detailed discussion in this specification is primarily written in terms of a 1:1 ratio of Z to D (m=1).

Antibodies that can be used in conjugates provided herein include those recognizing the following antigens: mesothelin, prostate specific membrane antigen (PSMA), CD19, CD22, CD30, CD70, B7H3, B7H4 (also known as O8E), protein tyrosine kinase 7 (PTK7), glypican-3, RG1, fucosyl-GM1, CTLA-4, LIV-1, and CD44. The antibody can be animal (e.g., murine), chimeric, humanized, or, preferably, human. The antibody preferably is monoclonal, especially a monoclonal human antibody. The preparation of human monoclonal antibodies against some of the aforementioned antigens is disclosed in Korman et al., U.S. Pat. No. 8,609,816 B2 (2013; B7H4, also known as O8E; in particular antibodies 2A7, 1G11, and 2F9); Rao-Naik et al., 8,097,703 B2 (2012; CD19; in particular antibodies 5G7, 13F1, 46E8, 21D4, 21D4a, 47G4, 27F3, and 3C10); King et al., U.S. Pat. No. 8,481,683 B2 (2013; CD22; in particular antibodies 12C5, 19A3, 16F7, and 23C6); Keler et al., U.S. Pat. No. 7,387,776 B2 (2008; CD30; in particular antibodies 5F11, 2H9, and 17G1); Terrett et al., U.S. Pat. No. 8,124,738 B2 (2012; CD70; in particular antibodies 2H5, 10B4, 8B5, 18E7, and 69A7); Korman et al., U.S. Pat. No. 6,984,720 B1 (2006; CTLA-4; in particular antibodies 10D1, 4B6, and 1E2); Korman et al., U.S. Pat. No. 8,008,449 B2 (2011; PD-1; in particular antibodies 17D8, 2D3, 4H1, 5C4, 4A11, 7D3, and 5F4); Huang et al., US 2009/0297438 A1 (2009; PSMA. in particular antibodies 1C3, 2A10, 2F5, 2C6); Cardarelli et al., U.S. Pat. No. 7,875,278 B2 (2011; PSMA; in particular antibodies 4A3, 7F12, 8C12, 8A11, 16F9, 2A10, 2C6, 2F5, and 1C3); Terrett et al., U.S. Pat. No. 8,222,375 B2 (2012; PTK7; in particular antibodies 3G8, 4D5, 12C6, 12C6a, and 7C8); Harkins et al., U.S. Pat. No. 7,335,748 B2 (2008; RG1; in particular antibodies A, B, C, and D); Terrett et al., U.S. Pat. No. 8,268,970 B2 (2012; mesothelin; in particular antibodies 3C10, 6A4, and 7B1); Xu et al., US 2010/0092484 A1 (2010; CD44; in particular antibodies 14G9.B8.B4, 2D1.A3.D12, and 1A9.A6.B9); Deshpande et al., U.S. Pat. No. 8,258,266 B2 (2012; IP10; in particular antibodies 1D4, 1E1, 2G1, 3C4, 6A5, 6A8, 7C10, 8F6, 10A12, 10A12S, and 13C4); Kuhne et al., U.S. Pat. No. 8,450,464 B2 (2013; CXCR4; in particular antibodies F7, F9, D1, and E2); and Korman et al., U.S. Pat. No. 7,943,743 B2 (2011; PD-L1; in particular antibodies 3G10, 12A4, 10A5, 5F8, 10H10, 1B12, 7H1, 11E6, 12B7, and 13G4); the disclosures of which are incorporated herein by reference. Preferably, the antibody is an anti-mesothelin antibody.

In addition to being an antibody, Z can also be an antibody fragment (such as Fab, Fab′, F(ab′)₂, Fd, or Fv) or antibody mimetic, such as an affibody, a domain antibody (dAb), a nanobody, a unibody, a DARPin, an anticalin, a versabody, a duocalin, a lipocalin, or an avimer.

Any one of several different reactive groups on Z can be a conjugation site, including F-amino groups in lysine residues, pendant carbohydrate moieties, carboxylic acid groups on aspartic or glutamic acid side chains, cysteine-cysteine disulfide groups, and cysteine thiol groups. For reviews on antibody reactive groups suitable for conjugation, see, e.g., Garnett, Adv. Drug Delivery Rev. 2001, 53, 171-216 and Dubowchik and Walker, Pharmacology & Therapeutics 1999, 83, 67-123, the disclosures of which are incorporated herein by reference.

Most antibodies have multiple lysine residues, which can be conjugated via their F-amino groups via amide, urea, thiourea, or carbamate bonds.

A thiol (—SH) group in the side chain of a cysteine can be used to form a conjugate by several methods. It can be used to form a disulfide bond between it and a thiol group on the linker. Another method is via its Michael addition to a maleimide group on the linker.

Typically, although antibodies have cysteine residues, they lack free thiol groups because all their cysteines are engaged in intra- or inter-chain disulfide bonds. To generate a free thiol group, a native disulfide group can be reduced. See, e.g., Packard et al., Biochemistry 1986, 25, 3548; King et al., Cancer Res. 1994, 54, 6176; and Doronina et al., Nature Biotechnol. 2003, 21, 778. Alternatively, a cysteine having a free —SH group can be introduced by mutating the antibody, substituting a cysteine for another amino acid or inserting one into the polypeptide chain. See, for example, Eigenbrot et al., U.S. Pat. No. 7,521,541 B2 (2009); Chilkoti et al., Bioconjugate Chem. 1994, 5, 504; Urnovitz et al., U.S. Pat. No. 4,698,420 (1987); Stimmel et al., J. Biol. Chem. 2000, 275, 30445; Barn et al., U.S. Pat. No. 7,311,902 B2 (2007); Kuan et al., J. Biol. Chem. 1994, 269, 7610; Poon et al., J. Biol. Chem. 1995, 270, 8571; Junutula et al., Nature Biotechnology 2008, 26, 925 and Rajpal et al., U.S. Provisional Application No. 62/270,245, filed Dec. 21, 2015. In yet another approach, a cysteine is added to the C-terminus of the heavy of light chain. See, e.g., Liu et al., U.S. Pat. No. 8,865,875 B2 (2014); Cumber et al., J. Immunol. 1992, 149, 120; King et al, Cancer Res. 1994, 54, 6176; Li et al., Bioconjugate Chem. 2002, 13, 985; Yang et al., Protein Engineering 2003, 16, 761; and Olafson et al., Protein Engineering Design & Selection 2004, 17, 21. The disclosures of the documents cited in this paragraph are incorporated herein by reference.

Linkers and their Components

As noted above, the linker comprises up to three elements: a cleavable group C and optional spacers X^{Z} and X^{D}.

Group C is cleavable under physiological conditions. Preferably it is relatively stable while the conjugate is in circulation in the blood, but is readily cleaved once the conjugate reaches its site of intended action.

A preferred group C is a peptide that is cleaved selectively by a protease inside the target cell, as opposed to by a protease in the serum. Typically, the peptide comprises from 1 to 20 amino acids, preferably from 1 to 6 amino acids, more preferably from 2 to 3 amino acids. The amino acid(s) can be natural and/or non-natural α-amino acids. Natural amino acids are those encoded by the genetic code, as well as amino acids derived therefrom, e.g., hydroxyproline, γ-carboxyglutamate, citrulline, and O-phosphoserine. In this specification, the term “amino acid” also includes amino acid analogs and mimetics. Analogs are compounds having the same general H₂N(R)CHCO₂H structure of a natural amino acid, except that the R group is not one found among the natural amino acids. Examples of analogs include homoserine, norleucine, methionine-sulfoxide, and methionine methyl sulfonium. An amino acid mimetic is a compound that has a structure different from the general chemical structure of an α-amino acid but functions in a manner similar to one. The amino acid can be of the “L” stereochemistry of the genetically encoded amino acids, as well as of the enantiomeric “D” stereochemistry.

Preferably, C contains an amino acid sequence that is a cleavage recognition sequence for a protease. Many cleavage recognition sequences are known in the art. See, e.g., Matayoshi et al. Science 247: 954 (1990); Dunn et al. Meth. Enzymol. 241: 254 (1994); Seidah et al. Meth. Enzymol. 244: 175 (1994); Thornberry, Meth. Enzymol. 244: 615 (1994); Weber et al. Meth. Enzymol. 244: 595 (1994); Smith et al. Meth. Enzymol. 244: 412 (1994); and Bouvier et al. Meth. Enzymol. 248: 614 (1995); the disclosures of which are incorporated herein by reference.

A group C can be chosen such that it is cleaved by a protease present in the extracellular matrix in the vicinity of a cancer, e.g., a protease released by nearby dying cancer cells or a tumor-associated protease secreted by cancer cells. Exemplary extracellular tumor-associated proteases are plasmin, matrix metalloproteases (MMP), thimet oligopeptidase (TOP) and CD10. See, e.g., Trouet et al., U.S. Pat. No. 7,402,556 B2 (2008); Dubois et al., U.S. Pat. No. 7,425,541 B2 (2008); and Bebbington et al., U.S. Pat. No. 6,897,034 B2 (2005). Cathepsin D, normally lysosomal enzyme found inside cells, is sometimes found in the environs of a tumor, possibly released by dying cancer cells.

For conjugates designed to be by an enzyme, C preferably comprises an amino acid sequence selected for cleavage by proteases such cathepsins B, C, D, H, L and S, especially cathepsin B. Exemplary cathepsin B cleavable peptides include Val-Ala, Val-Cit, Val-Lys, Lys-Val-Ala, Asp-Val-Ala, Val-Ala, Lys-Val-Cit, Ala-Val-Cit, Val-Gly, Val-Gln, and Asp-Val-Cit. (Herein, amino acid sequences are written in the N-to-C direction, as in H₂N-AA²-AA¹-CO₂H, unless the context clearly indicates otherwise.) See Dubowchik et al., Biorg. Med. Chem. Lett. 1998, 8, 3341; Dubowchik et al., Bioorg. Med. Chem. Lett. 1998, 8, 3347; and Dubowchik et al., Bioconjugate Chem. 2002, 13, 855; the disclosures of which are incorporated by reference.

Another enzyme that can be utilized for cleaving peptidyl linkers is legumain, a lysosomal cysteine protease that preferentially cleaves at Ala-Ala-Asn.

In one embodiment, Group C is a peptide comprising a two-amino acid sequence -AA²-AA¹- wherein AA¹ is lysine, arginine, or citrulline and AA² is phenylalanine, valine, alanine, leucine or isoleucine. In another embodiment, C consists of a sequence of one to three amino acids, selected from the group consisting of Val-Cit, Ala-Val, Val-Ala-Val, Lys-Lys, Ala-Asn-Val, Val-Leu-Lys, Cit-Cit, Val-Lys, Ala-Ala-Asn, Lys, Cit, Ser, and Glu. More preferably, it is a two to three amino acid peptide from the foregoing group.

The preparation and design of cleavable groups C consisting of a single amino acid is disclosed in Chen et al., U.S. Pat. No. 8,664,407 B2 (2014), the disclosure of which is incorporated herein by reference.

Group C can be bonded directly to Z or D; i.e. spacers X^{Z} or X^{D}, as the case may be, can be absent.

When present, spacer X^{Z} provides spatial separation between C and Z, lest the former sterically interfere with antigen binding by latter or the latter sterically interfere with cleavage of the former. Further, spacer X^{Z} can be used to confer increased solubility or decreased aggregation properties to conjugates. A spacer X^{Z} can comprise one or more modular segments, which can be assembled in any number of combinations. Examples of suitable segments for a spacer X^{Z} are:

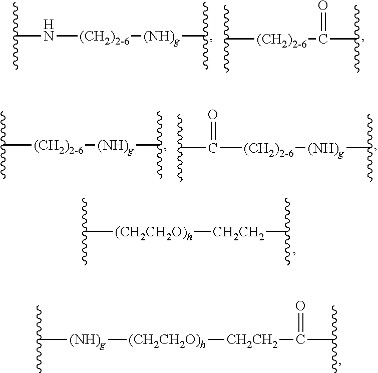

and combinations thereof,
where the subscript g is 0 or 1 and the subscript h is 1 to 24, preferably 2 to 4. These segments can be combined, such as illustrated below:

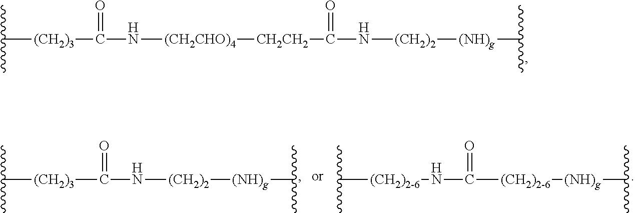

Spacer X^{D}, if present, provides spatial separation between C and D, lest the latter interfere sterically or electronically with cleavage of the former. Spacer X^{D} also can serve to introduce additional molecular mass and chemical functionality into a conjugate. Generally, the additional mass and functionality will affect the serum half-life and other properties of the conjugate. Thus, through judicious selection of spacer groups, the serum half-life of a conjugate can be modulated. Spacer X^{D} also can be assembled from modular segments, analogously to the description above for spacer X^{Z}.

Spacers X^{Z} and/or X^{D}, where present, preferably provide a linear separation of from 4 to 25 atoms, more preferably from 4 to 20 atoms, between Z and C or D and C, respectively.

The linker can perform other functions in addition to covalently linking the antibody and the drug. For instance, the linker can contain a poly(ethylene glycol) (“PEG”) group. Since the conjugation step typically involves coupling a drug-linker to an antibody in an aqueous medium, a PEG group many enhance the aqueous solubility of the drug-linker. Also, a PEG group may enhance the solubility or reduce aggregation in the resulting ADC. Where a PEG group is present, it may be incorporated into either spacer X^{Z} of X^{D}, or both. The number of repeat units in a PEG group can be from 2 to 20, preferably between 4 and 10.

Either spacer X^{Z} or X^{D}, or both, can comprise a self-immolating moiety. A self-immolating moiety is a moiety that (1) is bonded to C and either Z or D and (2) has a structure such that cleavage from group C initiates a reaction sequence resulting in the self-immolating moiety disbonding itself from Z or D, as the case may be. In other words, reaction at a site distal from Z or D (cleavage from group C) causes the X^{Z}—Z or the X^{D}-D bond to rupture as well. The presence of a self-immolating moiety is desirable in the case of spacer X^{D} because, if, after cleavage of the conjugate, spacer X^{D} or a portion thereof were to remain attached to D, the biological activity of D may be impaired. The use of a self-immolating moiety is especially desirable where cleavable group C is a polypeptide, in which instance the self-immolating moiety typically is located adjacent thereto, in order to prevent D from sterically or electronically interfering with peptide cleavage.

Exemplary self-immolating moieties (i)-(v) bonded to a hydroxyl or amino group of D are shown below:

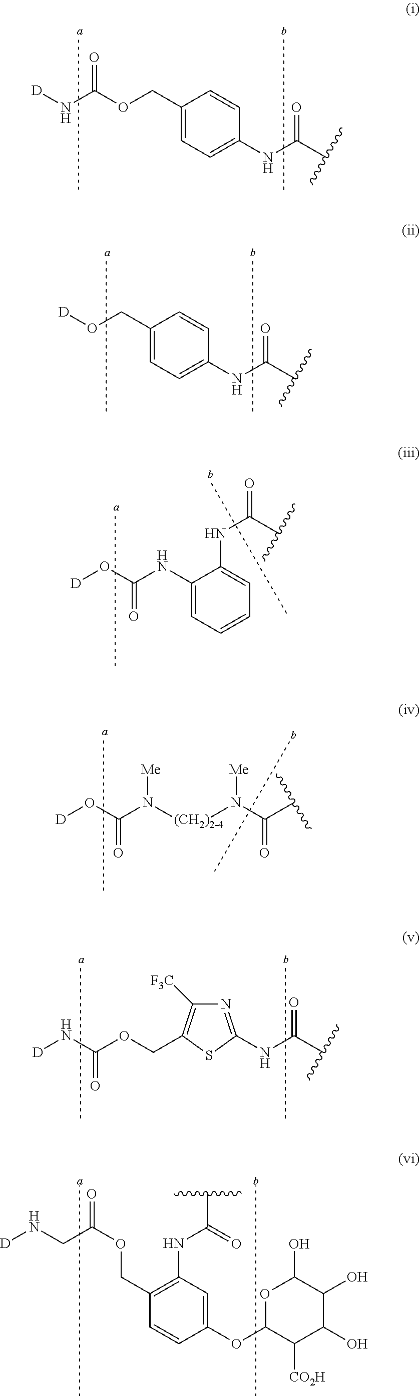

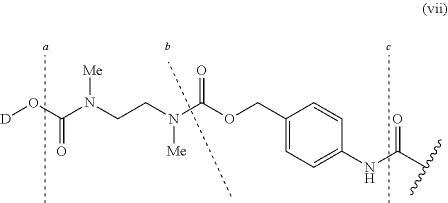

The self-immolating moiety is the structure between dotted lines a and b (or dotted lines b and c), with adjacent structural features shown to provide context. Self-immolating moieties (i) and (v) are bonded to a D-NH₂ (i.e., conjugation is via an amino group), while self-immolating moieties (ii), (iii), and (iv) are bonded to a D-OH (i.e., conjugation is via a hydroxyl or carboxyl group). Cleavage of the bond at dotted line b by an enzyme—a peptidase in the instance of structures (i)-(v) and a β-glucuronidase in the instance of structure (vi)—initiates a self-immolating reaction sequence that results in the cleavage of the bond at dotted line a and the consequent release of D-OH or D-NH₂, as the case may be. By way of illustration, self-immolating mechanisms for structures (i) and (iv) are shown below:

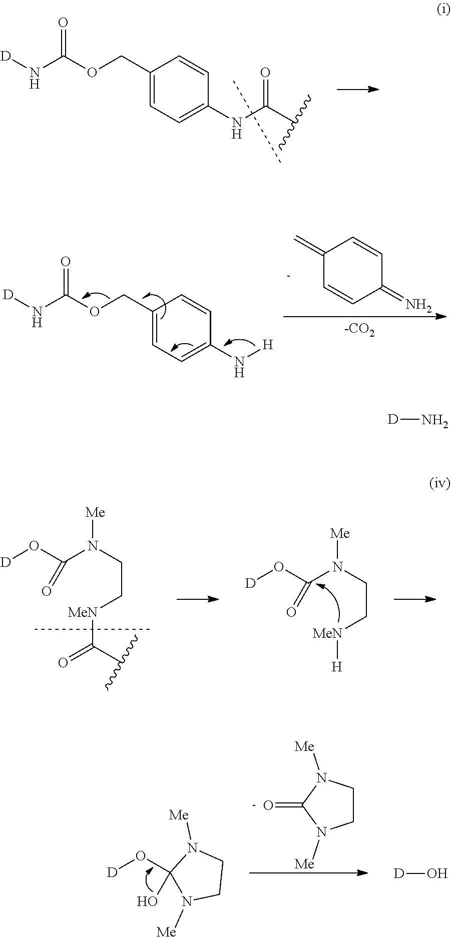

In other words, cleavage of a first chemical bond at one part of a self-immolating group initiates a sequence of steps that results in the cleavage of a second chemical bond—the one connecting the self-immolating group to the drug—at a different part of the self-immolating group, thereby releasing the drug.

In some instances, self-immolating groups can be used in tandem, as shown by structure (vii). In such case, cleavage at dotted line c triggers self-immolation of the moiety between dotted lines b and c by a 1,6-elimination reaction, followed by self-immolation of the moiety between dotted lines a and b by a cyclization-elimination reaction. For additional disclosures regarding self-immolating moieties, see Carl et al., J. Med. Chem. 1981, 24, 479; Carl et al., WO 81/01145 (1981); Dubowchik et al., Pharmacology & Therapeutics 1999, 83, 67; Firestone et al., U.S. Pat. No. 6,214,345 B1 (2001); Toki et al., J. Org. Chem. 2002, 67, 1866; Doronina et al., Nature Biotechnology 2003, 21, 778 (erratum, p. 941); Boyd et al., U.S. Pat. No. 7,691,962 B2; Boyd et al., US 2008/0279868 A1; Sufi et al., WO 2008/083312 A2; Feng, U.S. Pat. No. 7,375,078 B2; Jeffrey et al., U.S. Pat. No. 8,039,273; and Senter et al., US 2003/0096743 A1; the disclosures of which are incorporated by reference.

In another embodiment, Z and D are linked by a non-cleavable linker, i.e., C is absent. Metabolism of D eventually reduces the linker to a small appended moiety that does not interfere with the biological activity of D.

### Conjugation Techniques

Conjugates of TLR7 agonists disclosed herein preferably are made by first preparing a compound comprising D and linker (X^{D})ₐ(C)_{c}(X^{Z})_{b} (where X^{D}, C, X^{Z}, a, b, and c are as defined for Formula (VI)) to form drug-linker compound represented by Formula (VII):

D-(X^{D})ₐ(C)_{c}(X^{Z})_{b}—R³¹ (VII)
where R³¹ is a functional group suitable for reacting with a complementary functional group on Z to form the conjugate. Examples of suitable groups R³¹ include amino, azide, thiol, cyclooctyne,

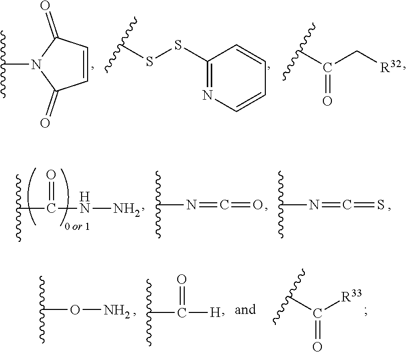

where R³² is Cl, Br, F, mesylate, or tosylate and R³³ is Cl, Br, I, F,
OH, —O—N-succinimidyl, —O-(4-nitrophenyl), —O-pentafluorophenyl, or —O-tetrafluorophenyl. Chemistry generally usable for the preparation of suitable moieties D-(X^{D})ₐC(X^{Z})_{b}—R³¹ is disclosed in Ng et al., U.S. Pat. No. 7,087,600 B2 (2006); Ng et al., U.S. Pat. No. 6,989,452 B2 (2006); Ng et al., U.S. Pat. No. 7,129,261 B2 (2006); Ng et al., WO 02/096910 A1; Boyd et al., U.S. Pat. No. 7,691,962 B2; Chen et al., U.S. Pat. No. 7,517,903 B2 (2009); Gangwar et al., U.S. Pat. No. 7,714,016 B2 (2010); Boyd et al., US 2008/0279868 A1; Gangwar et al., U.S. Pat. No. 7,847,105 B2 (2010); Gangwar et al., U.S. Pat. No. 7,968,586 B2 (2011); Sufi et al., U.S. Pat. No. 8,461,117 B2 (2013); and Chen et al., U.S. Pat. No. 8,664,407 B2 (2014); the disclosures of which are incorporated herein by reference.

Preferably reactive functional group —R³¹ is —NH₂, —OH, —CO₂H, —SH, maleimido, cyclooctyne, azido (—N₃), hydroxylamino (—ONH₂) or N-hydroxysuccinimido. Especially preferred functional groups —R³¹ are:

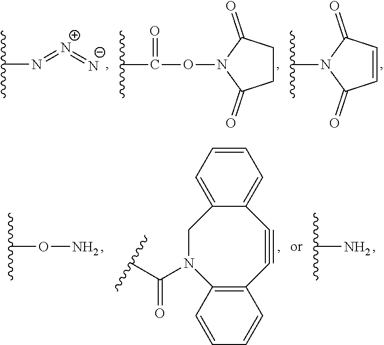

An —OH group can be esterified with a carboxy group on the antibody, for example, on an aspartic or glutamic acid side chain.

A —CO₂H group can be esterified with a —OH group or amidated with an amino group (for example on a lysine side chain) on the antibody.

An N-hydroxysuccinimide group is functionally an activated carboxyl group and can conveniently be amidated by reaction with an amino group (e.g., from lysine).

A maleimide group can be conjugated with an —SH group on the antibody (e.g., from cysteine or from the chemical modification of the antibody to introduce a sulfhydryl functionality), in a Michael addition reaction.

Where an antibody does not have a cysteine —SH available for conjugation, an F-amino group in the side chain of a lysine residue can be reacted with 2-iminothiolane or N-succinimidyl-3-(2-pyridyldithio)propionate (“SPDP”) to introduce a free thiol (—SH) group—creating a cysteine surrogate, as it were. The thiol group can react with a maleimide or other nucleophile acceptor group to effect conjugation. The mechanism if illustrated below with 2-iminothiolane.

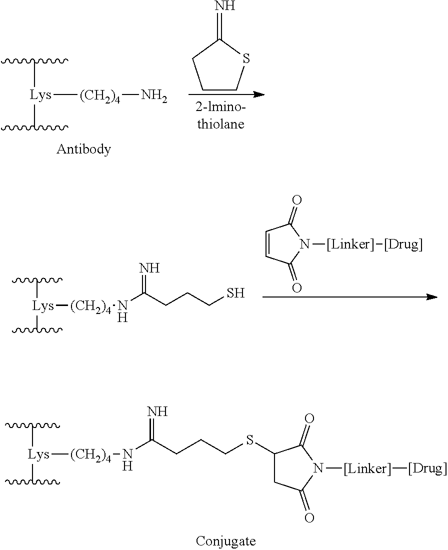

Typically, a thiolation level of two to three thiols per antibody is achieved. For a representative procedure, see Cong et al., U.S. Pat. No. 8,980,824 B2 (2015), the disclosure of which is incorporated herein by reference.

In a reversed arrangement, an antibody Z can be modified with N-succinimidyl 4-(maleimidomethyl)-cyclohexanecarboxylate (“SMCC”) or its sulfonated variant sulfo-SMCC, both of which are available from Sigma-Aldrich, to introduce a maleimide group thereto. Then, conjugation can be affected with a drug-linker compound having an —SH group on the linker.

An alternative conjugation method employs copper-free “click chemistry,” in which an azide group adds across a strained cyclooctyne to form an 1,2,3-triazole ring. See, e.g., Agard et al., J. Amer. Chem. Soc. 2004, 126, 15046; Best, Biochemistry 2009, 48, 6571, the disclosures of which are incorporated herein by reference. The azide can be located on the antibody and the cyclooctyne on the drug-linker moiety, or vice-versa. A preferred cyclooctyne group is dibenzocyclooctyne (DIBO). Various reagents having a DIBO group are available from Invitrogen/Molecular Probes, Eugene, Oregon. The reaction below illustrates click chemistry conjugation for the instance in which the DIBO group is attached to the antibody (Ab):

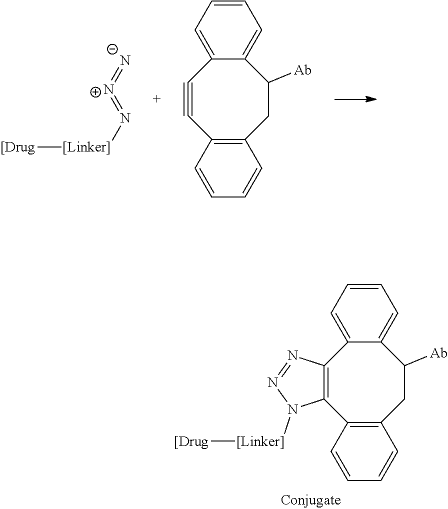

Yet another conjugation technique involves introducing a non-natural amino acid into an antibody, with the non-natural amino acid providing a functionality for conjugation with a reactive functional group in the drug moiety. For instance, the non-natural amino acid p-acetylphenylalanine can be incorporated into an antibody or other polypeptide, as taught in Tian et al., WO 2008/030612 A2 (2008). The ketone group in p-acetylphenyalanine can be a conjugation site via the formation of an oxime with a hydroxylamino group on the linker-drug moiety. Alternatively, the non-natural amino acid p-azidophenylalanine can be incorporated into an antibody to provide an azide functional group for conjugation via click chemistry, as discussed above. Non-natural amino acids can also be incorporated into an antibody or other polypeptide using cell-free methods, as taught in Goerke et al., US 2010/0093024 A1 (2010) and Goerke et al., Biotechnol. Bioeng. 2009, 102 (2), 400-416. The foregoing disclosures are incorporated herein by reference. Thus, in one embodiment, an antibody that is used for making a conjugate has one or more amino acids replaced by a non-natural amino acid, which preferably is p-acetylphenylalanine or p-azidophenylalanine, more preferably p-acetylphenylalanine.

Still another conjugation technique uses the enzyme transglutaminase (preferably bacterial transglutaminase from Streptomyces mobaraensis or BTG), per Jeger et al., Angew. Chem. Int. Ed. 2010, 49, 9995. BTG forms an amide bond between the side chain carboxamide of a glutamine (the amine acceptor) and an alkyleneamino group (the amine donor), which can be, for example, the F-amino group of a lysine or a 5-amino-n-pentyl group. In a typical conjugation reaction, the glutamine residue is located on the antibody, while the alkyleneamino group is located on the linker-drug moiety, as shown below:

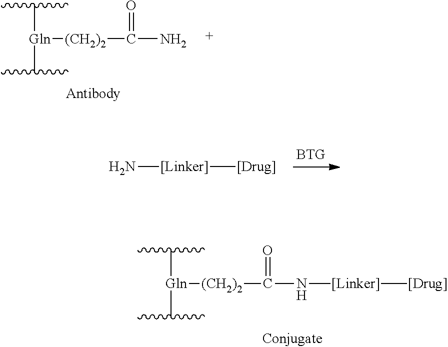

The positioning of a glutamine residue on a polypeptide chain has a large effect on its susceptibility to BTG mediated transamidation. None of the glutamine residues on an antibody are normally BTG substrates. However, if the antibody is deglycosylated—the glycosylation site being asparagine 297 (N297; numbering per EU index as set forth in Kabat et al., “Sequences of proteins of immunological interest,” 5th ed., Pub. No. 91-3242, U.S. Dept. Health & Human Services, NIH, Bethesda, Md., 1991; hereinafter “Kabat”) of the heavy chain—nearby glutamine 295 (Q295) is rendered BTG susceptible. An antibody can be deglycosylated enzymatically by treatment with PNGase F (Peptide-N-Glycosidase F). Alternatively, an antibody can be synthesized glycoside free by introducing an N297A mutation in the constant region, to eliminate the N297 glycosylation site. Further, it has been shown that an N297Q substitution not only eliminates glycosylation, but also introduces a second glutamine residue (at position 297) that too is an amine acceptor. Thus, in one embodiment, the antibody is deglycosylated. In another embodiment, the antibody has an N297Q substitution. Those skilled in the art will appreciate that deglycosylation by post-synthesis modification or by introducing an N297A mutation generates two BTG-reactive glutamine residues per antibody (one per heavy chain, at position 295), while an antibody with an N297Q substitution will have four BTG-reactive glutamine residues (two per heavy chain, at positions 295 and 297).

An antibody can also be rendered susceptible to BTG-mediated conjugation by introducing into it a glutamine containing peptide, or “tag,” as taught, for example, in Pons et al., US 2013/0230543 A1 (2013) and Rao-Naik et al., WO 2016/144608 A1.

In a complementary approach, the substrate specificity of BTG can be altered by varying its amino acid sequence, such that it becomes capable of reacting with glutamine 295 in an unmodified antibody, as taught in Rao-Naik et al., WO 2017/059158 A1 (2017).

While the most commonly available bacterial transglutaminase is that from S. mobaraensis, transglutaminase from other bacteria, having somewhat different substrate specificities, can be considered, such as transglutaminase from Streptoverticillium ladakanum (Hu et al., US 2009/0318349 A1 (2009), US 2010/0099610 A1 (2010), and US 2010/0087371 A1 (2010)).

### Pegylation

Attachment of a poly(ethylene glycol) (PEG) chain to a drug (“PEGylation”) can improve the latter's pharmacokinetic properties. The circulation half-life of the drug is increased, sometimes by over an order of magnitude, concomitantly reducing the dosage needed to achieve a desired therapeutic effect. PEGylation can also decrease metabolic degradation of a drug and reduce its immunogenicity. For a review, see Kolate et al., J. Controlled Release 2014, 192, 167.

Initially, PEGylation was applied to biologic drugs. As of 2016, over ten PEGylated biologics had been approved. Turecek et al., J. Pharmaceutical Sci. 2016, 105, 460. More recently, stimulated by the successful application of the concept to biologics, attention has turned towards its application to small molecule drugs. In addition to the aforementioned benefits, PEGylated small molecule drugs may have increased solubility and cause fewer toxic effects. Li et al. Prog. Polymer Sci. 2013, 38, 421.

The compounds disclosed herein can be PEGylated. Where a compound has an aliphatic primary or secondary amine or an aliphatic hydroxyl, it can be PEGylated via an ester, amide, carbonate, or carbamate group with a carboxy-containing PEG molecule utilizing conventional techniques such as dicyclohexylcarbodiimide, HATU, N-hydroxysuccinimide esters, and the like. Various other methods for PEGylating pharmaceutical molecules are disclosed in Alconcel et al., Polymer Chem. 2011, 2, 1442, the disclosure of which is incorporated herein by reference.

If desired, a TLR7 agonist disclosed herein can be PEGylated via an enzymatically cleavable linker comprising a self-immolating moiety, to allow release of the un-PEGylated agonist in a designed manner. Further, PEGylation can be combined with conjugation to a protein such as an antibody, if the PEG-containing molecule has a suitable functional group such as an amine for attachment to the protein. The protein can provide an additional therapeutic function or, if an antibody, can provide a targeting function. These concepts are illustrated in the following reaction sequence, where TLR7-NH—R generically represents a TLR7 agonist:

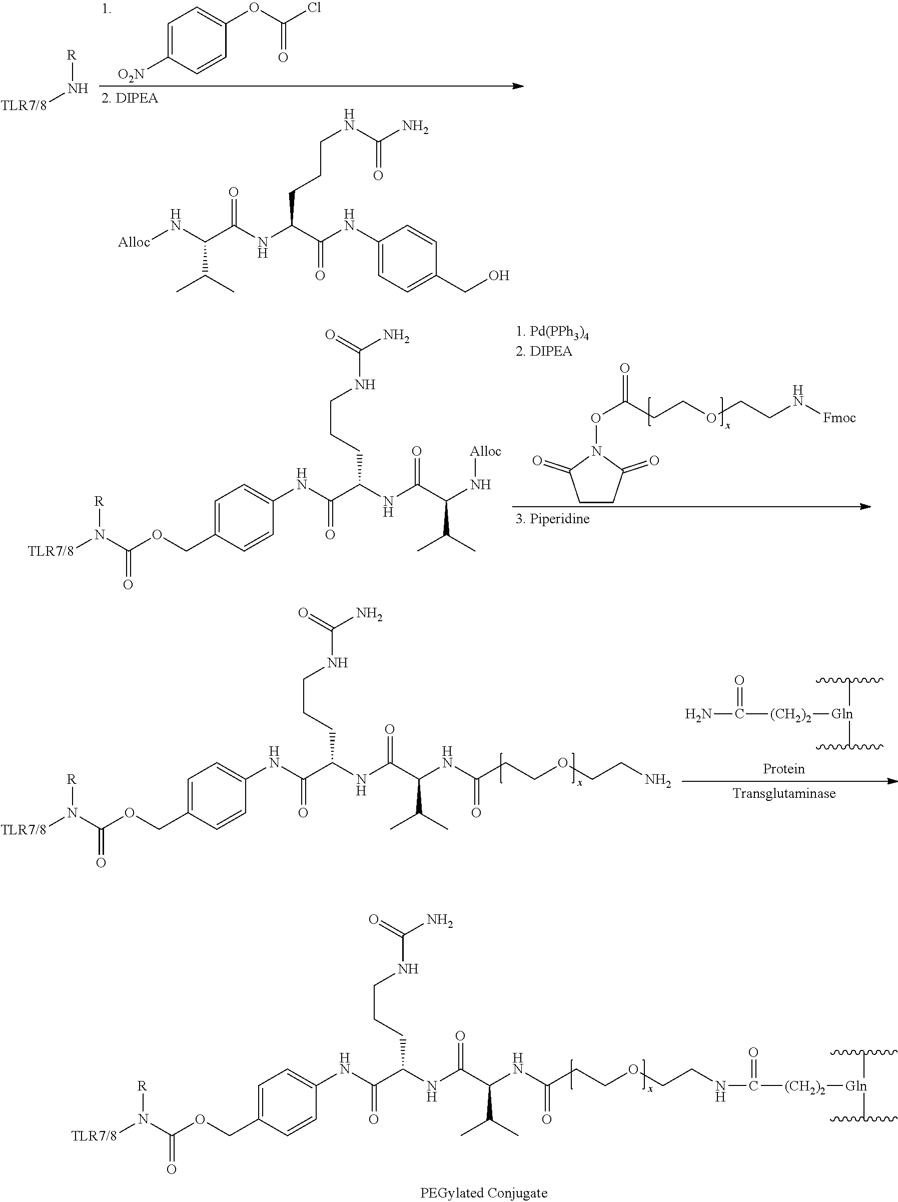

In the above reaction sequence, the valine-citrulline (Val-Cit) dipeptide is cleavable by the enzyme cathepsin B, with a p-aminobenzyl oxycarbonyl (PABC) group serving as a self-immolating spacer. The functional group for conjugation is an amine group, which is temporarily protected by an Fmoc group. Conjugation is effected by the enzyme transglutaminase, with a glutamine (Gln) side chain acting as the acyl acceptor. The subscript x, denoting the number of PEG repeat units, can vary widely, depending on the purpose of the PEGylation, as discussed below. For some purposes, x can be relatively small, such as 2, 4, 8, 12, or 24. For other purposes, x is large, for example between about 45 and about 910.

Those skilled in the art will understand that the sequence is illustrative and that other elements—peptide, self-immolating group, conjugation method, PEG length, etc.—may be employed, as is well known in the art. They will also understand that, while the above sequence combines PEGylation and conjugation, PEGylation does not require conjugation, and vice-versa.

Where the compound lacks aliphatic hydroxyl or aliphatic primary or secondary amine, it still can be PEGylated at the aromatic amine on the pyrimidine ring. A method for PEGylating at this position is disclosed by Zarraga, US 2017/0166384 A1 (2007), the disclosure of which is incorporated by reference.

In some embodiments, it may be desirable to have multiple PEGylated agonists linked in a single molecule. For instance, four PEGylated arms can be constructed on pentaerythritol (C(CH₂OH)₄) and a TLR7 agonist can be attached to each PEGylated arm. See Gao et al., US 2013/0028857 A1 (2013), the disclosure of which is incorporated by reference.

For modulating pharmacokinetics, it is generally preferred that the PEG moiety have a formula weight of between about 2 kDa (corresponding to about 45 —(CH₂CH₂O)— repeating units) and between about 40 kDa (corresponding to about 910 —(CH₂CH₂O)— repeating units), more preferably between about 5 kDa and about 20 kDa. That is, the range of the subscript x in the above formulae is from about 45 to about 910. It is to be understood that PEG compositions are not 100% homogeneous but, rather, exhibit a distribution of molecular weights. Thus, a reference to, for example, “20 kDa PEG” means PEG having an average molecular weight of 20 kDa.

PEGylation can also be used for improving the solubility of an agonist. In such instances a shorter PEG chain can be used, for example comprising 2, 4, 8, 12, or 24 repeating units.

### Pharmaceutical Compositions and Administration

In another aspect, there is provided a pharmaceutical composition comprising a compound of as disclosed herein, or of a conjugate thereof, formulated together with a pharmaceutically acceptable carrier or excipient. It may optionally contain one or more additional pharmaceutically active ingredients, such as a biologic or a small molecule drug. The pharmaceutical compositions can be administered in a combination therapy with another therapeutic agent, especially an anti-cancer agent.

The pharmaceutical composition may comprise one or more excipients. Excipients that may be used include carriers, surface active agents, thickening or emulsifying agents, solid binders, dispersion, or suspension aids, solubilizers, colorants, flavoring agents, coatings, disintegrating agents, lubricants, sweeteners, preservatives, isotonic agents, and combinations thereof. The selection and use of suitable excipients is taught in Gennaro, ed., Remington: The Science and Practice of Pharmacy, 20th Ed. (Lippincott Williams & Wilkins 2003).

Preferably, a pharmaceutical composition is suitable for intravenous, intramuscular, subcutaneous, parenteral, spinal or epidermal administration (e.g., by injection or infusion). Depending on the route of administration, the active compound may be coated in a material to protect it from the action of acids and other natural conditions that may inactivate it. The phrase “parenteral administration” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, the pharmaceutical composition can be administered via a non-parenteral route, such as a topical, epidermal, or mucosal route of administration, for example, intranasally, orally, vaginally, rectally, sublingually or topically.

Pharmaceutical compositions can be in the form of sterile aqueous solutions or dispersions. They can also be formulated in a microemulsion, liposome, or other ordered structure suitable to achieve high drug concentration. The compositions can also be provided in the form of lyophilates, for reconstitution in water prior to administration.

The amount of active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide a therapeutic response. For example, a single bolus may be administered, several divided doses may be administered over time, or the dose may be proportionally reduced or increased as indicated by the exigencies of the situation. It is especially advantageous to formulate parenteral compositions in dosage unit form for ease of administration and uniformity of dosage. “Dosage unit form” refers to physically discrete units suited as unitary dosages for the subjects to be treated; each unit containing a predetermined quantity of active compound calculated to produce the desired therapeutic response, in association with the required pharmaceutical carrier.

The dosage ranges from about 0.0001 to 100 mg/kg, and more usually 0.01 to 5 mg/kg, of the host body weight. For example, dosages can be 0.3 mg/kg body weight, 1 mg/kg body weight, 3 mg/kg body weight, 5 mg/kg body weight or 10 mg/kg body weight or within the range of 1-10 mg/kg, or alternatively 0.1 to 5 mg/kg. Exemplary treatment regimens are administration once per week, once every two weeks, once every three weeks, once every four weeks, once a month, once every 3 months, or once every three to 6 months. Preferred dosage regimens include 1 mg/kg body weight or 3 mg/kg body weight via intravenous administration, using one of the following dosing schedules: (i) every four weeks for six dosages, then every three months; (ii) every three weeks; (iii) 3 mg/kg body weight once followed by 1 mg/kg body weight every three weeks. In some methods, dosage is adjusted to achieve a plasma antibody concentration of about 1-1000 μg/mL and in some methods about 25-300 μg/mL.

A “therapeutically effective amount” of a compound provided herein preferably results in a decrease in severity of disease symptoms, an increase in frequency and duration of disease symptom-free periods, or a prevention of impairment or disability due to the disease affliction. For example, for the treatment of tumor-bearing subjects, a “therapeutically effective amount” preferably inhibits tumor growth by at least about 20%, more preferably by at least about 40%, even more preferably by at least about 60%, and still more preferably by at least about 80% relative to untreated subjects. A therapeutically effective amount of a therapeutic compound can decrease tumor size, or otherwise ameliorate symptoms in a subject, which is typically a human but can be another mammal. Where two or more therapeutic agents are administered in a combination treatment, “therapeutically effective amount” refers to the efficacy of the combination as a whole, and not each agent individually.

The pharmaceutical composition can be a controlled or sustained release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Therapeutic compositions can be administered via medical devices such as (1) needleless hypodermic injection devices; (2) micro-infusion pumps; (3) transdermal devices; (4) infusion devices; and (5) osmotic devices.

In certain embodiments, the pharmaceutical composition can be formulated to ensure proper distribution in vivo. For example, to ensure that the therapeutic compounds cross the blood-brain barrier, they can be formulated in liposomes, which may additionally comprise targeting moieties to enhance selective transport to specific cells or organs.

### INDUSTRIAL APPLICABILITY

TLR7 agonist compounds disclosed herein can be used for the treatment of a disease or condition that can be ameliorated by activation of TLR7.

In one embodiment, the TLR7 agonist is used in combination with an anti-cancer immunotherapy agent—also known as an immuno-oncology agent. An anti-cancer immunotherapy agent works by stimulating a body's immune system to attack and destroy cancer cells, especially through the activation of T cells. The immune system has numerous checkpoint (regulatory) molecules, to help maintain a balance between its attacking legitimate target cells and preventing it from attacking healthy, normal cells. Some are stimulators (up-regulators), meaning that their engagement promotes T cell activation and enhances the immune response. Others are inhibitors (down-regulators or brakes), meaning that their engagement inhibits T cell activation and abates the immune response. Binding of an agonistic immunotherapy agent to a stimulatory checkpoint molecule can lead to the latter's activation and an enhanced immune response against cancer cells. Reciprocally, binding of an antagonistic immunotherapy agent to an inhibitory checkpoint molecule can prevent down-regulation of the immune system by the latter and help maintain a vigorous response against cancer cells. Examples of stimulatory checkpoint molecules are B7-1, B7-2, CD28, 4-1BB (CD137), 4-1BBL, ICOS, CD40, ICOS-L, OX40, OX40L, GITR, GITRL, CD70, CD27, CD40, DR3 and CD28H. Examples of inhibitory checkpoint molecules are CTLA-4, PD-1, PD-L1, PD-L2, LAG-3, TIM-3, Galectin 9, CEACAM-1, BTLA, CD69, Galectin-1, CD113, GPR56, VISTA, 2B4, CD48, GARP, PD1H, LAIR1, TIM-1, CD96 and TIM-4.

Whichever the mode of action of an anti-cancer immunotherapy agent, its effectiveness can be increased by a general up-regulation of the immune system, such as by the activation of TLR7. Thus, in one embodiment, this specification provides a method of treating a cancer, comprising administering to a patient suffering from such cancer a therapeutically effective combination of an anti-cancer immunotherapy agent and a TLR7 agonist as disclosed herein. The timing of administration can be simultaneous, sequential, or alternating. The mode of administration can systemic or local. The TLR7 agonist can be delivered in a targeted manner, via a conjugate.

Cancers that could be treated by a combination treatment as described above include acute myeloid leukemia, adrenocortical carcinoma, Kaposi sarcoma, lymphoma, anal cancer, appendix cancer, teratoid/rhabdoid tumor, basal cell carcinoma, bile duct cancer, bladder cancer, bone cancer, brain cancer, breast cancer, bronchial tumor, carcinoid tumor, cardiac tumor, cervical cancer, chordoma, chronic lymphocytic leukemia, chronic myeloproliferative neoplasm, colon cancer, colorectal cancer, craniopharyngioma, bile duct cancer, endometrial cancer, ependymoma, esophageal cancer, esthesioneuroblastoma, Ewing sarcoma, eye cancer, fallopian tube cancer, gallbladder cancer, gastrointestinal carcinoid tumor, gastrointestinal stromal tumor, germ cell tumor, hairy cell leukemia, head and neck cancer, heart cancer, liver cancer, hypopharngeal cancer, pancreatic cancer, kidney cancer, laryngeal cancer, chronic myelogenous leukemia, lip and oral cavity cancer, lung cancer, melanoma, Merkel cell carcinoma, mesothelioma, mouth cancer, oral cancer, osteosarcoma, ovarian cancer, penile cancer, pharyngeal cancer, prostate cancer, rectal cancer, salivary gland cancer, skin cancer, small intestine cancer, soft tissue sarcoma, testicular cancer, throat cancer, thyroid cancer, urethral cancer, uterine cancer, vaginal cancer, and vulvar cancer.

Anti-cancer immunotherapy agents that can be used in combination therapies as disclosed herein include: AMG 557, AMP-224, atezolizumab, avelumab, BMS 936559, cemiplimab, CP-870893, dacetuzumab, durvalumab, enoblituzumab, galiximab, IN4P321, ipilimumab, lucatumumab, MEDI-570, ME1DJ-6383, ME1DJ-6469, muromonab-CD3, nivolumab, pembrolizumab, pidilizumab, spartalizumab, tremelimumab, urelumab, utomilumab, varlilumab, vonlerolizumab. Table A below lists their alternative name(s) (brand name, former name, research code, or synonym) and the respective target checkpoint molecule.

TABLE A

| Immunotherapy Agent | Alternative Name(s) | Target |
|---|---|---|
| AMG 557 | | B7RP-1 (ICOSL) |
| AMP-224 | | PD-1 |
| Atezolizumab | MPDL3280A, RO5541267, | PD-L1 |
| | TECENTRIQ ® | |
| Avelumab | BAVENCIO ® | PD-L1 |
| BMS 936559 | | PD-L1 |
| Cemiplimab | LIBTAYO ® | PD-1 |
| CP-870893 | | CD40 |
| Dacetuzumab | | CD40 |
| Durvalumab | IMFINZI ® | PD-L1 |
| Enoblituzumab | MGA271 | B7-H3 |
| Galiximab | | B7-1 (CD80) |
| IMP321 | | LAG-3 |
| Ipilimumab | YERVOY ® | CTLA-4 |
| Lucatumumab | | CD40 |
| MEDI-570 | | ICOS (CD278) |
| MEDI-6383 | | OX40 |
| MEDI-6469 | | OX40 |
| Muromonab-CD3 | | CD3 |
| Nivolumab | OPDIVO ® | PD-1 |
| Pembrolizumab | KEYTRUDA ® | PD-1 |
| Pidilizumab | MDV9300 | PD-1 |
| Spartalizumab | PDR001 | PD-1 |
| Tremelimumab | Ticilimumab, CP-675, CP- | CTLA-4 |
| | 675, 206 | |
| Urelumab | BMS-663513 | CD137 |
| Utomilumab | PF-05082566 | CD137 |
| Varlilumab | CDX 1127 | CD27 |
| Vonlerolizumab | RG7888, MOXR0916, | OX40 |
| | pogalizumab | |

In one embodiment of a combination treatment with a TLR7 agonist, the anti-cancer immunotherapy agent is an antagonistic anti-CTLA-4, anti-PD-1, or anti-PD-L1 antibody. The cancer can be lung cancer (including non-small cell lung cancer), pancreatic cancer, kidney cancer, head and neck cancer, lymphoma (including Hodgkin's lymphoma), skin cancer (including melanoma and Merkel skin cancer), urothelial cancer (including bladder cancer), gastric cancer, hepatocellular cancer, or colorectal cancer.

In another embodiment of a combination treatment with a TLR7 agonist, the anti-cancer immunotherapy agent is an antagonistic anti-CTLA-4 antibody, preferably ipilimumab.

In another embodiment of a combination treatment with a TLR7 agonist, the anti-cancer immunotherapy agent is an antagonistic anti-PD-1 antibody, preferably nivolumab or pembrolizumab.

The TLR7 agonists disclosed herein also are useful as vaccine adjuvants.

### EXAMPLES

The following Examples are presented by way of illustration, not limitation. Compounds are named using the automatic name generating tool provided in ChemBiodraw Ultra (Cambridgesoft), which generates systematic names for chemical structures, with support for the Cahn-Ingold-Prelog rules for stereochemistry. One skilled in the art can modify the procedures set forth in the illustrative examples to arrive at the desired products.

Salts of the compounds described herein can be prepared by standard methods, such as inclusion of an acid (for example TFA, formic acid, or HCl) in the mobile phases during chromatography purification, or stirring of the products after chromatography purification, with a solution of an acid (for example, aqueous HCl).

The following abbreviations may be relevant for the application.

Abbreviations

| | |
|---|---|
| AcOH, HOAc | Acetic acid |
| AIBN | Azobisisobutyronitrile |
| aq. | Aqueous |
| BnNH₂ | Benzyl amine |
| Boc | t-Butyloxycarbonyl |
| BOP | (Benzotriazol-1-yloxy)tris(dimethylamino)- |
| | phosphonium hexafluorophosphate (V) |
| DBU | 1,8-Diazabicyclo[5.4.0]undec-7-ene |
| DCM | Dichloromethane |
| DIAD | Diisopropyl azodicarboxylate |
| DIBAL-H | Diisobutylaluminium hydride |
| DIPEA, DIEA | N,N-diisopropylethylamine, also known as |
| | Hunig's base |
| DMF | N,N-dimethylformamide |
| DMSO | Dimethyl sulfoxide |
| EA, EtOAc | Ethyl acetate |
| Et₃N | Triethylamine |
| Fmoc | Fluorenylmethyloxycarbonyl |
| h | Hour(s) |
| HATU | Hexafluorophosphate Azabenzotriazole |
| | Tetramethyl Uronium; 1-[Bis(dimethylamino)- |
| | methylene]-1H-1,2,3-triazolo[4,5-b]pyridinium 3- |
| | oxide hexafluorophosphate |
| HPLC | High pressure liquid chromatography |
| Hunig's base | See DIPEA, DIEA |
| HX | Hexanes |
| LAH | Lithium aluminium hydride |
| LCMS, LC-MS, LC/MS | Liquid chromatography-mass spectrometry |
| LDBBA | Lithium diisobutyl-tert-butoxyaluminum hydride |
| MeCN | Acetonitrile |
| MeOH | Methanol |
| min | Minute(s) |
| MS | Mass spectrometry |
| MsCl | Methanesylfonyl chloride, mesyl chloride |
| NBS | N-Bromosuccinimide |
| NMR | Nuclear magnetic resonance |
| PEG | Poly(ethylene glycol) |
| pet. | Petroleum |
| PTFE | Poly(tetraflurorethylene) |
| PTSA | p-Toluenesulfonic acid |
| RT, rt, or r.t. (in context | Retention time, in min |
| of liquid chromatography) | |
| RT, rt, or r.t. (in the | Room (ambient) temperature, circa 25° C. |
| context of reaction conditions) | |
| Sat. | Saturated |
| SFC | Supercritical fluid chromatography |
| Soln | Solution |
| TBAF | Tetra-n-butylammonium fluoride |
| TBDPS | tert-Butyldiphenylsilyl |
| TEA | Triethylamine |
| TEAA | Triethylammonium acetate |
| TFA | Trifluoroacetic acid |
| THF | Tetrahydrofuran |
| wt. | Weight |

### SYNTHETIC EXAMPLES

The practice of this invention can be further understood by reference to the following examples, which are provided by way of illustration and not of limitation.

Generally, the procedures disclosed herein produce a mixture of regioisomers, alkylated at the 1H or 2H position of the pyrazolopyrimidine ring system (which are also referred to as N1 and N2 regioisomers, respectively, alluding to the nitrogen that is alkylated). In the figures, sometimes the N2 regioisomers are not shown for convenience, but it is to be understood that they are present in the initial product mixture and separated at a later time, for example by preparative HPLC.

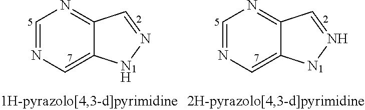 [SMILES: c1ncc2[nH]ncc2n1 ; c1ncc2[nH]ncc2n1]

The mixture of regioisomers can be separated at an early stage of the synthesis and the remaining synthetic steps carried out with the 1H regioisomer or, alternatively, the synthesis can be progressed carrying the mixture of regioisomers and separation effected at a later stage, as desired.

The foregoing detailed description includes passages that are chiefly or exclusively concerned with particular parts or aspects of the invention. It is to be understood that this is for clarity and convenience, that a particular feature may be relevant in more than just the passage in which it is disclosed, and that the disclosure herein includes all the appropriate combinations of information found in the different passages. Similarly, although the various figures and descriptions herein relate to specific embodiments of the invention, it is to be understood that where a specific feature is disclosed in the context of a particular figure or embodiment, such feature can also be used, to the extent appropriate, in the context of another figure or embodiment, in combination with another feature, or in the invention in general.

Further, while the present invention has been particularly described in terms of certain preferred embodiments, the invention is not limited to such preferred embodiments. Rather, the scope of the invention is defined by the appended claims.

### Example S1. 2-((5-Amino-2-(2-methoxy-4-(((4-methyltetrahydro-2H-pyran-4-yl) amino)methyl)benzyl)-2H-pyrazolo[4,3-d]pyrimidin-7-yl)(butyl)amino)ethan-1-ol (Compound 88)

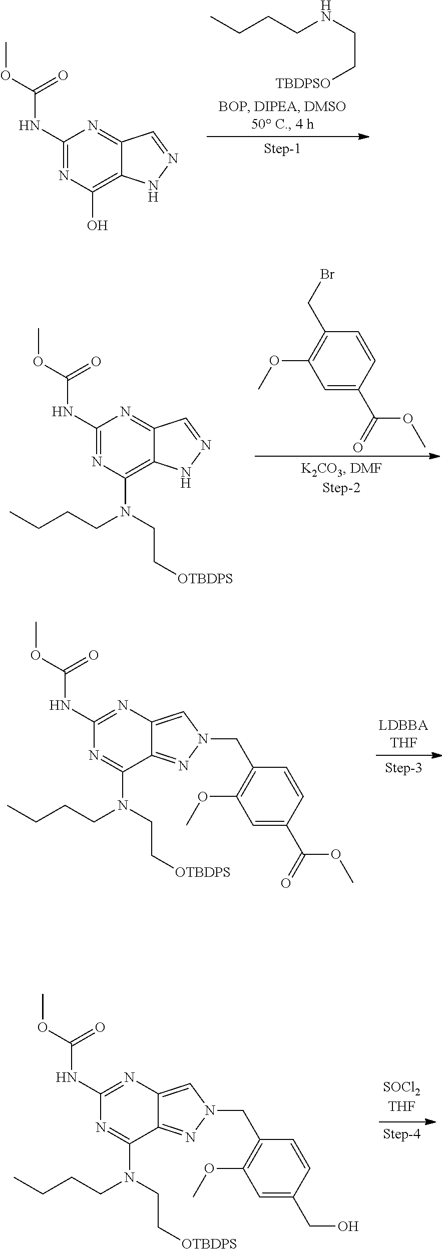

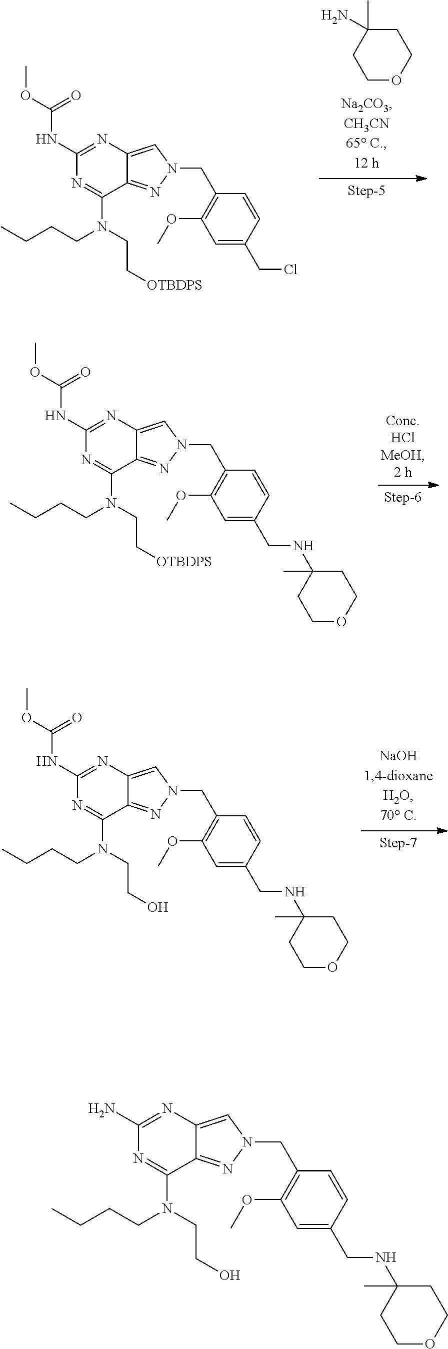

Step 1: Methyl (7-(butyl(2-((tert-butyldiphenylsilyl) oxy) ethyl) amino)-1H-pyrazolo[4,3-d]pyrimidin-5-yl) carbamate: To a stirred solution of methyl (7-hydroxy-1H-pyrazolo[4,3-d]pyrimidin-5-yl) carbamate (2 g, 9.56 mmol) in DMSO (12 mL), were added DIPEA (6.68 mL, 38.2 mmol), N-(2-((tert-butyldiphenylsilyl) oxy) ethyl) butan-1-amine (4.08 g, 11.47 mmol) and BOP (6.34 g, 14.34 mmol). The reaction mixture was stirred at 50° C. for 4 h. The reaction mixture was quenched by the addition of aq. sodium bicarbonate solution and extracted with ethyl acetate (2×20 mL). The combined organic layer was washed with water (20 mL) followed by brine solution (20 mL), dried over anhydrous sodium sulphate, filtered, and concentrated. The residue was purified by using CombiFlash column chromatography (40 g, silica gel column, 60% EA in pet. ether as an eluent) to afford the title compound (3 g, 5.49 mmol, 57.4% yield) as light brown semi solid. ¹H NMR (400 MHz, DMSO-d₆) δ 14.24-13.96 (m, 1H), 9.70-9.35 (m, 1H), 8.22-8.04 (m, 1H), 7.64-7.19 (m, 10H), 4.45-4.36 (m, 1H), 4.32-4.18 (m, 1H), 4.06-3.71 (m, 4H), 3.68-3.52 (m, 3H), 1.69-1.55 (m, 2H), 1.39-1.23 (m, 2H), 1.03-0.88 (m, 12H). LC-MS (ES): m/z=547.2 [M+H]⁺

Step 2: methyl 4-((7-(butyl(2-((tert-butyldiphenylsilyl)oxy)ethyl)amino)-5-((methoxycarbonyl)amino)-2H-pyrazolo[4,3-d]pyrimidin-2-yl)methyl)-3-methoxybenzoate: To a stirred solution of methyl (7-(butyl(2-((tert-butyldiphenylsilyl)oxy)ethyl)amino)-1H-pyrazolo[4,3-d]pyrimidin-5-yl)carbamate (4 g, 7.32 mmol) in anhydrous DMF (40 mL) at 0° C., were added K₂CO₃ (2.022 g, 14.63 mmol) and methyl 4-(bromomethyl)-3-methoxybenzoate (1.801 g, 6.95 mmol). The reaction mixture was stirred for 1.5 h at 0° C. The reaction mixture was quenched with ice-cold water and extracted with ethyl acetate. The aq. layer was re-extracted with ethyl acetate (2×50 mL). The combined organic layer was washed with water, brine solution, dried over anhydrous sodium sulphate, filtered, and concentrated. The crude was purified by flash chromatography (80 g, silic gel column, 30% ethyl acetate in pet. ether as eluent) to afford title compound (3.6 g, 4.97 mmol, 67.9% yield) as light brown semi solid. ¹H NMR (400 MHz, DMSO-d₆) δ 9.52-9.23 (m, 1H), 8.28-8.12 (m, 1H), 8.02-7.92 (m, 1H), 7.58-7.44 (m, 5H), 7.42-7.34 (m, 2H), 7.33-7.23 (m, 4H), 7.13-6.92 (m, 1H), 5.67-5.30 (m, 2H), 4.32-4.24 (m, 1H), 4.14-4.04 (m, 1H), 3.99-3.91 (m, 1H), 3.90-3.73 (m, 8H), 3.65-3.61 (m, 1H), 3.59-3.52 (m, 1H), 3.21-3.13 (m, 2H), 1.68-1.57 (m, 1H), 1.55-1.42 (m, 1H), 1.37-1.24 (m, 1H), 1.21-1.06 (m, 1H), 0.98-0.70 (m, 12H). LC-MS (ES): m/z=725.4 [M+H]⁺

Step 3: methyl (7-(butyl(2-((tert-butyldiphenylsilyl)oxy)ethyl)amino)-2-(4-(hydroxymethyl)-2-methoxybenzyl)-2H-pyrazolo[4,3-d]pyrimidin-5-yl)carbamate: To a solution of methyl 4-((7-(butyl(2-((tert-butyldiphenylsilyl)oxy)ethyl)amino)-5-((methoxycarbonyl)amino)-2H-pyrazolo[4,3-d]pyrimidin-2-yl)methyl)-3-methoxybenzoate (1 g, 1.379 mmol) in THE (10 mL), was added lithium diisobutyl-tert-butoxyaluminum hydride (38.6 mL, 9.66 mmol) at 0° C. After stirring for 10 min, the reaction suspension was allowed to warm to RT and stirred for 1 h. The reaction mixture was quenched with saturated aq. Rochelle salt solution dropwise over 5 min and stirred for 30 min. The reaction mixture was extracted with ethyl acetate. The aq. layer was re-extracted with ethyl acetate (2×20 mL). The combined organic layer was washed with water followed by brine solution, dried over anhydrous sodium sulphate, filtered, and concentrated. The crude was purified using CombiFlash (Redisep Rf 40 g silica gel column; 4% MeOH in DCM as eluent) to afford the title compound (300 mg, 0.430 mmol, 31.2% yield) as light yellow solid. ¹H NMR (400 MHz, DMSO-d₆) δ 9.49-9.26 (m, 1H), 8.15-7.97 (m, 1H), 7.61-7.51 (m, 4H), 7.44-7.22 (m, 6H), 7.03-6.93 (m, 2H), 6.86-6.81 (m, 1H), 6.77-6.65 (m, 1H), 5.54-5.33 (m, 2H), 5.26-5.10 (m, 1H), 4.51-4.40 (m, 2H), 4.35-4.28 (m, 1H), 4.17-4.06 (m, 1H), 3.99-3.91 (m, 1H), 3.90-3.72 (m, 6H), 3.65-3.51 (m, 3H), 1.68-1.47 (m, 2H), 1.35-1.18 (m, 2H), 1.03-0.78 (m, 12H). LC-MS (ES): m/z=697.25 [M+H]⁺

Step 4: Methyl (7-(butyl(2-((tert-butyldiphenylsilyl)oxy)ethyl)amino)-2-(4-(chloromethyl)-2-methoxybenzyl)-2H-pyrazolo[4,3-d]pyrimidin-5-yl)carbamate: To a stirred solution of methyl (7-(butyl(2-((tert-butyldiphenylsilyl)oxy)ethyl)amino)-2-(4-(hydroxymethyl)-2-methoxybenzyl)-2H-pyrazolo[4,3-d]pyrimidin-5-yl)carbamate (350 mg, 0.502 mmol) in THE (10 mL), SOCl₂ (0.220 mL, 3.01 mmol) was added at 0° C. The reaction mixture was stirred at same temperature for 1 h. The reaction mixture was concentrated under reduced pressure to afford the title compound (340 mg, 0.475 mmol, 95% yield) as light yellow semi-solid. LC-MS (ES): m/z=716.6 [M+H]⁺

Step 5: Methyl (7-(butyl(2-((tert-butyldiphenylsilyl)oxy)ethyl)amino)-2-(2-methoxy-4-(((4-methyltetrahydro-2H-pyran-4-yl)amino)methyl)benzyl)-2H-pyrazolo[4,3-d]pyrimidin-5-yl)carbamate: To a stirred solution of methyl (7-(butyl(2-((tert-butyldiphenylsilyl)oxy)ethyl)amino)-2-(4-(chloromethyl)-2-methoxybenzyl)-2H-pyrazolo[4,3-d]pyrimidin-5-yl) carbamate (100 mg, 0.140 mmol) in CH₃CN (3 mL), were added 4-methyltetrahydro-2H-pyran-4-amine hydrochloride (27.6 mg, 0.182 mmol), Na₂CO₃ (89 mg, 0.839 mmol) and KI (23.21 mg, 0.140 mmol). The reaction mixture was stirred at 65° C. for 12 h. The mixture was allowed to cool to room temperature and filtered through a syringe filter and concentrated under vacuum to afford the title compound (100 mg, 0.126 mmol, 90% yield) as a brown solid. LCMS (ES): m/z=794.2 [M+H]⁺

Step 6: Methyl (7-(butyl(2-hydroxyethyl)amino)-2-(2-methoxy-4-(((4-methyltetrahydro-2H-pyran-4-yl) amino)methyl)benzyl)-2H-pyrazolo[4,3-d]pyrimidin-5-yl) carbamate: To a solution of methyl (7-(butyl(2-((tert-butyldiphenylsilyl) oxy) ethyl) amino)-2-(2-methoxy-4-(((4-methyltetrahydro-2H-pyran-4-yl) amino) methyl) benzyl)-2H-pyrazolo[4,3-d]pyrimidin-5-yl) carbamate (100 mg, 0.126 mmol) in MeOH (5 mL), was added aqueous HCl (1.679 mL, 2.52 mmol) dropwise over 2 min. The mixture was stirred at 60° C. for 2 h and then concentrated under reduced pressure. The residue was triturated with diethyl ether (2×10 mL) and then dried to afford the title compound (65 mg, 0.110 mmol, 87% yield) as a light yellow semi-solid. LCMS (ES): m/z=556.2 [M+H]⁺

Step 7: 2-((5-Amino-2-(2-methoxy-4-(((4-methyltetrahydro-2H-pyran-4-yl)amino)methyl) benzyl)-2H-pyrazolo[4,3-d]pyrimidin-7-yl) (butyl)amino) ethan-1-ol: To a solution of methyl (7-(butyl(2-hydroxyethyl) amino)-2-(2-methoxy-4-(((4-methyltetrahydro-2H-pyran-4-yl)amino)methyl)benzyl)-2H-pyrazolo[4,3-d]pyrimidin-5-yl) carbamate hydrochloride (65 mg, 0.110 mmol) in dioxane (5 mL), was added an aqueous solution of NaOH (1.098 mL, 3.29 mmol) at room temperature. The reaction mixture was heated to 70° C. and stirred for 4 h. The organic layer from the reaction mixture was separated and concentrated under reduced pressure. The crude compound was purified by preparative LC-MS conditions (column: Waters XBridge C18, 19×150 mm, 5-μm particles; mobile phase A: 10-mM ammonium acetate; mobile phase B: acetonitrile; gradient: 15-55% B over 20 minutes, then a 5-minute hold at 100% B; flow: 15 mL/min). Fractions containing the desired product were combined and dried via centrifugal evaporation to afford the title compound (17.7 mg, 0.036 mmol, 32.4% yield) as an off-white solid.

| Cmpd. | | |
|---|---|---|
| No. | Structure | Analytical Data (¹H NMR; LC-MS) |
| 88 | 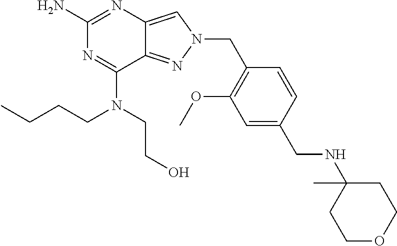 [SMILES: CCCCN(CCO)c1nc(N)nc2cn(Cc3ccc(CNC4(C)CCOCC4)cc3OC)nc12] | ¹H NMR (400 MHz, DMSO-d₆) δ = 7.74 (br s, 1H), 7.10 (s, 1H), 6.95 (s, 2H), 5.87 (s, 2H), 5.40 (s, 2H), 4.72 (s, 1H), 4.09 (br s, 2H), 3.84 (s, 3H), 3.76-3.63 (m, 9H), 3.47 (td, J = 3.8, 7.7 Hz, 3H), 1.66-1.44 (m, 5H), 1.19 (br s, 6H), 0.92 (br s, 3H); LC-MS (ES): m/z = 498.3 [M + H]⁺, RT (min) = 1.324; purity: 100% (LC-MS method A) |

### Example S2. 2-Methyltetrahydro-2H-pyran-4-amine (Amine Used in the Synthesis of Compound 122)

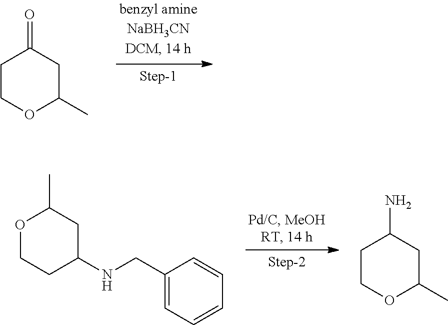

Step 1. N-Benzyl-2-methyltetrahydro-2H-pyran-4-amine: To a stirred solution of 2-methyltetrahydro-4H-pyran-4-one (2.0 g, 17.52 mmol) in MeOH (20 mL), were added benzylamine (2.297 mL, 21.03 mmol) and AcOH (2.006 mL, 35.0 mmol) under nitrogen atmosphere. The mixture was stirred for 4 h at RT. Then NaCNBH₃ (4.40 g, 70.1 mmol) was added. The mixture was stirred for 16 h and concentrated under reduced pressure. The residue was partitioned between saturated aq. NaHCO₃ solution and EtOAc. The organic layer was separated and the aqueous layer was extracted with EtOAc (2×20 mL). The combined organic layer was washed with water, brine solution, dried over sodium sulphate, filtered and concentrated. The residue was purified by CombiFlash (Redisep Rf 40 g silica gel column, eluting with 30-100% EtOAc/hexane) to afford the title product as two separable isomers (isomer-1: 550 mg, 2.68 mmol, 15.29% yield; isomer-2: 750 mg, 3.65 mmol, 20.85% yield) as a pale brown oil. ¹H NMR (500 MHz, DMSO-d₆) δ=7.34-7.23 (m, 5H), 3.82-3.74 (m, 1H), 3.68-3.58 (m, 2H), 3.31-3.16 (m, 2H), 2.53-2.51 (1H, m), 1.85-1.67 (m, 2H), 1.21-1.10 (m, 1H), 1.06-1.01 (m, 4H), 0.97-0.86 (m, 1H). LC-MS (ES): m/z=206.2 [M+H]⁺

Step 2. 2-Methyltetrahydro-2H-pyran-4-amine: A stirred solution of N-benzyl-2-methyltetrahydro-2H-pyran-4-amine (750 mg, 3.65 mmol; isomer-2 from the previous step) in MeOH (10 mL) was purged with N₂ gas at room temperature. Pd/C (778 mg, 0.731 mmol) was added. The mixture was degassed with vacuum and then stirred at room temperature under hydrogen gas (bladder) for 14 h. The black suspension was filtered through a Celite bed, which was washed with a mixture of DCM and methanol. The filtrate was concentrated under reduced pressure to afford the title compound (210 mg, 1.823 mmol, 74.9% yield) as a brown oil. LC-MS (ES): m/z=116.2 [M+H]⁺

### Example S3. 2,2-Dimethyltetrahydrofuran-3-amine (Amine Used in the Synthesis of Compound 153)

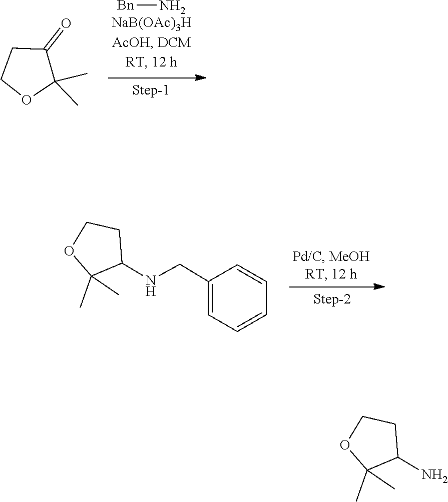

Step 1. N-Benzyl-2,2-dimethyltetrahydrofuran-3-amine: To a stirred solution of 2,2-dimethyldihydrofuran-3(2H)-one (1.5 g, 13.14 mmol) in DCM (25 mL), were added benzylamine (1.866 mL, 17.08 mmol), acetic acid (0.978 mL, 17.08 mmol) and sodium triacetoxyborohydride (9.75 g, 46.0 mmol). The reaction mixture was stirred at RT for 12 h. The reaction mixture was quenched with saturated aq. NaHCO₃ solution and extracted with DCM (2×20 mL). The combined organic layer was washed with brine solution, dried over anhydrous sodium sulphate, filtered, and concentrated. The residue was purified using CombiFlash column chromatography (24 g silica gel column, 20-30% ethyl acetate in pet. ether as an eluent) to afford the title compound (1 g, 4.87 mmol, 37.1% yield) as a light brown oil. ¹H NMR (400 MHz, DMSO-d₆) δ=7.27-7.37 (m, 5H) 7.18-7.25 (m, 1H) 3.74-3.80 (m, 1H) 3.64-3.73 (m, 2H) 3.53-3.62 (m, 1H) 2.70-2.77 (m, 1H) 2.06-2.15 (m, 1H) 1.61-1.70 (m, 1H) 1.11-1.15 (m, 3H) 1.02-1.05 (m, 3H). LC-MS (ES): m/z=206.25 [M+H]⁺

Step 2. 2,2-Dimethyltetrahydrofuran-3-amine: A stirred solution of N-benzyl-2,2-dimethyltetrahydrofuran-3-amine (1 g, 4.87 mmol) in methanol (20 mL) was purged with N₂ gas at room temperature. Pd—C (300 mg, 2.82 mmol) was added. The reaction mixture was degassed with vacuum and stirred at room temperature under hydrogen gas (balloon) for 12 h. The reaction mass was filtered through a Celite bed, which was washed with MeOH in DCM (1:1). The filtrate was concentrated under vacuum to afford the title compound (200 mg, 1.736 mmol, 35.6% yield) as a colorless oil. ¹H NMR (400 MHz, DMSO-d₆) δ=3.63-3.73 (m, 1H) 3.54-3.61 (m, 1H) 2.85-2.92 (m, 1H) 2.04-2.13 (m, 1H) 1.53-1.64 (m, 1H) 1.08-1.11 (m, 3H) 0.94-1.00 (m, 3H); LC-MS (ES): m/z=116.25 [M+H]⁺

### Example S4. 2,6-Dimethyltetrahydro-2H-pyran-4-amine (Amine Used in the Synthesis of Compound 166)

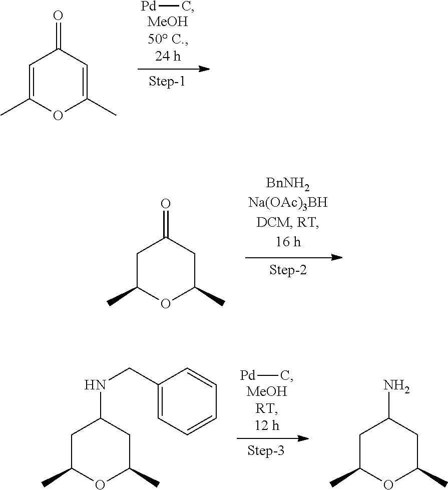

Step 1. 2,6-Dimethyltetrahydro-4H-pyran-4-one: 2,6-dimethyl-4H-pyran-4-one (5 g, 40.3 mmol) was dissolved in dry MeOH (60 mL) and charged into a Buchi-steel autoclave with mechanical stirring. Pd/C (2.143 g, 2.014 mmol) was added and hydrogenation was accomplished at 50° C. and 25 bar H₂ pressure for 24 h. The reaction mixture was filtered through a pad of Celite, and the filtrate was concentrated. The crude compound was purified via CombiFlash column chromatography (Redisep Rf 80 g silica gel column, 20% EtOAc in pet. ether as an eluent) to afford the title compound (1.5 g, 11.70 mmol, 29.1% yield) as a colorless oil. LCMS (ES): m/z=129.2 [M+H]⁺

Step 2. N-Benzyl-2,6-dimethyltetrahydro-2H-pyran-4-amine: To a stirred solution of 2,6-dimethyltetrahydro-4H-pyran-4-one (1.3 g, 10.14 mmol) in DCM (20 mL), were added benzyl amine (1.329 mL, 12.17 mmol) and AcOH (1.161 mL, 20.29 mmol) under nitrogen atmosphere. The mixture was stirred for 30 min. at room temperature. Then sodium triacetoxyborohydride (6.45 g, 30.4 mmol) was added. The reaction mixture was stirred for 16 h and then concentrated under reduced pressure. The residue was partitioned between saturated aq. NaHCO₃ solution and EtOAc. The organic layer was separated, and the aqueous layer was extracted with EtOAc (2×20 mL). The combined organic layer was washed with water, brine solution, dried over sodium sulphate, filtered, and concentrated. The residue was purified by CombiFlash column chromatography (Redisep Rf 40 g silica gel column, eluting with 30% EtOAc/hexane) to afford the title compound (1.1 g, 5.02 mmol, 49.4% yield) as a pale brown oil. ¹H NMR (500 MHz, DMSO-d₆) δ=7.38-7.27 (m, 4H), 7.23-7.17 (m, 1H), 3.89-3.80 (m, 2H), 3.71-3.64 (m, 2H), 2.92-2.84 (m, 1H), 2.09-2.02 (m, 1H), 1.63-1.54 (m, 2H), 1.23-1.14 (m, 2H), 1.03-0.98 (m, 6H). LCMS (ES): m/z=220.2 [M+H]⁺

Step 3. 2,6-Dimethyltetrahydro-2H-pyran-4-amine: The stirred solution of N-benzyl-2,6-dimethyltetrahydro-2H-pyran-4-amine (1.1 g, 5.02 mmol) in an anhydrous methanol (25 mL) was purged with N₂ gas at room temperature. Pd—C (550 mg, 0.517 mmol) was then added. The reaction mixture was degassed with vacuum and stirred at room temperature under hydrogen gas (bladder) for 12 h. The reaction mixture was filtered through a Celite bed, which was washed with excess of MeOH in DCM (1:1). The filtrate was concentrated under vacuum to afford the title compound (500 mg, 3.87 mmol, 77% yield) as a colorless oil. ¹H NMR (400 MHz, DMSO-d₆) δ=3.79-3.88 (m, 2H) 2.68-2.75 (m, 1H) 1.34-1.45 (m, 2H) 1.19-1.29 (m, 2H) 1.01 (d, J=6.25 Hz, 6H). LCMS (ES): m/z=130.20 [M+H]⁺

The following compounds were prepared using the amines described in Examples S2-S4 or commercially available amines following the experimental procedure for Compound 88.

| Cmpd | | Analytical Data (¹H NMR 500 MHz, DMSO- |
|---|---|---|
| No. | Structure | d₆; LC-MS) |
| 122 | 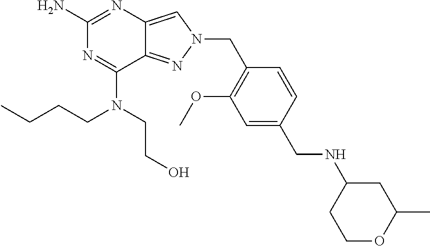 [SMILES: CCCCN(CCO)c1nc(N)nc2cn(Cc3ccc(CNC4CCOC(C)C4)cc3OC)nc12] | ¹H NMR (400 MHz, DMSO-d₆) δ = 7.69 (br s, 1H), 7.05 (s, 1H), 6.93 (br d, J = 3.1 Hz, 1H), 6.90-6.82 (m, 1H), 5.51 (br s, 2H), 5.37 (s, 2H), 4.70 (br s, 1H), 4.10 (br s, 1H), 3.83 (s, 4H), 3.76-3.61 (m, 9H), 3.25 (br s, 2H), 2.60 (br s, 1H), 1.85 (td, J = 1.8, 12.4 Hz, 1H), 1.79-1.74 (m, 1H), 1.66-1.43 (m, 1H), 1.29 (br s, 1H), 1.17 (dd, J = 4.4, 11.6 Hz, 1H), 1.07 (d, J = 6.3 Hz, 5H), 0.98-0.79 (m, 4H) LC-MS (ES): m/z = 498.3 [M + H]⁺, RT (min) = 1.28 |
| 153 | 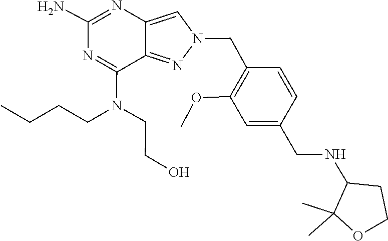 [SMILES: CCCCN(CCO)c1nc(N)nc2cn(Cc3ccc(CNC4CCOC4(C)C)cc3OC)nc12] | ¹H NMR (400 MHz, DMSO-d₆) δ = 7.88-7.77 (m, 1H), 7.08 (s, 1H), 7.01 (br s, 1H), 6.90 (d, J = 7.8 Hz, 1H), 6.68 (br s, 2H), 5.42 (s, 2H), 4.77 (br s, 1H), 4.21-3.99 (m, 2H), 3.81 (s, 3H), 3.78-3.62 (m, 7H), 3.57 (br d, J = 8.0 Hz, 2H), 2.73 (t, J = 7.8 Hz, 1H), 2.15-2.05 (m, 1H), 1.72-1.49 (m, 3H), 1.37-1.20 (m, 2H), 1.12 (s, 3H), 1.04 (s, 3H), 0.85 (br s, 3H); LC-MS (ES): m/z = 498.2 [M + H]⁺, RT (min) = 1.49 |
| 166 | 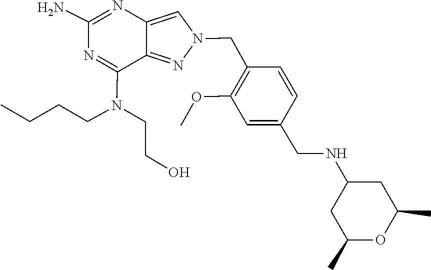 [SMILES: CCCCN(CCO)c1nc(N)nc2cn(Cc3ccc(CNC4C[C@@H](C)O[C@@H](C)C4)cc3OC)nc12] | ¹H NMR (400 MHz, DMSO-d₆) δ = 7.74 (br s, 1H), 7.06 (s, 1H), 7.01-6.92 (m, 1H), 6.91- 6.83 (m, 1H), 5.91 (br s, 2H), 5.39 (s, 2H), 4.72 (br s, 1H), 4.13 (br s, 2H), 3.87-3.79 (m, 6H), 3.75-3.60 (m, 8H), 2.89 (br d, J = 1.3 Hz, 1H), 1.66-1.49 (m, 4H), 1.34-1.11 (m, 4H), 1.00 (d, J = 6.3 Hz, 6H), 0.88 (m, 3H); LC- MS (ES): m/z = 512.3 [M + H]⁺, RT (min) = 0.91 |
| 3 | 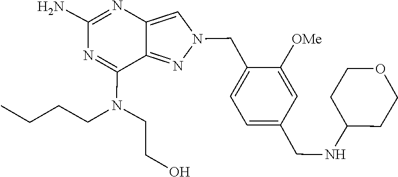 [SMILES: CCCCN(CCO)c1nc(N)nc2cn(Cc3ccc(CNC4CCOCC4)cc3OC)nc12] | δ 7.68 (s, 1H), 7.05 (s, 1H), 6.94 (s, 1H), 6.87 (d, J = 7.8 Hz, 1H), 5.54 (s, 1H), 5.37 (s, 2H), 4.10 (s, 1H), 3.80 (s, 3H), 3.72 (s, 2H), 3.64 (s, 1H), 3.28-3.19 (m, 2H), 2.55 (s, 5H), 1.90 (s, 4H), 1.76 (d, J = 13.1 Hz, 2H), 1.56 (s, 1H), 1.27 (d, J = 9.8 Hz, 4H), 0.87 (s, 3H). 2 protons were not observed, which is attributed to the water suppression used. LC-MS (ES): m/z = 483.9 [M + H]⁺, RT (min) = 1.33 |
| 4 | 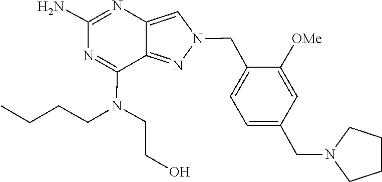 [SMILES: CCCCN(CCO)c1nc(N)nc2cn(Cc3ccc(CN4CCCC4)cc3OC)nc12] | δ 7.70 (s, 1H), 6.97 (s, 1H), 6.93 (s, 1H), 6.84 (d, J = 7.7 Hz, 1H), 5.55 (s, 1H), 5.38 (s, 2H), 4.09 (s, 1H), 3.82 (s, 3H), 2.55 (s, 2H), 2.42 (d, J = 5.8 Hz, 4H), 1.90 (s, 5H), 1.68 (q, J = 3.4 Hz, 4H), 1.55 (s, 1H), 1.26 (s, 1H), 0.85 (s, 3H). 4 protons were not observed, which is attributed to the water suppression used. LC- MS (ES): m/z = 453.9 [M + H]⁺, RT (min) = 1.12 |
| 5 | 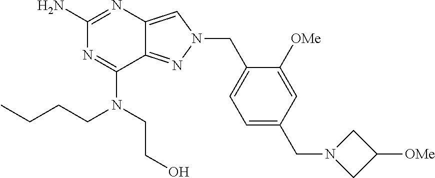 [SMILES: CCCCN(CCO)c1nc(N)nc2cn(Cc3ccc(CN4CC(OC)C4)cc3OC)nc12] | δ 8.10 (d, J = 14.2 Hz, 1H), 8.04 (s, 0H), 7.78 (s, 2H), 7.21 (s, 1H), 7.19-7.12 (m, 1H), 7.07 (dd, J = 15.9, 10.3 Hz, 2H), 5.57-5.48 (m, 2H), 4.36 (s, 2H), 4.27-4.18 (m, 3H), 4.15 (t, J = 7.9 Hz, 1H), 3.95 (s, 2H), 3.88-3.78 (m, 4H), 3.72 (d, J = 5.7 Hz, 1H), 3.65 (s, 1H), 2.55 (s, 4H), 1.69-1.62 (m, 1H), 1.58 (s, 1H), 1.34 (q, J = 7.5 Hz, 1H), 1.25 (q, J = 7.5 Hz, 1H), 0.94 (t, J = 7.4 Hz, 1H), 0.86 (dt, J = 12.2, 7.3 Hz, 3H). LC-MS (ES): m/z = 470.2 [M + H]⁺, RT (min) = 1.19 |
| 6 | 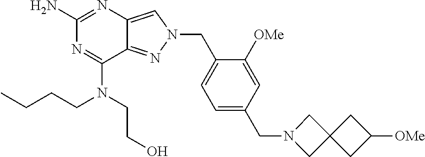 [SMILES: CCCCN(CCO)c1nc(N)nc2cn(Cc3ccc(CN4CC5(CC(OC)C5)C4)cc3OC)nc12] | δ 7.69 (s, 1H), 6.90 (s, 2H), 6.77 (d, J = 7.7 Hz, 1H), 5.60 (s, 1H), 5.36 (s, 3H), 4.09 (s, 1H), 3.81 (s, 3H), 3.68 (t, J = 6.9 Hz, 2H), 3.47 (s, 1H), 3.12 (s, 3H), 3.07 (s, 6H), 2.55 (s, 4H), 2.32 (ddd, J = 10.2, 7.1, 3.1 Hz, 3H), 1.92-1.84 (m, 4H), 1.55 (s, 1H), 1.25 (s, 1H), 0.85 (s, 2H). LC-MS (ES): m/z = 509.9 [M + H]⁺, RT (min) = 1.13 |
| 7 | 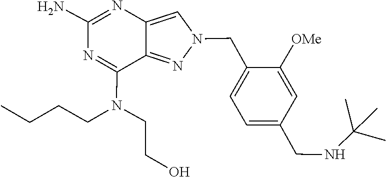 [SMILES: CCCCN(CCO)c1nc(N)nc2cn(Cc3ccc(CNC(C)(C)C)cc3OC)nc12] | δ 8.06 (s, 1H), 8.01 (s, 0H), 7.80 (s, 2H), 7.28 (s, 1H), 7.21 (d, J = 7.8 Hz, 1H), 7.15 (d, J = 7.8 Hz, OH), 7.08 (t, J = 6.2 Hz, 1H), 5.51 (d, J = 2.7 Hz, 2H), 4.24-4.13 (m, 2H), 4.11 (s, 2H), 3.89-3.80 (m, 4H), 2.55 (s, 4H), 1.62 (s, 2H), 1.60 (d, J = 7.3 Hz, 0H), 1.36 (s, 9H), 1.30 (dt, J = 14.8, 6.9 Hz, 1H), 0.91 (dt, J = 21.6, 7.3 Hz, 3H). 2 protons were not observed, which is attributed to the water suppression used. LC-MS (ES): m/z = 456.0 [M + H]⁺, RT (min) = 1.13 |
| 11 | 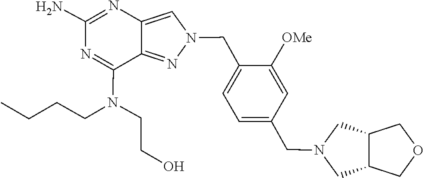 [SMILES: CCCCN(CCO)c1nc(N)nc2cn(Cc3ccc(CN4C[C@H]5COC[C@H]5C4)cc3OC)nc12] | δ 7.71 (s, 1H), 6.95 (d, J = 18.4 Hz, 2H), 6.83 (d, J = 7.7 Hz, 1H), 5.57 (s, 1H), 5.38 (s, 2H), 4.10 (s, 1H), 3.82 (s, 3H), 3.71 (d, J = 15.2 Hz, 1H), 3.71 (s, 2H), 3.64 (s, 1H), 2.68 (s, 2H), 2.55 (s, 3H), 2.49 (s, 1H), 2.29 (dd, J = 9.3, 2.8 Hz, 2H), 1.91 (s, 4H), 1.55 (s, 3H), 1.26 (s, 3H), 0.86 (s, 1H). 3 protons were not observed, which is attributed to the water suppression used. LC-MS (ES): m/z = 496.3 [M + H]⁺, RT (min) = 1.5 |
| 12 | 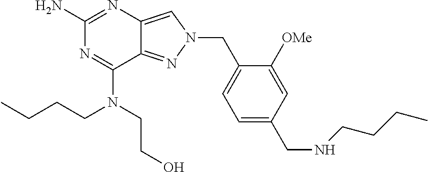 [SMILES: CCCCNCc1ccc(Cn2cc3nc(N)nc(N(CCO)CCCC)c3n2)c(OC)c1] | δ 8.07 (s, 1H), 8.02 (s, OH), 7.79 (s, 2H), 7.24 (s, 1H), 7.15 (dd, J = 27.8, 7.8 Hz, 1H), 7.05 (d, J = 7.4 Hz, 1H), 5.51 (d, J = 3.0 Hz, 2H), 4.21 (s, 1H), 4.14 (s, 3H), 3.84 (t, J = 11.6 Hz, 5H), 3.72 (s, 1H), 3.67 (s, 1H), 2.90 (s, 2H), 1.60 (t, J = 8.0 Hz, 4H), 1.32 (dt, J = 13.4, 6.3 Hz, 3H), 1.30-1.23 (m, 1H), 0.97-0.83 (m, 6H). 1 proton was not observed, which is attributed to the water suppression used. LC-MS (ES): m/z = 456.2 [M + H]⁺, RT (min) = 1.17 |
| 18 | 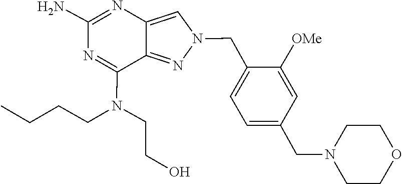 [SMILES: CCCCN(CCO)c1nc(N)nc2cn(Cc3ccc(CN4CCOCC4)cc3OC)nc12] | δ 7.71 (s, 1H), 6.97 (s, 1H), 6.93 (s, 1H), 6.85 (d, J = 7.4 Hz, 1H), 5.56 (s, 1H), 5.38 (s, 2H), 4.09 (s, 1H), 3.82 (s, 3H), 3.55 (s, 2H), 2.55 (s, 3H), 2.34 (s, 4H), 1.91 (s, 3H), 1.55 (s, 3H), 1.25 (s, 2H), 0.85 (s, 3H). 4 protons was not observed, which is attributed to the water suppression used. LC-MS (ES): m/z = 470.3 [M + H]⁺, RT (min) = 1.11 |
| 21 | 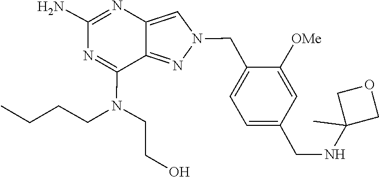 [SMILES: CCCCN(CCO)c1nc(N)nc2cn(Cc3ccc(CNC4(C)COC4)cc3OC)nc12] | δ 7.76 (s, 1H), 7.07 (s, 1H), 6.98 (s, 1H), 6.90 (d, J = 7.6 Hz, 1H), 6.13 (s, 2H), 5.40 (s, 2H), 4.45 (d, J = 5.7 Hz, 2H), 4.16 (d, J = 5.6 Hz, 2H), 3.83 (s, 3H), 3.67 (d, J = 14.2 Hz, 5H), 2.54 (d, J = 3.2 Hz, 3H), 1.91 (d, J = 3.2 Hz, 1H), 1.41 (s, 3H), 1.28 (s, 3H), 0.88 (s, 4H). 1 proton was not observed, which is attributed to the water suppression used. LC-MS (ES): m/z = 470.2 [M + H]⁺, RT (min) = 1.1 |
| 22 | 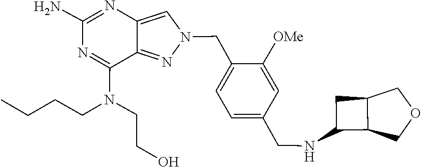 | δ 7.74 (s, 1H), 7.01 (s, 1H), 6.96 (s, 1H), 6.84 (d, J = 7.6 Hz, 1H), 5.96 (s, 1H), 5.39 (s, 2H), 4.12 (s, 2H), 4.05 (d, J = 9.6 Hz, 1H), 3.71 (s, 2H), 3.65 (s, 2H), 3.61-3.51 (m, 2H), 3.24 (dd, J = 8.8, 4.4 Hz, 1H), 3.18 (q, J = 8.3 Hz, 1H), 2.90 (d, J = 7.5 Hz, 1H), 2.66-2.60 (m, 1H), 2.55 (d, J = 0.8 Hz, 6H), 2.27 (d, J = 9.8 Hz, 1H), 1.91 (d, J = 0.8 Hz, 2H), 1.57 (s, 3H), 1.37-1.25 (m, 2H), 0.87 (s, 3H). LC-MS (ES): m/z = 496.5 [M + H]⁺, RT (min) = 1.16 |
| 24 | 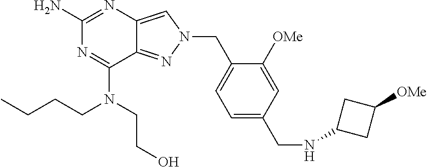 | δ 7.68 (s, 1H), 7.01 (s, 1H), 6.93 (s, 1H), 6.84 (d, J = 7.7 Hz, 1H), 5.58 (s, 1H), 5.37 (s, 2H), 4.10 (s, 1H), 3.96-3.90 (m, 1H), 3.64 (s, 1H), 3.28-3.21 (m, 1H), 3.10 (d, J = 1.3 Hz, 3H), 2.57-2.48 (m, 6H), 1.99-1.92 (m, 4H), 1.91 (d, J = 1.3 Hz, 3H), 1.57 (s, 3H), 1.28 (s, 3H), 0.88 (s, 3H). 1 proton was not observed, which is attributed to the water suppression used. LC-MS (ES): m/z = 484.3 [M + H]⁺, RT (min) = 1.16 |
| 25 | 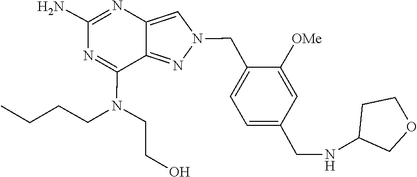 [SMILES: CCCCN(CCO)c1nc(N)nc2cn(Cc3ccc(CNC4CCOC4)cc3OC)nc12] | δ 7.68 (s, 1H), 7.03 (s, 1H), 6.86 (d, J = 7.6 Hz, 1H), 5.55 (s, 1H), 5.37 (s, 2H), 3.82 (s, 3H), 3.75 (d, J = 7.5 Hz, 1H), 3.71-3.61 (m, 3H), 2.57-2.48 (m, 6H), 1.90 (s, 3H), 1.95-1.85 (m, 1H), 1.66 (dq, J = 12.2, 5.9 Hz, 1H), 1.56 (s, 3H), 1.27 (s, 3H), 0.87 (s, 3H). 2 protons were not observed, which is attributed to the water suppression used. LC-MS (ES): m/z = 470.2 [M + H]⁺, RT (min) = 1.03 |
| 28 | 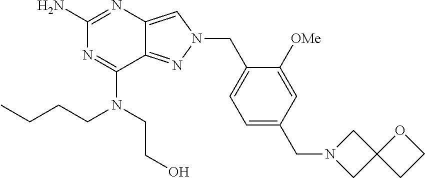 [SMILES: CCCCN(CCO)c1nc(N)nc2cn(Cc3ccc(CN4CC5(CCO5)C4)cc3OC)nc12] | δ 7.70 (s, 1H), 6.91 (s, 2H), 6.78 (d, J = 7.7 Hz, 1H), 5.60 (s, 1H), 5.37 (s, 2H), 4.35 (t, J = 7.5 Hz, 2H), 4.10 (s, 1H), 3.69 (s, 1H), 3.64 (s, 1H), 3.09-3.03 (m, 2H), 2.74 (t, J = 7.5 Hz, 2H), 2.55 (d, J = 1.1 Hz, 4H), 1.91 (s, 3H), 1.56 (s, 3H), 1.26 (s, 3H), 0.86 (s, 3H). 3 protons were not observed, which is attributed to the water suppression used. LC-MS (ES): m/z = 482.2 [M + H]⁺, RT (min) = 1.19 |
| 31 | 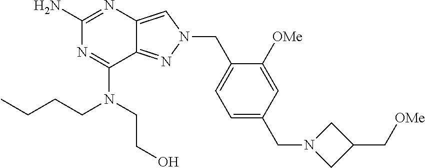 [SMILES: CCCCN(CCO)c1nc(N)nc2cn(Cc3ccc(CN4CC(COC)C4)cc3OC)nc12] | δ 8.06 (s, 1H), 8.01 (s, 1H), 7.75 (s, 2H), 7.19- 7.08 (m, 3H), 7.02 (s, 2H), 5.49 (d, J = 2.4 Hz, 3H), 4.35 (s, 2H), 4.29 (s, 2H), 4.20 (s, 1H), 4.13 (t, J = 7.8 Hz, 2H), 3.91-3.77 (m, 4H), 3.51 (s, 2H), 2.89 (s, 1H), 2.73 (s, 1H), 2.55 (s, 1H), 1.67-1.60 (m, 1H), 1.35-1.28 (m, 1H), 1.23 (q, J = 7.6 Hz, 2H), 0.92 (t, J = 7.3 Hz, 2H), 0.83 (t, J = 7.3 Hz, 3H). LC-MS (ES): m/z = 482.2 [M + H]⁺, RT (min) = 1.19 |
| 33 | 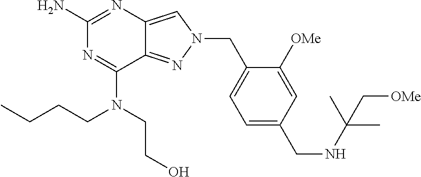 [SMILES: CCCCN(CCO)c1nc(N)nc2cn(Cc3ccc(CNC(C)(C)COC)cc3OC)nc12] | δ 7.67 (s, 1H), 7.02 (s, 1H), 6.95 (s, 1H), 6.87 (d, J = 7.6 Hz, 1H), 5.55 (s, 1H), 5.37 (s, 2H), 4.10 (s, 1H), 3.82 (s, 3H), 3.70 (s, 1H), 3.65 (s, 2H), 3.18 (d, J = 1.7 Hz, 2H), 2.55 (d, J = 1.6 Hz, 4H), 1.91 (d, J = 1.6 Hz, 3H), 1.56 (s, 3H), 1.27 (s, 3H), 1.03 (d, J = 1.7 Hz, 6H), 0.88 (s, 4H). LC-MS (ES): m/z = 486.3 [M + H]⁺, RT (min) = 1.27 |
| 53 | 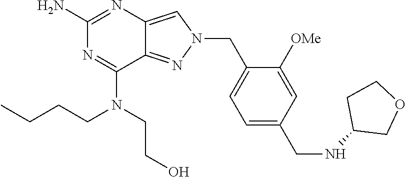 [SMILES: CCCCN(CCO)c1nc(N)nc2cn(Cc3ccc(CN[C@@H]4CCOC4)cc3OC)nc12] | δ 7.03 (s, 1H), 6.95 (s, 2H), 6.86 (d, J = 7.2 Hz, 1H), 5.62 (s, 1H), 5.37 (s, 2H), 3.82 (s, 3H), 3.77-3.59 (m, 5H), 2.55 (s, 3H), 1.97- 1.87 (m, 1H), 1.91 (s, 4H), 1.67 (dt, J = 11.9, 6.2 Hz, 1H), 1.56 (s, 3H), 1.27 (s, 3H), 0.87 (s, 5H). LC-MS (ES): m/z = 470.2 [M + H]⁺, RT (min) = 1.09 |
| 44 | 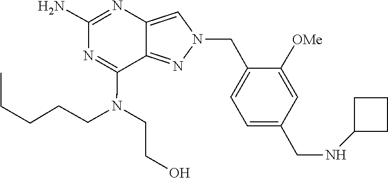 [SMILES: CCCCCN(CCO)c1nc(N)nc2cn(Cc3ccc(CNC4CCC4)cc3OC)nc12] | δ 7.71 (s, 1H), 7.05 (s, 1H), 6.93 (s, 1H), 6.86 (d, J = 7.9 Hz, 1H), 5.66 (s, 1H), 5.39 (s, 2H), 4.10 (s, 1H), 3.83 (s, 3H), 3.21 (s, 1H), 2.55 (s, 3H), 2.06 (d, J = 9.0 Hz, 3H), 1.91 (s, 2H), 1.80-1.72 (m, 2H), 1.63 (d, J = 9.4 Hz, 1H), 1.62-1.52 (m, 6H), 1.26 (s, H), 0.86 (d, J = 9.1 Hz, 3H). LC-MS (ES): m/z = 468.5 [M + H]⁺, RT (min) = 1.27 |

### Example S5. 2-((5-Amino-2-(4-(((2,2-dimethyltetrahydro-2H-pyran-4-yl)amino)methyl)-2-methoxybenzyl)-2H-pyrazolo[4,3-d] pyrimidin-7-yl)(butyl)amino) ethan-1-ol (Compound 103 (racemate), Compound 111 (Enantiomer-1), and Compound 112 (Enantiomer-2))

Compound 103 (racemate) was prepared following the experimental procedure for Compound 88. The enantiomers of Compound 103 were separated by chiral SFC method to obtain the individual enantiomers, Compound 111 and Compound 112.

| Cmpd | | Analytical Data (¹H NMR 400 MHz, DMSO- |
|---|---|---|
| No. | Structure | d₆; LC-MS) |
| 103 | 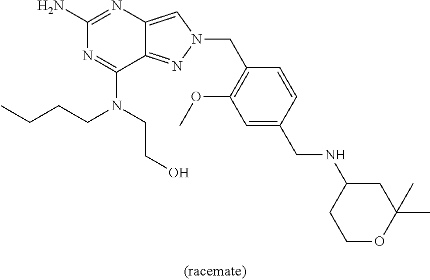 [SMILES: CCCCN(CCO)c1nc(N)nc2cn(Cc3ccc(CNC4CCOC(C)(C)C4)cc3OC)nc12] | δ = 7.67 (br d, J = 1.8 Hz, 1H), 7.03 (s, 1H), 6.97-6.90 (m, 1H), 6.89-6.82 (m, 1H), 5.43 (s, 2H), 5.37 (s, 2H), 4.82-4.56 (m, 1H), 4.20-3.98 (m, 2H), 3.82 (s, 3H), 3.71 (s, 2H), 3.67-3.57 (m, 8H), 3.47 (br d, J = 2.0 Hz, 1H), 2.79-2.70 (m, 1H), 1.91 (s, 1H), 1.82-1.69 (m, 2H), 1.64- 1.49 (m, 2H), 1.36-1.20 (m, 2H), 1.12 (s, 3H), 1.08 (s, 4H), 0.95-0.81 (m, 3H). LC-MS (ES): m/z = 512.3 [M + H]⁺, RT (min) = 1.29 |
| 111 | 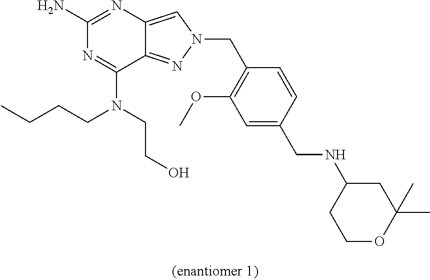 [SMILES: CCCCN(CCO)c1nc(N)nc2cn(Cc3ccc(CNC4CCOC(C)(C)C4)cc3OC)nc12] | δ = 7.77 (br s, 1H), 7.11 (s, 1H), 7.00 (br s, 1H), 6.95-6.84 (m, 1H), 6.04 (br s, 2H), 5.41 (s, 2H), 4.76 (br s, 1H), 4.11 (br dd, J = 2.4, 4.8 Hz, 2H), 3.94-3.81 (m, 6H), 3.76-3.62 (m, 6H), 3.51 (br d, J = 12.0 Hz, 2H), 2.99 (br s, 1H), 1.90-1.76 (m, 2H), 1.60 (br s, 2H), 1.34- 1.22 (m, 3H), 1.19-1.12 (m, 4H), 1.10 (s, 3H), 0.88 (br d, J = 6.3 Hz, 3H). LC-MS (ES): m/z = 512.3 [M + H]⁺, RT (min) = 1.34 |
| 112 | 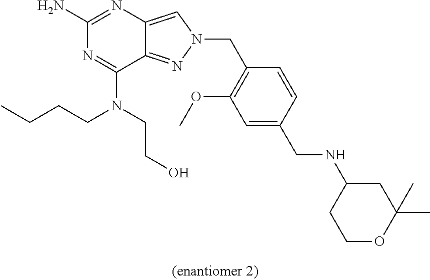 [SMILES: CCCCN(CCO)c1nc(N)nc2cn(Cc3ccc(CNC4CCOC(C)(C)C4)cc3OC)nc12] | δ = 7.76 (br s, 1H), 7.10 (s, 1H), 7.03-6.80 (m, 2H), 5.97 (br d, J = 8.3 Hz, 2H), 5.41 (s, 2H), 4.74 (br s, 1H), 4.20-4.01 (m, 2H), 3.88-3.81 (m, 5H), 3.77-3.60 (m, 6H), 3.49 (br s, 2H), 3.00-2.92 (m, 3H), 1.88-1.73 (m, 2H), 1.67- 1.43 (m, 2H), 1.35-1.20 (m, 3H), 1.12 (d, J = 18.3 Hz, 7H), 0.97-0.72 (m, 3H). LC-MS (ES): m/z = 512.3 [M + H]⁺, RT (min) = 1.38 |

### Example S6. 2-((5-Amino-2-(2-methoxy-4-(((tetrahydro-2H-pyran-4-yl) amino) methyl) benzyl)-3-methyl-2H-pyrazolo[4,3-d]pyrimidin-7-yl) (butyl)amino) ethan-1-ol (Compound 212)

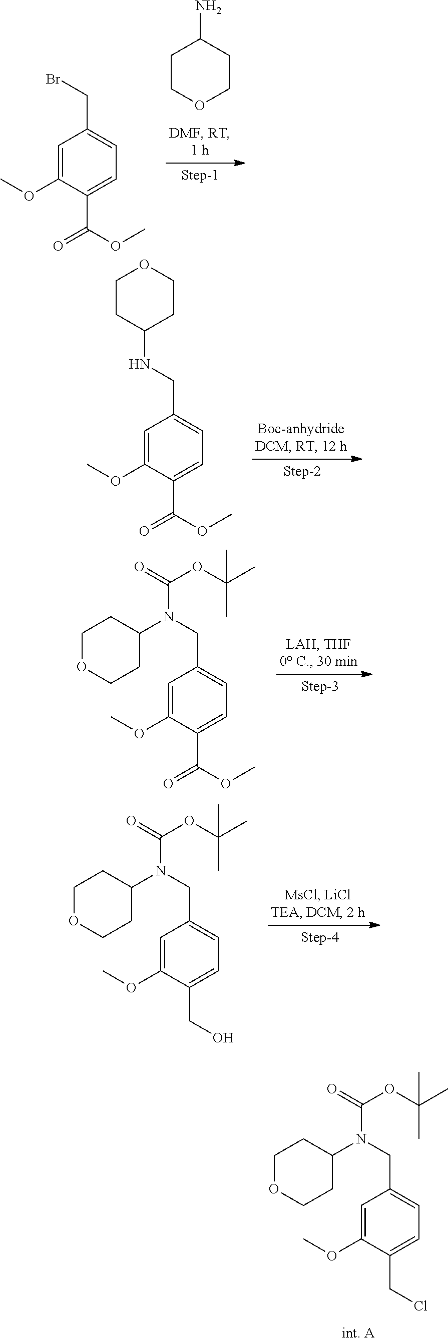

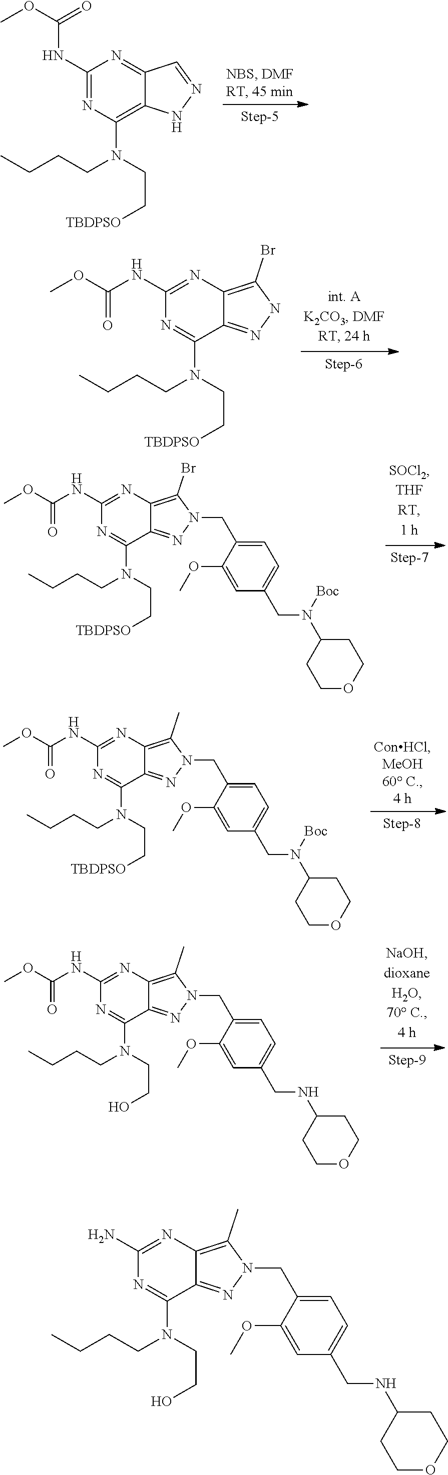

Step 1. Methyl 2-methoxy-4-(((tetrahydro-2H-pyran-4-yl) amino)methyl) benzoate: To a stirred solution of methyl 4-(bromomethyl)-2-methoxybenzoate (10 g, 38.6 mmol) in DMF (30 mL), was added tetrahydro-2H-pyran-4-amine (9.76 g, 96 mmol). The reaction mixture was stirred at RT for 1 h. The reaction mixture was concentrated to get the title compound. ¹H NMR (400 MHz, DMSO-d₆) δ=7.99-7.95 (m, 1H), 7.64-7.55 (m, 1H), 7.20-7.13 (m, 1H), 7.03-6.94 (m, 1H), 3.92-3.71 (m, 8), 3.39-3.12 (m, 5H), 2.65-2.53 (m, 1H), 1.56-1.19 (m, 4H). LC-MS (ES): m/z=280.2 [M+H]⁺

Step 2. Methyl 4-(((tert-butoxycarbonyl)(tetrahydro-2H-pyran-4-yl)amino)methyl)-2-methoxy benzoate: To a stirred solution of methyl 2-methoxy-4-(((tetrahydro-2H-pyran-4-yl) amino) methyl) benzoate (10 g, 35.8 mmol) in DCM (100 mL) at 0° C., were added DIPEA (18.76 mL, 107 mmol) and Boc-anhydride (16.62 mL, 71.6 mmol) dropwise over 10 min. The reaction mixture was stirred at RT for 12 h and concentrated under reduced pressure. The residue was purified using CombiFlash (Redisep Rf 120 g silica gel column; 40% ethyl acetate in pet. ether as an eluent) to afford the title compound (9.3 g, 24.51 mmol, 68.5% yield) as a colorless oil. ¹H NMR (500 MHz, DMSO-d₆) δ=7.67-7.55 (m, 1H), 7.01-6.97 (m, 1H), 6.91-6.81 (m, 1H), 4.45-4.35 (m, 2H), 3.87-3.71 (m, 8H), 3.37-3.22 (m, 2H), 2.65-2.61 (m, 1H), 1.72-1.58 (m, 2H), 1.51-1.20 (m, 11H). LC-MS (ES): m/z=324.2 [M−56]⁺

Step 3. tert-Butyl (4-(hydroxymethyl)-3-methoxybenzyl) (tetrahydro-2H-pyran-4-yl) carbamate: To a stirred solution of methyl 4-(((tert-butoxycarbonyl) (tetrahydro-2H-pyran-4-yl) amino) methyl)-2-methoxybenzoate (9.3 g, 24.51 mmol) in THE (90 mL) at 0° C., was added LAH (30.6 mL, 73.5 mmol) drop wise over 2 min. The reaction mixture was stirred at RT for 0.5 h. The reaction mixture was quenched by dropwise addition of saturated aq. NaOH solution until the effervescence ceased. The reaction mixture was filtered through a Celite bed, which was washed with ethyl acetate. The filtrate was dried over sodium sulphate, filtered, and concentrated to get the title compound (7.8 g, 22.19 mmol, 91% yield) as a colorless oil. ¹H NMR (500 MHz, DMSO-d₆) δ=7.35-7.27 (m, 1H), 6.87-6.80 (m, 2H), 4.97-4.89 (m, 1H), 4.52-4.47 (m, 2H), 4.44-4.37 (m, 2H), 4.15-4.00 (m, 1H), 3.89-3.81 (m, 2H), 3.79-3.74 (m, 3H), 3.33-3.22 (m, 2H), 1.76-1.61 (m, 2H), 1.55-1.30 (m, 11H). LC-MS (ES): m/z=278.2 [M−56]⁺

Step 4. tert-Butyl (4-(chloromethyl)-3-methoxybenzyl) (tetrahydro-2H-pyran-4-yl) carbamate: To a stirred solution of tert-butyl (4-(hydroxymethyl)-3-methoxybenzyl) (tetrahydro-2H-pyran-4-yl) carbamate (4 g, 11.38 mmol) in DCM (40 mL) at 0° C., were added triethylamine (6.35 mL, 45.5 mmol), MsCl (2.66 mL, 34.1 mmol) and lithium chloride (0.965 g, 22.76 mmol). The reaction mixture was stirred at RT for 2 h, diluted with saturated aq. NaHCO₃ solution and extracted with DCM (2×50 mL). The combined organic layer was washed with water and brine solution, dried over sodium sulphate, filtered, and concentrated. The crude was purified using CombiFlash (Redisep Rf 40 g silica gel column; 30% ethyl acetate in pet. ether as an eluent) to afford the title compound (2.1 g, 5.68 mmol, 49.9% yield) as colorless oil. ¹H NMR (500 MHz, DMSO-d₆) δ=7.38-7.25 (m, 1H), 6.93-6.70 (m, 2H), 4.72-4.62 (m, 2H), 4.41-4.30 (m, 2H), 3.87-3.70 (m, 5H), 3.38-3.21 (m, 3H), 1.72-1.59 (m, 2H), 1.52-1.16 (m, 11H). LC-MS (ES): m/z=314.2 [M−56]⁺

Step 5. Methyl (3-bromo-7-(butyl(2-((tert-butyldiphenylsilyl)oxy)ethyl)amino)-1H-pyrazolo[4,3-d]pyrimidin-5-yl) carbamate: To a stirred solution of methyl (7-(butyl(2-((tert-butyldiphenylsilyl) oxy) ethyl) amino)-1H-pyrazolo[4,3-d]pyrimidin-5-yl) carbamate (3 g, 5.49 mmol) in DMF (30 mL), was added NBS (1.172 g, 6.58 mmol). The reaction mixture was stirred at RT for 45 min, diluted with ice cold water and stirred for 15 min. The precipitated solid was filtered and dried to afford the title compound (1.6 g, 2.56 mmol, 46.6% yield) as a brown solid. LC-MS (ES): m/z=625.2 [M+H]⁺

Step 6. tert-Butyl (4-((3-bromo-7-(butyl(2-((tert-butyldiphenylsilyl) oxy) ethyl) amino)-5-((methoxycarbonyl)amino)-2H-pyrazolo[4,3-d]pyrimidin-2-yl) methyl)-3-methoxybenzyl) (tetrahydro-2H-pyran-4-yl) carbamate: To a stirred solution of methyl (3-bromo-7-(butyl(2-((tert-butyldiphenylsilyl)oxy)ethyl)amino)-1H-pyrazolo[4,3-d]pyrimidin-5-yl) carbamate (1.5 g, 2.398 mmol) in DMF (20 mL), were added K₂CO₃ (0.994 g, 7.19 mmol) and tert-butyl (4-(chloromethyl)-3-methoxybenzyl) (tetrahydro-2H-pyran-4-yl) carbamate (1.064 g, 2.88 mmol) at 0° C. The reaction mixture was allowed to warm to RT and stirred for 24 h. The reaction was diluted with ice cold water. The precipitated solid was filtered and dried to afford the title compound (1.6 g, 1.668 mmol, 69.6% yield) as a yellow solid. LC-MS (ES): m/z=958.3 [M+H]⁺

Step 7. tert-Butyl (4-((7-(butyl(2-((tert-butyldiphenylsilyl) oxy) ethyl) amino)-5-((methoxycarbonyl)amino)-3-methyl-2H-pyrazolo[4,3-d]pyrimidin-2-yl) methyl)-3-methoxybenzyl) (tetrahydro-2H-pyran-4-yl) carbamate: To a solution of tert-butyl (4-((3-bromo-7-(butyl(2-((tert-butyldiphenylsilyl) oxy) ethyl) amino)-5-((methoxycarbonyl)amino)-2H-pyrazolo[4,3-d]pyrimidin-2-yl) methyl)-3-methoxybenzyl) (tetrahydro-2H-pyran-4-yl) carbamate (250 mg, 0.261 mmol) in a mixture of dioxane (10 mL) and water (1 mL), were added trimethylboroxine (0.182 mL, 1.303 mmol), K₂CO₃ (108 mg, 0.782 mmol) and PdCl₂(dppf)-CH₂Cl₂ (63.9 mg, 0.078 mmol). The reaction mixture was heated to 110° C. under N₂ gas for 12 h. The reaction mixture was allowed to cool to RT and filtered through a Celite pad, which was washed with MeOH. The filtrate was concentrated under reduced pressure. The residue was partitioned between water and ethyl acetate. The organic layer was washed with water and brine solution, dried over sodium sulphate, filtered, and concentrated. The crude was purified using CombiFlash (Redisep Rf 40 g silica gel column; 5% MeOH in DCM as an eluent) to afford the title compound (145 mg, 0.162 mmol, 62.2% yield) as a brown solid. LC-MS (ES): m/z=894.2 [M+H]⁺

Step 8. Methyl (7-(butyl(2-hydroxyethyl) amino)-2-(2-methoxy-4-(((tetrahydro-2H-pyran-4-yl) amino) methyl) benzyl)-3-methyl-2H-pyrazolo[4,3-d]pyrimidin-5-yl) carbamate hydrochloride: To a solution containing tert-butyl (4-((7-(butyl(2-((tert-butyldiphenylsilyl)oxy)ethyl)amino)-5-((methoxycarbonyl)amino)-3-methyl-2H-pyrazolo[4,3-d]pyrimidin-2-yl)methyl)-3-methoxybenzyl) (tetrahydro-2H-pyran-4-yl) carbamate (80 mg, 0.089 mmol) in MeOH (5 mL), was added HCl in dioxane (4M, 1.193 mL, 1.789 mmol) dropwise over 2 min. The mixture was stirred at RT for 4 h. The reaction mixture was concentrated under reduced pressure. The residue was triturated with diethyl ether (2×10 mL), and dried to afford the title compound (48 mg, 0.081 mmol, 91% yield) as a light yellow solid. LC-MS (ES): m/z=556.2 [M+H]⁺

Step 9. 2-((5-Amino-2-(2-methoxy-4-(((tetrahydro-2H-pyran-4-yl) amino) methyl) benzyl)-3-methyl-2H-pyrazolo[4,3-d]pyrimidin-7-yl) (butyl)amino) ethan-1-ol: To a solution containing methyl (7-(butyl(2-hydroxyethyl)amino)-2-(2-methoxy-4-(((tetrahydro-2H-pyran-4-yl)amino)methyl)benzyl)-3-methyl-2H-pyrazolo[4,3-d]pyrimidin-5-yl)carbamate hydrochloride (48 mg, 0.081 mmol) in dioxane (5 mL), was added an aqueous solution of NaOH (0.811 mL, 2.432 mmol) at room temperature. The reaction mixture was heated to 70° C. and stirred for 4 h. The organic layer was separated and concentrated under reduced pressure. The crude compound was purified by preparative LC-MS (Column: Waters XBridge C18, 250 mm×19 mm, 5-μm particles; mobile phase A: 5:95 acetonitrile:water with 0.05% TFA; mobile phase B: 95:5 acetonitrile:water with 0.05% TFA; gradient: a 0-minute hold at 12% B, 12-34% B over 25 minutes, then a 5-minute hold at 100% B; flow rate: 20 mL/min; column temperature: 25° C.). Fractions containing the desired product were combined and dried via centrifugal evaporation to afford the title compound (23.2 mg, 0.044 mmol, 53.9% yield).

| Cmpd | | Analytical Data (¹H NMR 400 MHz, DMSO- |
|---|---|---|
| No. | Structure | d₆; LC-MS) |
| 212 | 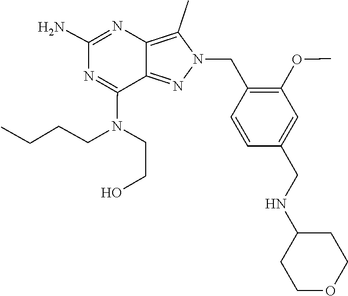 [SMILES: CCCCN(CCO)c1nc(N)nc2c(C)n(Cc3ccc(CNC4CCOCC4)cc3OC)nc12] | δ = 8.89 (br s, 2H), 7.60 (br s, 1H), 7.33-7.21 (m, 1H), 7.13-6.78 (m, 2H), 5.45 (d, J = 3.8 Hz, 2H), 4.83-4.81 (m, 1H), 4.92-4.68 (m, 1H), 4.27-4.10 (m, 4H), 3.98-3.90 (m, 2H), 3.88-3.76 (m, 5H), 3.76-3.61 (m, 3H), 3.28- 3.23 (m, 1H), 2.46-2.39 (m, 4H), 2.07-1.93 (m, 2H), 1.73-1.50 (m, 4H), 1.40-1.19 (m, 2H), 1.01-0.81 (m, 3H). LC-MS (ES): m/z = 498.3 [M + H]⁺, RT (min) = 1.56 |

### Example S7. 2-((5-amino-2-(2-methoxy-4-(piperidin-1-ylmethyl)benzyl)-2H-pyrazolo[4,3-d]pyrimidin-7-yl)(butyl)amino)propan-1-ol (Compound 49 (Racemic), Compound 49-1 (Enantiomer 1), and Compound 49-2 (Enantiomer 2))

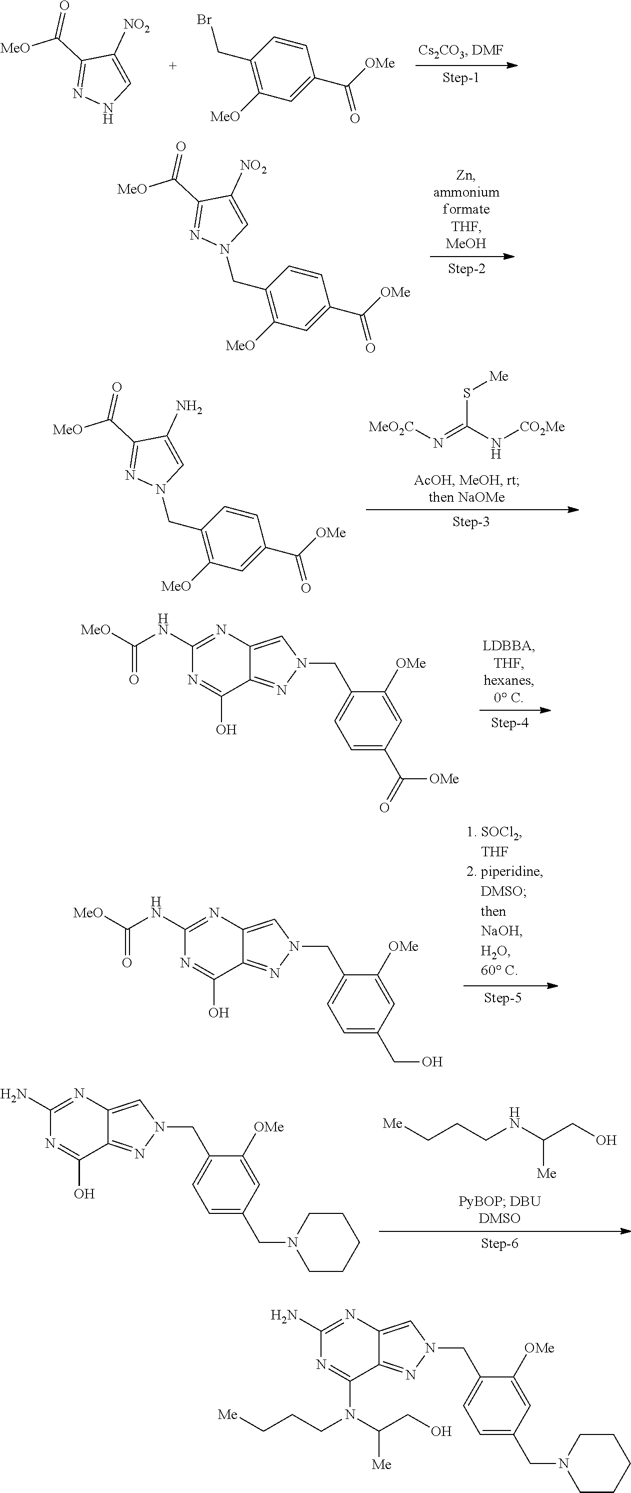

Step 1. Methyl 1-(2-methoxy-4-(methoxycarbonyl)benzyl)-4-nitro-1H-pyrazole-3-carboxylate: A biphasic mixture of methyl 4-nitro-1H-pyrazole-3-carboxylate (1 g, 5.84 mmol), methyl 4-(bromomethyl)-3-methoxybenzoate (1.514 g, 5.84 mmol), and cesium carbonate (3.81 g, 11.69 mmol) in DMF (5.84 mL) was stirred at rt under ambient atmosphere for 10 min. The reaction mixture was diluted with water and extracted with EtOAc. The combined organic layers were washed with a 10% aqueous solution of LiCl, washed with brine, dried over MgSO₄, filtered, and concentrated under reduced pressure. The crude was purified using CombiFlash (Redisep Rf 40 g silica gel column; 0-50% EtOAc in hexanes as an eluent) to afford the title compound (1.6383 g, 4.6903 mmol, 80% yield) as a white solid. ¹H NMR (400 MHz, DMSO-d₆) δ 9.05 (s, 1H), 7.58 (dd, J=7.9, 1.5 Hz, 1H), 7.53 (d, J=1.3 Hz, 1H), 7.28 (d, J=7.7 Hz, 1H), 5.47 (s, 2H), 3.89 (s, 3H), 3.87 (s, 3H), 3.86 (s, 3H). LC-MS (ES): m/z=350.2 [M+H]⁺.

Step 2. Methyl 4-amino-1-(2-methoxy-4-(methoxycarbonyl)benzyl)-1H-pyrazole-3-carboxylate: To a mixture of methyl 1-(2-methoxy-4-(methoxycarbonyl)benzyl)-4-nitro-1H-pyrazole-3-carboxylate (1.6383 g, 4.69 mmol) and ammonium formate (1.183 g, 18.76 mmol) in MeOH (9.38 mL) and THE (9.38 mL) was added zinc (0.767 g, 11.73 mmol). The reaction mixture was stirred at rt under ambient atmosphere for 1 h. Additional portions of zinc (0.767 g, 11.73 mmol) and ammonium formate (1.183 g, 18.76 mmol) were added and the reaction mixture was stirred for an additional 15 minutes. The reaction mixture was filtered through Celite, rinsed with MeOH, and the filtrate was concentrated under reduced pressure to give the title compound (3.1470 g with ammonium formate impurities) as a white residue, which was used in the next step without any further purification. ¹H NMR (400 MHz, DMSO-d₆) δ 7.60-7.48 (m, 2H), 7.21 (s, 1H), 6.97 (d, J=7.7 Hz, 1H), 5.26 (s, 2H), 3.90 (s, 3H), 3.86 (s, 3H), 3.74 (s, 3H). LC-MS (ES): m/z=320.2 [M+H]⁺.

Step 3. Methyl 4-((7-hydroxy-5-((methoxycarbonyl)amino)-2H-pyrazolo[4,3-d]pyrimidin-2-yl)methyl)-3-methoxybenzoate: To a mixture of methyl 4-amino-1-(2-methoxy-4-(methoxycarbonyl)benzyl)-1H-pyrazole-3-carboxylate (3.1470 g) and 1,3-bis(methoxycarbonyl)-2-methyl-2-thiopseudourea (1.064 g, 5.16 mmol) in MeOH (18.76 mL) was added AcOH (1.074 mL, 18.76 mmol). The white slurry was stirred at rt under ambient atmosphere for 15 h. Additional MeOH (18.76 mL) was added to the solidified reaction mixture, which was then broken up to form a suspension. An additional portion of AcOH (1.074 mL, 18.76 mmol) was added and the reaction mixture was stirred for 1 h, after which additional portions of MeOH (18.76 mL) and AcOH (1.074 mL, 18.76 mmol) were added. The reaction mixture was stirred for 1 h and then diluted with MeOH to a total volume of ˜250 mL. The slurry was stirred for 2.5 h, and then an additional portion of AcOH (1.074 mL, 18.76 mmol) was added and the slurry was stirred for an additional 2.5 h. A 25 wt % solution of sodium methoxide in MeOH (25.7 mL, 113 mmol) was added and the reaction mixture solubilized. The reaction mixture was stirred for 1 h and then acidified to pH=ca. 5 with ˜10 mL AcOH. The resultant white precipitate was collected by filtration and rinsed with water, MeOH, and Et₂O to give the title compound (1.3910 g, 3.591 mmol, 77% over 2 steps) as a fluffy, white solid. ¹H NMR (400 MHz, DMSO-d₆) δ 8.17 (s, 1H), 7.57-7.52 (m, 2H), 7.07 (d, J=7.5 Hz, 1H), 5.54 (s, 2H), 3.91 (s, 3H), 3.86 (s, 3H), 3.72 (s, 3H). LC-MS (ES): m/z=388.3 [M+H]⁺.

Step 4. Methyl (7-hydroxy-2-(4-(hydroxymethyl)-2-methoxybenzyl)-2H-pyrazolo[4,3-d]pyrimidin-5-yl)carbamate: To a suspension of methyl 4-((7-hydroxy-5-((methoxycarbonyl)amino)-2H-pyrazolo[4,3-d]pyrimidin-2-yl)methyl)-3-methoxybenzoate (0.8739 g, 2.256 mmol) in THE (22.6 mL) under N₂ was added a 0.25 M solution of lithium diisobutyl-tert-butoxyaluminum hydride solution in THF/hexanes (63.2 mL, 15.79 mmol) dropwise at 0° C. The reaction mixture was stirred for 20 min at 0° C. under N₂, then quenched with MeOH, acidified to pH=ca. 3 with 1 M HCl, and then concentrated under reduced pressure. The crude was purified using CombiFlash (Redisep Rf 24 g silica gel column; 0-50% MeOH in DCM as an eluent) to afford the title compound (1.9320 g with aluminum salt impurities) as a white solid, which was used in the next step without any further purification. ¹H NMR (400 MHz, DMSO-d₆) δ 8.08 (s, 1H), 7.05 (d, J=7.7 Hz, 1H), 7.01 (s, 1H), 6.88 (d, J=7.7 Hz, 1H), 5.44 (s, 2H), 5.24 (t, J=5.7 Hz, 1H), 4.49 (d, J=5.7 Hz, 2H), 3.82 (s, 3H), 3.73 (s, 3H). LC-MS (ES): m/z=360.3 [M+H]⁺.

Step 5. 5-Amino-2-(2-methoxy-4-(piperidin-1-ylmethyl)benzyl)-2H-pyrazolo[4,3-d]pyrimidin-7-ol, TFA: To a solution of methyl (7-hydroxy-2-(4-(hydroxymethyl)-2-methoxybenzyl)-2H-pyrazolo[4,3-d]pyrimidin-5-yl)carbamate (1.8583 g) in THE (100 mL) under N₂ was added thionyl chloride (4.53 mL, 62.1 mmol). The reaction mixture was stirred at rt for 15 h and then concentrated under reduced pressure. The residue was dissolved in DMSO (25 mL) and then piperidine (2.56 mL, 25.9 mmol) was added. The reaction mixture was stirred for 15 min at rt under ambient atmosphere, and then water (17 mL) was added, followed by NaOH, 10 M in H₂O (5.17 mL, 51.7 mmol). The reaction mixture was heated at 60° C. for 20 min, and then an additional portion of NaOH, 10 M in H₂O (2.6 mL, 26 mmol) was added. The reaction mixture was heated at 60° C. for 40 min, and then an additional portion of NaOH, 10 M in H₂O (2.6 mL, 26 mmol) was added. The reaction mixture was stirred for 45 min, cooled to 0° C., and acidified to pH=ca. 5 with ˜121 mL 1 M HCl and held for 4 days at ˜0° C. The mixture was concentrated under reduced pressure and filtered through Celite. The filter cake was rinsed with MeCN and the filtrate was concentrated under reduced pressure to give a thick oil. The crude was purified using CombiFlash (Redisep Rf 100 g C18 AQ column; 0-50% MeCN in H₂O with TFA modifier as an eluent) to afford the title compound (840.7 mg, 1.743 mmol) as a white solid. ¹H NMR (400 MHz, DMSO-d₆) δ 9.49 (br s, 1H), 8.01 (s, 1H), 7.84 (br s, 1H), 7.22 (d, J=1.1 Hz, 1H), 7.16-7.10 (m, 1H), 7.08-7.02 (m, 1H), 5.47 (s, 2H), 4.27 (br d, J=4.6 Hz, 2H), 3.87 (s, 3H), 3.32 (br d, J=12.1 Hz, 2H), 2.97-2.80 (m, 2H), 1.88-1.77 (m, 2H), 1.73-1.30 (m, 4H). ¹⁹F NMR (376 MHz, DMSO-d₆) δ −74.10 (s, 3F). LC-MS (ES): m/z=369.4 [M+H]⁺.

Step 6. 2-((5-Amino-2-(2-methoxy-4-(piperidin-1-ylmethyl)benzyl)-2H-pyrazolo[4,3-d]pyrimidin-7-yl)(butyl)amino)propan-1-ol: To a solution of 5-amino-2-(2-methoxy-4-(piperidin-1-ylmethyl)benzyl)-2H-pyrazolo[4,3-d]pyrimidin-7-ol, TFA (15 mg, 0.031 mmol), 2-(butylamino)propan-1-ol (8.16 mg, 0.062 mmol), and PyBOP (19.42 mg, 0.037 mmol) in DMSO (311 μL), was added DBU (18.75 μL, 0.124 mmol). The reaction mixture was stirred at room temperature under ambient atmosphere for 2 hours. The reaction mixture was then diluted with 1.7 mL DMSO, filtered, and purified via preparative LC/MS with the following conditions: column: XBridge C18, 200 mm×19 mm, 5-μm particles; mobile phase A: 5:95 acetonitrile:water with ammonium acetate; mobile phase B: 95:5 acetonitrile:water with ammonium acetate; gradient: a 0-minute hold at 14% B, 14-54% B over 20 minutes, then a 0-minute hold at 100% B; flow Rate: 20 mL/min; column temperature: 25° C. Fraction collection was triggered by MS signals. Fractions containing the desired product were combined and dried via centrifugal evaporation to give Compound 49 (14.4 mg, 96%). The racemate was separated via chrial SFC with the following conditions: column: chiral OD, 30×250 mm. 5 micron; mobile phase: 70% CO2/30% IPA w/0.1% DEA; flow conditions: 100 mL/min; detector wavelength: 220 nM. Fractions containing the separate enantiomers were combined and dried via centrifugal evaporation to give Compound 49-1 (1.8 mg, 12%) and Compound 49-2 (1.5 mg, 10%) with >95% chiral purity each.

| Cmpd | | Analytical Data (¹H NMR 500 MHz, DMSO- |
|---|---|---|
| No. | Structure | d₆; LC-MS) |
| 49 | 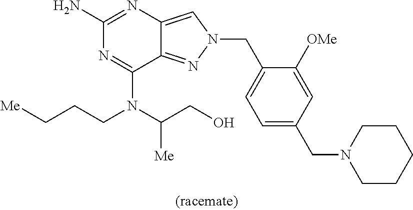 [SMILES: CCCCN(c1nc(N)nc2cn(Cc3ccc(CN4CCCCC4)cc3OC)nc12)C(C)CO] | 7.73 (br s, 1H), 6.96 (s, 2H), 6.83 (br d, J = 7.6 Hz, 1H), 5.63 (br s, 2H), 5.38 (s, 2H), 4.46- 4.20 (m, 1H), 4.14-3.85 (m, 2H), 3.84-3.83 (m, 1H), 3.81 (s, 2H), 2.31 (br s, 4H), 1.48 (br d, J = 5.2 Hz, 6H), 1.38 (br s, 2H), 1.31-1.18 (m, 2H), 1.12-0.97 (m, 3H), 0.92-0.71 (m, 3H). 4 N—CH₂ protons were not observed, which is attributed to the water suppression used. LC- MS (ES): m/z = 482.4 [M + H]⁺, RT (min) = 1.31 |
| 49-1 | 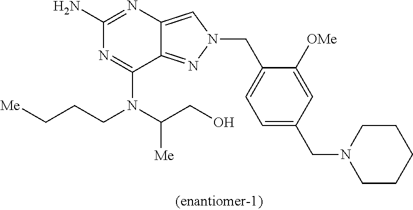 [SMILES: CCCCN(c1nc(N)nc2cn(Cc3ccc(CN4CCCCC4)cc3OC)nc12)C(C)CO] | 7.78 (s, 1H), 6.97 (s, 2H), 6.84 (br d, J = 7.6 Hz, 1H), 6.16-5.85 (m, 2H), 5.39 (s, 2H), 4.47- 4.24 (m, 1H), 4.08-3.86 (m, 2H), 3.81 (s, 3H), 2.32 (br s, 4H), 1.64-1.45 (m, 6H), 1.39 (br d, J = 4.6 Hz, 2H), 1.31-1.18 (m, 2H), 1.12-0.97 (m, 3H), 0.94-0.77 (m, 3H). 4 N—CH₂ protons were not observed, which is attributed to the water suppression used. LC-MS (ES): m/z = 482.4 [M + H]⁺, RT (min) = 1.31 |
| 49-2 | 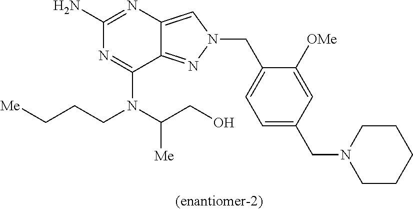 [SMILES: CCCCN(c1nc(N)nc2cn(Cc3ccc(CN4CCCCC4)cc3OC)nc12)C(C)CO] | δ 7.79 (s, 1H), 6.97 (s, 2H), 6.84 (d, J = 7.6 Hz, 1H), 6.29-5.85 (m, 2H), 5.40 (s, 2H), 4.48-4.21 (m, 1H), 4.13-3.87 (m, 2H), 3.81 (s, 3H), 2.32 (br s, 3H), 1.69-1.45 (m, 6H), 1.38 (br s, 2H), 1.33-1.20 (m, 2H), 1.13-0.97 (m, 3H), 0.95- 0.76 (m, 3H). 4 N—CH₂ protons were not observed, which is attributed to the water suppression used. LC-MS (ES): m/z = 482.4 [M + H]⁺, RT (min) = 1.31 |

The following compound was prepared following a similar experimental procedure for Compound 49.

| Cmpd | | Analytical Data (¹H NMR 500 MHz, DMSO- |
|---|---|---|
| No. | Structure | d₆; LC-MS) |
| 86 | 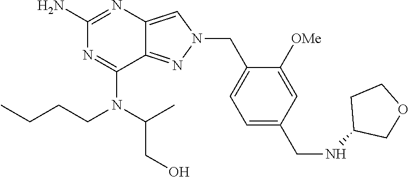 [SMILES: CCCCN(c1nc(N)nc2cn(Cc3ccc(CN[C@@H]4CCOC4)cc3OC)nc12)C(C)CO] | δ 7.92 (s, 1H), 7.10 (d, J = 10.9 Hz, 3H), 6.94 (d, J = 7.6 Hz, 1H), 5.45 (s, 2H), 4.37 (s, 1H), 4.08 (s, 1H), 3.95 (s, 1H), 3.85-3.76 (m, 6H), 3.71 (dd, J = 9.1, 6.0 Hz, 1H), 3.66-3.59 (m, 1H), 2.55 (s, 6H), 2.01 (dd, J = 13.1, 6.9 Hz, 1H), 1.78 (d, J = 6.0 Hz, 0H), 1.61 (s, 1H), 1.56 (s, 1H), 1.24 (s, 2H), 1.11 (s, 2H), 1.04 (s, 1H), 0.92 (s, 2H), 0.86 (s, 3H). LC-MS (ES): m/z = 484.3 [M + H]⁺, RT (min) = 1.42 |

### Example S8. 2-((5-amino-2-(2-fluoro-4-(1-(2-hydroxyethyl)piperidin-4-yl)-6-methoxybenzyl)-2H-pyrazolo[4,3-d]pyrimidin-7-yl)(butyl)amino)ethan-1-ol (Compound 34)

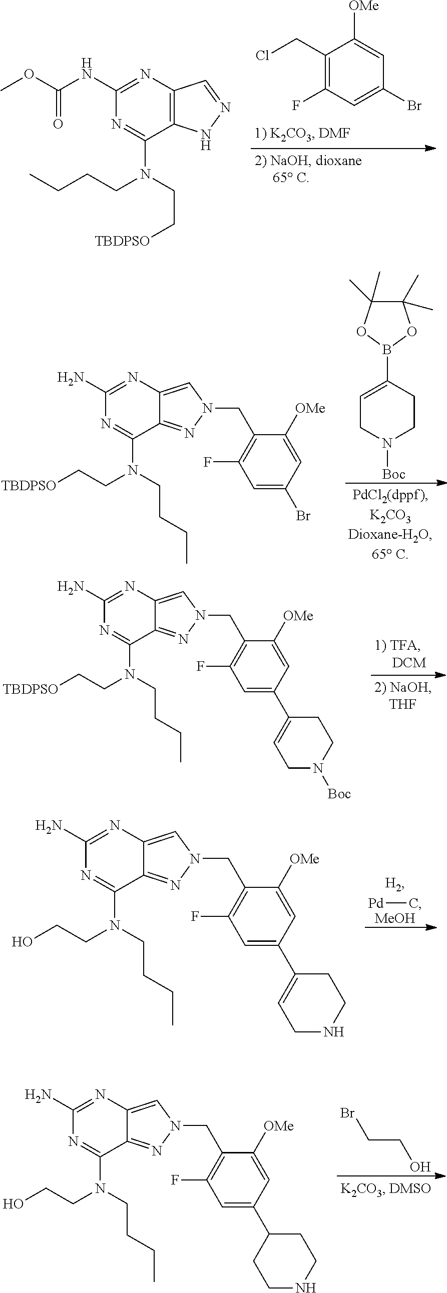

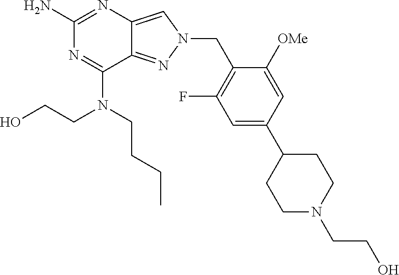 [SMILES: CCCCN(CCO)c1nc(N)nc2cn(Cc3c(F)cc(C4CCN(CCO)CC4)cc3OC)nc12]

Step 1. 2-((5-Amino-2-(2-fluoro-6-methoxy-4-(piperidin-4-yl)benzyl)-2H-pyrazolo[4,3-d]pyrimidin-7-yl)(butyl)amino)ethan-1-ol: A mixture of 5-bromo-2-(chloromethyl)-1-fluoro-3-methoxybenzene (306 mg, 1.207 mmol) in DMF (5 mL) was treated with K₂CO₃ (278 mg, 2.012 mmol) followed by 5-bromo-2-(chloromethyl)-1-fluoro-3-methoxybenzene (306 mg, 1.207 mmol). The resulting mixture was allowed to stir at ambient temperature for 45 min and quenched with saturated NH₄Cl solution. The mixture was extracted with EtOAc (3×100 ml). The combined organic layer was dried over Na₂SO₄, filtered and concentrated giving methyl (2-(4-bromo-2-fluoro-6-methoxybenzyl)-7-(butyl(2-((tert-butyldiphenylsilyl)oxy)ethyl)amino)-2H-pyrazolo[4,3-d]pyrimidin-5-yl)carbamate, which was used without purification. LC-MS (ES, m/z): [M+H]⁺=763.2, 765.2.

The above crude material was suspended in dioxane (5 mL) and treated with NaOH 5.0 N (1.207 mL, 6.04 mmol) and the mixture was stirred at 65° C. for 24 h. Upon cooling, reaction was quenched with saturated NH₄Cl solution and the mixture was extracted with EtOAc (3×100 mL). The combined organic layer was dried over Na₂SO₄, filtered and concentrated. The crude material was purified using flash chromatography (ISCO, 40 g SiO₂ column, loaded in DCM, 0 to 50% (20% MeOH in DCM) in DCM over 25 minutes). The desired fraction was concentrated to yield 2-(4-bromo-2-fluoro-6-methoxybenzyl)-N7-butyl-N7-(2-((tert-butyldiphenylsilyl)oxy)ethyl)-2H-pyrazolo[4,3-d]pyrimidine-5,7-diamine (488 mg, 0.691 mmol, 68.7% yield). ¹H NMR @90° C. (400 MHz, DMSO-d₆) δ 7.66-7.56 (m, 5H), 7.47-7.29 (m, 6H), 7.11-7.01 (m, 2H), 5.39 (s, 2H), 5.04 (br s, 2H), 4.08-3.94 (m, 2H), 3.94-3.85 (m, 4H), 3.82 (s, 3H), 1.63-1.43 (m, 2H), 1.31-1.12 (m, 2H), 0.99 (s, 9H), 0.87 (t, J=7.4 Hz, 3H); LC-MS (ES, m/z): [M+H]⁺=705.3, 707.3

Step 2. tert-Butyl 4-(4-((5-amino-7-(butyl(2-((tert-butyldiphenylsilyl)oxy)ethyl)amino)-2H-pyrazolo[4,3-d]pyrimidin-2-yl)methyl)-3-fluoro-5-methoxyphenyl)-3,6-dihydropyridine-1(2H)-carboxylate: A mixture of 2-(4-bromo-2-fluoro-6-methoxybenzyl)-N7-butyl-N7-(2-((tert-butyldiphenylsilyl)oxy)ethyl)-2H-pyrazolo[4,3-d]pyrimidine-5,7-diamine (488 mg, 0.691 mmol) in dioxane (4 mL) and water (1 mL) was treated with tert-butyl 4-(4,4,5,5-tetramethyl-1,3,2-dioxaborolan-2-yl)-3,6-dihydropyridine-1(2H)-carboxylate (257 mg, 0.830 mmol) and K₂CO₃ (287 mg, 2.074 mmol). Then a stream of N₂ was bubbled into the mixture for 2 min, PdCl₂(dppf)-CH₂Cl₂ adduct (56.5 mg, 0.069 mmol) was added, a stream of N₂ was bubbled into the mixture for another 2 min, then sealed, and the mixture was stirred at 60° C. for 2 h. Upon cooling, the EtOAc (10 mL) was added. The catalyst was removed by filtration and the filtrate was concentrated. The crude material was purified using flash chromatography (ISCO, 40 g SiO₂ column) by eluting with EA:HX=0-100%, followed by 20% MeOH in DCM:DCM=0-100%. The desired fractions were concentrated to yield tert-butyl 4-(4-((5-amino-7-(butyl(2-((tert-butyldiphenylsilyl)oxy)ethyl)amino)-2H-pyrazolo[4,3-d]pyrimidin-2-yl)methyl)-3-fluoro-5-methoxyphenyl)-3,6-dihydropyridine-1(2H)-carboxylate (417.5 mg, 0.517 mmol, 74.7% yield). LC-MS (ES, m/z): [M+H]⁺=808.5

Step 3. 2-((5-amino-2-(2-fluoro-6-methoxy-4-(1,2,3,6-tetrahydropyridin-4-yl)benzyl)-2H-pyrazolo[4,3-d]pyrimidin-7-yl)(butyl)amino)ethan-1-ol: The mixture of tert-butyl 4-(4-((5-amino-7-(butyl(2-((tert-butyldiphenylsilyl)oxy)ethyl)amino)-2H-pyrazolo[4,3-d]pyrimidin-2-yl)methyl)-3-fluoro-5-methoxyphenyl)-3,6-dihydropyridine-1(2H)-carboxylate (450 mg, 0.557 mmol) in DCM (5 mL) was treated with TFA (1 mL, 12.98 mmol) and stirred at ambient temperature for 4 h. The reaction was concentrated to dryness. This crude material was then suspended into dioxane (5 mL), and treated with NaOH (5.0 N, 2.227 mL, 11.14 mmol). The mixture was then stirred at 80° C. for 16 h. Upon cooling, reaction mixture was concentrated to yield 2-((5-amino-2-(2-fluoro-6-methoxy-4-(1,2,3,6-tetrahydropyridin-4-yl)benzyl)-2H-pyrazolo[4,3-d]pyrimidin-7-yl)(butyl)amino)ethan-1-ol, which was used without purification. 20 mg of this crude material was purified via preparative LC/MS [column: XBridge C18, 200 mm×19 mm, 5-μm particles; mobile phase A: 5:95 acetonitrile:water with ammonium acetate; mobile phase B: 95:5 acetonitrile:water with ammonium acetate; Gradient: a 0-minute hold at 7% B, 7-47% B over 20 minutes, then a 0-minute hold at 100% B; flow rate: 20 mL/min; column temperature: 25 C]. Fraction collection was triggered by MS signals. Fractions containing the desired product were combined and dried via centrifugal evaporation to provide 3.8 mg of 2-((5-amino-2-(2-fluoro-6-methoxy-4-(1,2,3,6-tetrahydropyridin-4-yl)benzyl)-2H-pyrazolo[4,3-d]pyrimidin-7-yl)(butyl)amino)ethan-1-ol. ¹H NMR (500 MHz, DMSO-d₆) δ 7.74-7.51 (m, 1H), 6.97-6.83 (m, 2H), 6.34 (br s, 1H), 5.48 (br s, 2H), 5.40 (s, 2H), 4.15-3.96 (m, 2H), 3.87 (br s, 3H), 3.74-3.56 (m, 2H), 2.93 (br t, J=5.5 Hz, 2H), 2.35 (br s, 2H), 1.68-1.43 (m, 2H), 1.36-1.13 (m, 2H), 0.98-0.72 (m, 3H). Four protons were not visible due to water suppression, and two exchangeable protons were not visible either. LC-MS (ES, m/z): [M+H]⁺=470.4.

Step 4. 2-((5-amino-2-(2-fluoro-6-methoxy-4-(1,2,3,6-tetrahydropyridin-4-yl)benzyl)-2H-pyrazolo[4,3-d]pyrimidin-7-yl)(butyl)amino)ethan-1-ol: A Parr-shaker bottle containing mixture of 2-((5-amino-2-(2-fluoro-6-methoxy-4-(1,2,3,6-tetrahydropyridin-4-yl)benzyl)-2H-pyrazolo[4,3-d]pyrimidin-7-yl)(butyl)amino)ethan-1-ol (250 mg, 0.494 mmol) in MeOH (10 mL) was purged with N₂ and then Pd—C (52.6 mg, 0.494 mmol) was added. The reaction mixture was shaken under H₂ (45 psi) for 16 hours. Then the catalyst was removed by filtration. The filtrate was concentrated to yield 2-((5-amino-2-(2-fluoro-6-methoxy-4-(piperidin-4-yl)benzyl)-2H-pyrazolo[4,3-d]pyrimidin-7-yl)(butyl)amino)ethan-1-ol, which was used without purification. 25 mg of this crude material was purified via preparative LC/MS with the following conditions: column: XBridge C18, 200 mm×19 mm, 5-μm particles; Mobile Phase A: 5:95 acetonitrile:water with ammonium acetate; mobile phase B: 95:5 acetonitrile:water with ammonium acetate; gradient: a 0-minute hold at 6% B, 6-46% B over 20 minutes, then a 0-minute hold at 100% B; flow Rate: 20 mL/min; column Temperature: 25° C. Fraction collection was triggered by MS and UV signals. Fractions containing the desired product were combined and dried via centrifugal evaporation and yield of 11.7 mg of 2-((5-amino-2-(2-fluoro-6-methoxy-4-(piperidin-4-yl)benzyl)-2H-pyrazolo[4,3-d]pyrimidin-7-yl)(butyl)amino)ethan-1-ol. LC-MS (ES, m/z): [M+H]⁺=472.5. ¹H NMR (500 MHz, DMSO-d₆) δ 7.77-7.50 (m, 1H), 6.77 (br s, 1H), 6.70 (br d, J=10.4 Hz, 1H), 5.49 (br s, 2H), 5.44-5.27 (m, 2H), 4.02 (br d, J=7.0 Hz, 2H), 3.84 (br s, 4H), 3.77-3.58 (m, 2H), 3.12 (br d, J=11.6 Hz, 2H), 2.69 (br t, J=11.6 Hz, 2H), 1.76 (br d, J=11.6 Hz, 2H), 1.69-1.37 (m, 4H), 1.37-1.12 (m, 2H), 0.98-0.73 (m, 3H). Three protons were not visible due to water suppression and the overlap with DMSO-d₆ peak. One exchangeable proton was not visible either.

Step 5. 2-((5-amino-2-(2-fluoro-6-methoxy-4-(1-(2-methoxyethyl)piperidin-4-yl)benzyl)-2H-pyrazolo[4,3-d]pyrimidin-7-yl)(butyl)amino)ethan-1-ol: A mixture of 2-((5-amino-2-(2-fluoro-6-methoxy-4-(piperidin-4-yl)benzyl)-2H-pyrazolo[4,3-d]pyrimidin-7-yl)(butyl)amino)ethan-1-ol (25 mg, 0.049 mmol) in DMSO (0.5 mL) was treated with K₂CO₃ (40.8 mg, 0.295 mmol), followed by 1-bromo-2-methoxyethane (20.52 mg, 0.148 mmol). The resulting mixture was allowed to stir at ambient temperature for 16 hours. The solid was filtered off and filtrate was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 9% B, 9-49% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation and yield 2-((5-amino-2-(2-fluoro-6-methoxy-4-(1-(2-methoxyethyl)piperidin-4-yl)benzyl)-2H-pyrazolo[4,3-d]pyrimidin-7-yl)(butyl)amino)ethan-1-ol (2.3 mg, 4.22 μmol, 8.58% yield).

| Cmpd | | Analytical Data (¹H NMR 500 MHz, DMSO- |
|---|---|---|
| No. | Structure | d₆; LC-MS) |
| 34 | 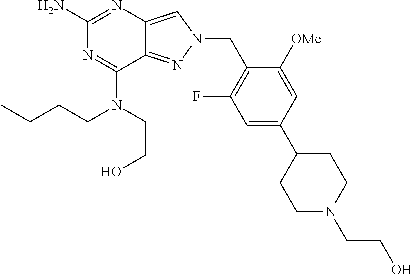 | δ 7.95-7.70 (m, 1H), 6.90-6.67 (m, 4H), 5.43 (s, 2H), 4.19-3.95 (m, 2H), 3.85 (br s, 3H), 3.80-3.56 (m, 4H), 3.49 (br t, J = 5.6 Hz, 1H), 3.07 (br d, J = 10.4 Hz, 2H), 2.68-2.56 (m, 3H), 2.21 (br dd, J-9.9, 8.1 Hz, 2H), 1.85-1.64 (m, 4H), 1.65-1.44 (m, 2H), 1.37-1.14 (m, 2H), 0.98-0.74 (m, 3H). Five protons were not visible due to water suppression and the overlap with DMSO-d₆ peak. LC-MS (ES): m/z = 530.6 [M + H]⁺, RT (min) = 1.31 |

### Example S9. 2-((5-amino-2-(2-fluoro-6-methoxy-4-(1-(tetrahydro-2H-pyran-4-yl)piperidin-4-yl)benzyl)-2H-pyrazolo[4,3-d]pyrimidin-7-yl)(butyl)amino)ethan-1-ol (Compound 37)

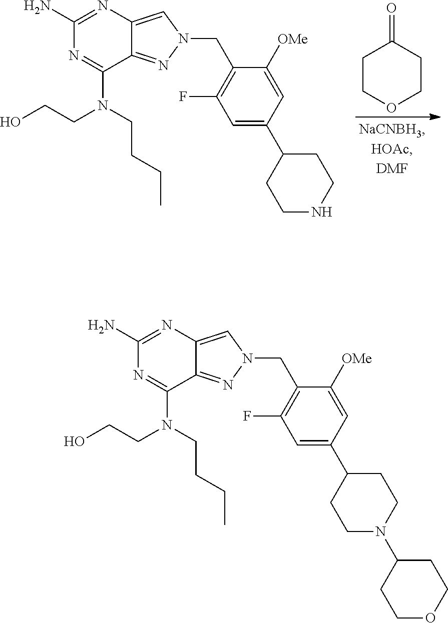 [SMILES: CCCCN(CCO)c1nc(N)nc2cn(Cc3c(F)cc(C4CCNCC4)cc3OC)nc12 ; O=C1CCOCC1 ; CCCCN(CCO)c1nc(N)nc2cn(Cc3c(F)cc(C4CCN(C5CCOCC5)CC4)cc3OC)nc12]

To a solution of 2-((5-amino-2-(2-fluoro-6-methoxy-4-(piperidin-4-yl)benzyl)-2H-pyrazolo[4,3-d]pyrimidin-7-yl)(butyl)amino)ethan-1-ol (25 mg, 0.049 mmol) in DMSO (0.5 mL) was treated with tetrahydro-4H-pyran-4-one (24.63 mg, 0.246 mmol), 1 drop of HOAc, followed by sodium cyanoborohydride (15.46 mg, 0.246 mmol) The reaction mixture was stirred at ambient temperature for 16 hours. LCMS indicated that reaction was completed. The mixture was purified via preparative LC/MS [Column: XBridge C18, 200 mm×19 mm, 5-μm particles; mobile phase A: 5:95 acetonitrile:water with ammonium acetate; mobile phase B: 95:5 acetonitrile:water with ammonium acetate; gradient: a 0-minute hold at 12% B, 12-52% B over 20 minutes, then a 0-minute hold at 100% B; flow rate: 20 mL/min; column Temperature: 25° C.]. Fraction collection was triggered by MS signals. Fractions containing the desired product were combined and dried via centrifugal evaporation to yield 2-((5-amino-2-(2-fluoro-6-methoxy-4-(1-(tetrahydro-2H-pyran-4-yl)piperidin-4-yl)benzyl)-2H-pyrazolo[4,3-d]pyrimidin-7-yl)(butyl)amino)ethan-1-ol (7.3 mg, 0.013 mmol, 26.7% yield).

| Cmpd | | Analytical Data (¹H NMR 500 MHz, DMSO- |
|---|---|---|
| No. | Structure | d₆; LC-MS) |
| 37 | 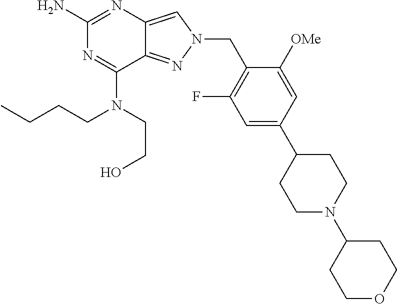 [SMILES: CCCCN(CCO)c1nc(N)nc2cn(Cc3c(F)cc(C4CCN(C5CCOCC5)CC4)cc3OC)nc12] | δ 7.72-7.46 (m, 1H), 6.79 (s, 1H), 6.73 (br d, J = 9.6 Hz, 1H), 5.49 (s, 2H), 5.37 (s, 2H), 3.73-3.49 (m, 3H), 3.28 (br t, J = 11.3 Hz, 2H), 2.98 (br d, J = 11.4 Hz, 2H), 2.49-2.37 (m, 3H), 2.18 (br t, J = 10.9 Hz, 2H), 1.81-1.39 (m, 10H), 1.35-1.12 (m, 2H), 0.98-0.71 (m, 3H). Eight protons were not visible due to water suppression and the overlap with DMSO-d₆ peak. LC-MS (ES): m/z = 556.3 [M + H]⁺, RT (min) = 1.55 |

In the following table, Compound 36, Compound 41, and Compound 42 were prepared following the procedure for Compound 34. Compound 38, Compound 39, Compound 40, Compound 47, Compound 110, Compound 164, and Compound 179 were prepared following the experimental procedure for Compound 37.

| Cmpd | | Analytical Data (¹H NMR 500 MHz, DMSO- |
|---|---|---|
| No. | Structure | d₆; LC-MS) |
| 36 | 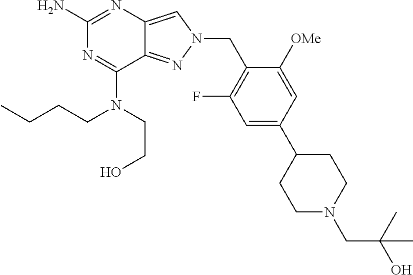 [SMILES: CCCCN(CCO)c1nc(N)nc2cn(Cc3c(F)cc(C4CCN(CC(C)(C)O)CC4)cc3OC)nc12] | δ 7.66 (br s, 1H), 6.78 (s, 1H), 6.73 (br d, J = 10.4 Hz, 1H), 5.53 (br s, 2H), 5.38 (s, 2H), 3.85 (s, 3H), 3.03 (br d, J = 11.6 Hz, 2H), 2.51- 2.41 (m, 2H), 2.27-2.11 (m, 4H), 1.68 (br d, J = 1.8 Hz, 4H), 1.61-1.44 (m, 2H), 1.37-1.18 (m, 2H), 1.10 (s, 6H), 0.85 (br s, 3H), Five protons were not visible due to water suppression and the overlap with the DMSO-d₆ peak, two exchangeable protons were not visible either. LC-MS (ES): m/z = 544.4 [M + H]⁺, RT (min) = 1.41 |
| 38 | 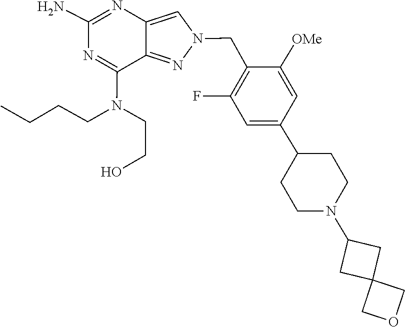 [SMILES: CCCCN(CCO)c1nc(N)nc2cn(Cc3c(F)cc(C4CCN(C5CC6(COC6)C5)CC4)cc3OC)nc12] | δ 7.72-7.49 (m, 1H), 6.78 (br s, 1H), 6.71 (br d, J = 10.1 Hz, 1H), 5.37 (s, 2H), 4.58 (s, 2H), 4.44 (s, 2H), 4.03 (br d, J = 7.6 Hz, 1H), 3.83 (br s, 3H), 3.73-3.48 (m, 2H), 2.85 (br d, J = 11.0 Hz, 2H), 2.50-2.44 (m, 2H), 2.36-2.24 (m, 2H), 1.95-1.91 (m, 2H), 1.82-1.65 (m, 4H), 1.65-1.43 (m, 4H), 1.38-1.15 (m, 2H), 0.98- 0.73 (m, 3H). Six protons were not visible due to water suppression and the overlap with DMSO-d₆ peak. LC-MS (ES): m/z = 568.4 [M + H]⁺, RT (min) = 1.46 |
| 39 | 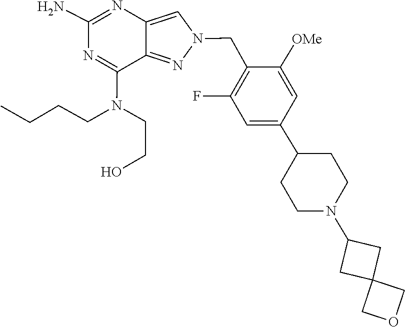 [SMILES: CCCCN(CCO)c1nc(N)nc2cn(Cc3c(F)cc(C4CCN(C5CC6(COC6)C5)CC4)cc3OC)nc12] | δ 7.67 (br s, 1H), 7.61-7.45 (m, 1H), 6.79 (br s, 1H), 6.72 (br d, J = 10.1 Hz, 1H), 5.59 (br s, 2H), 5.38 (s, 2H), 4.12-3.94 (m, 2H), 3.84 (br s, 3H), 3.74-3.54 (m, 3H), 2.85 (br d, J = 10.7 Hz, 2H), 2.50-2.44 (m, 2H), 2.14-2.05 (m, 2H), 1.98 (br t, J = 7.2 Hz, 2H), 1.88-1.83 (m, 2H), 1.83-1.76 (m, 2H), 1.76-1.69 (m, 4H), 1.67-1.41 (m, 4H), 1.35-1.12 (m, 2H), 0.99- 0.74 (m, 3H.) Three protons were not visible due to water suppression and the overlap with DMSO-d₆ peak. LC-MS (ES): m/z = 566.5 [M + H]⁺, RT (min) = 1.81 |
| 40 | 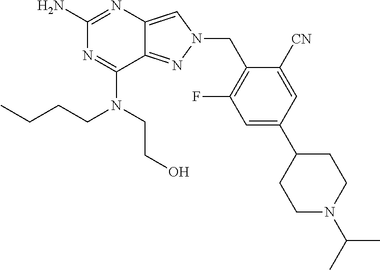 | δ 7.74-7.59 (m, 1H), 7.59-7.45 (m, 1H), 6.78 (br s, 1H), 6.72 (br d, J = 9.9 Hz, 1H), 5.40 (br s, 2H), 5.37 (s, 2H), 4.14-3.94 (m, 2H), 3.84 (br s, 3H), 3.72-3.60 (m, 2H), 2.86 (br d, J = 11.3 Hz, 2H), 2.77-2.61 (m, 1H), 2.49-2.41 (m, 1H), 2.18 (br t, J = 11.4 Hz, 2H), 1.78-1.67 (m, 2H), 1.67-1.35 (m, 4H), 1.36-1.14 (m, 2H), 0.98 (br d, J = 6.6 Hz, 6H), 0.84 (br dd, J = 8.5, 3.7 Hz, 3H). Two protons were not visible due to water suppression. LC-MS (ES): m/z = 514.3 [M + H]⁺, RT (min) = 1.32 |
| 41 | 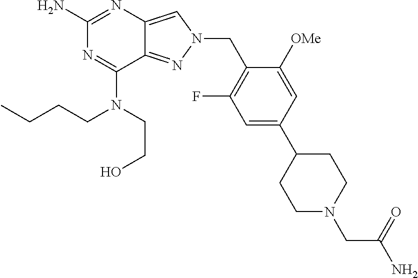 [SMILES: CCCCN(CCO)c1nc(N)nc2cn(Cc3c(F)cc(C4CCN(CC(N)=O)CC4)cc3OC)nc12] | δ 7.75-7.50 (m, 1H), 7.21 (br s, 1H), 7.12 (br s, 1H), 6.80 (br s, 1H), 6.74 (br d, J = 10.4 Hz, 1H), 5.46 (br s, 2H), 5.38 (s, 2H), 4.03 (br dd, J = 8.5, 6.5 Hz, 1H), 3.84 (br s, 3H), 3.75-3.56 (m, 2H), 2.94-2.79 (m, 4H), 2.21-2.09 (m, 2H), 1.81-1.65 (m, 4H), 1.66-1.41 (m, 2H), 1.35-1.11 (m, 2H), 0.98-0.70 (m, 3H). Five protons were not visible due to water suppression and the overlap with DMSO-d₆ peak. LC-MS (ES): m/z = 529.3 [M + H]⁺, RT (min) = 1.46 |
| 42 | 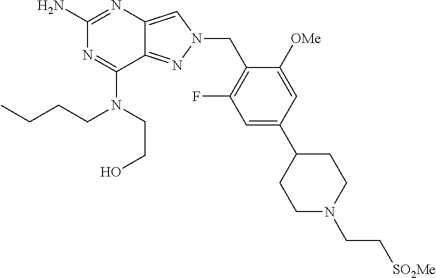 [SMILES: CCCCN(CCO)c1nc(N)nc2cn(Cc3c(F)cc(C4CCN(CCS(C)(=O)=O)CC4)cc3OC)nc12] | δ 7.74-7.51 (m, 1H), 6.79 (s, 1H), 6.74 (br d, J = 9.8 Hz, 1H), 5.47 (br s, 2H), 5.37 (s, 2H), 4.15-3.97 (m, 1H), 3.84 (s, 3H), 3.74-3.52 (m, 2H), 3.29 (br t, J = 6.6 Hz, 2H), 3.04 (s, 3H), 3.03-2.92 (m, 2H), 2.72 (br t, J = 6.7 Hz, 2H), 2.05 (br t, J = 11.0 Hz, 2H), 1.75 (br d, J = 12.0 Hz, 2H), 1.69-1.39 (m, 4H), 1.34-1.10 (m, 2H), 0.98-0.70 (m, 3H). Five protons were not visible due to water suppression and the overlap with DMSO-d₆ peak. LC-MS (ES): m/z = 578.2 [M + H]⁺, RT (min) = 1.56 |
| 47 | 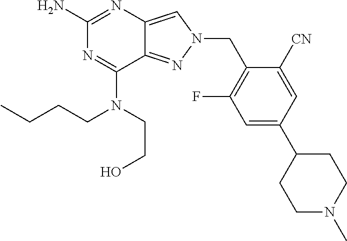 | δ 7.77-7.51 (m, 1H), 6.80 (br s, 1H), 6.73 (br d, J = 10.4 Hz, 1H), 5.61 (br d, J = 2.1 Hz, 2H), 5.39 (s, 2H), 4.20-3.96 (m, 2H), 3.85 (br s, 3H), 3.74-3.55 (m, 2H), 2.89 (br d, J = 11.9 Hz, 2H), 2.48 (br s, 1H), 2.22 (s, 3H), 1.99 (br t, J = 10.5 Hz, 2H), 1.81-1.60 (m, 4H), 1.61- 1.42 (m, 2H), 1.36-1.13 (m, 2H), 0.98-0.75 (m, 3H). Two protons were not visible due to water suppression and one exchangeable proton was not visible either. LC-MS (ES): m/z = 486.5 [M + H]⁺, RT (min) = 1.24 |
| 110 | 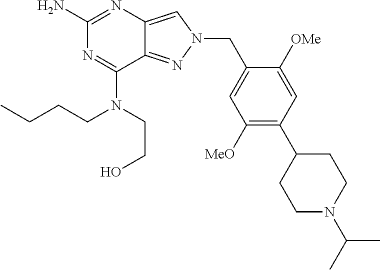 [SMILES: CCCCN(CCO)c1nc(N)nc2cn(Cc3cc(OC)c(C4CCN(C(C)C)CC4)cc3OC)nc12] | δ 7.72 (br s, 2H), 7.02-6.91 (m, 1H), 6.81 (br d, J = 4.6 Hz, 1H), 5.54-5.44 (m, 2H), 4.40- 3.95 (m, 4H), 3.92-3.62 (m, 8H), 3.65-3.31 (m, 4H), 3.24-3.14 (m, 2H), 3.15-3.02 (m, 2H), 2.13-1.81 (m, 4H), 1.72-1.49 (m, 2H), 1.40-1.16 (m, 8H), 0.99-0.72 (m, 3H); LC- MS (ES): m/z = 526.2 [M + H]⁺, RT (min) = 1.44 |

The following compounds were made by following a similar procedure for Compound 37.

| Cmpd | | Analytical Data (¹H NMR 500 MHz, DMSO- |
|---|---|---|
| No. | Structure | d₆; LC-MS) |
| 164 | 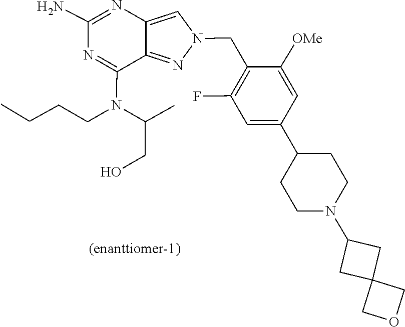 [SMILES: CCCCN(c1nc(N)nc2cn(Cc3c(F)cc(C4CCN(C5CC6(COC6)C5)CC4)cc3OC)nc12)C(C)CO] | δ 7.65 (br s, 1H), 6.78 (s, 1H), 6.72 (br d, J = 10.4 Hz, 1H), 5.45 (br s, 2H), 5.37 (br s, 2H), 4.58 (s, 2H), 4.45 (s, 2H), 3.84 (s, 3H), 2.86 (br d, J = 11.0 Hz, 2H), 2.50-2.41 (m, 2H), 2.36- 2.24 (m, 2H), 1.97-1.91 (m, 2H), 1.80-1.67 (m, 4H), 1.67-1.39 (m, 4H), 1.36-1.13 (m, 3H), 1.13-0.69 (m, 7H). Four protons were not visible due to water suppression and the overlap with DMSO-d₆ peak. LC-MS (ES): m/z = 582.4 [M + H]⁺, RT (min) = 1.55 |
| 179 | 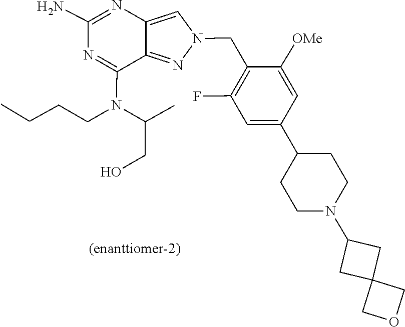 [SMILES: CCCCN(c1nc(N)nc2cn(Cc3c(F)cc(C4CCN(C5CC6(COC6)C5)CC4)cc3OC)nc12)C(C)CO] | δ 7.65 (br s, 1H), 6.78 (s, 1H), 6.72 (br d, J = 10.4 Hz, 1H), 5.45 (br s, 2H), 5.37 (br s, 2H), 4.58 (s, 2H), 4.45 (s, 2H), 3.84 (s, 3H), 2.86 (br d, J = 11.0 Hz, 2H), 2.50-2.41 (m, 2H), 2.36- 2.24 (m, 2H), 1.97-1.91 (m, 2H), 1.80-1.67 (m, 4H), 1.67-1.39 (m, 4H), 1.36-1.13 (m, 3H), 1.13-0.69 (m, 7H). Four protons were not visible due to water suppression and the overlap with DMSO-d₆ peak. LC-MS (ES): m/z = 582.4 [M + H]⁺, RT (min) = 1.55 |

The following compounds were prepared following the experimental procedure for Example S1.

| Cmpd | | Analytical Data (¹H NMR 500 MHz, DMSO- |
|---|---|---|
| No. | Structure | d₆; LC-MS) |
| 72 | 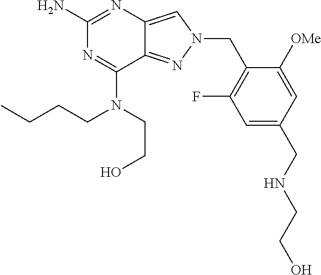 [SMILES: CCCCN(CCO)c1nc(N)nc2cn(Cc3c(F)cc(CNCCO)cc3OC)nc12] | δ 8.33-8.18 (m, 1H), 7.72 (br s, 1H), 6.94 (br s, 1H), 6.87 (br d, J = 9.8 Hz, 1H), 6.03-5.83 (m, 2H), 5.42 (s, 2H), 4.15-4.00 (m, 2H), 3.84 (br s, 3H), 3.79 (s, 2H), 3.73-3.56 (m, 2H), 2.62 (br t, J = 5.8 Hz, 2H), 1.69-1.45 (m, 2H), 1.36-1.12 (m, 2H), 1.00-0.66 (m, 3H). LC- MS (ES): m/z = 462.2 [M + H]⁺, RT (min) = 1.20 |
| 73 | 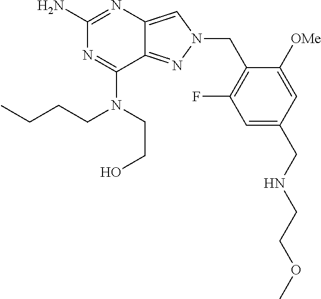 [SMILES: CCCCN(CCO)c1nc(N)nc2cn(Cc3c(F)cc(CNCCOC)cc3OC)nc12] | δ 8.18-7.92 (m, 1H), 7.79 (br s, 2H), 7.14 (s, 1H), 7.05 (br d, J = 9.8 Hz, 1H), 5.61-5.41 (m, 2H), 4.23-4.12 (m, 3H), 4.12-4.06 (m, 1H), 3.94-3.83 (m, 3H), 3.82-3.74 (m, 2H), 3.72- 3.65 (m, 1H), 3.65-3.50 (m, 2H), 3.09 (br d, J = 4.9 Hz, 2H), 1.69-1.59 (m, 1H), 1.59-1.49 (m, 2H), 1.38-1.28 (m, 1H), 1.27-1.17 (m, 2H), 0.92 (t, J = 7.3 Hz, 2H), 0.86 (t, J = 7.5 Hz, 3H). Two protons were not visible due to water suppression. LC-MS (ES): m/z = 476.2 [M + H]⁺, RT (min) = 1.51 |
| 75 | 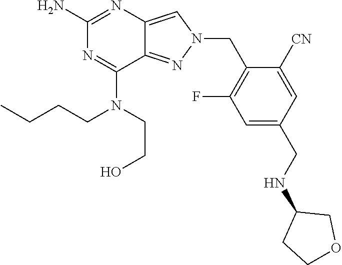 [SMILES: CCCCN(CCO)c1nc(N)nc2cn(Cc3c(F)cc(CN[C@@H]4CCOC4)cc3C#N)nc12] | δ 7.67 (br s, 1H), 6.91 (br s, 1H), 6.85 (br d, J = 10.4 Hz, 1H), 5.58 (br s, 2H), 5.40 (s, 2H), 4.27-3.94 (m, 2H), 3.85 (s, 3H), 3.80-3.49 (m, 6H), 3.23 (br d, J = 4.3 Hz, 1H), 1.99-1.92 (m, 1H), 1.73-1.63 (m, 1H), 1.63-1.39 (m, 2H), 1.38-1.10 (m, 2H), 0.97-0.67 (m, 3H). Six protons were not visible due to water suppression and the overlap with DMSO-d₆ peak. |
| 82 | 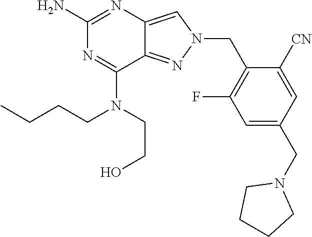 [SMILES: CCCCN(CCO)c1nc(N)nc2cn(Cc3c(F)cc(CN4CCCC4)cc3C#N)nc12] | δ 8.17-7.96 (m, 1H), 7.78 (br s, 2H), 7.21- 6.99 (m, 2H), 5.64-5.44 (m, 2H), 4.46-4.25 (m, 2H), 4.21-4.00 (m, 2H), 3.98-3.83 (m, 4H), 3.83-3.74 (m, 1H), 3.73-3.53 (m, 1H), 2.16-1.75 (m, 4H), 1.68-1.44 (m, 2H), 1.38- 1.12 (m, 2H), 0.96-0.73 (m, 3H). Six protons were not visible due to water suppression and the overlap with DMSO-d₆ peak |
| 84 | 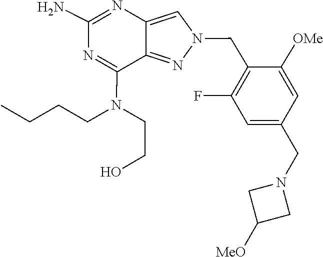 [SMILES: CCCCN(CCO)c1nc(N)nc2cn(Cc3c(F)cc(CN4CC(OC)C4)cc3OC)nc12] | δ 7.82-7.61 (m, 1H), 6.81 (br s, 1H), 6.75 (br d, J = 9.8 Hz, 1H), 6.09 (br s, 2H), 5.41 (s, 2H), 4.18-3.93 (m, 3H), 3.83 (br s, 3H), 3.76-3.61 (m, 2H), 3.14 (s, 3H), 2.86 (br t, J = 6.7 Hz, 2H), 1.71-1.41 (m, 2H), 1.38-1.11 (m, 2H), 1.02- 0.66 (m, 3H). Seven protons were not visible due to water suppression and the overlap with DMSO-d₆ peak |
| 161 | 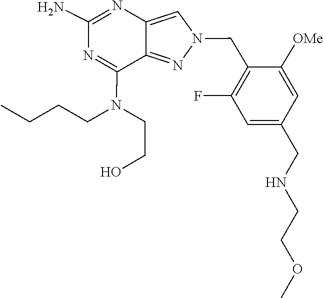 | δ 7.83 (br d, J = 5.2 Hz, 1H), 7.42-7.14 (m, 4H), 5.64 (br s, 2H), 5.43 (s, 2H), 4.24-4.03 (m, 1H), 3.81-3.58 (m, 3H), 3.22 (s, 3H), 2.65 (br t, J = 5.5 Hz, 2H), 1.67-1.48 (m, 2H), 1.39- 1.22 (m, 2H), 1.00-0.68 (m, J = 3.1 Hz, 3H). Eight protons were not visible due to water suppression and the overlap with DMSO-d₆ peak. LC-MS (ES): m/z = 428.2 [M + H]⁺, RT (min) = 1.15 |

### Example S10. 2-((5-Amino-2-((5-((cyclobutylamino)methyl)quinolin-8-yl)methyl)-2H-pyrazolo[4,3-d]pyrimidin-7-yl)(butyl)amino)ethan-1-ol (Compound 46)

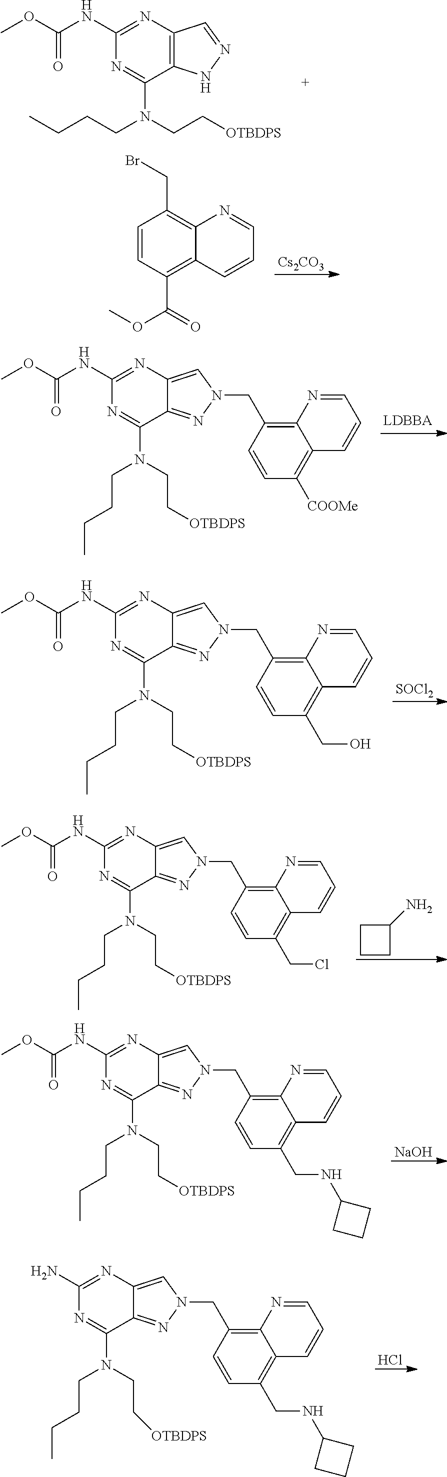

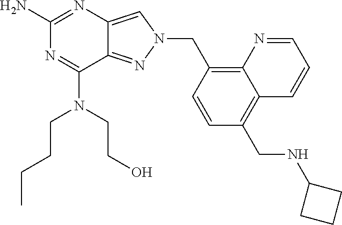 [SMILES: CCCCN(CCO)c1nc(N)nc2cn(Cc3ccc(CNC4CCC4)c4cccnc34)nc12]

Step 1. methyl 8-((7-(butyl(2-((tert-butyldiphenylsilyl)oxy)ethyl)amino)-5-((methoxycarbonyl)amino)-2H-pyrazolo[4,3-d]pyrimidin-2-yl)methyl)quinoline-5-carboxylate: To a solution of methyl (7-(butyl(2-((tert-butyldiphenylsilyl)oxy)ethyl)amino)-1H-pyrazolo[4,3-d]pyrimidin-5-yl)carbamate (371 mg, 0.679 mmol) and methyl 8-(bromomethyl)quinoline-5-carboxylate (190 mg, 0.679 mmol) in DMF (3 mL), cesium carbonate (442 mg, 1.357 mmol) was added. The reaction mixture was stirred at RT for 2 h. LCMS analysis showed the reaction completed (2.462 min at 3 min acidic run, M+H/z=746.5). The reaction mixture was purified by preparative HPLC with the following conditions: Xbridge C18, 19×150 mm, 5 μm particles; mobile phase A: water with 0.05% TFA; mobile phase B: acetonitrile with 0.05% TFA; gradient: 0-2 min at 50% B, 50-90% B over 25 minutes, then a 6-minute hold at 100% B; flow Rate: 18.8 mL/min; column temperature: 25° C. The fractions containing product were combined and freeze-dried to yield methyl 8-((7-(butyl(2-((tert-butyldiphenylsilyl)oxy)ethyl)amino)-5-((methoxycarbonyl)amino)-2H-pyrazolo[4,3-d]pyrimidin-2-yl)methyl)quinoline-5-carboxylate (351 mg, 0.471 mmol, 69.3% yield).

Step 2. methyl (7-(butyl(2-((tert-butyldiphenylsilyl)oxy)ethyl)amino)-2-((5-(hydroxymethyl)quinolin-8-yl)methyl)-2H-pyrazolo[4,3-d]pyrimidin-5-yl)carbamate: To a solution of this compound (170 mg, 0.228 mmol) in THE (1 mL), LDBBA in THF/hexanes (5.47 mL, 1.367 mmol) was added. The reaction mixture was stirred at RT for 1 h. LCMS analysis showed the reaction 80% completed (2.305 at 3 min acidic run, M+H/z=718.5). The reaction mixture was stirred with a solution of Rochelle salt and worked up with EtOAc, water and brine. The organic layer was concentrated. The residue was purified with preparative HPLC with following conditions: Xbridge C18, 19×150 mm, 5 μm particles; mobile phase A: water with 0.05% TFA; mobile phase B: acetonitrile with 0.05% TFA; gradient: 0-2 min at 50% B, 50-90% B over 25 minutes, then a 6-minute hold at 100% B; flow rate: 18.8 mL/min; column temperature: 25° C. The fractions containing product were combined and freeze-dried to yield methyl (7-(butyl(2-((tert-butyldiphenylsilyl)oxy)ethyl)amino)-2-((5-(hydroxymethyl)quinolin-8-yl)methyl)-2H-pyrazolo[4,3-d]pyrimidin-5-yl)carbamate (101 mg, 0.141 mmol, 61.7% yield).

Step 3. methyl (7-(butyl(2-((tert-butyldiphenylsilyl)oxy)ethyl)amino)-2-((5-(chloromethyl)quinolin-8-yl)methyl)-2H-pyrazolo[4,3-d]pyrimidin-5-yl)carbamate: To a solution of methyl (7-(butyl(2-((tert-butyldiphenylsilyl)oxy)ethyl)amino)-2-((5-(hydroxymethyl)quinolin-8-yl)methyl)-2H-pyrazolo[4,3-d]pyrimidin-5-yl)carbamate (60 mg) in THE (1 mL), thionyl chloride (0.100 mL, 1.367 mmol) was added. The reaction mixture was stirred at RT for 15 mins. LCMS analysis showed the reaction completed (2.395 min at 3 min acidic run, M+H/z=736.5. The reaction was concentrated and co-evaporated with DCM (2×5 mL) and dried under high vacuum for 15 min to yield crude methyl (7-(butyl(2-((tert-butyldiphenylsilyl)oxy)ethyl)amino)-2-((5-(chloromethyl)quinolin-8-yl)methyl)-2H-pyrazolo[4,3-d]pyrimidin-5-yl)carbamate (68 mg). The material was used for the next reaction without further purification.

Step 4. methyl (7-(butyl(2-((tert-butyldiphenylsilyl)oxy)ethyl)amino)-2-((5-((cyclobutylamino)methyl)quinolin-8-yl)methyl)-2H-pyrazolo[4,3-d]pyrimidin-5-yl)carbamate: To a solution of methyl (7-(butyl(2-((tert-butyldiphenylsilyl)oxy)ethyl)amino)-2-((5-(chloromethyl)quinolin-8-yl)methyl)-2H-pyrazolo[4,3-d]pyrimidin-5-yl)carbamate (20 mg, 0.027 mmol) and cyclobutanamine (29.0 mg, 0.407 mmol) in DMF (0.5 mL), DIPEA (0.014 mL, 0.081 mmol) was added. The reaction mixture was stirred at RT for 4 hr. LCMS analysis showed the reaction completed (1.920 at 3 min acidic run, M+H/z=771.6). The reaction mixture was freeze-dried with ACN and water to yield crude methyl (7-(butyl(2-((tert-butyldiphenylsilyl)oxy)ethyl)amino)-2-((5-((cyclobutylamino)methyl)quinolin-8-yl)methyl)-2H-pyrazolo[4,3-d]pyrimidin-5-yl)carbamate (24 mg).

Step 5. N7-butyl-N7-(2-((tert-butyldiphenylsilyl)oxy)ethyl)-2-((5-((cyclobutylamino)methyl)quinolin-8-yl)methyl)-2H-pyrazolo[4,3-d]pyrimidine-5,7-diamine: To a solution of crude methyl (7-(butyl(2-((tert-butyldiphenylsilyl)oxy)ethyl)amino)-2-((5-((cyclobutylamino)methyl)quinolin-8-yl)methyl)-2H-pyrazolo[4,3-d]pyrimidin-5-yl)carbamate (24 mg) in 1,4-dioxane (0.5 mL), NaOH (10 N, 0.3 mL) was added. The reaction was stirred at 70° C. for overnight. LCMS analysis showed the reaction completed (2.137 min at 3 min acidic run, M+H/z=713.5). The reaction mixture was neutralized with acetic acid and freeze-dried with ACN and water to yield crude N7-butyl-N7-(2-((tert-butyldiphenylsilyl)oxy)ethyl)-2-((5-((cyclobutylamino)methyl)quinolin-8-yl)methyl)-2H-pyrazolo[4,3-d]pyrimidine-5,7-diamine.

Step 6. 2-((5-amino-2-((5-((cyclobutylamino)methyl)quinolin-8-yl)methyl)-2H-pyrazolo[4,3-d]pyrimidin-7-yl)(butyl)amino)ethan-1-ol: N7-butyl-N7-(2-((tert-butyldiphenylsilyl)oxy)ethyl)-2-((5-((cyclobutylamino)methyl)quinolin-8-yl)methyl)-2H-pyrazolo[4,3-d]pyrimidine-5,7-diamine was dissolved in methanol (0.5 mL). HCl (12 N, 0.3 mL) was added. The reaction mixture was stirred at RT for 30 min. LCMS analysis showed the reaction completed (1.057 min at 3 min acidic run, M+H/z=475.4). The crude material was purified via preparative LC/MS with the following conditions: column: XBridge C18, 200 mm×19 mm, 5 μm particles; mobile phase A: 5:95 acetonitrile:water with ammonium acetate; mobile phase B: 95:5 acetonitrile:water with ammonium acetate; gradient: a 0-minute hold at 20% B, 20-60% B over 20 minutes, then a 0-minute hold at 100% B; flow Rate: 20 mL/min; column temperature: 25° C. Fraction collection was triggered by MS signals. Fractions containing the desired product were combined and dried via centrifugal evaporation to yield 2-((5-amino-2-((5-((cyclobutylamino)methyl)quinolin-8-yl)methyl)-2H-pyrazolo[4,3-d]pyrimidin-7-yl)(butyl)amino)ethan-1-ol (4.6 mg, 0.01 mmol, 33.6% for 3 steps).

| Cmpd | | Analytical Data (¹H NMR 500 MHz, DMSO- |
|---|---|---|
| No. | Structure | d₆; LC-MS) |
| 46 | 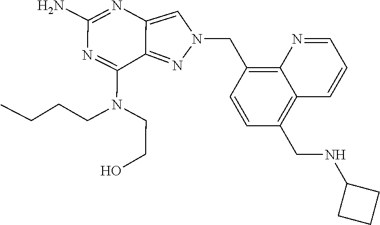 [SMILES: CCCCN(CCO)c1nc(N)nc2cn(Cc3ccc(CNC4CCC4)c4cccnc34)nc12] | δ 9.00 (s, 1H), 8.71-8.65 (m, 1H), 7.86 (s, 1H), 7.63 (dd, J = 8.7, 4.1 Hz, 1H), 7.50 (d, J = 7.3 Hz, 1H), 7.28 (s, 1H), 6.08 (s, 2H), 5.50 (s, 1H), 4.05 (s, 2H), 3.19 (s, 1H), 2.08 (d, J = 8.7 Hz, 2H), 1.90 (s, 5H), 1.70 (t, J = 9.4 Hz, 2H), 1.62 (t, J = 9.5 Hz, 1H), 1.52 (d, J = 10.1 Hz, 3H), 1.26 (s, 1H), 1.19 (s, 1H), 0.93 (s, 1H), 0.80 (s, 3H). LC-MS (ES): m/z = 475.4 [M + H] , RT (min) = 1.44 |

The following compounds were prepared following the procedure for Compound 46.

| Cmpd | | Analytical Data (¹H NMR 500 MHz, DMSO-d₆; |
|---|---|---|
| No. | Structure | LC-MS) |
| 45 | 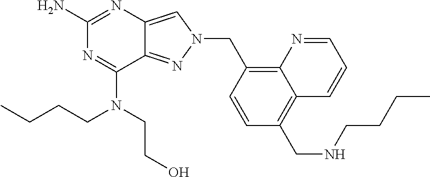 [SMILES: CCCCNCc1ccc(Cn2cc3nc(N)nc(N(CCO)CCCC)c3n2)c2ncccc12] | δ 9.00 (s, 1H), 8.70 (d, J = 8.8 Hz, 1H), 7.88 (s, 1H), 7.63 (dd, J = 8.4, 4.1 Hz, 1H), 7.54 (d, J = 7.3 Hz, 1H), 7.31 (s, 1H), 6.08 (s, 2H), 5.65 (s, 1H), 4.16 (s, 2H), 4.07 (s, 1H), 2.59 (t, J = 6.9 Hz, 2H), 1.91 (s, 4H), 1.51 (s, 2H), 1.43 (p, J = 7.2 Hz, 2H), 1.29 (q, J = 7.4 Hz, 2H), 1.17 (s, 2H), 0.92 (s, 1H), 0.85 (t, J = 7.3 Hz, 3H), 0.78 (s, 3H). LC-MS (ES): m/z = 477.6 [M + H]⁺, RT (min) = 1.45 |
| 48 | 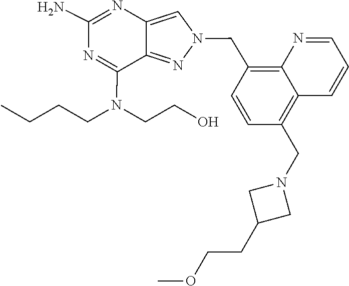 [SMILES: CCCCN(CCO)c1nc(N)nc2cn(Cc3ccc(CN4CC(CCOC)C4)c4cccnc34)nc12] | δ 9.00 (s, 1H), 8.66 (d, J = 8.5 Hz, 1H), 7.88 (s, 1H), 7.63 (dd, J = 8.7, 4.2 Hz, 1H), 7.46 (d, J = 7.3 Hz, 1H), 7.24 (s, 1H), 6.07 (s, 2H), 5.51 (s, 1H), 4.08 (s, 1H), 3.93 (d, J = 16.6 Hz, 2H), 3.30 (d, J = 7.2 Hz, 1H), 3.24 (t, J = 6.5 Hz, 1H), 3.18 (s, 2H), 2.80 (t, J = 6.8 Hz, 2H), 2.43-2.36 (m, 1H), 1.92 (s, 4H), 1.72 (q, J = 6.7 Hz, 2H), 1.51 (s, 2H), 1.32 (s, 1H), 1.17 (s, 2H), 0.92 (s, 2H), 0.77 (s, 3H). LC-MS (ES): m/z = 519.6 [M + H]⁺, RT (min) = 1.40 |
| 58 | 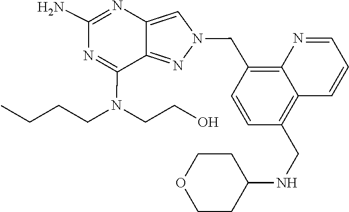 [SMILES: CCCCN(CCO)c1nc(N)nc2cn(Cc3ccc(CNC4CCOCC4)c4cccnc34)nc12] | δ 9.00 (s, 1H), 8.70 (d, J = 8.4 Hz, 1H), 7.90 (s, 1H), 7.63 (dd, J = 8.5, 4.3 Hz, 1H), 7.57 (d, J = 7.3 Hz, 1H), 7.33 (s, 1H), 6.08 (s, 2H), 5.80 (s, 2H), 4.19 (s, 2H), 4.08 (s, 1H), 3.83 (d, J = 11.5 Hz, 2H), 3.64 (s, 2H), 3.28 (t, J = 11.4 Hz, 1H), 3.18 (s, 1H), 2.71 (s, 1H), 1.92 (s, 4H), 1.84 (d, J = 12.9 Hz, 2H), 1.52 (s, 2H), 1.32 (d, J = 12.5 Hz, 3H), 1.18 (s, 2H), 0.92 (s, 1H), 0.79 (s, 2H). LC- MS (ES): m/z = 505.1 [M + H]⁺, RT (min) = 1.56 |
| 63 | 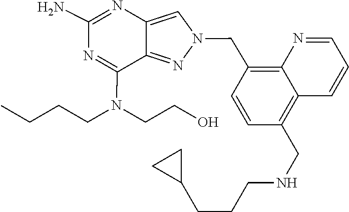 [SMILES: CCCCN(CCO)c1nc(N)nc2cn(Cc3ccc(CNCCCC4CC4)c4cccnc34)nc12] | δ 9.05 (s, 1H), 8.75 (d, J = 8.4 Hz, 1H), 7.92 (s, 1H), 7.68 (dd, J = 8.5, 4.3 Hz, 1H), 7.58 (d, J = 7.3 Hz, 1H), 7.34 (s, 1H), 6.13 (s, 2H), 5.62 (s, 1H), 4.18 (s, 2H), 4.13 (s, 1H), 2.64 (t, J = 7.1 Hz, 2H), 1.96 (s, 5H), 1.61-1.55 (m, 4H), 1.35 (s, 2H), 1.24 (q, J = 7.1 Hz, 3H), 0.97 (s, 2H), 0.84 (s, 3H), 0.68 (s, 1H), 0.39 (dd, J = 7.8, 1.8 Hz, 2H). LC-MS (ES): m/z = 503.1 [M + H]⁺, RT (min) = 1.74 |
| 60 | 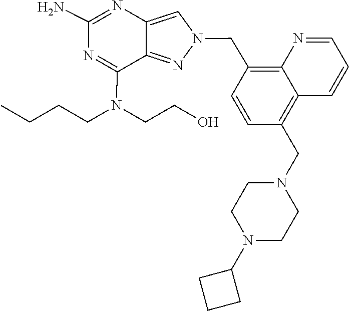 [SMILES: CCCCN(CCO)c1nc(N)nc2cn(Cc3ccc(CN4CCN(C5CCC5)CC4)c4cccnc34)nc12] | δ 8.99 (s, 1H), 8.73 (d, J = 8.5 Hz, 1H), 7.96 (s, 1H), 7.63 (dd, J = 8.6, 4.3 Hz, 1H), 7.45 (d, J = 7.3 Hz, 1H), 7.28 (s, 1H), 7.23 (s, 1H), 6.08 (s, 2H), 5.97(s, 1H), 4.12 (s, 1H), 4.06 (s, 1H), 3.85 (s, 2H), 3.64 (s, 1H), 2.69-2.62 (m, 1H), 2.40 (s, 4H), 2.18 (s, 3H), 1.91 (d, J = 0.8 Hz, 3H), 1.74 (t, J = 9.8 Hz, 2H), 1.60 (s, 4H), 1.48 (s, 2H), 1.32 (s, 1H), 1.13(s, 2H), 0.92 (s, 1H), 0.73 (s, 3H). LC-MS (ES): m/z = 544.3 [M + H]⁺, RT (min) = 1.61 |

### Example S11. 2-((5-Amino-2-((7-(((tetrahydro-2H-pyran-4-yl)amino)methyl)benzo[d]thiazol-4-yl)methyl)-2H-pyrazolo[4,3-d]pyrimidin-7-yl)(butyl)amino)ethan-1-ol (Compound 180)

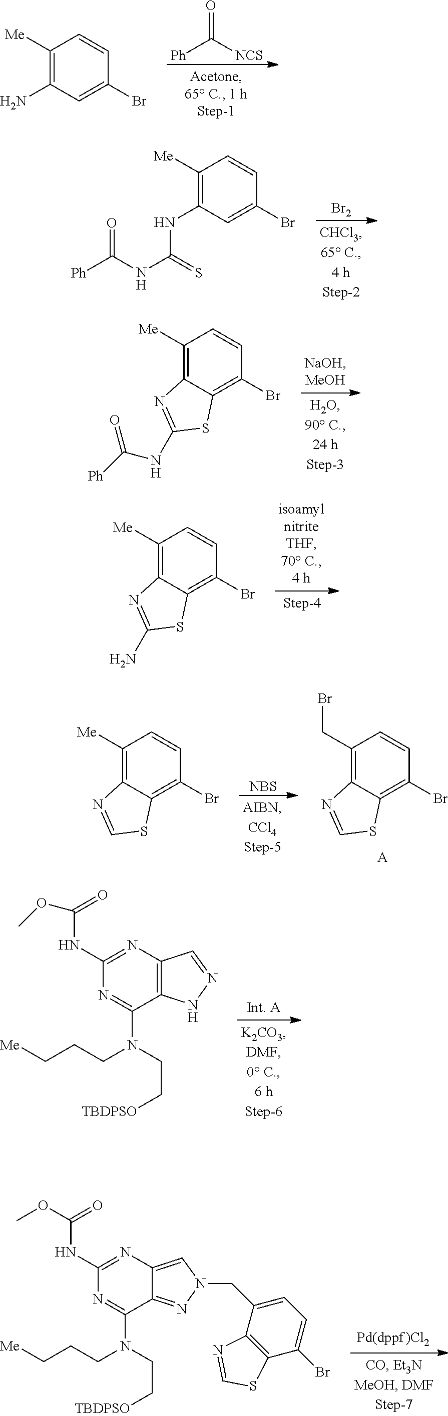

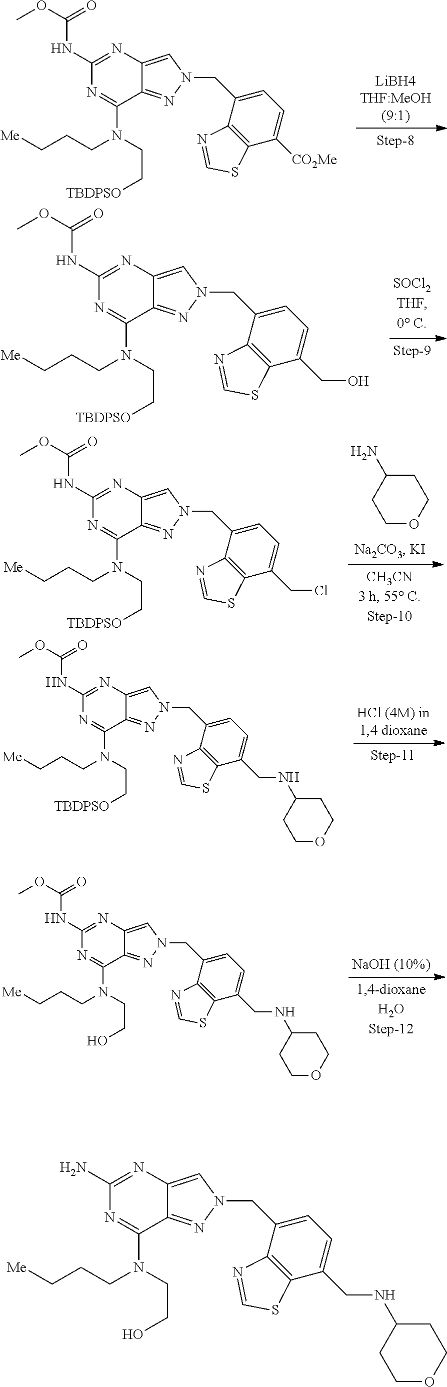

Step 1. N-((5-Bromo-2-methylphenyl)carbamothioyl)benzamide: To a stirred solution of 5-bromo-2-methylaniline (5 g, 26.9 mmol) in acetone (80 mL), was added benzoyl isothiocyanate (3.62 mL, 26.9 mmol) at RT. The reaction mixture was stirred at 65° C. for 1 h. The reaction mixture was allowed to cool to RT and concentrated under reduced pressure to afford title compound (9 g, 25.8 mmol, 96% yield) as a light yellow solid. ¹H NMR (500 MHz, DMSO-d₆) δ=12.36 (s, 1H), 11.71 (s, 1H), 8.00 (br d, J=7.6 Hz, 2H), 7.90 (s, 1H), 7.71-7.62 (m, 1H), 7.58-7.51 (m, 2H), 7.42 (br d, J=8.2 Hz, 1H), 7.28 (d, J=7.9 Hz, 1H), 2.24 (s, 3H). LC-MS (ES): m/z=349.05 [M+H]⁺

Step 2. N-(7-Bromo-4-methylbenzo[d]thiazol-2-yl)benzamide: To a stirred solution of N-((5-bromo-2-methylphenyl)carbamothioyl)benzamide (5 g, 14.32 mmol) in chloroform (120 mL) at 0° C., was added Br₂ (1.991 mL, 38.7 mmol) as CHCl₃ solution (20 mL) via a dropping funnel. The reaction mixture was warmed to RT, stirred at 65° C. for 3 h and concentrated under the reduced pressure. The residue was washed with ethyl acetate. The precipitated solid was filtered and was washed with pet. ether to afford the title compound (3.2 g, 9.22 mmol, 64.4% yield) as a light yellow solid. ¹H NMR (500 MHz, DMSO-d₆) δ=13.68-12.56 (m, 1H), 8.16 (d, J=7.3 Hz, 2H), 7.72-7.64 (m, 1H), 7.57 (t, J=7.6 Hz, 2H), 7.45 (d, J=7.9 Hz, 1H), 7.27 (d, J=7.6 Hz, 1H), 2.59 (s, 3H). LC-MS (ES): m/z=349.05 [M+H]⁺.

Step 3. 7-Bromo-4-methylbenzo[d]thiazol-2-amine: To a stirred solution of N-(7-bromo-4-methylbenzo[d]thiazol-2-yl)benzamide (3.1 g, 8.93 mmol) in methanol (40 mL) and water (40 mL), was added NaOH (3.57 g, 89 mmol). The reaction mixture was heated at 90° C. for 24 h. After evaporating the solvent, the residue was partitioned between DCM and water. The organic layer was washed with brine solution, dried over anhydrous Na₂SO₄, filtered, and concentrated under vacuum to afford the title compound (1.7 g, 6.99 mmol, 78% yield) as a white solid. ¹H NMR (500 MHz, DMSO-d₆) δ=7.72 (s, 2H), 7.09 (d, J=7.9 Hz, 1H), 7.03-6.95 (m, 1H), 2.36 (s, 3H). LC-MS (ES): m/z=245.00 [M+H]⁺.

Step 4. 7-Bromo-4-methylbenzo[d]thiazole: To a stirred solution of 7-bromo-4-methylbenzo[d]thiazol-2-amine (1.7 g, 6.99 mmol) in THE (30 mL), was added isoamyl nitrite (1.412 mL, 10.49 mmol) under N₂ atmosphere. The mixture was stirred at 65° C. for 4 h and then partitioned between water and EtOAc. The organic layer was washed with brine solution, dried over anhydrous Na₂SO₄, filtered and concentrated under vacuum to afford the title compound (1.5 g, 6.58 mmol, 94% yield) as a light yellow solid. ¹H NMR (400 MHz, DMSO-d₆) δ=9.46 (s, 1H), 7.61 (d, J=7.8 Hz, 1H), 7.34 (d, J=7.8 Hz, 1H), 2.65 (s, 3H). LC-MS (ES): m/z=228.00 [M+H]⁺

Step 5. 7-Bromo-4-(bromomethyl)benzo[d]thiazole: To a stirred solution of 7-bromo-4-methylbenzo[d]thiazole (1.5 g, 6.58 mmol) in carbon tetrachloride (50 mL), was added NBS (1.756 g, 9.86 mmol) and AIBN (0.324 g, 1.973 mmol). After stirring at 70° C. under N₂ for 24 h, the reaction mixture was filtered through a Celite pad and the filtrate was concentrated under reduced pressure. The residue was purified using CombiFlash column chromatography (Redisep Rf silica gel 20 g column; 10% EtOAc in pet. ether as an eluent) to afford the title compound (1 g, 3.26 mmol, 49.5% yield) as a light yellow solid. ¹H NMR (400 MHz, DMSO-d₆) δ=9.59 (s, 1H), 7.75 (d, J=7.9 Hz, 1H), 7.64 (d, J=8.1 Hz, 1H), 5.12 (s, 2H). LC-MS (ES): m/z=307.90 [M+H]⁺.

Step 6. Methyl (2-((7-bromobenzo[d]thiazol-4-yl)methyl)-7-(butyl(2-((tert-butyldiphenylsilyl)oxy)ethyl)amino)-2H-pyrazolo[4,3-d]pyrimidin-5-yl)carbamate: To a stirred solution of methyl (7-(butyl(2-((tert-butyldiphenylsilyl)oxy)ethyl)amino)-1H-pyrazolo[4,3-d]pyrimidin-5-yl)carbamate (2.0 g, 2.74 mmol) and 7-bromo-4-(bromomethyl)benzo[d]thiazole (1.011 g, 3.29 mmol) in dry DMF (20 mL), was added potassium carbonate (0.758 g, 5.49 mmol) at 0° C. The reaction mixture was stirred for 3 h at RT and then partitioned between water and EtOAc. The organic layer was washed with brine solution, dried over anhydrous Na₂SO₄, filtered, and concentrated under vacuum. The residue was purified using CombiFlash column chromatography (Redisep Rf silica gel 20 g column; 5% methanol in DCM as an eluent) to afford the title compound (1.20 g, 1.553 mmol, 56.6% yield) as a light brown foam. ¹H NMR (400 MHz, DMSO-d₆) δ=9.57-9.49 (m, 1H), 9.49-9.30 (m, 1H), 8.40-8.17 (m, 1H), 7.56-7.44 (m, 4H), 7.41-7.32 (m, 3H), 7.32-7.22 (m, 5H), 6.16-5.84 (m, 2H), 4.32-4.23 (m, 1H), 4.07-3.99 (m, 1H), 3.92 (br d, J=5.5 Hz, 1H), 3.85-3.71 (m, 3H), 3.65-3.60 (m, 1H), 3.54-3.54 (m, 1H), 3.59-3.52 (m, 1H), 1.70-1.55 (m, 1H), 1.46-1.35 (m, 1H), 1.35-1.25 (m, 1H), 1.08-1.00 (m, 1H), 0.95-0.86 (m, 7H), 0.84-0.80 (m, 3H), 0.73-0.64 (m, 2H). LC-MS (ES): m/z=774.45 [M+H]⁺.

Step 7. Methyl 4-((7-(butyl(2-((tert-butyldiphenylsilyl)oxy)ethyl)amino)-5-((methoxycarbonyl)amino)-2H-pyrazolo[4,3-d]pyrimidin-2-yl)methyl)benzo[d]thiazole-7-carboxylate: To a stirred solution of methyl (2-((7-bromobenzo[d]thiazol-4-yl)methyl)-7-(butyl(2-((tert-butyldiphenylsilyl)oxy)ethyl)amino)-2H-pyrazolo[4,3-d]pyrimidin-5-yl)carbamate (0.650 g, 0.841 mmol) in dry methanol (10 mL) and DMF (10 mL), was added Et₃N (0.586 mL, 4.21 mmol) and PdCl₂(dppf)-CH₂Cl₂ adduct (0.206 g, 0.252 mmol). The reaction mixture was heated at 95° C. under CO gas (10 Kg) in tiny clave for 16 h. The reaction mixture was filtered through a pad of Celite, which was washed with 5% MeOH in DCM. The filtrate was concentrated. The residue was purified using CombiFlash column chromatography (Redisep Rf silica gel 10 g column; 5% methanol in DCM as an eluent) to afford the title compound (0.400 g, 0.532 mmol, 63.2% yield) as a light brown foam. ¹H NMR (400 MHz, DMSO-d₆) δ=9.62-9.52 (m, 1H), 8.43-8.26 (m, 1H), 8.14-7.92 (m, 1H), 7.63-7.59 (m, 1H), 7.60-7.41 (m, 4H), 7.40-7.31 (m, 3H), 7.30-7.18 (m, 4H), 6.28-6.02 (m, 2H), 4.34-4.21 (m, 1H), 4.08-3.99 (m, 1H), 3.94-3.88 (m, 1H), 3.85-3.70 (m, 3H), 3.66-3.51 (m, 3H), 2.94-2.84 (m, 1H), 2.78-2.71 (m, 1H), 1.71-1.56 (m, 1H), 1.46-1.36 (m, 1H), 1.36-1.25 (m, 1H), 1.09-0.97 (m, 1H), 0.97-0.77 (m, 11H), 0.70-0.56 (m, 2H). LC-MS (ES): m/z=753.05 [M+H]⁺

Step 8. Methyl (7-(butyl(2-((tert-butyldiphenylsilyl)oxy)ethyl)amino)-2-((7-(hydroxymethyl)benzo[d]thiazol-4-yl)methyl)-2H-pyrazolo[4,3-d]pyrimidin-5-yl)carbamate: To a stirred solution of methyl 4-((7-(butyl(2-((tert-butyldiphenylsilyl)oxy)ethyl)amino)-5-((methoxycarbonyl)amino)-2H-pyrazolo[4,3-d]pyrimidin-2-yl)methyl)benzo[d]thiazole-7-carboxylate (0.650 g, 0.864 mmol) in tetrahydrofuran (10 mL) and methanol (1 mL), was added a solution of LiBH₄ (6.48 mL, 12.97 mmol, 2M in THF) at 0° C. The mixture was stirred at RT for 1.5 h. After quenching the reaction mixture with NaHCO₃, the reaction mixture was partitioned between EtOAc and water. The organic layer was dried over Na₂SO₄ and concentrated. The residue was purified using CombiFlash column chromatography (Redisep Rf silica gel 10 g column; 5% methanol in DCM as an eluent) to afford the title compound (0.375 g, 0.518 mmol, 59.9% yield) as a light brown foam. LC-MS (ES): m/z=724.50 [M+H]⁺

Step 9. Methyl (7-(butyl(2-((tert-butyldiphenylsilyl)oxy)ethyl)amino)-2-((7-(chloromethyl)benzo[d]thiazol-4-yl)methyl)-2H-pyrazolo[4,3-d]pyrimidin-5-yl)carbamate: To a stirred solution of methyl (7-(butyl(2-((tert-butyldiphenylsilyl)oxy)ethyl)amino)-2-((7-(hydroxymethyl)benzo[d]thiazol-4-yl)methyl)-2H-pyrazolo[4,3-d]pyrimidin-5-yl)carbamate (0.080 g, 0.111 mmol) in THE (2.0 mL), was added SOCl₂ (0.040 mL, 0.553 mmol) at 0° C. After stirring at the same temperature for 30 min, the mixture was concentrated under reduced pressure. The residue was triturated with diethyl ether (2×5 mL) and dried to afford title compound (0.080 g, 0.107 mmol, 98.0% yield) as a light brown foam. LC-MS (ES): m/z=742.50 [M+H]⁺

Step 10. Methyl (7-(butyl(2-((tert-butyldiphenylsilyl)oxy)ethyl)amino)-2-((7-(((tetrahydro-2H-pyran-4-yl)amino)methyl)benzo[d]thiazol-4-yl)methyl)-2H-pyrazolo[4,3-d]pyrimidin-5-yl)carbamate: To a stirred solution of methyl (7-(butyl(2-((tert-butyldiphenylsilyl)oxy)ethyl)amino)-2-((7-(chloromethyl)benzo[d]thiazol-4-yl)methyl)-2H-pyrazolo[4,3-d]pyrimidin-5-yl)carbamate (0.080 g, 0.108 mmol) in CH₃CN (1.5 mL), were added Na₂CO₃ (0.029 g, 0.269 mmol), KI (1.789 mg, 10.78 μmol) and tetrahydro-2H-pyran-4-amine (0.022 g, 0.216 mmol). The reaction mixture was stirred at 55° C. for 3 h. The reaction mixture was allowed to cool to RT and partitioned between water and EtOAc. The aqueous layer was extracted with EtOAc. The combined organic extracts were washed with brine solution, dried over anhydrous Na₂SO₄, filtered, and concentrated under vacuum to afford title compound (0.085 g, 0.105 mmol, 98.0% yield) as a light brown oil. LC-MS (ES): m/z=807.6 [M+H]⁺

Step 11. Methyl (7-(butyl(2-hydroxyethyl)amino)-2-((7-(((tetrahydro-2H-pyran-4-yl)amino)methyl)benzo[d]thiazol-4-yl)methyl)-2H-pyrazolo[4,3-d]pyrimidin-5-yl)carbamate: To a stirred solution of methyl (7-(butyl(2-((tert-butyldiphenylsilyl)oxy)ethyl)amino)-2-((7-(((tetrahydro-2H-pyran-4-yl)amino)methyl)benzo[d]thiazol-4-yl)methyl)-2H-pyrazolo[4,3-d]pyrimidin-5-yl)carbamate (0.135 g, 0.167 mmol) in dichloromethane (1.5 mL) at 0° C., was added 4M HCl in 1,4-dioxane (2.091 mL, 8.36 mmol). The reaction was warmed to RT, stirred for 3 h and concentrated under reduced pressure. The residue was triturated with diethyl ether (2×5 mL) and dried to afford title compound (0.090 g, 0.158 mmol, 95.0% yield) as a light brown oil. LC-MS (ES): m/z=569.5 [M+H]⁺

Step 12. 2-((5-Amino-2-((7-(((tetrahydro-2H-pyran-4-yl)amino)methyl)benzo[d]thiazol-4-yl)methyl)-2H-pyrazolo[4,3-d]pyrimidin-7-yl)(butyl)amino)ethan-1-ol: To a stirred suspension of methyl (7-(butyl(2-hydroxyethyl)amino)-2-((7-(((tetrahydro-2H-pyran-4-yl)amino)methyl)benzo[d]thiazol-4-yl)methyl)-2H-pyrazolo[4,3-d]pyrimidin-5-yl)carbamate (0.090 g, 0.158 mmol) in dioxane (3 mL), was added an aqueous solution of 10% NaOH (2.59 mL, 7.76 mmol) at RT. The reaction mixture was heated to 90° C. and stirred for 3 h. The organic layer from the reaction mixture was separated and concentrated under reduced pressure. The residue was purified via preparative LC/MS (conditions: column: Waters XBridge C18, 19×150 mm, 5-μm particles; mobile phase A: 10 mM ammonium acetate; mobile phase B: acetonitrile; gradient: 12-54% B over 25 minutes, then a 5-minute hold at 100% B; flow rate: 20 mL/min). Fractions containing the desired product were combined and dried via centrifugal evaporation. The residue was dissolved in a mixture of MeCN and water. The resultant mixture was frozen and lyophilized for 12 h to afford the title compound (12 mg, 0.023 mmol, 14.2% yield) as a white solid.

| Cmpd | | Analytical Data (¹H NMR 400 MHz, DMSO-d₆; |
|---|---|---|
| No. | Structure | LC-MS) |
| 180 | 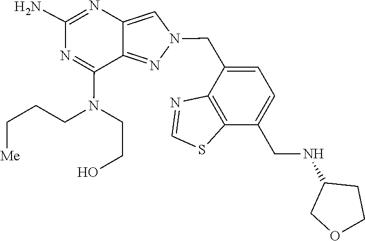 | δ = 9.41 (s, 1H), 7.96-7.78 (m, 1H), 7.37 (d, J = 7.5 Hz, 1H), 7.29-7.12 (m, 1H), 6.02-5.91 (m, 2H), 5.89-5.67 (m, 2H), 5.07-4.45 (m, 1H), 4.19-3.96 (m, 4H), 3.83-3.77 (m, 2H), 3.72-3.59 (m, 4H), 3.22-3.18 (m, 3H), 1.84- 1.73 (m, 2H), 1.63-1.42 (m, 3H), 1.32-1.15 (m, 4H), 0.92-0.75 (m, 3H). LC-MS (ES): m/z = 511.2 [M + H]⁺, RT (min) = 1.37 |

The following compounds were prepared by following the procedure for Compound 180.

| Cmpd | | Analytical Data (¹H NMR 400 MHz, DMSO- |
|---|---|---|
| No. | Structure | d₆; LC-MS) |
| 183 | 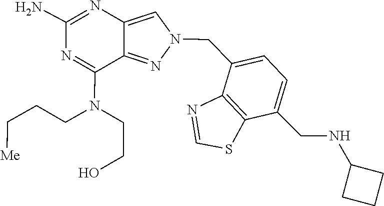 [SMILES: CCCCN(CCO)c1nc(N)nc2cn(Cc3ccc(CNC4CCC4)c4scnc34)nc12] | δ = 9.42 (s, 1H), 8.04-7.88 (m, 1H), 7.45- 7.34 (m, 1H), 7.33-7.20 (m, 1H), 6.57-6.11 (m, 2H), 6.04-5.90 (m, 2H), 4.85-4.61 (m, 1H), 4.17-4.03 (m, 2H), 3.97 (d, J = 6.3 Hz, 2H), 3.81-3.74 (m, 2H), 3.69-3.60 (m, 5H), 3.47 (br d, J = 4.1 Hz, 1H), 2.69-2.62 (m, 1H), 2.35-2.29 (m, 1H), 1.98-1.85 (m, 1H), 1.76- 1.66 (m, 1H), 1.65-1.41 (m, 2H), 1.36- 1.25 (m, 1H), 1.21-1.10 (m, 1H), 0.94-0.75 (m, 3H). LC-MS (ES): m/z = 497.2 [M + H]⁺, RT (min) = 1.34 |
| 184 | 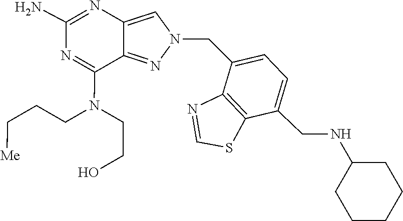 [SMILES: CCCCN(CCO)c1nc(N)nc2cn(Cc3ccc(CNC4CCCCC4)c4scnc34)nc12] | ¹H NMR (400 MHz, DMSO-d₆) δ = 9.41 (s, 1H), 8.04-7.82 (m, 1H), 7.35 (d, J = 7.6 Hz, 1H), 7.30-7.17 (m, 1H), 6.53-6.08 (m, 2H), 5.97 (s, 2H), 4.82-4.61 (m, 1H), 4.24-4.01 (m, 2H), 3.89 (s, 2H), 3.72-3.62 (m, 3H), 2.73-2.63 (m, 1H), 2.38-2.28 (m, 1H), 2.10- 1.99 (m, 2H), 1.91 (s, 1H), 1.78-1.65 (m, 2H), 1.65-1.55 (m, 2H), 1.54-1.45 (m, 2H), 1.36-1.22 (m, 1H), 1.21-1.09 (m, 1H), 0.95- 0.74 (m, 3H). LC-MS (ES): m/z = 481.2 [M + H]⁺, RT (min) = 1.61 |
| 202 | 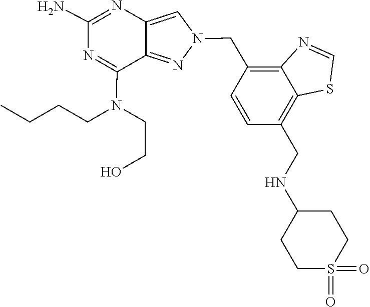 | ¹H NMR (400 MHz, DMSO-d₆) δ = 9.41 (s, 1H), 8.07-7.83 (m, 1H), 7.37 (d, J = 7.6 Hz, 1H), 7.30-7.17 (m, 1H), 6.54-6.48 (m, 1H), 6.40-6.21 (m, 1H), 5.97 (s, 2H), 4.86-4.54 (m, 1H), 4.17-4.05 (m, 2H), 4.02 (br d, J = 5.4 Hz, 2H), 3.81-3.76 (m, 1H), 3.70 (br s, 1H), 3.63 (br dd, J = 6.9, 4.1 Hz, 4H), 3.06 (br dd, J = 10.9, 8.7 Hz, 1H), 2.43-2.29 (m, 2H), 1.97- 1.86 (m, 2H), 1.64-1.47 (m, 3H), 1.40- 1.23 (m, 3H), 1.20-1.09 (m, 1H), 0.95-0.87 (m, 1H), 0.82-0.69 (m, 2H). LC-MS (ES): m/z = 511.3 [M + H]⁺, RT (min) = 0.94 |
| 213 | 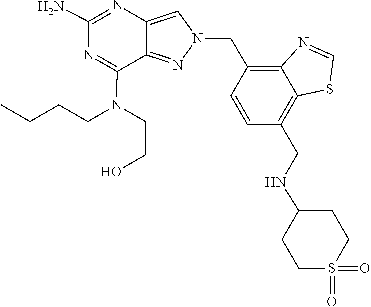 [SMILES: CCCCN(CCO)c1nc(N)nc2cn(Cc3ccc(CNC4CCS(=O)(=O)CC4)c4scnc34)nc12] | ¹H NMR (400 MHz, DMSO-d₆) δ = 12.36- 11.98 (m, 1H), 9.68-9.21 (m, 2H), 8.28- 8.06 (m, 1H), 7.79 (br s, 2H), 7.53-7.39 (m, 1H), 6.08 (s,2H), 4.94-4.30 (m, 1H), 4.26- 4.06 (m, 2H), 3.85-3.76 (m, 4H), 3.71-3.62 (m, 5H), 3.23 (br s, 4H), 2.10 (br s, 2H), 1.70- 1.46 (m, 4H), 1.41-1.08 (m, 3H), 1.00-0.68 (m, 3H). LC-MS (ES): m/z = 559.2 [M + H]⁺, RT (min) = 1.25 |

### Example S12. (S)-2-((5-Amino-2-((5-(((tetrahydrofuran-3-yl)amino)methyl)chroman-8-yl) methyl)-2H-pyrazolo[4,3-d]pyrimidin-7-yl)(butyl)amino) ethan-1-ol (Compound 136)

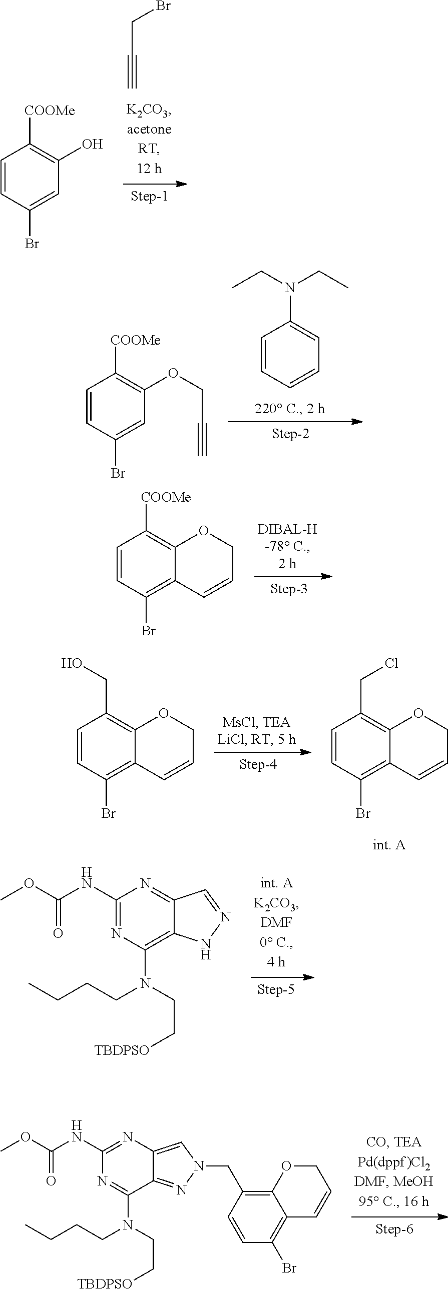

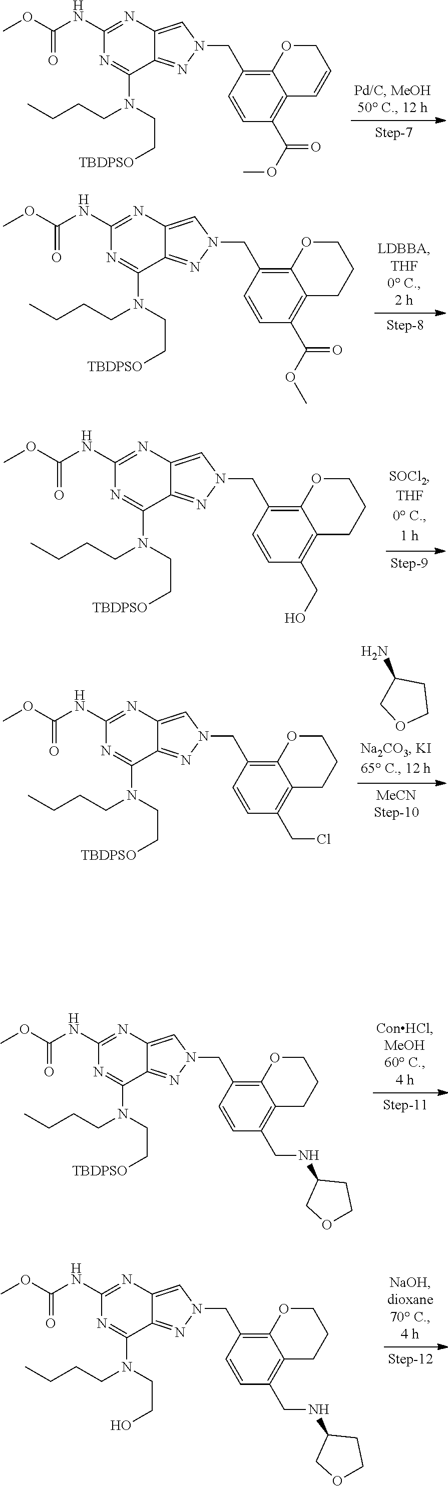

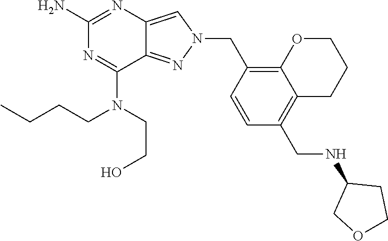 [SMILES: CCCCN(CCO)c1nc(N)nc2cn(Cc3ccc(CN[C@H]4CCOC4)c4c3OCCC4)nc12]

Step 1. Methyl 4-bromo-2-(prop-2-yn-1-yloxy) benzoate: To a stirred solution of methyl 4-bromo-2-hydroxybenzoate (10 g, 43.3 mmol) in acetone (100 mL), were added K₂CO₃ (11.96 g, 87 mmol) and 3-bromoprop-1-yne (5.60 ml, 51.9 mmol) at 0° C. The reaction mixture was allowed to warm to room temperature and stirred for 12 h. The reaction mixture was concentrated under reduced pressure and the crude was partitioned between water (100 mL) and EtOAc (100 mL). The organic layer was separated, washed with water and brine solution, filtered and concentrated. The crude was purified via ComiFlash column chromatography (Redisep Rf 80 g, silica gel column, 30% EtOAc in pet. ether as an eluent) to afford the title compound (8.8 g, 32.7 mmol, 76% yield) as an off-white colored solid. ¹H NMR (400 MHz, DMSO-d₆) δ=7.66-7.58 (m, 1H), 7.47-7.41 (m, 1H), 7.32-7.25 (m, 1H), 4.99-4.88 (m, 2H), 3.83-3.76 (m, 3H), 3.66-3.60 (m, 1H). LC-MS (ES): m/z=270.2 [M+H]⁺

Step 2. Methyl 5-bromo-2H-chromene-8-carboxylate: A suspension of methyl 4-bromo-2-(prop-2-yn-1-yloxy) benzoate (1 g, 3.72 mmol) in diethylaniline (10 mL) was heated at 220° C. with stirring for 2 h in a 100 mL pressure round bottom flask. The reaction mixture was allowed to cool to room temperature and diluted with ethyl acetate (100 mL). The resultant solution was washed with 1.5M aqueous HCl (100 mL×2) and brine, dried over anhydrous Na₂SO₄, filtered and concentrated. The crude was purified via CombiFlash column chromatography (Redisep Rf 40 g, silica gel column, 30% EtOAc in pet. ether as an eluent) to afford the title compound (720 mg, 2.68 mmol, 72.0% yield) as a light yellow colored solid. ¹H NMR (500 MHz, DMSO-d₆) δ=7.46-7.39 (m, 1H), 7.27-7.21 (m, 1H), 6.71-6.62 (m, 1H), 6.18-6.09 (m, 1H), 4.89-4.80 (m, 2H), 3.80-3.73 (m, 3H). LC-MS (ES): m/z=271.1 [M+H]⁺

Step 3. (5-Bromo-2H-chromen-8-yl)methanol: To a solution of methyl 5-bromo-2H-chromene-8-carboxylate (1.5 g, 5.57 mmol) in THF (20 mL) at −78° C., a solution of DIBAL-H (16.72 mL, 16.72 mmol) was added dropwise over 10 min. The mixture was stirred at same temperature for 2 h and then quenched with dropwise addition of 1.5N HCl solution. After being stirred for 2 h, EtOAc was added. The organic layer was separated and the aqueous layer was extracted with EtOAc (2×20 mL). The combined organic layer was washed with saturated ammonium chloride solution, water and brine solution, dried over sodium sulphate, filtered and concentrated under reduced pressure to afford the title compound (1.2 g, 4.98 mmol, 89% yield) as an off-white solid. ¹H NMR (500 MHz, DMSO-d₆) δ=7.20-7.09 (m, 2H), 6.68-6.58 (m, 1H), 6.09-5.99 (m, 1H), 5.19-5.05 (m, 1H), 4.80-4.71 (m, 2H), 4.46-4.33 (m, 2H). LC-MS (ES): m/z=241.2 [M+H]⁺

Step 4. 5-Bromo-8-(chloromethyl)-2H-chromene: To a stirred solution of (5-bromo-2H-chromen-8-yl)methanol (1.0 g, 4.15 mmol) in DCM (10 mL), were added TEA (1.734 mL, 12.44 mmol), MsCl (0.646 mL, 8.30 mmol) and lithium chloride (0.352 g, 8.30 mmol) at 0° C. The reaction mixture was stirred at same temperature for 30 minutes and then at room temperature for 5 h. The reaction mixture was diluted with DCM (20 mL) and saturated aq. NaHCO₃ solution (20 mL). The organic layer was separated, and the aq. layer was extracted with DCM (2×20 mL). The combined organic extracts were dried over sodium sulphate, filtered, and concentrated to afford the title compound (800 mg, 3.08 mmol, 74.3% yield) as an off-white solid. ¹H NMR (400 MHz, DMSO-d₆) δ 7.22-7.15 (m, 2H), 6.70-6.60 (m, 1H), 6.19-6.06 (m, 1H), 4.90-4.78 (m, 2H), 4.67-4.55 (m, 2H). LC-MS (ES): m/z=259.2 [M+H]⁺

Step 5. Methyl (2-((5-bromo-2H-chromen-8-yl) methyl)-7-(butyl(2-((tert-butyldiphenylsilyl) oxy) ethyl) amino)-2H-pyrazolo[4,3-d]pyrimidin-5-yl) carbamate: To a stirred solution of methyl (7-(butyl(2-((tert-butyldiphenylsilyl)oxy)ethyl)amino)-1H-pyrazolo[4,3-d]pyrimidin-5-yl) carbamate (2.8 g, 5.12 mmol) in DMF (30 mL), was added K₂CO₃ (1.416 g, 10.24 mmol). To this mixture 5-bromo-8-(chloromethyl)-2H-chromene (1.728 g, 6.66 mmol) was added portion wise over 2 min at 0° C. The reaction mixture was stirred at 0° C. for 4 h. Ice-cold water was added. The precipitated solid was filtered, washed with water followed by pet. ether and dried under vacuum. The crude compound was purified by using Combiflash column chromatography (80 g, silica gel column, 60% ethyl acetate in pet. ether as an eluent) to afford the title compound (2.4 g, 3.12 mmol, 60.9% yield) as a light yellow solid. LC-MS (ES): m/z=569.2 [M+H]⁺

Step 6. Methyl 8-((7-(butyl(2-((tert-butyldiphenylsilyl)oxy)ethyl)amino)-5-((methoxycarbonyl)amino)-2H-pyrazolo[4,3-d]pyrimidin-2-yl)methyl)-2H-chromene 5-carboxylate: To a solution of methyl (2-((5-bromo-2H-chromen-8-yl)methyl)-7-(butyl(2-((tert-butyldiphenylsilyl) oxy)ethyl)amino)-2H-pyrazolo[4,3-d]pyrimidin-5-yl) carbamate (1.0 g, 1.299 mmol) in a mixture of dry DMF (25 mL) and MeOH (25 mL), were added Et₃N (0.905 mL, 6.50 mmol) and PdCl₂(dppf)-CH₂Cl₂ (0.212 g, 0.260 mmol). The reaction mixture was heated at 95° C. under CO gas pressure (10 Kg) in tiny clave for 16 h. The reaction mixture was allowed to cool to room temperature and then filtered through a Celite bed, which was washed with excess of EtOAc. The filtrate was concentrated in vacuo at 45° C. The residue was purified by CombiFlash column chromatography (Redisep Rf 40 g silica gel column, 10-50% ethyl acetate in pet. ether as an eluent) to get the title compound (0.74 g, 0.988 mmol, 76% yield) as a light brown semi-solid. LC-MS (ES): m/z=749.2 [M+H]⁺

Step 7. Methyl 8-((7-(butyl(2-((tert-butyldiphenylsilyl)oxy)ethyl)amino)-5-((methoxycarbonyl)amino)-2H-pyrazolo[4,3-d]pyrimidin-2-yl)methyl)chromane 5-carboxylate: A stirred solution of methyl 8-((7-(butyl(2-((tert-butyldiphenylsilyl)oxy)ethyl)amino)-5-((methoxycarbonyl)amino)-2H-pyrazolo[4,3-d]pyrimidin-2-yl)methyl)-2H-chromene-5-carboxylate (1.4 g, 1.869 mmol) in a mixture of MeOH (25 mL) and AcOH (4 mL) in an autoclave was purged with N₂ gas at room temperature. Pd/C (0.995 g, 0.935 mmol) was added. The mixture was degassed with vacuum and then heated to 50° C. under 10 Kg hydrogen gas pressure for 12 h. The reaction mixture was allowed to cool to room temperature and the black suspension was filtered through a Celite bed, which was washed with ethyl acetate. The filtrate was concentrated under reduced pressure to afford the title compound (1.2 g, 1.598 mmol, 85% yield) as a brown semi-solid. LCMS (ES): m/z=852.3 [M+H]⁺

Step 8. Methyl (7-(butyl(2-((tert-butyldiphenylsilyl)oxy)ethyl)amino)-2-((5-(hydroxymethyl)chroman-8-yl)methyl)-2H-pyrazolo[4,3-d]pyrimidin-5-yl) carbamate: To a stirred solution of methyl 8-((7-(butyl(2-((tert-butyldiphenylsilyl)oxy)ethyl)amino)-5-((methoxycarbonyl)amino)-2H-pyrazolo[4,3-d]pyrimidin-2-yl)methyl)chromane 5-carboxylate (500 mg, 0.666 mmol) in THE (5 mL) at 0° C., was added a solution of lithium diisobutyl-tert-butoxyaluminum hydride (0.25 M in THF/hexane, 18.64 mL, 4.66 mmol) dropwise over 10 min under nitrogen atmosphere. The reaction mixture was allowed to warm to RT and stirred for 2 h. The reaction mixture was partitioned between aq. sodium potassium tartrate solution and EtOAc. The organic layer was separated, washed with water (30 mL) and brine (30 mL), dried over anhydrous sodium sulphate, filtered, and concentrated. The residue was purified by CombiFlash column chromatography (12 g Redisep silica gel column 60% ethyl acetate in DCM as an eluent) to afford the title compound (420 mg, 0.581 mmol, 87% yield) as an off-white solid. LCMS (ES): m/z=723.2 [M+H]⁺

Step 9. Methyl (7-(butyl(2-((tert-butyldiphenylsilyl) oxy)ethyl)amino)-2-((5-(chloromethyl)chroman-8-yl)methyl)-2H-pyrazolo[4,3-b]pyridin-5-yl) carbamate: To a stirred solution of methyl (7-(butyl(2-((tert-butyldiphenylsilyl)oxy)ethyl)amino)-2-((5-(hydroxymethyl)chroman-8-yl)methyl)-2H-pyrazolo[4,3-b]pyridin-5-yl) carbamate (200 mg, 0.277 mmol) in THE (8 mL), SOCl₂ (0.121 mL, 1.662 mmol) was added at 0° C. The reaction mixture was stirred at the same temperature for 1 h. The mixture was concentrated under reduced pressure to afford the title compound (200 mg, 0.270 mmol, 98% yield) as an off-white colored solid. LCMS (ES): m/z=741.3 [M+H]⁺

Step 10. Methyl (S)-(7-(butyl(2-((tert-butyldiphenylsilyl) oxy) ethyl) amino)-2-((5-(((tetrahydrofuran-3-yl)amino)methyl)chroman-8-yl)methyl)-2H-pyrazolo[4,3-d]pyrimidin-5-yl) carbamate: To a stirred solution of methyl (7-(butyl(2-((tert-butyldiphenylsilyl)oxy)ethyl)amino)-2-((5-(chloromethyl)chroman-8-yl)methyl)-2H-pyrazolo[4,3-d]pyrimidin-5-yl) carbamate (100 mg, 0.135 mmol) in CH₃CN (6 mL), were added (S)-tetrahydrofuran-3-amine hydrochloride (21.67 mg, 0.175 mmol), Na₂CO₃ (86 mg, 0.809 mmol) and KI (22.39 mg, 0.135 mmol). The reaction mixture was stirred at 65° C. for 12 h. The reaction mixture was allowed to cool to RT and then filtered by syringe filter. The filtrate was concentrated under vacuum to afford title compound (100 mg, 0.126 mmol, 94% yield) as a brown solid. LCMS (ES): m/z=792.2 [M+H]⁺

Step 11. Methyl (S)-(7-(butyl(2-hydroxyethyl) amino)-2-((5-(((tetrahydrofuran-3-yl) amino) methyl) chroman-8-yl)methyl)-2H-pyrazolo[4,3-d]pyrimidin-5-yl) carbamate hydrochloride: To a solution containing methyl (S)-(7-(butyl(2-((tert-butyldiphenylsilyl)oxy)ethyl)amino)-2-((5-(((tetrahydrofuran-3-yl)amino)methyl) chroman-8-yl)methyl)-2H-pyrazolo[4,3-d]pyrimidin-5-yl) carbamate (100 mg, 0.126 mmol) in MeOH (5 mL), was added aqueous HCl (1.683 mL, 2.53 mmol) dropwise over 2 min. The mixture was stirred at 60° C. for 4 h. The reaction mixture was concentrated under reduced pressure. The residue was triturated with diethyl ether (2×10 mL) and pet. ether (1×10 mL) and dried to afford the title compound (70 mg, 0.119 mmol, 94% yield) as a light yellow solid. LCMS (ES): m/z=554.2 [M+H]⁺

Step 12. (S)-2-((5-Amino-2-((5-(((tetrahydrofuran-3-yl) amino) methyl) chroman-8-yl) methyl)-2H-pyrazolo[4,3-d]pyrimidin-7-yl) (butyl)amino) ethan-1-ol: To a solution containing methyl (S)-(7-(butyl(2-hydroxyethyl) amino)-2-((5-(((tetrahydrofuran-3-yl) amino)methyl)chroman-8-yl)methyl)-2H-pyrazolo[4,3-d]pyrimidin-5-yl) carbamate hydrochloride (80 mg, 0.136 mmol) in dioxane (5 mL), was added an aqueous solution of NaOH (1.356 mL, 4.07 mmol) at room temperature. The reaction mixture was heated to 70° C. and stirred for 4 h. The organic layer was separated and concentrated under reduced pressure to get the residue. The crude compound was purified by following preparative LC-MS (Column: Waters XBridge C18, 19×150 mm, 5-μm particles; mobile phase A: 10 mM ammonium acetate; mobile phase B: acetonitrile; gradient: 10⁻³³% B over 21 minutes, then a 5-minute hold at 100% B; flow: 20 mL/min). Fractions containing the desired product were combined and dried via centrifugal evaporation to afford title compound (13.9 mg, 0.028 mmol, 20.69% yield).

| Cmpd | | Analytical Data (¹H NMR 400 MHz, DMSO- |
|---|---|---|
| No. | Structure | d₆; LC-MS) |
| 136 | 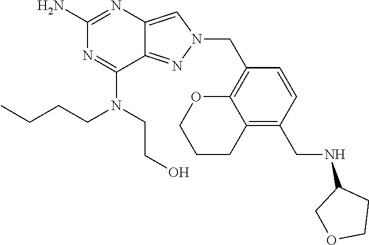 | δ = 7.70 (br s, 1H), 6.88-6.81 (m, 1H), 6.76 (br s, 1H), 5.71 (br s, 2H), 5.34 (s, 2H), 4.68 (s, 1H), 4.20-4.07 (m, 2H), 3.77-3.56 (m, 12H), 3.43 (br d, J = 4.5 Hz, 4H), 2.76 (t, J = 6.4 Hz, 2H), 1.99-1.89 (m, H), 1.68 (br d, J = 5.4 Hz, 1H), 1.58 (br s, 2H), 1.30 (br d, J = 6.0 Hz, 2H), 0.90 (br s, 3H). LC-MS (ES): m/z = 496.2 [M + H]⁺, RT (min) = 1.35 |

### Example S13. methyl (R)-3-aminopiperidine-1-carboxylate (Amine Used in the Synthesis of Compound 185)

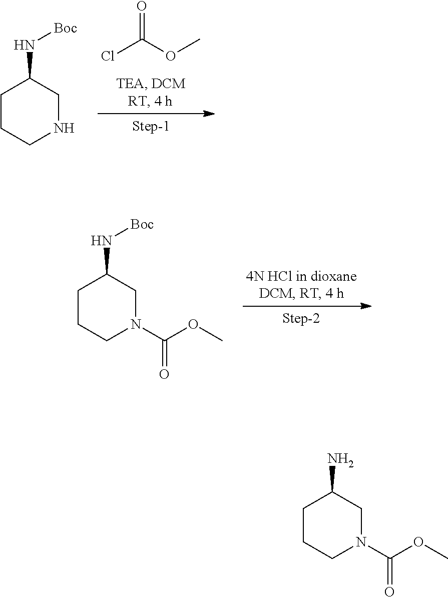

Step 1. Methyl (R)-3-((tert-butoxycarbonyl) amino) piperidine-1-carboxylate: To a solution containing tert-butyl (R)-piperidin-3-ylcarbamate (500 mg, 2.496 mmol) in chloroform (5 mL) and Et₃N (1.044 mL, 7.49 mmol) at 0° C., was added methyl chloroformate (307 mg, 3.25 mmol) dropwise over 2 min. The mixture was stirred at room temperature for 4 h. The mixture was diluted with chloroform (20 mL) and the resultant solution was washed with brine solution (1×20 mL), dried over Na₂SO₄, filtered, and concentrated to afford the title compound (600 mg, 2.323 mmol, 93% yield) as a brown semi-solid. ¹H NMR (300 MHz, CD₃OD) δ=3.96-3.70 (m, 3H), 3.49-3.27 (m, 2H), 3.05-2.80 (m, 2H), 1.97-1.81 (m, 2H), 1.77-1.61 (m, 2H), 1.50-1.39 (m, 9H). LC-MS (ES): m/z=259.2 [M+H]⁺

Step 2. Methyl (R)-3-aminopiperidine-1-carboxylate: To a stirred solution of methyl (R)-3-((tert-butoxycarbonyl) amino) piperidine-1-carboxylate (400 mg, 1.548 mmol) in DCM (10 mL), was added 4M HCl in dioxane (1.936 mL, 7.74 mmol) at 0° C. and the reaction mixture was stirred at RT for 12 h. The reaction mixture was concentrated under reduced pressure. The residue was triturated with diethyl ether (2×10 mL), and dried to afford the title compound (220 mg, 1.130 mmol, 73.0% yield) as an off-white solid. LC-MS (ES): m/z=159.2 [M+H]⁺

### Example S14. Methyl (R)-3-(((8-((5-amino-7-(butyl(2-hydroxyethyl) amino)-2H-pyrazolo[4,3-d]pyrimidin-2-yl)methyl)quinolin-5-yl)methyl)amino) piperidine-1-carboxylate (Compound 185)

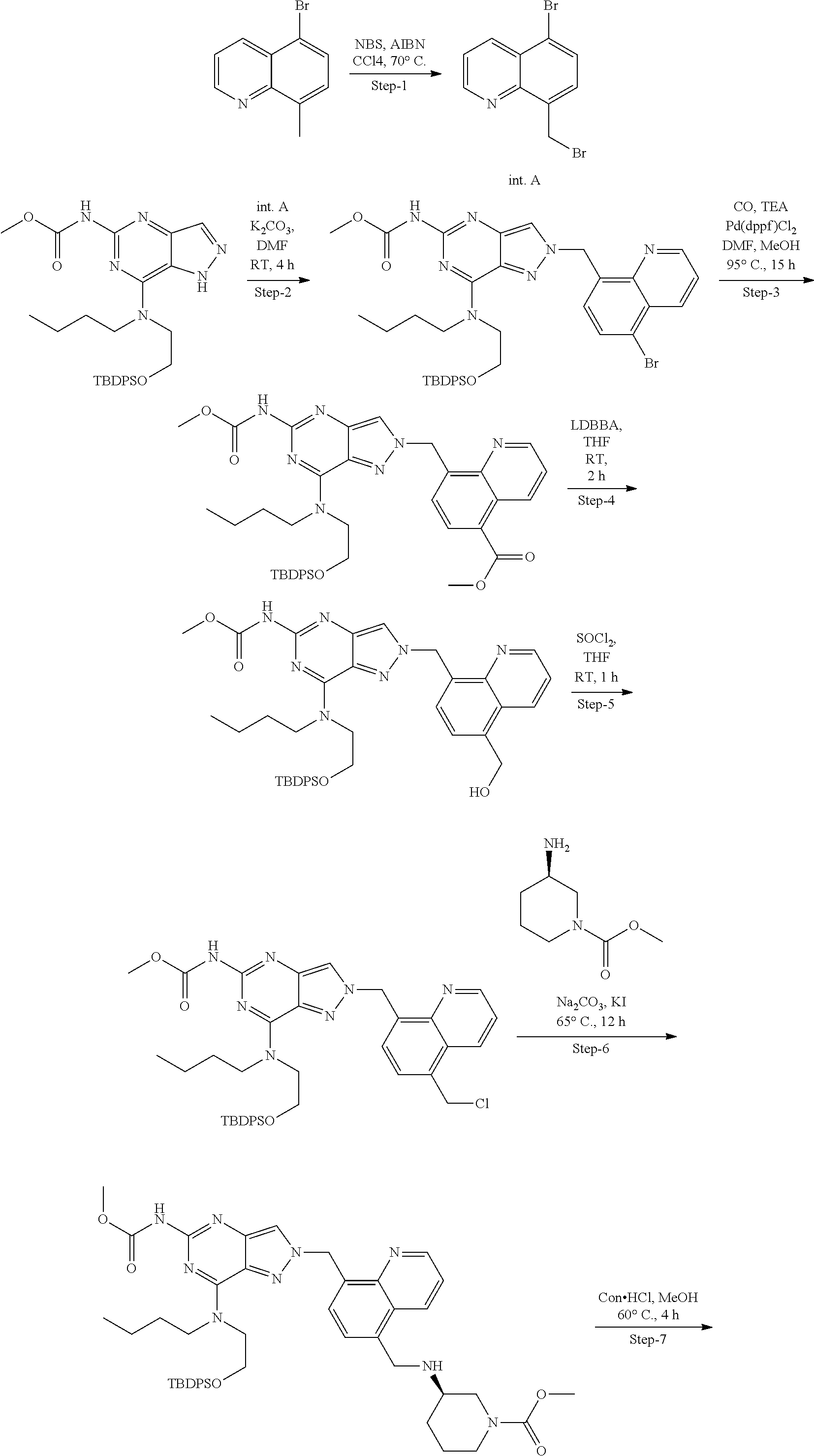

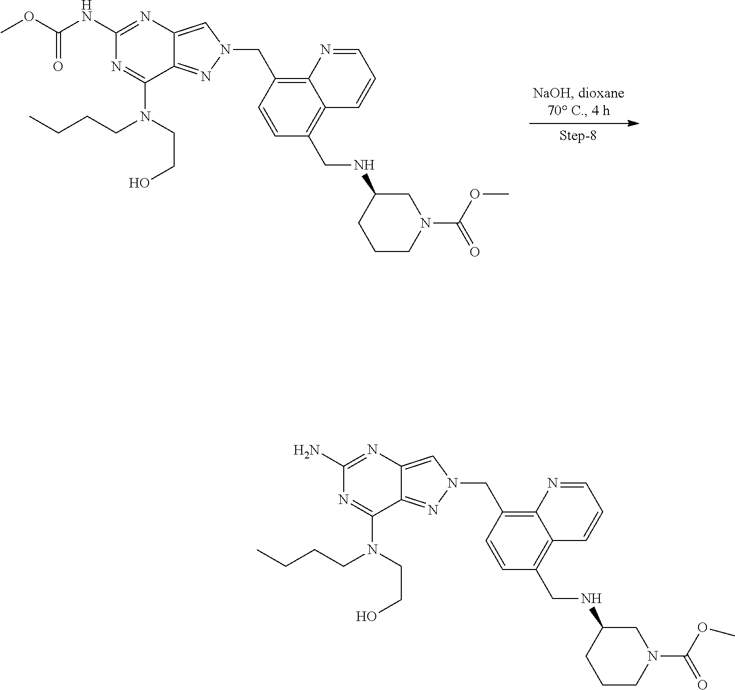

Step 1. 5-Bromo-8-(bromomethyl)quinoline: To a stirred solution of 5-bromo-8-methylquinoline (5 g, 22.51 mmol) in CCl₄ (50 mL), was added NBS (4.81 g, 27.0 mmol) followed by AIBN (0.739 g, 4.50 mmol). The reaction mixture was stirred at 70° C. for 12 h. The reaction mixture was cooled to room temperature and concentrated under high vacuum. The residue was partitioned between ethyl acetate and water. The aq. layer was extracted with ethyl acetate (2×50 mL). The combined organic layer was washed with water and brine solution, dried over anhydrous sodium sulphate, filtered and concentrated. The residue was purified using CombiFlash (Redisep Rf 80 g silica gel column; 15% ethyl acetate in pet. ether as an eluent) to afford the title compound (3.6 g, 11.96 mmol, 53.1% yield) as a white solid. ¹H NMR (500 MHz, DMSO-d₆) δ=8.27-8.22 (m, 1H), 7.76-7.69 (m, 1H), 7.17-7.13 (m, 1H), 7.08-7.04 (m, 1H), 6.96-6.88 (m, 1H), 4.47-4.38 (m, 2H). LC-MS (ES): m/z=301.9 [M+2H]⁺

Step 2. 5-Bromo-8-(bromomethyl)quinoline: To a stirred solution of methyl (7-(butyl(2-((tert-butyldiphenylsilyl) oxy) ethyl) amino)-1H-pyrazolo[4,3-d]pyrimidin-5-yl) carbamate (3.0 g, 5.49 mmol) in DMF (30 mL), were added K₂CO₃ (1.517 g, 10.97 mmol) and 5-bromo-8-(bromomethyl)quinoline (2.147 g, 7.13 mmol) at 0° C. The reaction mixture was allowed to warm to room temperature and stirred for 4 h. EtOAc and water were added. and the mixture was stirred for 10 min. The organic layer was separated, and the aqueous layer was extracted with EtOAc (2×20 mL). The combined organic layer was washed with water (2×20 mL), brine solution, dried over sodium sulphate, filtered, and concentrated. The residue was purified via CombiFlash chromatography (Redisep Rf 40 g silica gel column, 40% EtOAc in DCM as an eluent) to afford the title compound (3.1 g, 4.04 mmol, 73.7% yield) as a light yellow solid. LC-MS (ES): m/z=766.5 [M+H]⁺

Step 3. Methyl 8-((7-(butyl(2-((tert-butyldiphenylsilyl)oxy)ethyl)amino)-5-((methoxycarbonyl)amino)-2H-pyrazolo[4,3-d]pyrimidin-2-yl)methyl) quinoline-5-carboxylate: To a solution of methyl (2-((5-bromoquinolin-8-yl)methyl)-7-(butyl(2-((tert-butyldiphenylsilyl)oxy) ethyl)amino)-2H-pyrazolo[4,3-d]pyrimidin-5-yl) carbamate (3.0 g, 3.91 mmol) in a mixture of dry MeOH (60 mL) and DMF (60 mL), were added Et₃N (2.73 mL, 19.56 mmol) and PdCl₂(dppf)-CH₂Cl₂ (0.639 g, 0.782 mmol). The reaction mixture was heated at 95° C. under CO gas (10 Kg pressure) in auto clave for 16 h. The reaction mixture was allowed to cool to room temperature and then filtered through a Celite bed, which was washed with EtOAc. The filtrate was concentrated in vacuo at 45° C. The residue was purified by CombiFlash chromatography (Redisep Rf 40 g silica gel column, 10-50% ethyl acetate in pet. ether as an eluent) to get the title compound (2.4 g, 3.22 mmol, 82% yield) as a light brown semi-solid. LC-MS (ES): m/z=746.2 [M+2H]⁺

Step 4. Methyl (7-(butyl(2-((tert-butyldiphenylsilyl)oxy)ethyl)amino)-2-((5-(hydroxymethyl)quinolin-8-yl)methyl)-2H-pyrazolo[4,3-d]pyrimidin-5-yl) carbamate: To a solution of methyl 8-((7-(butyl(2-((tert-butyldiphenylsilyl)oxy)ethyl)amino)-5-((methoxycarbonyl) amino)-2H-pyrazolo[4,3-d]pyrimidin-2-yl)methyl) quinoline-5-carboxylate (500 mg, 0.670 mmol) in THE (5 mL) at 0° C., was added lithium diisobutyl-tert-butoxyaluminum hydride solution (0.25 M in THF/hexane, 18.77 mL, 4.69 mmol) dropwise over 10 min under nitrogen atmosphere. The reaction mixture was allowed to warm to RT and stirred for 2 h. The reaction mixture was partitioned between aq. sodium potassium tartrate solution and EtOAc. The organic layer was separated, washed with water (30 mL) and brine (30 mL), dried over anhydrous sodium sulphate and concentrated. The residue was purified by CombiFlash chromatography (Redisep Rf 60-120 silica gel; 60% ethyl acetate in DCM as an eluent) to afford the title compound (394 mg, 0.549 mmol, 82% yield) as an off-white solid. LC-MS (ES): m/z=718.2 [M+H]⁺

Step 5. Methyl (7-(butyl(2-((tert-butyldiphenylsilyl) oxy)ethyl)amino)-2-((5-(chloromethyl)quinolin-8-yl)methyl)-2H-pyrazolo[4,3-b]pyridin-5-yl) carbamate: To a stirred solution of methyl (7-(butyl(2-((tert-butyldiphenylsilyl)oxy)ethyl)amino)-2-((5-(hydroxymethyl)quinolin-8-yl)methyl)-2H-pyrazolo[4,3-b]pyridin-5-yl) carbamate (125 mg, 0.174 mmol) in THE (8 mL), SOCl₂ (0.076 mL, 1.046 mmol) was added at 0° C. The reaction mixture was stirred at the same temperature for 1 h. The reaction mixture was concentrated under reduced pressure to afford the title compound (120 mg, 0.163 mmol, 94% yield) as an off-white colored solid. LC-MS (ES): m/z=735.3 [M+H]⁺

Step 6. Methyl (R)-3-(((8-((7-(butyl(2-((tert-butyldiphenylsilyl)oxy)ethyl)amino)-5-((methoxycarbonyl)amino)-2H-pyrazolo[4,3-d]pyrimidin-2-yl)methyl)quinolin-5-yl)methyl) amino) piperidine-1-carboxylate: To a stirred solution of methyl (7-(butyl(2-((tert-butyldiphenylsilyl)oxy)ethyl)amino)-2-((5-(chloromethyl)quinolin-8-yl)methyl)-2H-pyrazolo[4,3-d]pyrimidin-5-yl) carbamate (100 mg, 0.136 mmol) in CH₃CN (10 mL), were added methyl (R)-3-aminopiperidine-1-carboxylate (25.8 mg, 0.163 mmol), Na₂CO₃ (86 mg, 0.815 mmol) and KI (22.54 mg, 0.136 mmol). The reaction mixture was stirred at 65° C. for 12 h. The reaction mixture was allowed to cool to RT and then filtered through a pad of syringe. The filtrate was concentrated under vacuum to afford the title compound (102 mg, 0.119 mmol, 88% yield) as a brown semi-solid. LC-MS (ES): m/z=858.2 [M+H]⁺

Step 7. Methyl (R)-3-(((8-((7-(butyl(2-hydroxyethyl)amino)-5-((methoxycarbonyl)amino)-2H-pyrazolo[4,3-d]pyrimidin-2-yl)methyl)quinolin-5-yl)methyl) amino)piperidine-1-carboxylate hydrochloride: To a solution of methyl (R)-3-(((8-((7-(butyl(2-((tert-butyldiphenylsilyl)oxy)ethyl)amino)-5-((methoxycarbonyl)amino)-2H-pyrazolo[4,3-d]pyrimidin-2-yl)methyl)quinolin-5-yl)methyl)amino) piperidine-1-carboxylate (100 mg, 0.117 mmol) in MeOH (5 mL), was added aqueous HCl (1.55 mL, 2.331 mmol) dropwise over 2 min. The mixture was stirred at 60° C. for 4 h. The reaction mixture was concentrated under reduced pressure. The residue was triturated with diethyl ether (2×10 mL) and dried to afford title compound (65 mg, 0.099 mmol, 85% yield) as a light yellow solid. LC-MS (ES): m/z=620.2 [M+H]⁺

Step 8. Methyl (R)-3-(((8-((5-amino-7-(butyl(2-hydroxyethyl) amino)-2H-pyrazolo[4,3-d]pyrimidin-2-yl) methyl) quinolin-5-yl) methyl) amino) piperidine-1-carboxylate: To a solution containing methyl (R)-3-(((8-((7-(butyl(2-hydroxyethyl)amino)-5-((methoxycarbonyl)amino)-2H-pyrazolo[4,3-d]pyrimidin-2-yl)methyl)quinolin-5-yl)methyl)amino)piperidine-1-carboxylate hydrochloride (65 mg, 0.099 mmol) in dioxane (5 mL), was added an aqueous solution of NaOH (0.990 mL, 2.97 mmol) at room temperature. The reaction mixture was heated to 70° C. and stirred for 4 h. The organic layer was separated and concentrated under reduced pressure. The crude compound was purified by preparative LC-MS (column: Waters XBridge C18, 19×150 mm, 5-μm particles; mobile phase A: 10 mM ammonium acetate; mobile phase b: acetonitrile; gradient: 12-38% B over 25 minutes, then a 5-minute hold at 100% B; flow: 20 mL/min). Fractions containing the desired product were combined and dried via centrifugal evaporation to afford the title compound (21.3 mg, 0.038 mmol, 38.3% yield).

| Cmpd | | Analytical Data (¹H NMR 400 MHz, DMSO-d₆; |
|---|---|---|
| No. | Structure | LC-MS) |
| 185 | 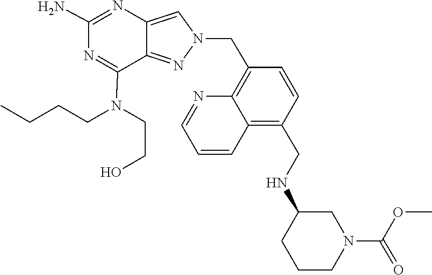 [SMILES: CCCCN(CCO)c1nc(N)nc2cn(Cc3ccc(CN[C@@H]4CCCN(C(=O)OC)C4)c4cccnc34)nc12] | δ = 9.05-8.87 (m, 1H), 8.69 (dd, J = 1.6, 8.6 Hz, 1H), 7.87 (br s, 1H), 7.62 (dd, J = 4.2, 8.6 Hz, 1H), 7.53 (d, J = 7.3 Hz, 1H), 7.26 (br s, 1H), 6.07 (s, 2H), 5.58 (br s, 2H), 4.71 (br s, 1H), 4.23- 3.98 (m, 5H), 3.87 (br s, 2H), 3.63 (br d, J = 4.6 Hz, 7H), 3.55 (br s, 4H), 2.96 (br s, 1H), 2.80 (dd, J = 8.8, 12.9 Hz, 1H), 1.64 (br s, 3H), 1.39- 1.14 (m, 5H), 0.78 (br d, J = 1.0 Hz, 3H). LC- MS (ES): m/z = 562.3 [M + H]⁺, RT (min) = 0.89 |

### Example S15. (R)-1-(methylsulfonyl)piperidin-3-amine (Amine Used in the Synthesis of Compound 200)

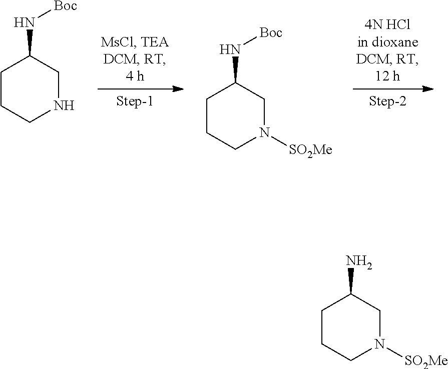 [SMILES: CC(C)(C)OC(=O)N[C@@H]1CCCNC1 ; CC(C)(C)OC(=O)N[C@@H]1CCCN(S(C)(=O)=O)C1 ; CS(=O)(=O)N1CCC[C@@H](N)C1]

Step 1. tert-Butyl-(R)-(1-(methylsulfonyl)piperidin-3-yl) carbamate: To a solution containing tert-butyl (R)-piperidin-3-yl carbamate (500 mg, 2.496 mmol) in chloroform (5 mL) and TEA (1.044 mL, 7.49 mmol) at 0° C., was added methanesulfonyl chloride (372 mg, 3.25 mmol) over 2 min. The mixture was stirred at room temperature for 4 h, diluted with chloroform (20 mL), washed with brine solution (1×20 mL), dried over Na₂SO₄, filtered, and concentrated to afford the title compound (610 mg, 2.191 mmol, 88% yield) as a brown semi-solid. LC-MS (ES): m/z=279.2 [M+H]⁺

Step 2. (R)-1-(Methylsulfonyl)piperidin-3-amine: To a stirred solution of tert-butyl (R)-(1-(methylsulfonyl) piperidin-3-yl) carbamate (400 mg, 1.437 mmol) in DCM (10 mL), was added HCl in dioxane (1.796 mL, 7.18 mmol) at 0° C. and the reaction mixture was stirred at RT for 12 h. The reaction mixture was concentrated under reduced pressure. The residue was triturated with diethyl ether (2×10 mL) and dried to afford the title product (220 mg, 1.025 mmol, 71.3% yield) as a brown solid. LC-MS (ES): m/z=179.2 [M+H]⁺

### Example S16. (R)-2-((5-Amino-2-((5-(((1-(methylsulfonyl)piperidin-3-yl)amino)methyl) quinolin-8-yl)methyl)-2H-pyrazolo[4,3-d]pyrimidin-7-yl)(butyl)amino) ethan-1-ol (Compound 200)

The title compound was prepared from methyl (7-(butyl(2-((tert-butyldiphenylsilyl)oxy)ethyl)amino)-2-((5-(chloromethyl)quinolin-8-yl)methyl)-2H-pyrazolo[4,3-b]pyridin-5-yl) carbamate and (R)-1-(methyl sulfonyl) piperidin-3-amine following three-step procedure for Compound 185.

| Cmpd | | Analytical Data (¹H NMR 400 MHz, DMSO-d₆; |
|---|---|---|
| No. | Structure | LC-MS) |
| 200 | 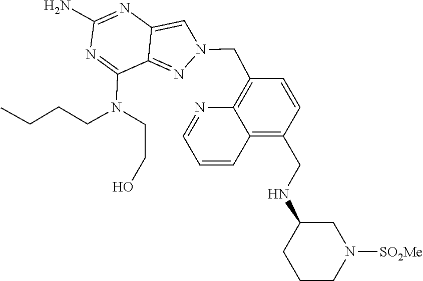 [SMILES: CCCCN(CCO)c1nc(N)nc2cn(Cc3ccc(CN[C@@H]4CCCN(S(C)(=O)=O)C4)c4cccnc34)nc12] | δ = 8.98 (br d, J = 4.3 Hz, 1H), 8.70 (d, J = 8.8 Hz, 1H), 7.87 (br s, 1H), 7.61 (dd, J = 4.0, 8.0 Hz, 1H), 7.53 (d, J = 7.5 Hz, 1H), 7.32 (s, 1H), 6.07 (s, 2H), 5.65 (br s, 2H), 4.70 (s, 1H), 4.17 (br s, 4H), 3.77-3.55 (m, 10H), 2.84 (s, 3H), 2.76-2.71 (m, 1H), 2.66-2.56 (m, 1H), 1.91 (s, 1H), 1.77 (br s, 1H), 1.49 (br s, 3H), 1.24 (br d, J = 1.0 Hz, 3H), 0.95-0.68 (m, 3H). LC-MS (ES): m/z = 582.3 [M + H]⁺, RT (min) = 1.44 |

### Example S17. (R)-2-((5-Amino-2-((5-(((tetrahydrofuran-3-yl)amino)methyl) imidazo[1,2-a]pyridin-8-yl)methyl)-2H-pyrazolo[4,3-d]pyrimidin-7-yl)(butyl)amino) ethan-1-ol (Compound 187)

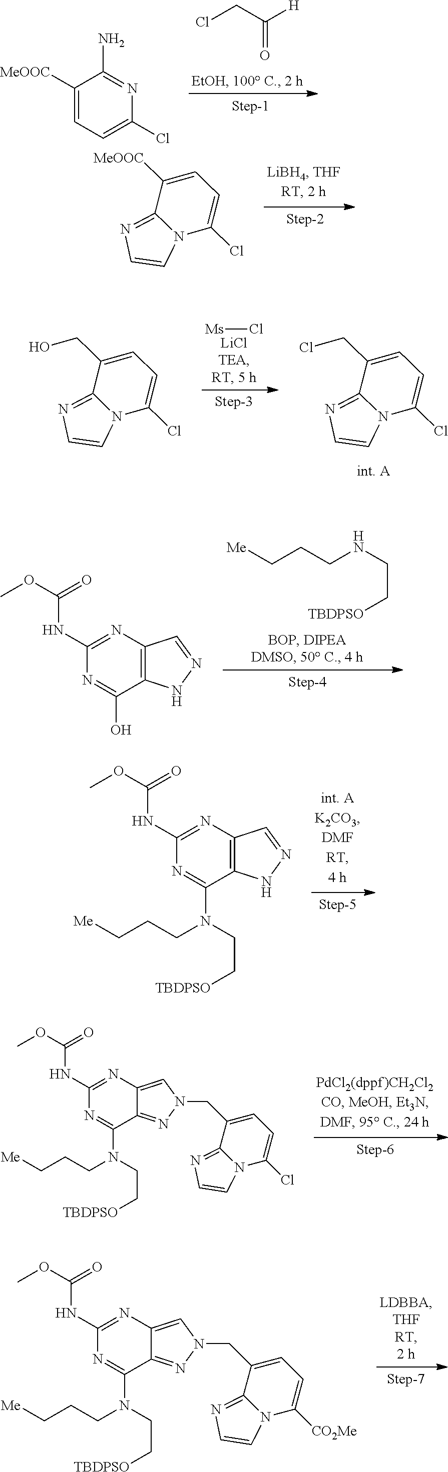

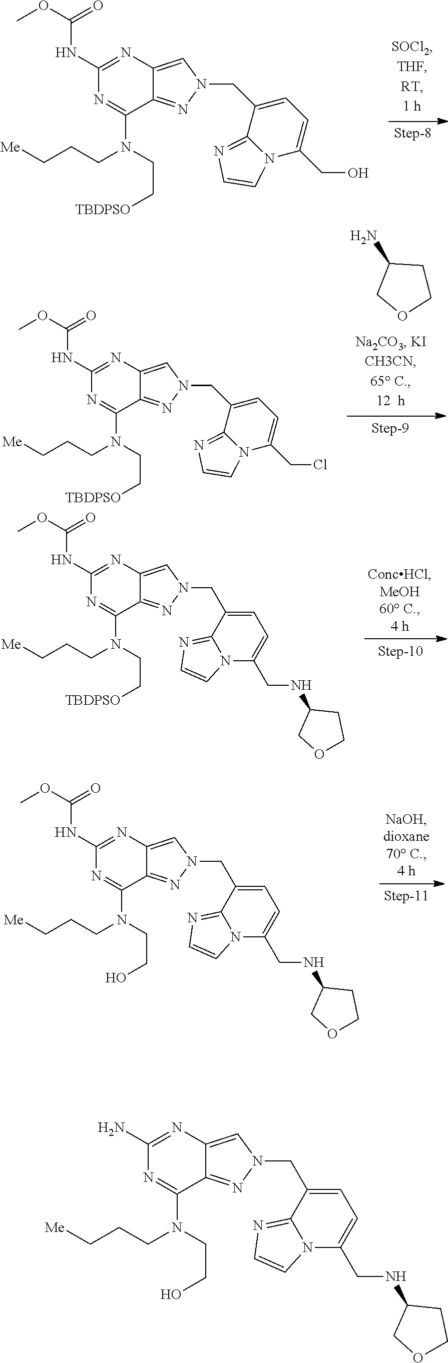

Step 1. Methyl 5-chloroimidazo[1,2-a]pyridine-8-carboxylate: A 100 mL pressure tube was charged with methyl 2-amino-6-chloronicotinate (4 g, 21.44 mmol) and 2-chloroacetaldehyde (50% wt. solution in water) (13.61 mL, 107 mmol) in ethanol (30 mL) and the resultant mixture was heated at 100° C. for 2 h under closed condition. The reaction mixture was allowed to cool to room temperature and concentrated under reduced pressure. The residue was partitioned between ethyl acetate and saturated aqueous sodium bicarbonate. The organic phase was separated, and the aqueous phase was extracted with ethyl acetate. The combined organic extracts were dried (Na₂SO₄), filtered and concentrated in vacuo. The residue was purified via CombiFlash column chromatography (Redisep Rf silica gel 40 g column; 70% ethyl acetate in pet. ether as an eluent) to afford the title compound (2.6 g, 12.34 mmol, 57.6% yield) as a light yellow solid. LC-MS (ES): m/z=211.2 [M+H]⁺

Step 2. (5-Chloroimidazo[1,2-a]pyridin-8-yl) methanol: To a stirred solution of methyl 5-chloroimidazo[1,2-a]pyridine-8-carboxylate (1.5 g, 7.12 mmol) in THE (15 mL) at 0° C., was added a solution of LiBH₄ (17.80 mL, 35.6 mmol) dropwise over 5 min. The reaction mixture was stirred at RT for 2 h and partitioned between ammonium chloride solution and EtOAc. The organic layer was separated, and the aqueous layer was extracted with EtOAc (2×100 mL). The combined organic layer was washed with saturated brine (100 mL), dried over anhydrous sodium sulphate, filtered, and concentrated. The residue was purified by CombiFlash chromatography (Redisep Rf 40 g silica gel column; 40% ethyl acetate in pet ether as an eluent) to afford the title compound (600 mg, 3.29 mmol, 46.1% yield) as an off-white solid. LC-MS (ES): m/z=183.2 [M+H]⁺

Step 3. 5-Chloro-8-(chloromethyl)imidazo[1,2-a]pyridine: To a stirred solution of (5-chloroimidazo[1,2-a]pyridin-8-yl)methanol (600 mg, 3.29 mmol) in DCM (10 mL), TEA (1.374 mL, 9.86 mmol), MsCl (0.512 mL, 6.57 mmol) and lithium chloride (279 mg, 6.57 mmol) were sequentially added at 0° C. The reaction mixture was stirred at same temperature for 30 minutes and then at room temperature for 5 h. The reaction mixture was partitioned between DCM (20 mL) and saturated aq. NaHCO₃ solution (20 mL). The organic layer was separated, and the aq. layer was extracted with DCM (2×20 mL). The combined organic extracts were dried over sodium sulphate, filtered, and concentrated to afford the title compound (550 mg, 2.74 mmol, 83% yield) as a pale brown colored solid. LC-MS (ES): m/z=200.9 [M+H]⁺

Step 4. Methyl (7-(butyl(2-((tert-butyldiphenylsilyl)oxy)ethyl)amino)-1H-pyrazolo[4,3-d]pyrimidin-5-yl) carbamate: To a stirred solution of methyl (7-hydroxy-1H-pyrazolo[4,3-d]pyrimidin-5-yl) carbamate (5 g, 23.90 mmol) in DMSO (30 mL), were added DIPEA (16.70 mL, 96 mmol), N-(2-((tert-butyldiphenylsilyl)oxy) ethyl)butan-1-amine (10.20 g, 28.7 mmol) and BOP (15.86 g, 35.9 mmol). The reaction mixture was stirred at 50° C. for 4 h. The reaction mixture was diluted with ethyl acetate and washed with ice cold water (3×100 mL) followed by brine solution. The organic layer was dried over anhydrous sodium sulphate, filtered, and concentrated. The residue was purified by using Combiflash chromatography (120 g, silica gel column, 40% ethyl acetate in DCM as an eluent) to afford the title compound (4.8 g, 8.78 mmol, 36.7% yield) as light brown semi-solid. LC-MS (ES): m/z=547.2 [M+H]⁺

Step 5. 5-Chloro-8-(chloromethyl)imidazo[1,2-a]pyridine methyl (7-(butyl(2-((tert-butyldiphenylsilyl) oxy) ethyl) amino)-2-((5-chloroimidazo[1,2-a]pyridin-8-yl)methyl)-2H-pyrazolo[4,3-d]pyrimidin-5-yl)carbamate: To a stirred solution of methyl (7-(butyl(2-((tert-butyldiphenylsilyl)oxy)ethyl)amino)-1H-pyrazolo[4,3-d]pyrimidin-5-yl)carbamate (1.360 g, 2.487 mmol) in DMF (15 mL), were added K₂CO₃ (0.687 g, 4.97 mmol) and 5-chloro-8-(chloromethyl)imidazo[1,2-a]pyridine (0.5 g, 2.487 mmol) at 0° C. The reaction mixture was allowed to warm to room temperature and stirred for 4 h. The reaction mixture was partitioned between EtOAc and water. The organic layer was separated, and the aqueous layer was extracted with EtOAc (2×20 mL). The combined organic layer was washed with water (2×20 mL), brine solution, dried over sodium sulphate, filtered, and concentrated. The crude material was purified via CombiFlash chromatography (Redisep Rf 40 g silica gel column, 40% EtOAc in pet. ether as an eluent) to afford the title compound (800 mg, 1.125 mmol, 45.2% yield) as a light yellow solid. LC-MS (ES): m/z=711.5 [M+H]⁺

Step 6. Methyl 8-((7-(butyl(2-((tert-butyldiphenylsilyl)oxy)ethyl)amino)-5-((methoxycarbonyl)amino)-2H-pyrazolo[4,3-d]pyrimidin-2-yl) methyl) imidazo[1,2-a]pyridine-5-carboxylate: To a solution of methyl (7-(butyl(2-((tert-butyldiphenylsilyl)oxy)ethyl)amino)-2-((5-chloroimidazo[1,2-a]pyridin-8-yl)methyl)-2H-pyrazolo[4,3-d]pyrimidin-5-yl) carbamate (1.0 g, 1.406 mmol) in a mixture of dry MeOH (15 mL) and DMF (15 mL), were added Et₃N (0.980 mL, 7.03 mmol) and PdCl₂(dppf)-CH₂Cl₂ (0.230 g, 0.281 mmol). The reaction mixture was heated at 95° C. under CO gas (15 Kg pressure) in tiny clave for 24 h. The reaction mixture was allowed to cool to room temperature, filtered through a Celite bed, which was washed with EtOAc. The filtrate was concentrated in vacuo at 45° C. The residue was purified by CombiFlash chromatography (Redisep Rf 40 g silica gel column, 10-50% ethyl acetate in pet. ether as an eluent) to get the title compound (620 mg, 0.844 mmol, 60.0% yield) as a light brown semi-solid. LC-MS (ES): m/z=735.2 [M+H]⁺

Step 7. Methyl (7-(butyl(2-((tert-butyldiphenylsilyl)oxy)ethyl)amino)-2-((5-(hydroxymethyl) imidazo[1,2-a]pyridin-8-yl)methyl)-2H-pyrazolo[4,3-d]pyrimidin-5-yl) carbamate: To a solution of methyl 8-((7-(butyl(2-((tert-butyldiphenylsilyl)oxy)ethyl)amino)-5-((methoxycarbonyl)amino)-2H-pyrazolo[4,3-d]pyrimidin-2-yl)methyl)imidazole[1,2-a]pyridine-5-carboxylate (600 mg, 0.408 mmol) in THF (10 mL) at 0° C., was added lithium diisobutyl-tert-butoxyaluminum hydride solution (0.25 M in THF/hexane, 11.43 mL, 2.86 mmol) dropwise over 10 min under nitrogen atmosphere. The reaction mixture was allowed to warm to RT and stirred for 2 h. The reaction mixture was partitioned between aq. sodium potassium tartrate solution and EtOAc. The separated organic layer was washed with water (30 mL) and brine (30 mL), dried over anhydrous sodium sulphate and concentrated. The residue was purified by CombiFlash chromatography (24 g Redisep silica gel column 60% ethyl acetate in DCM as an eluent) to afford the title compound (202 mg, 0.286 mmol, 70.0% yield) as an off-white solid. LC-MS (ES): m/z=707.5 [M+H]⁺

Step 8. Methyl (7-(butyl(2-((tert-butyldiphenylsilyl) oxy) ethyl) amino)-2-((5-(chloromethyl) imidazo[1,2-a]pyridin-8-yl) methyl)-2H-pyrazolo[4,3-d]pyrimidin-5-yl) carbamate: To a stirred solution of methyl (7-(butyl(2-((tert-butyldiphenylsilyl)oxy)ethyl)amino)-2-((5-(hydroxymethyl)imidazo[1,2-a]pyridin-8-yl)methyl)-2H-pyrazolo[4,3-d]pyrimidin-5-yl) carbamate (150 mg, 0.212 mmol) in THE (8 mL), SOCl₂ (0.093 mL, 1.273 mmol) was added at 0° C. The reaction mixture was stirred at the same temperature for 1 h. The reaction mixture was concentrated under reduced pressure to afford the title compound (152 mg, 0.210 mmol, 99% yield) as an off-white colored solid. LC-MS (ES): m/z=725.3 [M+H]⁺

Step 9. Methyl (R)-(7-(butyl(2-((tert-butyldiphenylsilyl)oxy)ethyl)amino)-2-((5-(((tetrahydrofuran-3-yl) amino)methyl)imidazo[1,2-a]pyridin-8-yl)methyl)-2H-pyrazolo[4,3-d]pyrimidin-5-yl) carbamate: To a stirred solution of methyl (7-(butyl(2-((tert-butyldiphenylsilyl)oxy)ethyl)amino)-2-((5-(chloromethyl)imidazo[1,2-a]pyridin-8-yl)methyl)-2H-pyrazolo[4,3-d]pyrimidin-5-yl) carbamate (70 mg, 0.097 mmol) in MeCN (5 mL), were added (R)-tetrahydrofuran-3-amine hydrochloride (14.31 mg, 0.116 mmol), Na₂CO₃ (61.4 mg, 0.579 mmol) and KI (16.02 mg, 0.097 mmol). The reaction mixture was stirred at 65° C. for 12 h. The reaction mixture was allowed to cool to RT and then filtered through a syringe pad. The filtrate was concentrated under vacuum to afford the title compound (70 mg, 0.090 mmol, 93% yield) as a brown colored semi-solid. LC-MS (ES): m/z=776.2 [M+H]⁺

Step 10. Methyl (R)-(7-(butyl(2-hydroxyethyl) amino)-2-((5-(((tetrahydrofuran-3-yl)amino)methyl) imidazo[1,2-a]pyridin-8-yl)methyl)-2H-pyrazolo[4,3-d]pyrimidin-5-yl) carbamate: To a solution containing methyl (R)-(7-(butyl(2-((tert-butyldiphenylsilyl)oxy)ethyl)amino)-2-((5-(((tetrahydrofuran-3-yl)amino)methyl)imidazo[1,2-a]pyridin-8-yl)methyl)-2H-pyrazolo[4,3-d]pyrimidin-5-yl) carbamate (70 mg, 0.090 mmol) in MeOH (5 mL), was added aqueous HCl (1.203 mL, 1.804 mmol) dropwise over 2 min. The mixture was stirred at 60° C. for 4 h. The reaction mixture was concentrated under reduced pressure. The residue was triturated with diethyl ether (2×10 mL) and pet. ether (1×10 mL) and dried to afford the title compound (45 mg, 0.078 mmol, 87% yield) as a light yellowish semi-solid. LC-MS (ES): m/z=538.2 [M+H]⁺

Step 11. (R)-2-((5-Amino-2-((5-(((tetrahydrofuran-3-yl)amino)methyl)imidazo[1,2-a]pyridin-8-yl) methyl)-2H-pyrazolo[4,3-d]pyrimidin-7-yl) (butyl)amino) ethan-1-ol: To a solution containing methyl (R)-(7-(butyl(2-hydroxyethyl) amino)-2-((5-(((tetrahydrofuran-3-yl) amino) methyl) imidazo[1,2-a]pyridin-8-yl) methyl)-2H-pyrazolo[4,3-d]pyrimidin-5-yl) carbamate HCl (45 mg, 0.078 mmol) in dioxane (5 mL), was added an aqueous solution of NaOH (0.784 mL, 2.352 mmol) at room temperature. The reaction mixture was heated to 70° C. and stirred for 4 h. The separated organic layer was concentrated under reduced pressure. The residue was purified via preparative LC/MS (Column: Waters XBridge C18, 150 mm×19 mm, 5-μm particles; mobile phase A: 5:95 acetonitrile:water with 10 mM ammonium acetate; mobile phase B: 95:5 acetonitrile:water with 10 mM ammonium acetate; gradient: a 0-minute hold at 10% B, 10-25% B over 20 minutes, then a 5-minute hold at 100% B; flow rate: 20 mL/min; column temperature: 25° C.). Fractions containing the desired product were combined and dried via centrifugal evaporation to afford the title compound (6.1 mg, 0.013 mmol, 16.23% yield).

| Cmpd | | Analytical Data (¹H NMR 400 MHz, DMSO-d₆; |
|---|---|---|
| No. | Structure | LC-MS) |
| 187 | 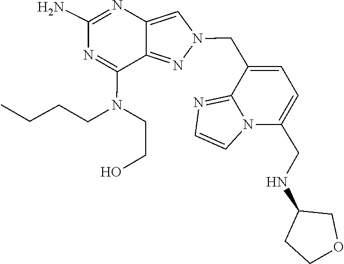 [SMILES: CCCCN(CCO)c1nc(N)nc2cn(Cc3ccc(CN[C@@H]4CCOC4)n4ccnc34)nc12] | δ = 8.10-7.93 (m, 2H), 7.66 (s, 1H), 7.03-6.96 (m, 1H), 6.90 (d, J = 7.1 Hz, 1H), 6.16 (br s, 2H), 5.80 (s, 2H), 4.74 (br s, 1H), 4.19-4.05 (m, 2H), 4.04-3.94 (m, 2H), 3.78-3.60 (m, 8H), 3.45 (br s, 4H), 1.99-1.92 (m, 1H), 1.73-1.44 (m, 3H), 1.24 (s, 2H), 0.96-0.72 (m, 3H). LC-MS (ES): m/z = 480.2 [M + H]⁺, RT (min) = 1.35 |

### Example S18. 2-((5-Amino-2-((5-((cyclobutylamino)methyl)-[1,2,4]triazolo[1,5-a]pyridin-8-yl)methyl)-2H-pyrazolo[4,3-d]pyrimidin-7-yl)(butyl)amino)ethan-1-ol (Compound 175)

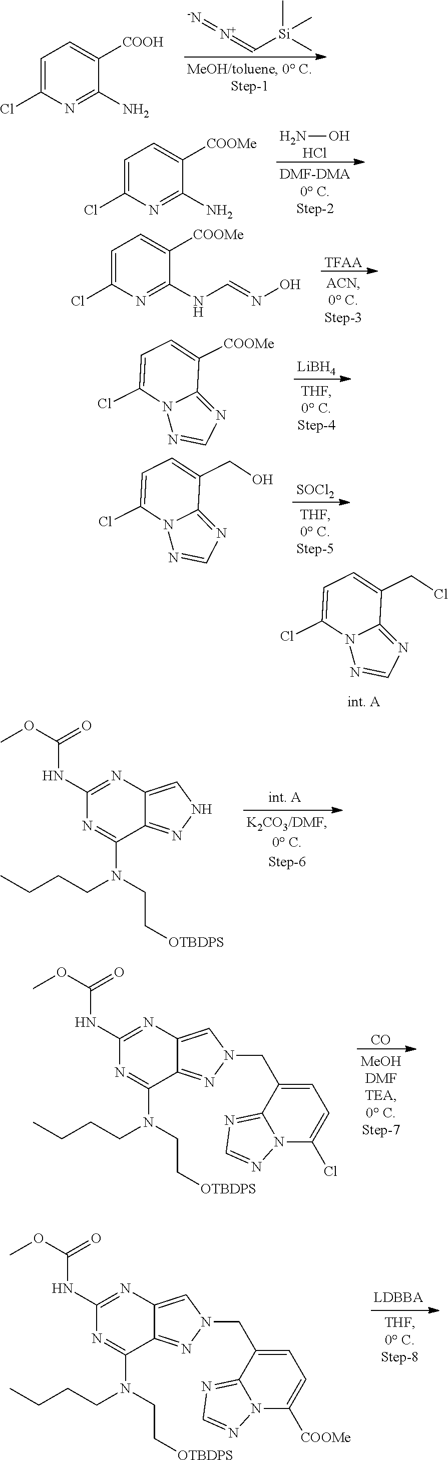

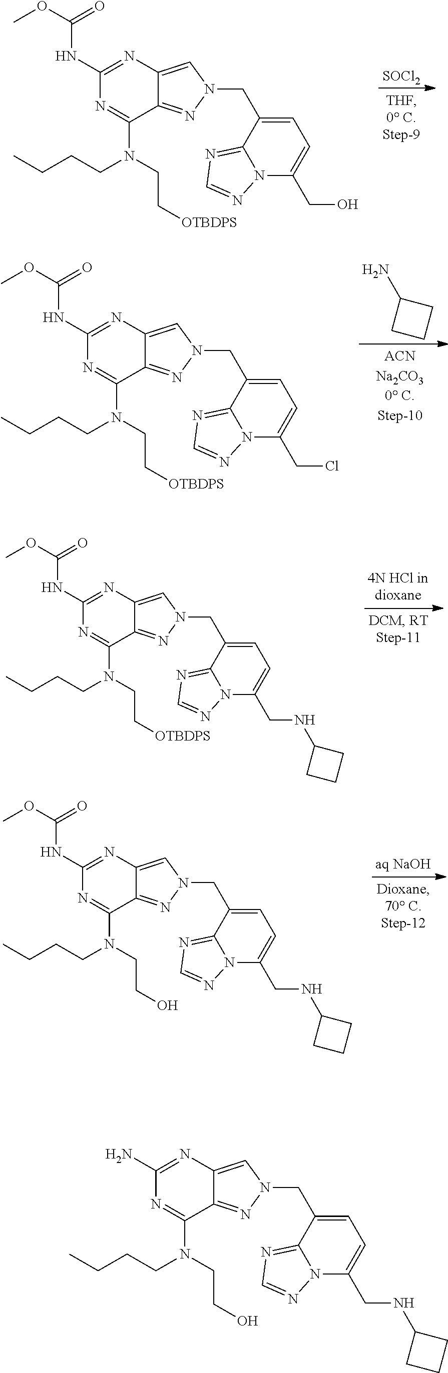

Step 1. Methyl 2-amino-6-chloronicotinate: To a solution of 2-amino-6-chloronicotinic acid (5 g, 29.0 mmol) in anhydrous toluene (60 mL) and MeOH (40 mL) at 0° C., was added trimethylsilyl)diazomethane (2M solution in hexanes, 29.0 mL, 57.9 mmol). After being stirred at room temperature for 2 h under nitrogen atmosphere, the reaction mixture was concentrated in vacuo at 40° C. The residue was partitioned between ethyl acetate and water. The organic layer was washed with brine, dried over anhydrous sodium sulphate and concentrated to get the crude product, which was purified by CombiFlash chromatography (Redisep Rf silica gel 40 g 60-120 silica gel; 10-15% ethyl acetate in pet. ether as an eluent) to afford the title compound (4.5 g, 24.12 mmol, 83% yield) as an off-white solid. ¹H NMR (500 MHz, DMSO-d₆) δ=8.05 (d, J=8.24 Hz, 1H) 7.47-7.60 (m, 2H) 6.66 (d, J=7.93 Hz, 1H) 3.81 (s, 3H). LC/MS (ES): m/z=187.0 [M+H]⁺.

Step 2. Methyl 6-chloro-2-(N′-hydroxyformimidamido)nicotinate: To a solution of methyl 2-amino-6-chloronicotinate (4.5 g, 24.12 mmol) in dry 2-propanol (25 mL), DMF-DMA (3.87 mL, 28.9 mmol) was added. The reaction mixture was stirred at 70° C. for 3 h. Hydroxylamine hydrochloride (2.51 g, 36.2 mmol) was added. The reaction mixture was stirred at RT for 4 h and then concentrated in vacuo. The residue was partitioned between EtOAc and water. The organic layer was washed with water and brine, dried over anhydrous Na₂SO₄, and concentrated to afford the title compound (5 g, 21.78 mmol, 90% yield). ¹H NMR (500 MHz, CDCl₃) δ=10.83 (br s, 1H) 8.15-8.25 (m, 2H) 6.89 (d, J=8.24 Hz, 1H) 3.95 (s, 3H). LC/MS (ES): m/z=230.0 [M+H]⁺.

Step 3. Methyl 5-chloro-[1,2,4]triazolo[1,5-a]pyridine-8-carboxylate: To a solution of methyl 6-chloro-2-(N-hydroxyformimidamido)nicotinate (5.0 g, 21.78 mmol) in dry acetonitrile (100 mL), was added TFAA (6.15 mL, 43.6 mmol) at 0° C. After being stirred for 16 h under nitrogen atmosphere, the reaction mixture was concentrated in vacuo to afford the residue, which was purified by CombiFlash chromatography (Redisep Rf silica gel 40 g 60-120 silica gel; 10-30% ethyl acetate in pet. ether as eluent) to afford the title compound (3 g, 14.18 mmol, 65.1% yield) as a colorless solid. ¹H NMR (500 MHz, CDCl₃) δ=8.56 (s, 1H) 8.33 (d, J=7.93 Hz, 1H) 7.24-7.30 (m, 2H) 4.09 (s, 3H). LC/MS (ES): m/z=212.1 [M+H]⁺.

Step 4. (5-Chloro-[1,2,4]triazolo[1,5-a]pyridin-8-yl)methanol: To a solution of ethyl 5-chloro-[1,2,4]triazolo[1,5-a]pyridine-8-carboxylate (2.5 g, 11.08 mmol) in anhydrous tetrahydrofuran (60 mL) at 0° C., was added a solution of LiBH₄ in THE (55.4 mL, 111 mmol). After being stirred at room temperature for 2 h under nitrogen atmosphere, the reaction mixture was quenched with ice cold water and was filtered through Celite bed, which was washed with EtOAc. The filtrate was washed with water and brine, dried over anhydrous sodium sulphate, and concentrated in vacuo. The crude product was purified by CombiFlash chromatography (Redisep Rf silica gel 40 g 60-120 silica gel; 20-60% ethyl acetate in pet. ether as an eluent) to afford the title compound (1.6 g, 8.71 mmol, 79% yield) as a light yellow solid. ¹H NMR (500 MHz, CDCl₃) δ=8.42 (s, 1H) 7.52 (d, J=7.63 Hz, 1H) 7.16 (d, J=7.63 Hz, 1H) 5.07 (s, 2H), LC/MS (ES): m/z=184.1 [M+H]⁺.

Step 5. 5-Chloro-8-(chloromethyl)-[1,2,4]triazolo[1,5-a]pyridine: To a solution of (5-chloro-[1,2,4]triazolo[1,5-a]pyridin-8-yl)methanol (1.5 g, 8.17 mmol) in dry tetrahydrofuran (5 mL), was added SOCl₂ (2.98 mL, 40.9 mmol) at 0° C. for 2 h under nitrogen atmosphere. The reaction mixture was concentrated in vacuo to afford the title compound (1.3 g, 6.43 mmol, 79% yield). LC/MS (ES): m/z=202.0 [M+H]⁺.

Step 6. Methyl (7-(butyl(2-((tert-butyldiphenylsilyl) oxy) ethyl) amino)-2-((5-chloro-[1,2,4]triazolo[1,5-a]pyridin-8-yl)methyl)-2H-pyrazolo[4,3-d]pyrimidin-5-yl)carbamate: To a solution of methyl (7-(butyl(2-((tert-butyldiphenylsilyl) oxy) ethyl) amino)-2H-pyrazolo[4,3-d]pyrimidin-5-yl) carbamate (3 g, 5.49 mmol) in dry DMF (5 mL), were added K₂CO₃ (1.517 g, 10.97 mmol) and 5-chloro-8-(chloromethyl)-[1,2,4]triazolo[1,5-a]pyridine (1.109 g, 5.49 mmol). After being stirred at 45° C. for 12 h under nitrogen atmosphere, the reaction mixture was partitioned between ethyl acetate and ice cold water. The organic layer was washed with water and brine, dried over anhydrous sodium sulphate, and concentrated in vacuo. The crude product was purified by CombiFlash chromatography (Redisep Rf silica gel 40 g 60-120 silica gel; 2-5% MeOH in CHCl₃ as an eluent) to afford the title compound (1.5 g, 2.106 mmol, 38.4% yield) as a brown oil. LC-MS (ES): m/z=712.0 [M+H]⁺.

Step 7. Methyl 8-((7-(butyl(2-((tert-butyldiphenylsilyl) oxy) ethyl) amino)-5-((methoxycarbonyl)amino)-2H-pyrazolo[4,3-d]pyrimidin-2-yl) methyl)-[1,2,4]triazolo[1,5-a]pyridine-5-carboxylate: To a solution of methyl (7-(butyl(2-((tert-butyldiphenylsilyl) oxy) ethyl) amino)-2-((5-chloro-[1,2,4]triazolo[1,5-a]pyridin-8-yl)methyl)-2H-pyrazolo[4,3-d]pyrimidin-5-yl)carbamate (1.2 g, 1.685 mmol) in dry MeOH (15 mL), were added DMF (15 mL), TEA (1.174 mL, 8.42 mmol) and PdCl₂(dppf)-CH₂Cl₂ (0.275 g, 0.337 mmol). The reaction mixture was heated at 100° C. under CO (100 psi pressure) in a mini-clave for 16 h, cooled to RT, filtered through a Celite bed and the bed was washed with EtOAc. The filtrate was concentrated in vacuo. The crude product was purified by CombiFlash chromatography (Redisep Rf silica gel 40 g 60-120 silica gel; 10-100% ethyl acetate in pet. ether as eluent) to afford the title compound (1.1 g, 1.495 mmol, 89% yield) as a light brown oil. LC/MS (ES): m/z=736.0 [M+H]⁺.

Step 8. Methyl (7-(butyl(2-((tert-butyldiphenylsilyl) oxy) ethyl) amino)-2-((5-(hydroxymethyl)-[1,2,4]triazolo[1,5-a]pyridin-8-yl) methyl)-2H-pyrazolo[4,3-d]pyrimidin-5-yl) carbamate: To a solution of methyl 8-((7-(butyl(2-((tert-butyldiphenylsilyl) oxy) ethyl) amino)-5-((methoxycarbonyl)amino)-2H-pyrazolo[4,3-d]pyrimidin-2-yl) methyl)-[1,2,4]triazolo[1,5-a]pyridine-5-carboxylate (1.1 g, 1.495 mmol) in tetrahydrofuran (10 mL), was added Lithium diisobutyl-tert-butoxyaluminum hydride solution (0.25 M in THF/hexane, 59.8 mL, 14.95 mmol) at 0° C. and stirred for 12 h. The reaction mixture was quenched with Rochelle's salt solution and extracted with ethyl acetate. The organic layer was washed with water and brine, dried over anhydrous sodium sulphate, and concentrated to get crude product, which was purified by CombiFlash chromatography (Redisep Rf silica gel 24 g 60-120 silica gel; 1-10% MeOH in CHCl₃ as an eluent) to afford the title compound (800 mg, 1.130 mmol, 76% yield) as a brown solid. LC-MS (ES): m/z=707.0 [M+H]⁺.

Step 9. Methyl (7-(butyl(2-((tert-butyldiphenylsilyl) oxy) ethyl) amino)-2-((5-(chloromethyl)-[1,2,4]triazolo[1,5-a]pyridin-8-yl)methyl)-2H-pyrazolo[4,3-d]pyrimidin-5-yl) carbamate: To a stirred solution of methyl (7-(butyl(2-((tert-butyldiphenylsilyl) oxy) ethyl) amino)-2-((5-(hydroxymethyl)-[1,2,4]triazolo[1,5-a]pyridin-8-yl)methyl)-2H-pyrazolo[4,3-d]pyrimidin-5-yl)carbamate (200 mg, 0.283 mmol) in tetrahydrofuran (5 mL) at 0° C. under nitrogen atmosphere, was added SOCl₂ (0.103 mL, 1.413 mmol). The reaction mixture was stirred at 0° C. for 0.5 h under nitrogen atmosphere and subsequently concentrated in vacuo to afford the title compound (201 mg, 0.277 mmol, 98% yield) as a light yellow solid. LC-MS (ES): m/z=726.4 [M+H]⁺.

Step 10. Methyl (7-(butyl(2-((tert-butyldiphenylsilyl)oxy)ethyl)amino)-2-((5-((cyclobutylamino) methyl)-[1,2,4]triazolo[1,5-a]pyridin-8-yl)methyl)-2H-pyrazolo[4,3-d]pyrimidin-5-yl)carbamate: To a solution of methyl (7-(butyl(2-((tert-butyldiphenylsilyl) oxy) ethyl) amino)-2-((5-(chloromethyl)-[1,2,4]triazolo[1,5-a]pyridin-8-yl)methyl)-2H-pyrazolo[4,3-d]pyrimidin-5-yl)carbamate (120 mg, 0.165 mmol) in acetonitrile (2 mL) at RT, were added cyclobutanamine (23.50 mg, 0.330 mmol), potassium iodide (0.549 mg, 3.30 μmol) and Na₂CO₃ (52.5 mg, 0.496 mmol). The reaction mixture was stirred at 60° C. for 2 h, diluted with DCM and washed with water and brine. The organic layer was dried over anhydrous sodium sulphate and concentrated to afford the title compound (125 mg, 0.164 mmol, 99% yield) as a light brown oil. LC-MS (ES): m/z=761.5 [M+H]⁺.

Step 11. Methyl (7-(butyl(2-hydroxyethyl) amino)-2-((5-((cyclobutylamino)methyl)-[1,2,4]triazolo[1,5-a]pyridin-8-yl)methyl)-2H-pyrazolo[4,3-d]pyrimidin-5-yl)carbamate hydrochloride: To a stirred solution of methyl (7-(butyl(2-((tert-butyldiphenylsilyl) oxy) ethyl) amino)-2-((5-((cyclobutylamino)methyl)-[1,2,4]triazolo[1,5-a]pyridin-8-yl)methyl)-2H-pyrazolo[4,3-d]pyrimidin-5-yl)carbamate (120 mg, 0.158 mmol) in dry CH₂Cl₂ (1 mL), was added 4N HCl in dioxane (1.5 mL, 6.00 mmol). The reaction mixture was stirred at RT for 2 h. The solvent was concentrated in vacuo to afford the title compound (88 mg, 0.157 mmol, 100% yield) as a light brown oil. LC-MS (ES): m/z=523.5 [M+H]⁺.

Step 12. 2-((5-Amino-2-((5-((cyclobutylamino)methyl)-[1,2,4]triazolo[1,5-a]pyridin-8-yl)methyl)-2H-pyrazolo[4,3-d]pyrimidin-7-yl)(butyl)amino)ethan-1-ol: To a stirred solution of methyl (7-(butyl(2-hydroxyethyl) amino)-2-((5-((cyclobutylamino)methyl)-[1,2,4]triazolo[1,5-a]pyridin-8-yl)methyl)-2H-pyrazolo[4,3-d]pyrimidin-5-yl)carbamate hydrochloride (81 mg, 0.145 mmol) in 1,4-dioxane (1 mL), was added a solution of NaOH (3 mL, 0.145 mmol). The reaction mixture was stirred at 70° C. for 1 h and then cooled to room temperature. The organic layer was separated, dried over Na₂SO₄, filtered, and concentrated under reduced pressure. The residue was purified by reversed phase preparative LC/MS (Column: Waters XBridge C18, 19×150 mm, 5-μm particles; mobile phase A: 10 mM ammonium acetate; mobile phase B: acetonitrile; gradient: 10⁻⁴5% B over 20 minutes, then a 5-minute hold at 100% B; flow rate: 15 mL/min). The fraction collection was triggered by MS and UV signals. Fractions containing the desired product were combined and dried via centrifugal evaporation using Genevac to afford the title compound (20.5 mg, 0.044 mmol, 30.2% yield).

Compound 176 was prepared by following the procedures described above in Compound 175.

| Cmpd | | Analytical Data (¹H NMR 400 MHz, DMSO- |
|---|---|---|
| No. | Structure | d₆; LC-MS) |
| 175 | 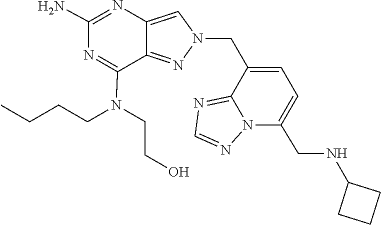 [SMILES: CCCCN(CCO)c1nc(N)nc2cn(Cc3ccc(CNC4CCC4)n4ncnc34)nc12] | δ = 8.55 (s, 1H), 7.93 (br s, 1H), 7.42 (br d, J = 5.9 Hz, 1H), 7.20 (d, J = 7.3 Hz, 1H), 5.81 (s, 2H), 5.63 (br s, 2H),4.68 (br s, 1H), 4.09 (s, 3H), 3.76-3.59 (m, 4H), 3.23-3.18 (m, 2H), 2.06-1.94 (m, 2H), 1.70-1.42 (m, 6H), 1.36- 1.02 (m, 2H), 0.73 (br s, 3H). LC-MS (ES): m/z = 465.2 [M + H]⁺, RT (min) = 1.28 |

### Example S19. 2-((5-Amino-2-((5-((tert-butylamino) methyl)-[1,2,4]triazolo[1,5-a]pyridin-8-yl)methyl)-2H-pyrazolo[4,3-d]pyrimidin-7-yl)(butyl)amino)ethan-1-ol (Compound 189)

The title compound was prepared from methyl (7-(butyl(2-((tert-butyldiphenylsilyl) oxy) ethyl) amino)-2-((5-(chloromethyl)-[1,2,4]triazolo[1,5-a]pyridin-8-yl)methyl)-2H-pyrazolo[4,3-d]pyrimidin-5-yl) carbamate and 2-methylpropan-2-amine following three-step procedure for Compound 175.

| Cmpd | | Analytical Data (¹H NMR 400 MHz, DMSO-d₆; |
|---|---|---|
| No. | Structure | LC-MS) |
| 189 | 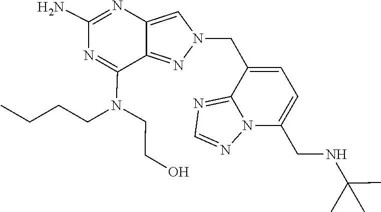 [SMILES: CCCCN(CCO)c1nc(N)nc2cn(Cc3ccc(CNC(C)(C)C)n4ncnc34)nc12] | δ = 8.56 (br s, 1H), 8.06 (br d, J = 2.4 Hz, 1H), 7.52 (br d, J = 2.0 Hz, 1H), 7.31 (d, J = 7.4 Hz, 1H), 6.40 (s, 1H),5.85 (s, 2H), 4.74 (br s, 1H), 4.18 (br s, 5H), 3.78-3.58 (m, 6H), 1.67-1.26 (m, 2H), 1.13 (s, 9H), 0.96-0.58 (m, 3H). LC- MS (ES): m/z = 467.3 [M + H]⁺, RT (min) = 1.61 |

### Example S20. 2-((5-amino-2-((7-((((1r,3r)-3-methoxycyclobutyl)amino)methyl)-2-methylbenzo[d]oxazol-4-yl)methyl)-2H-pyrazolo[4,3-d]pyrimidin-7-yl)(butyl)amino)ethan-1-ol (Compound 141)

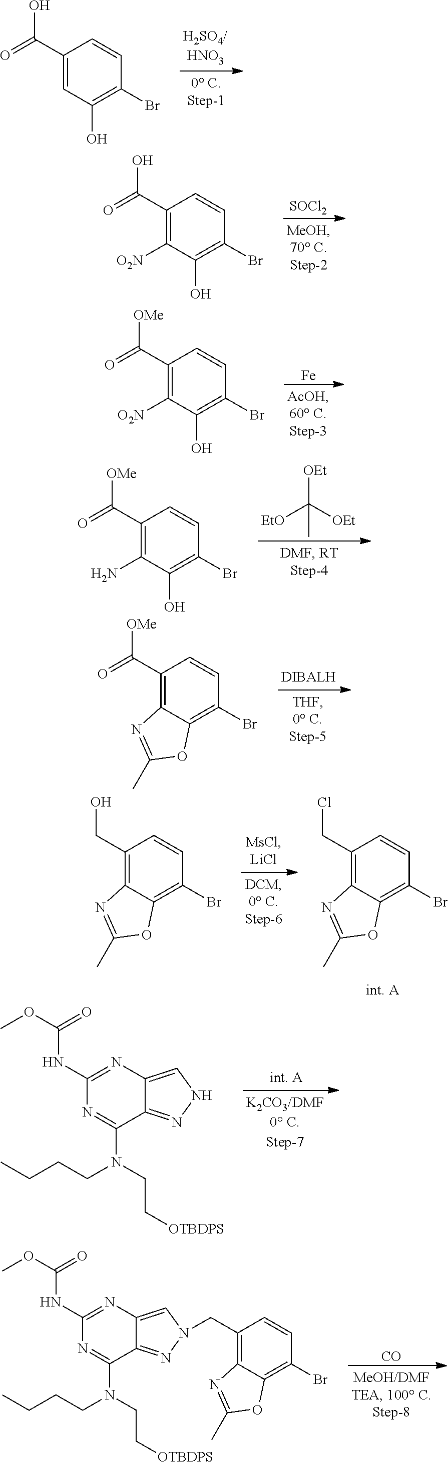

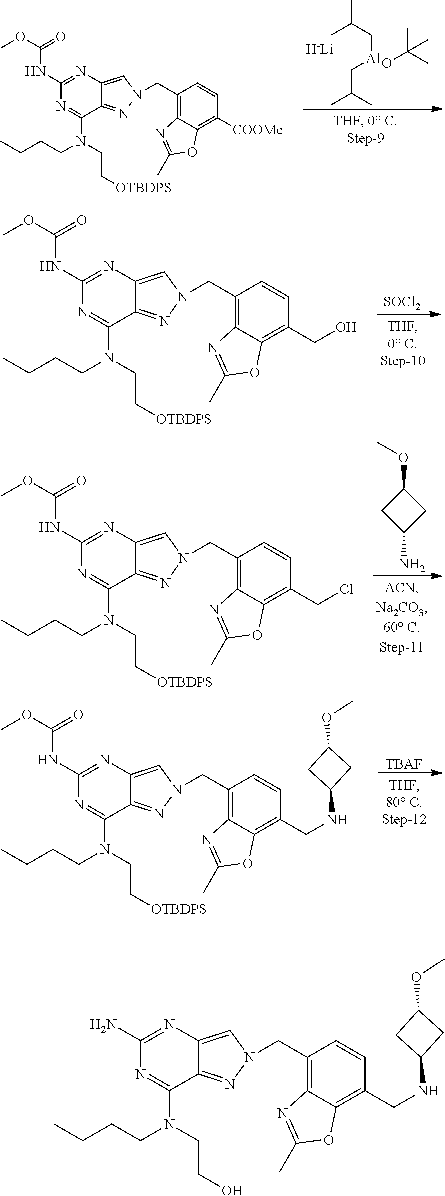

Step 1. 4-Bromo-3-hydroxy-2-nitrobenzoic acid: A solution of 4-bromo-3-hydroxybenzoic acid (20 g, 92 mmol) in concentrated sulphuric acid (460 mL, 8630 mmol) was stirred at 20° C. for 30 min and cooled to 0° C. A chilled solution of fuming nitric acid (4.53 mL, 101 mmol) and sulfuric acid (460 mL, 8630 mmol) dropwise under nitrogen atmosphere. The reaction mixture was cooled to 0° C. for 0.5 h, poured over ice, and extracted with ethyl acetate. The combined organic extracts were washed with brine, dried over Na₂SO₄ and concentrated under reduced pressure to afford the title compound (18 g, 68.7 mmol, 74.5% yield) as a light yellow solid.

Step 2. Methyl 4-bromo-3-hydroxy-2-nitrobenzoate: To a solution of 4-bromo-3-hydroxy-2-nitrobenzoic acid (18 g, 68.7 mmol) in MeOH (150 mL), thionyl chloride (25.07 mL, 343 mmol) at 0° C., the reaction mixture was stirred at 70° C. under N₂ for 16 h, cooled to RT and concentrated in vacuo at 40° C. The crude product was purified by CombiFlash chromatography (60-120 silica gel; 10-70% ethyl acetate in pet. ether as an eluent) to afford the title compound (8 g, 29.0 mmol, 42.2% yield) as a light brown oil. LC-MS (ES): m/z=275.80 [M+H]⁺.

Step 3. Methyl 2-amino-4-bromo-3-hydroxybenzoate: To a stirred solution of methyl 4-bromo-3-hydroxy-2-nitrobenzoate (8 g, 29.0 mmol) in AcOH (200 mL), was added iron powder (4.86 g, 87 mmol). The reaction mixture was stirred at 60° C. for 1 h. Ethyl acetate was added. The insoluble material was filtered off, and the filtrate was concentrated under reduced pressure. The residue was partitioned between ethyl acetate and saturated aqueous 10% NaHCO₃ solution. The organic layer was washed with water and brine, dried over anhydrous Na₂SO₄ and concentrated under reduced pressure. The residue was purified by CombiFlash chromatography (Redisep Rf silica gel 40 g 60-120 silica gel; 10-30% ethyl acetate in pet. ether) to afford the title compound (6 g, 24.38 mmol, 84% yield) as a light brown oil. ¹H NMR (400 MHz, DMSO-d₆) δ=7.22 (d, J=9.01 Hz, 1H) 6.69 (d, J=9.01 Hz, 1H) 3.79 (s, 3H); LC-MS (ES): m/z=248.05 [M+H]⁺

Step 4. Methyl 7-bromo-2-methylbenzo[d]oxazole-4-carboxylate: To a solution of methyl 2-amino-4-bromo-3-hydroxybenzoate (8 g, 32.5 mmol) in DMF (100 mL), were added triethyl orthoacetate (39.6 mL, 215 mmol) and PTSA (0.618 g, 3.25 mmol). After being stirred at room temperature for 12 h under nitrogen atmosphere, the reaction mixture was partitioned between ethyl acetate and water. The organic layer was washed with water and brine, dried over anhydrous Na₂SO₄ and concentrated. The residue was purified by CombiFlash chromatography (Redisep Rf silica gel 40 g 60-120 silica gel; 10-50% ethyl acetate in pet. ether an eluent) to afford title the compound (6 g, 22.22 mmol, 68.3% yield) as a light yellow solid. ¹H NMR (400 MHz, CDCl₃) δ=7.87 (d, J=8.24 Hz, 1H) 7.52 (d, J=8.55 Hz, 1H) 4.03 (s, 3H) 2.77 (s, 3H), LC-MS (ES): m/z=270.0 [M+H]⁺

Step 5. (7-Bromo-2-methylbenzo[d]oxazol-4-yl) methanol: To a solution of methyl 7-bromo-2-methylbenzo[d]oxazole-4-carboxylate (6 g, 22.22 mmol) in anhydrous tetrahydrofuran (300 mL) at −78° C., was added 1M DIBAL-H (133 mL, 133 mmol). After being stirred at room temperature for 2 h under nitrogen atmosphere, the reaction mixture was partitioned between ethyl acetate and ice cold water. The organic layer was washed with water and brine, dried over anhydrous Na₂SO₄ and concentrated in vacuo at 45° C. The crude product was purified by CombiFlash chromatography (Redisep Rf silica gel 40 g 60-120 silica gel; 20-60% ethyl acetate in pet. ether as eluent) to afford the title compound (4.5 g, 18.59 mmol, 84% yield) as a light yellow solid. ¹H NMR (400 MHz, CDCl₃) δ=7.45 (d, J=8.00 Hz, 1H) 7.20 (d, J=8.01 Hz, 1H) 5.01 (s, 2H) 2.72 (s, 3H); LC-MS (ES): m/z=243.7 [M+H]⁺

Step 6. 7-Bromo-4-(chloromethyl)-2-methylbenzo[d]oxazole: To a solution of (7-bromo-2-methylbenzo[d]oxazol-4-yl) methanol (4.0 g, 16.52 mmol) in dry CH₂Cl₂ (20 mL), were added TEA (4.61 mL, 33.0 mmol), MsCl (2.58 mL, 33.0 mmol) and lithium chloride (1.401 g, 33.0 mmol) at 0° C. for 16 h under nitrogen atmosphere. The reaction mixture was concentrated in vacuo at 30° C. The residue was partitioned between ethyl acetate and water. The organic layer was washed with brine, dried over anhydrous Na₂SO₄ and concentrated in vacuo to get crude product, which was purified by CombiFlash chromatography (Redisep Rf silica gel 40 g 60-120 silica gel; 10-30% ethyl acetate in pet. ether to afford the title compound (3.8 g, 14.59 mmol, 88% yield) as an off-white solid. ¹H NMR (400 MHz, CDCl₃) δ=7.45 (d, J=8.00 Hz, 1H) 7.20 (d, J=8.01 Hz, 1H) 5.01 (s, 2H) 2.72 (s, 3H); LC-MS (ES): m/z=261.6 [M+H]⁺

Step 7. Methyl (2-((7-bromo-2-methylbenzo[d]oxazol-4-yl) methyl)-7-(butyl(2-((tert-butyldiphenylsilyl) oxy) ethyl) amino)-2H-pyrazolo[4,3-d]pyrimidin-5-yl) carbamate: To a solution of methyl (7-(butyl(2-((tert-butyldiphenylsilyl) oxy) ethyl) amino)-2H-pyrazolo[4,3-d]pyrimidin-5-yl) carbamate (3 g, 5.49 mmol) in dry DMF (25 mL), were added K₂CO₃ (1.517 g, 10.97 mmol) and 7-bromo-4-(chloromethyl)-2-methylbenzo[d]oxazole (1.429 g, 5.49 mmol) at 0° C. for 16 h. After being stirred at 0° C. for 16 h under nitrogen atmosphere. The reaction mixture was partitioned between ethyl acetate and ice cold water. The organic layer was washed with water and brine, dried over anhydrous Na₂SO₄ and concentrated in vacuo at 45° C. The crude product was purified by CombiFlash chromatography (Redisep Rf silica gel 40 g 60-120 silica gel; 2% MeOH in CHCl₃ as eluent) to afford the title compound (3.8 g, 4.93 mmol, 90% yield) as an off-white solid; LC-MS (ES): m/z=772.6 [M+H]⁺

Step 8. Methyl 4-((7-(butyl(2-((tert-butyldiphenylsilyl) oxy) ethyl) amino)-5-((methoxycarbonyl) amino)-2H-pyrazolo[4,3-d]pyrimidin-2-yl)methyl)-2-methylbenzo[d]oxazole-7-carboxylate: To a solution of methyl (2-((7-bromo-2-methylbenzo[d]oxazol-4-yl) methyl)-7-(butyl(2-((tert-butyldiphenylsilyl) oxy) ethyl) amino)-2H-pyrazolo[4,3-d]pyrimidin-5-yl) carbamate (2.0 g, 2.59 mmol) in dry MeOH (15 mL), were added DMF (15 mL), TEA (1.808 mL, 12.97 mmol) and PdCl₂(dppf)-CH₂Cl₂ adduct (0.424 g, 0.519 mmol). The reaction mixture was heated at 100° C. under CO (100 Psi pressure) in mini-clave for 16 h, filtered through a Celite bed and the bed was washed with EtOAc. The filtrate was concentrated in vacuo at 40° C. The crude product was purified by CombiFlash chromatography (Redisep Rf silica gel 40 g 60-120 silica gel; 2-3% MeOH in CHCl₃ as eluent) to afford the title compound (1.2 g, 1.600 mmol, 61.7% yield) as a brown solid. ¹H NMR (400 MHz, DMSO-d₆+D₂O) δ=8.21-8.33 (m, 1H) 7.77-7.83 (m, 1H) 7.45-7.56 (m, 3H) 7.30-7.43 (m, 3H) 7.18-7.28 (m, 3H) 6.84-6.91 (m, 1H) 5.88-5.96 (m, 1H) 5.82 (s, 1H) 5.69 (s, 1H) 4.22-4.28 (m, 1H) 3.98-4.07 (m, 1H) 3.91 (s, 4H) 3.87 (s, 3H) 3.72-3.77 (m, 3H) 2.65 (d, J=11.60 Hz, 3H) 1.57-1.64 (m, 1H) 1.37-1.44 (m, 1H) 1.25-1.33 (m, 1H) 1.00-1.07 (m, 1H) 0.75-0.93 (m, 12H). LC-MS (ES): m/z=750.3 [M+H]⁺

Step 9. Methyl (7-(butyl(2-((tert-butyldiphenylsilyl) oxy) ethyl) amino)-2-((7-(hydroxymethyl)-2-methylbenzo[d]oxazol-4-yl) methyl)-2H-pyrazolo[4,3-d]pyrimidin-5-yl) carbamate: To a solution of methyl 4-((7-(butyl(2-((tert-butyldiphenylsilyl) oxy) ethyl) amino)-5-((methoxycarbonyl)amino)-2H-pyrazolo[4,3-d]pyrimidin-2-yl) methyl)-2-methylbenzo[d]oxazole-7-carboxylate (1.4 g, 1.867 mmol) in tetrahydrofuran (5 mL), was added lithium diisobutyl-tert-butoxyaluminum hydride solution (0.25 M in THF/hexane, 74.7 mL, 18.67 mmol) at 0° C. After being stirred for 12 h, the reaction mixture was quenched with Rochelle's salt solution and extracted with ethyl acetate. The organic layer was washed with water and brine, dried over anhydrous sodium sulphate and concentrated. The residue was purified by CombiFlash chromatography (Redisep Rf silica gel 40 g, 60-120 silica gel; 1-10% MeOH in CHCl₃ as eluent) to afford the title compound (0.8 g, 1.108 mmol, 59.4% yield) as an off-white solid. ¹H NMR (400 MHz, DMSO-d₆) δ=9.27-9.54 (m, 1H) 8.13-8.33 (m, 1H) 7.49-7.58 (m, 4H) 7.13-7.43 (m, 7H) 6.89-6.95 (m, 1H) 5.70-5.88 (m, 2H) 5.30-5.41 (m, 1H) 4.64-4.77 (m, 2H) 4.27-4.38 (m, 1H) 4.04-4.16 (m, 1H) 3.89-3.99 (m, 1H) 3.72-3.87 (m, 3H) 3.55 (s, 3H) 2.62 (m, 3H) 1.41-1.69 (m, 2H) 1.14-1.35 (m, 2H) 0.67-0.99 (m, 12H). LC-MS (ES): m/z=722.2 [M+H]⁺

Step 10. Methyl (7-(butyl(2-((tert-butyldiphenylsilyl)oxy)ethyl)amino)-2-((7-(chloromethyl)-2-methylbenzo[d]oxazol-4-yl) methyl)-2H-pyrazolo[4,3-d]pyrimidin-5-yl) carbamate: To a stirred solution of methyl (7-(butyl(2-((tert-butyldiphenylsilyl) oxy) ethyl) amino)-2-((7-(hydroxymethyl)-2-methylbenzo[d]oxazol-4-yl) methyl)-2H-pyrazolo[4,3-d]pyrimidin-5-yl) carbamate (600 mg, 0.831 mmol) in tetrahydrofuran (5 mL) at 0° C. under nitrogen atmosphere, was added thionyl chloride (0.303 mL, 4.16 mmol). The reaction mixture was stirred at 0° C. for 0.5 h under nitrogen atmosphere and subsequently concentrated in vacuo to afford the title compound (610 mg, 0.824 mmol, 99% yield) as a light yellow solid. LC-MS (ES): m/z=740.05 [M+H]⁺

Step 11. Methyl (7-(butyl(2-((tert-butyldiphenylsilyl) oxy) ethyl) amino)-2-((7-((((1r,3r)-3-methoxycyclobutyl) amino) methyl)-2-methylbenzo[d]oxazol-4-yl) methyl)-2H-pyrazolo[4,3-d]pyrimidin-5-yl) carbamate: To a solution of methyl (7-(butyl(2-((tert-butyldiphenylsilyl) oxy)ethyl)amino)-2-((7-(chloromethyl)-2-methylbenzo[d]oxazol-4-yl) methyl)-2H-pyrazolo[4,3-d]pyrimidin-5-yl) carbamate (200 mg, 0.270 mmol) in acetonitrile (2 mL) at RT, were added trans-3-methoxycyclobutan-1-amine hydrochloride (74.3 mg, 0.540 mmol), potassium iodide (0.897 mg, 5.40 μmol) and Na₂CO₃ (86 mg, 0.810 mmol). The reaction mixture was stirred at 60° C. for 2 h, cooled to RT and partitioned between DCM and water. The organic layer was dried over anhydrous sodium sulphate and concentrated to afford the title compound (210 mg, 0.261 mmol, 97% yield) as a light brown oil. LC-MS (ES): m/z=805.5 [M+H]⁺

Step 12. 2-((5-Amino-2-((7-((((1r,3r)-3-methoxycyclobutyl) amino) methyl)-2-methylbenzo[d]oxazol-4-yl) methyl)-2H-pyrazolo[4,3-d]pyrimidin-7-yl) (butyl) amino) ethan-1-ol: To the suspension of methyl (7-(butyl(2-((tert-butyldiphenylsilyl) oxy) ethyl) amino)-2-((7-((((1r,3r)-3-methoxycyclobutyl) amino) methyl)-2-methylbenzo[d]oxazol-4-yl) methyl)-2H-pyrazolo[4,3-d]pyrimidin-5-yl) carbamate (220 mg, 0.273 mmol) in tetrahydrofuran (1.0 mL), was added a solution of TBAF in THE (0.820 mL, 0.820 mmol). The reaction mixture heated at 80° C. After being stirred at 80° C. for 16 h, the reaction mixture was concentrated in vacuo. The crude product was purified by reversed phase preparative LC-MS (Column: Waters XBridge C18, 19×150 mm, 5-μm particles; mobile phase A: 10 mM ammonium acetate; mobile phase B: acetonitrile; gradient: 10⁻⁴5% B over 20 minutes, then a 5-minute hold at 100% B; flow rate: 15 mL/min). The fraction collection was triggered by MS and UV signals. Fractions containing the desired product were combined and dried via centrifugal evaporation using Genevac to afford the title compound (1 mg, 1.966 μmol, 0.71900 yield).

| Cmpd | | Analytical Data (¹H NMR 400 MHz, DMSO-d₆; |
|---|---|---|
| No. | Structure | LC-MS) |
| 141 | 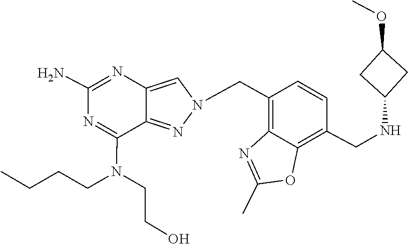 [SMILES: CCCCN(CCO)c1nc(N)nc2cn(Cc3ccc(CN[C@H]4C[C@H](OC)C4)c4oc(C)nc34)nc12] | δ = 8.23-8.05 (m, 1H), 7.61 (br s, 2H), 7.39 (br d, J = 7.6 Hz, 1H), 7.31-7.19 (m, 1H), 5.84 (s, 2H), 4.82 (br d, J = 4.0 Hz, 1H), 4.23-4.05 (m, 4H), 3.98 (br s, 1H), 3.83-3.74 (m, 2H), 3.71- 3.53 (m, 3H), 3.12 (s, 3H), 2.70-2.63 (m, 2H), 2.30-2.01 (m, 4H), 1.68-1.43 (m, 2H), 1.36- 1.10 (m, 3H), 0.97-0.72 (m, 3H), LC-MS (ES). LC-MS (ES): m/z = 509.2 [M + H]⁺, RT (min) = 1.28 |

### Example S21. 2-((5-Amino-2-((7-((cyclobutylamino)methyl)-2-methylbenzo[d]oxazol-4-yl) methyl)-2H-pyrazolo-[4,3-d] pyrimidin-7-yl) (butyl)amino) ethan-1-ol (Compound 140)

The title compound was prepared from methyl (7-(butyl(2-((tert-butyldiphenylsilyl) oxy) ethyl) amino)-2-((7-(chloromethyl)-2-methylbenzo[d]oxazol-4-yl) methyl)-2H-pyrazolo[4,3-d]pyrimidin-5-yl) carbamate and cyclobutylamine following three-step procedure for Compound 141.

| Cmpd | | Analytical Data (¹H NMR 400 MHz, DMSO-d₆; |
|---|---|---|
| No. | Structure | LC-MS) |
| 140 | 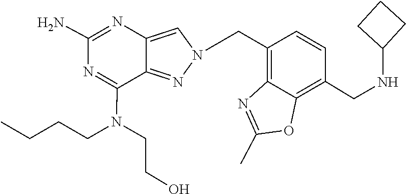 [SMILES: CCCCN(CCO)c1nc(N)nc2cn(Cc3ccc(CNC4CCC4)c4oc(C)nc34)nc12] | δ = 7.92 (br s, 1H), 7.30 (d, J = 7.8 Hz, 1H), 7.13 (d, J = 7.8 Hz, 1H), 6.10 (br s, 2H), 5.74 (s, 2H), 4.68 (br s, 1H), 4.07 (br s, 2H), 3.89 (s, 2H), 3.64 (br s, 4H), 3.22 (br d, J = 7.9 Hz, 3H), 2.66 (s, 3H), 2.11-1.99 (m, 2H), 1.76-1.46 (m, 6H), 1.24 (br s, 2H), 0.80 (brs, 3H). LC-MS (ES): m/z = 479.2 [M + H]⁺, RT (min) = 1.34 |

The following compounds were prepared by following the procedures described above in Example S1-Example S21.

| | | Analytical Data (¹H NMR | |
|---|---|---|---|
| Cmpd | | 500 MHz, DMSO-d₆, unless | Example |
| No. | Structure | otherwise stated; LC-MS) | No. |
| 1 | 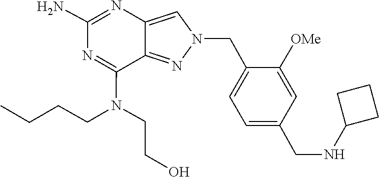 [SMILES: CCCCN(CCO)c1nc(N)nc2cn(Cc3ccc(CNC4CCC4)cc3OC)nc12] | δ 8.07 (s, 1H), 8.02 (s, 1H), 7.79 (s, 2H), 7.23 (s, 1H), 7.15 (dd, J = 25.8, 7.7 Hz, 1H), 7.04 (d, J = 7.4 Hz, 1H), 5.51 (d, J = 3.0 Hz, 2H), 4.41-4.08 (m, 2H), 4.03 (s, 2H), 3.94-3.78 (m, 4H), 3.76-3.58 (m, 2H), 2.52 (s, 3H), 2.15 (t, J = 8.3 Hz, 4H), 1.92-1.71 (m, 1H), 1.63 (d, J = 28.3 Hz, 1H), 1.31 (dq, J = 32.4, 7.4 | S1 |
| | | Hz, 2H), 0.94 (t, J = 7.4 Hz, | |
| | | 1H), 0.87 (t, J = 7.4 Hz, | |
| | | 3H). LC-MS (ES): m/z = | |
| | | 454.2 [M + H]+, RT (min) = | |
| | | 1.13 | |
| 2 | 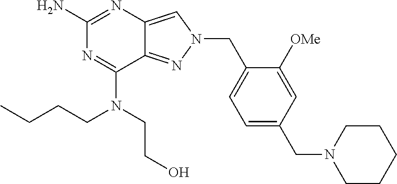 [SMILES: CCCCN(CCO)c1nc(N)nc2cn(Cc3ccc(CN4CCCCC4)cc3OC)nc12] | δ 7.70 (br s, 1H), 7.00-6.88 (m, 2H), 6.82 (br d, J-7.6 Hz, 1H), 5.48 (s, 2H), 5.37 (s, 2H), 4.16-4.00 (m, 1H), 3.81 (s, 3H), 3.72-3.60 (m, 1H), 3.53-3.40 (m, 1H), 3.08-2.98 (m, 1H), 2.29 (br s, 4H), 1.62-1.43 (m, 6H), 1.39-1.20 (m, 4H), 0.94- 0.76 (m, 3H). LC-MS (ES): m/z = 468.0 [M + H]+, RT (min) = 1.49 | S1 |
| 8 | 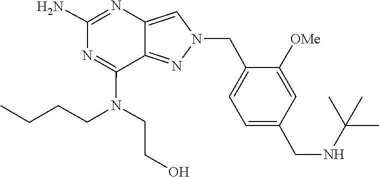 [SMILES: CCCCN(CCO)c1nc(N)nc2cn(Cc3ccc(CNC(C)(C)C)cc3OC)nc12] | δ 8.03 (d, J = 22.9 Hz, 1H), 7.80 (s, 2H), 7.38-6.98 (m, 3H), 5.51 (d, J = 2.7 Hz, 2H), 4.27-4.14 (m, 2H), 4.11 (s, 2H), 3.87 (s, 1H), 3.85 (s, 2H), 3.81 (d, J = 6.3 Hz, 2H), 3.70 (d, J = 19.1 Hz, 2H), 2.51 (d, J = 1.9 Hz, 1H), 1.83-1.56 (m, 2H), 1.36 (m, 10H), 0.91 (dt, J = 21.6, 7.3 Hz, 3H). 2 proton was not observed, which is | S1 |
| | | attributed to the water | |
| | | suppression used. LC-MS | |
| | | (ES): m/z = 456.0 [M + H]+, | |
| | | RT (min) = 1.13 | |
| 9 | 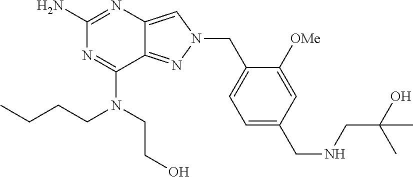 [SMILES: CCCCN(CCO)c1nc(N)nc2cn(Cc3ccc(CNCC(C)(C)O)cc3OC)nc12] | δ 7.70 (s, 1H), 7.06 (s, 1H), 6.95 (s, 1H), 6.87 (d, J = 7.6 Hz, 1H), 5.60 (s, 1H), 5.38 (s, 2H), 4.14 (d, J = 41.2 Hz, 1H), 3.82 (s, 3H), 3.74 (s, 2H), 3.64 (s, 1H), 2.51 (t, J = 1.8 Hz, 3H), 2.37 (s, 2H), 1.91 (s, 2H), 1.56 (s, 3H), 1.41-1.15 (m, 3H), 1.09 (s, 7H), 0.87 (s, 3H). LC-MS (ES): m/z = 472.3 [M + H]+, RT (min) = 1.11 | S1 |
| 10 | 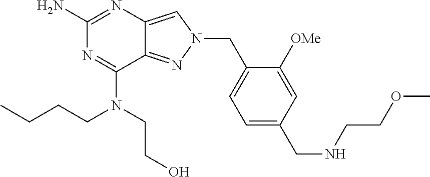 [SMILES: CCCCN(CCO)c1nc(N)nc2cn(Cc3ccc(CNCCOC)cc3OC)nc12] | δ 7.69 (s, 1H), 7.03 (s, 1H), 7.00-6.93 (m, 1H), 6.85 (d, J = 7.7 Hz, 1H), 5.66 (s, 1H), 5.37 (s, 2H), 3.81 (s, 2H), 3.70 (s, 1H), 3.59 (s, 1H), 3.39 (t, J = 5.6 Hz, 2H), 3.22 (s, 3H), 2.64 (t, J = 5.6 Hz, 2H), 2.55 (s, 2H), 2.53-2.44 (m, 2H), 1.89 (s, 6H), 1.54 (s, 3H), 1.25 (s, 2H), 0.85 (s, 2H). LC-MS (ES): m/z = 458.2 [M + H]+, RT (min) = 1.17 | S1 |
| 13 | 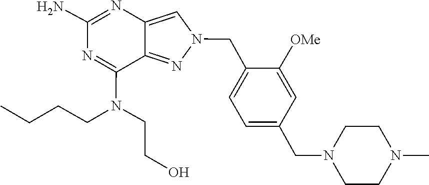 [SMILES: CCCCN(CCO)c1nc(N)nc2cn(Cc3ccc(CN4CCN(C)CC4)cc3OC)nc12] | δ 7.71 (s, 1H), 7.14-6.63 (m, 3H), 5.59 (s, 1H), 5.38 (s, 2H), 4.09 (s, 1H), 3.82 (s, 3H), 3.64 (s, 2H), 2.54- 2.41 (m, 5H), 2.33 (s, 6H), 2.14 (s, 3H), 1.91 (s, 2H), 1.55 (s, 3H), 1.25 (s, 3H), 0.85 (s, 3H). LC-MS (ES): m/z = 483.3 [M + H]+, RT (min) = 1.06 | S1 |
| 15 | 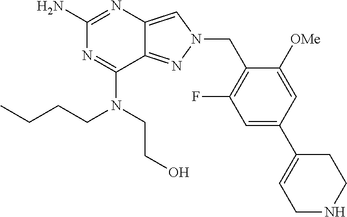 [SMILES: CCCCN(CCO)c1nc(N)nc2cn(Cc3c(F)cc(C4=CCNCC4)cc3OC)nc12] | δ 7.67 (s, 1H), 6.89 (d, J = 17.4 Hz, 2H), 6.34 (s, 1H), 5.48 (s, 1H), 5.40 (s, 3H), 4.02 (s, 1H), 3.87 (s, 3H), 3.62 (s, 2H), 2.93 (t, J = 5.7 Hz, 2H), 2.51 (d, J = 1.8 Hz, 3H), 2.35 (s, 2H), 1.88 (s, 4H), 1.53 (d, J = 47.3 Hz, 3H), 1.20 (s, 2H), 0.83 (s, 3H). LC-MS (ES): m/z = 470.3 [M + H]+, RT (min) = 1.19 | S8 |
| 16 | 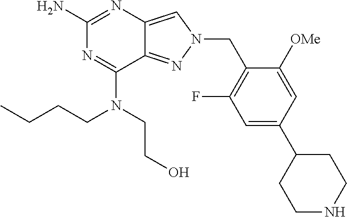 [SMILES: CCCCN(CCO)c1nc(N)nc2cn(Cc3c(F)cc(C4CCNCC4)cc3OC)nc12] | δ 7.66 (s, 1H), 7.01-6.43 (m, 2H), 5.49 (s, 1H), 5.38 (s, 2H), 4.03 (s, 1H), 3.84 (s, 2H), 3.62 (s, 1H), 3.25- 3.00 (m, 2H), 2.69 (t, J = 12.2 Hz, 3H), 2.51 (s, 2H), 1.87 (s, 6H), 1.76 (d, J = 12.8 Hz, 2H), 1.67-1.55 (m, 2H), 1.48 (s, 1H), 1.20 (s, 2H), 0.87 (d, J = 32.2 Hz, 4H). LC-MS (ES): m/z = 472.5 [M + H]+, RT (min) = 1.14 | S8 |
| 17 | 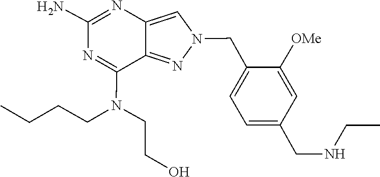 [SMILES: CCCCN(CCO)c1nc(N)nc2cn(Cc3ccc(CNCC)cc3OC)nc12] | δ 7.68 (s, 1H), 7.05 (s, 1H), 7.01-6.79 (m, 2H), 5.52 (s, 1H), 5.37 (s, 2H), 4.10 (s, 2H), 3.83 (s, 3H), 3.70 (s, 2H), 3.64 (s, 1H), 2.51 (d, J = 1.8 Hz, 2H), 2.00-1.80 (m, 4H), 1.56 (s, 3H), 1.27 (s, 3H), 1.03 (t, J = 7.1 Hz, 3H), 0.87 (s, 3H). LC-MS (ES): m/z = 428.3 [M + H]+, RT (min) = 1.12 | S1 |
| 19 | 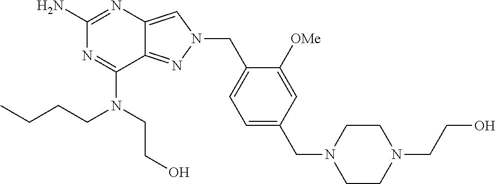 [SMILES: CCCCN(CCO)c1nc(N)nc2cn(Cc3ccc(CN4CCN(CCO)CC4)cc3OC)nc12] | δ 7.71 (s, 2H), 6.94 (d, J = 13.8 Hz, 3H), 6.83 (d, J = 7.7 Hz, 1H), 5.60 (s, 2H), 5.38 (s, 3H), 4.09 (s, 2H), 3.82 (s, 4H), 3.66 (d, J = 24.5 Hz, 3H), 2.51 (s, 2H), 2.36 (t, J = 6.3 Hz, 4H), 1.91 (s, 5H), 1.55 (s, 4H), 1.25 (s, 2H), 0.85 (s, 3H). ). LC-MS (ES): m/z = 513.4 [M + H]+, RT (min) = 1.0 | S1 |
| 20 | 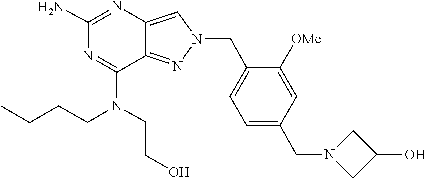 [SMILES: CCCCN(CCO)c1nc(N)nc2cn(Cc3ccc(CN4CC(O)C4)cc3OC)nc12] | δ 7.69 (s, 1H), 6.91 (s, 2H), 6.78 (d, J = 7.6 Hz, 1H), 5.51 (s, 1H), 5.36 (s, 2H), 4.18 (t, J = 6.1 Hz, 1H), 4.09 (s, 1H), 3.82 (s, 3H), 3.66 (d, J = 21.2 Hz, 4H), 3.52 (s, 0H), 2.82-2.61 (m, 3H), 2.54-2.38 (m, 2H), 1.90 (s, 3H), 1.55 (s, 4H), 1.26 (s, 2H), 0.85 (s, 3H). LC-MS (ES): m/z = 456.2 [M + H]+, RT (min) = 1.1 | S1 |
| 23 | 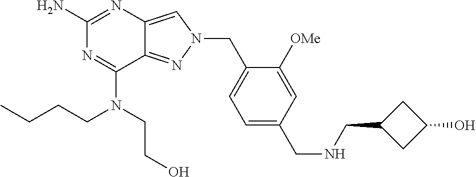 | δ 7.69 (s, 1H), 7.04 (s, 1H), 6.93 (s, 2H), 5.54 (s, 1H), 5.37 (s, 2H), 4.12 (t, J = 7.0 Hz, 1H), 3.82 (s, 2H), 2.51 (s, 1H), 2.48 (d, J = 7.6 Hz, 2H), 2.17 (s, 1H), 1.94 (d, J = 7.4 Hz, 1H), 1.89 (s, 6H), 1.56 (s, 3H), 1.27 (s, 3H), 0.87 (s, 3H). 3 proton was not observed, which is attributed to the water suppression used. LC-MS | S1 |
| | | (ES): m/z = 484.4 [M + H]+, | |
| | | RT (min) = 1.1 | |
| 26 | 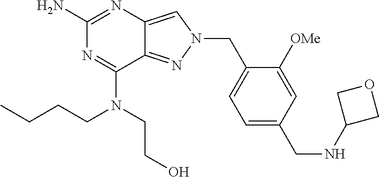 [SMILES: CCCCN(CCO)c1nc(N)nc2cn(Cc3ccc(CNC4COC4)cc3OC)nc12] | δ 7.70 (s, 1H), 7.01 (s, 1H), 6.94 (s, 1H), 6.83 (d, J = 7.7 Hz, 1H), 5.72 (s, 1H), 5.38 (s, 2H), 4.53 (t, J = 6.5 Hz, 2H), 4.28 (t, J = 6.2 Hz, 2H), 4.10 (s, 2H), 3.82 (s, 4H), 3.62 (d, J = 21.4 Hz, 4H), 2.55 (s, 3H), 1.91 (s, 2H), 1.57 (s, 2H), 1.27 (s, 2H), 0.88 (s, 3H). LC-MS (ES): m/z = 456.2 [M + H]+, RT (min) = 1.1 | S1 |
| 27 | 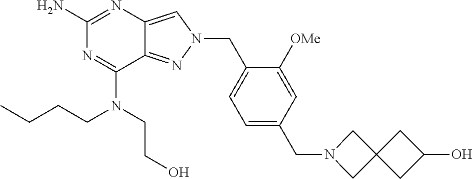 [SMILES: CCCCN(CCO)c1nc(N)nc2cn(Cc3ccc(CN4CC5(CC(O)C5)C4)cc3OC)nc12] | δ 7.69 (s, 1H), 6.89 (s, 2H), 6.77 (d, J = 7.7 Hz, 1H), 5.52 (s, 1H), 5.36 (s, 2H), 4.09 (s, 2H), 3.91 (q, J = 7.0 Hz, 1H), 3.81 (s, 3H), 3.64 (s, 2H), 3.09 (s, 2H), 3.04 (s, 2H), 2.51 (d, J = 2.9 Hz, 4H), 2.30 (t, J = 9.0 Hz, 2H), 1.91 (s, 3H), 1.85 (t, J = 9.7 Hz, 2H), 1.55 (s, 2H), 1.26 (s, 2H), 0.86 (s, 3H). LC-MS (ES): m/z = 496.2 [M + H]+, RT (min) = 1.1 | S1 |
| 29 | 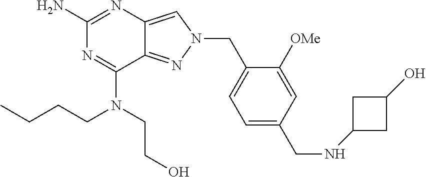 [SMILES: CCCCN(CCO)c1nc(N)nc2cn(Cc3ccc(CNC4CC(O)C4)cc3OC)nc12] | δ 7.71 (s, 1H), 7.04 (s, 1H), 6.96 (s, 1H), 6.86 (d, J = 7.7 Hz, 1H), 5.71 (s, 2H), 5.38 (s, 2H), 4.25 (d, J = 8.1 Hz, 1H), 4.11 (s, 1H), 3.82 (s, 3H), 3.67 (d, J = 29.3 Hz, 4H), 2.51 (d, J = 1.7 Hz, 5H), 2.03 (d, J = 8.1 Hz, 2H), 1.97-1.78 (m, 4H), 1.57 (s, 2H), 1.27 (s, 2H), 0.87 (s, 3H). LC-MS (ES): m/z = 470.2 [M + H]+, RT (min) = 1.1 | S1 |
| 30 | 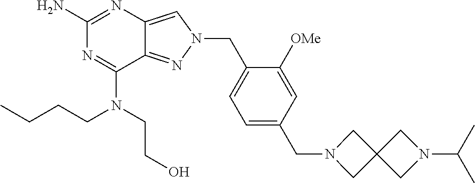 [SMILES: CCCCN(CCO)c1nc(N)nc2cn(Cc3ccc(CN4CC5(C4)CN(C(C)C)C5)cc3OC)nc12] | δ 7.69 (s, 1H), 6.90 (s, 2H), 6.77 (d, J = 7.7 Hz, 1H), 5.51 (s, 1H), 5.36 (s, 2H), 4.09 (s, 1H), 3.81 (s, 2H), 3.66 (d, J = 22.4 Hz, 2H), 3.16 (s, 3H), 3.11 (s, 3H), 2.51 (s, 4H), 2.26-2.11 (m, 2H), 1.90 (s, 4H), 1.55 (s, 2H), 1.26 (s, 2H), 0.86 (s, 4H), 0.81 (s, 3H), 0.79 (s, 3H). LC-MS (ES): m/z = 523.2 [M + H]+, RT (min) = 1.0 | S1 |
| 32 | 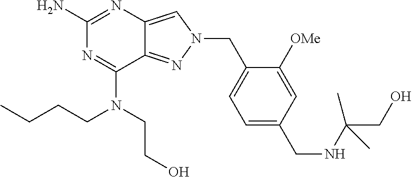 [SMILES: CCCCN(CCO)c1nc(N)nc2cn(Cc3ccc(CNC(C)(C)CO)cc3OC)nc12] | δ 7.67 (s, 1H), 7.22-6.59 (m, 3H), 5.57 (s, 1H), 5.37 (s, 2H), 3.83 (s, 2H), 3.25 (s, 2H), 2.51 (t, J = 1.8 Hz, 3H), 1.90 (s, 7H), 1.57 (s, 2H), 1.28 (s, 3H), 1.01 (s, 6H), 0.88 (s, 3H). 2 proton was not observed, which is attributed to the water suppression used. LC-MS (ES): m/z = 472.3 [M + H]+, RT (min) = 1.2 | S1 |
| 35 | 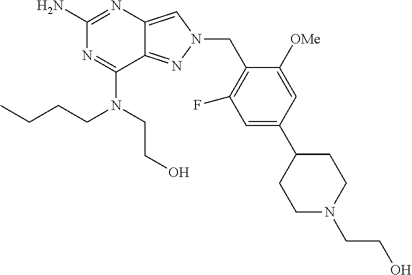 [SMILES: CCCCN(CCO)c1nc(N)nc2cn(Cc3c(F)cc(C4CCN(CCO)CC4)cc3OC)nc12] | δ 7.65 (s, 1H), 6.93-6.46 (m, 2H), 5.40 (d, J = 26.2 Hz, 4H), 4.03 (s, 2H), 3.84 (s, 3H), 3.62 (s, 1H), 2.96 (d, J = 11.0 Hz, 2H), 2.51 (s, 3H), 2.40 (t, J = 6.4 Hz, 2H), 2.07-1.99 (m, 2H), 1.90 (s, 4H), 1.68 (q, J = 12.7, 12.0 Hz, 4H), 1.49 (s, 2H), 1.21 (s, 3H), 0.84 (s, 3H). LC-MS (ES): m/z = 516.2 [M + H]+, RT (min) = 1.1 | S8 |
| 43 | 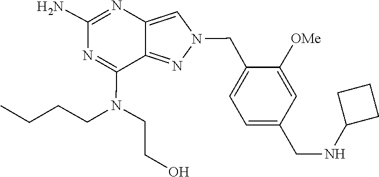 | δ 7.71 (s, 1H), 7.29-6.69 (m, 4H), 5.78 (s, 1H), 5.40 (s, 2H), 4.06 (s, 1H), 3.83 (s, 3H), 3.69 (s, 2H), 3.65 (s, 1H), 2.51 (s, 2H), 2.07 (d, J = 8.7 Hz, 3H), 1.92 (s, 3H), 1.79 (t, J = 9.8 Hz, 2H), 1.69-1.49 (m, 5H), 0.85 (s, 3H). LC-MS (ES): m/z = 440.3 [M + H]+, RT (min) = 1.1 | S7 |
| 50 | 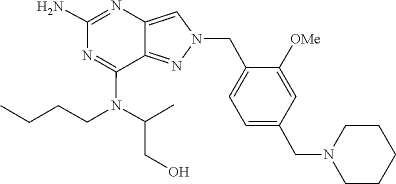 [SMILES: CCCCN(c1nc(N)nc2cn(Cc3ccc(CN4CCCCC4)cc3OC)nc12)C(C)CO] | δ 7.73 (s, 1H), 6.96 (s, 3H), 6.83 (d, J = 7.7 Hz, 1H), 5.63 (s, 2H), 5.38 (s, 3H), 3.81 (s, 3H), 2.55 (s, 3H), 2.31 (s, 4H), 1.91 (s, 4H), 1.49 (t, J = 5.7 Hz, 5H), 1.38 (s, 2H), 1.25 (s, 2H), 1.05 (d, J = 38.2 Hz, 3H), 0.87 (d, J = 33.4 Hz, 3H). LC-MS (ES): m/z = 482.3 [M + H]+, RT (min) = 1.2 | S7 |
| 52 | 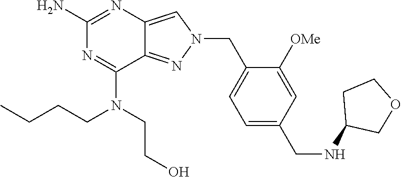 [SMILES: CCCCN(CCO)c1nc(N)nc2cn(Cc3ccc(CN[C@H]4CCOC4)cc3OC)nc12] | δ 7.69 (s, 2H), 7.04 (s, 1H), 6.95 (s, 2H), 6.86 (d, J = 7.2 Hz, 2H), 5.62 (s, 1H), 5.38 (s, 2H), 4.10 (s, 1H), 3.82 (s, 3H), 3.75 (q, J = 7.5 Hz, 1H), 3.71-3.58 (m, 4H), 2.51 (s, 2H), 1.91 (s, 4H), 1.67 (dq, J = 12.1, 5.7 Hz, 1H), 1.56 (s, 3H), 1.28 (s, 3H), 0.87 (s, 3H). LC-MS (ES): m/z = 470.2 [M + H]+, RT (min) = 1.0 | S1 |
| 54 | 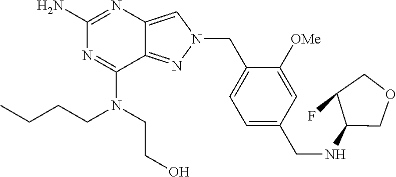 [SMILES: CCCCN(CCO)c1nc(N)nc2cn(Cc3ccc(CN[C@@H]4COC[C@@H]4F)cc3OC)nc12] | δ 7.68 (s, 1H), 7.05 (s, 1H), 7.01-6.75 (m, 2H), 5.50 (s, 1H), 5.38 (s, 2H), 5.15 (s, 1H), 5.04 (s, 1H), 4.09 (s, 1H), 3.93 (d, J = 3.7 Hz, 1H), 3.86 (d, J = 2.8 Hz, 1H), 3.83 (s, 3H), 3.79 (s, 2H), 3.73 (s, 1H), 3.69 (d, J = 12.7 Hz, 0H), 3.64 (s, 1H), 3.43-3.29 (m, 1H), 2.51 (s, 4H), 1.91 (s, 3H), 1.56 (s, 2H), 1.27 (s, 2H), | S1 |
| | | 0.86 (s, 3H). LC-MS (ES): | |
| | | m/z = 488.2 [M + H]+, RT | |
| | | (min) = 1.0 | |
| 55 | 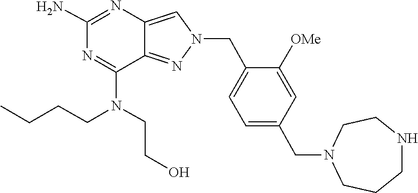 [SMILES: CCCCN(CCO)c1nc(N)nc2cn(Cc3ccc(CN4CCCNCC4)cc3OC)nc12] | δ 7.70 (s, 1H), 7.01 (s, 1H), 6.97-6.57 (m, 2H), 5.46 (s, 1H), 4.09 (s, 1H), 3.83 (s, 3H), 3.63 (d, J = 11.1 Hz, 3H), 3.00 (s, 0H), 2.96 (t, J = 5.9 Hz, 2H), 2.88 (d, J = 5.6 Hz, 1H), 2.61 (d, J = 11.1 Hz, 4H), 2.51 (t, J = 1.8 Hz, 7H), 1.89 (s, 3H), 1.79-1.68 (m, 2H), 1.56 (s, 2H), 1.26 (s, 2H), 0.85 (s, 2H). LC-MS (ES): m/z = 483.3 [M + H]+, RT (min) = 1.0 | S1 |
| 56 | 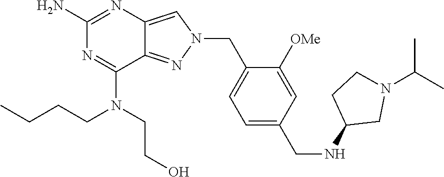 [SMILES: CCCCN(CCO)c1nc(N)nc2cn(Cc3ccc(CN[C@H]4CCN(C(C)C)C4)cc3OC)nc12] | δ 7.68 (s, 1H), 7.03 (s, 1H), 6.99-6.73 (m, 3H), 5.51 (s, 1H), 5.37 (s, 2H), 4.10 (s, 1H), 3.82 (s, 3H), 3.67 (d, J = 15.8 Hz, 4H), 3.16 (s, 0H), 2.80 (t, J = 8.3 Hz, 1H), 2.72-2.58 (m, 1H), 2.51 (s, 3H), 2.48-2.26 (m, 1H), 2.00-1.80 (m, 3H), 1.55 (d, J = 12.5 Hz, 4H), 1.28 (s, 3H), 1.02 (dd, J = 6.3, 3.2 Hz, 7H), 0.88 (s, | S1 |
| | | 3H). LC-MS (ES): m/z = | |
| | | 511.3 [M + H]+, RT (min) = | |
| | | 1.0 | |
| 57 | 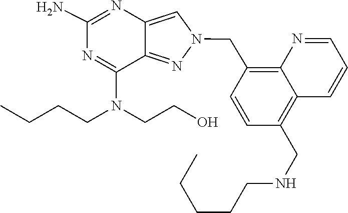 [SMILES: CCCCCNCc1ccc(Cn2cc3nc(N)nc(N(CCO)CCCC)c3n2)c2ncccc12] | δ 9.05 (s, 1H), 8.75 (d, J = 8.6 Hz, 1H), 8.12 (s, 1H), 7.71 (d, J = 7.7 Hz, 2H), 7.49 (s, 1H), 7.42 (s, 1H), 7.02 (s, 1H), 6.16 (s, 2H), 4.52 (s, 2H), 4.17 (s, 1H), 4.10 (s, 1H), 3.76 (s, 2H), 2.91 (s, 1H), 1.92 (s, 1H), 1.59 (s, 3H), 1.52 (s, 1H), 1.31-1.26 (m, 5H), 1.16 (s, 3H), 0.93 (s, 1H), 0.90- 0.83 (m, 4H), 0.78 (s, 3H). LC-MS(ES): m/z = 491.2 [M + H]+, RT(min) = 1.82 | S10 |
| 59 | 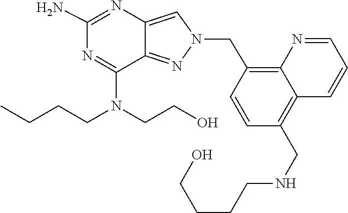 [SMILES: CCCCN(CCO)c1nc(N)nc2cn(Cc3ccc(CNCCCCO)c4cccnc34)nc12] | δ 9.01 (s, 1H), 8.70 (d, J = 8.7 Hz, 1H), 7.91 (s, 1H), 7.68-7.62 (m, 1H), 7.57 (d, J = 7.2 Hz, 1H), 7.34 (s, 1H), 6.09 (s, 2H), 5.84 (s, 2H), 4.23 (s, 2H), 4.08 (s, 1H), 3.64 (s, 2H), 3.18 (s, 2H), 2.66 (s, 2H), 1.91 (s, 4H), 1.50 (d, J = 7.0 Hz, 4H), 1.44 (t, J = 7.1 Hz, 2H), 1.18 (s, 2H), 0.92 (s, 2H), 0.78 (s, 3H). LC- MS(ES): m/z = 493.2 [M + H]+, RT(min) = 1.21 | S10 |
| 61 | 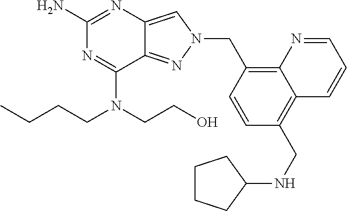 [SMILES: CCCCN(CCO)c1nc(N)nc2cn(Cc3ccc(CNC4CCCC4)c4cccnc34)nc12] | δ 8.99 (s, 1H), 8.72-8.66 (m, 1H), 7.87 (s, 1H), 7.63 (dd, J = 8.4, 4.1 Hz, 1H), 7.55 (d, J = 7.3 Hz, 1H), 7.31 (s, 1H), 6.07 (s, 2H), 5.63 (s, 1H), 4.15 (s, 2H), 4.07 (s, 1H), 3.18 (s, 2H), 3.11 (s, 1H), 1.91 (s, 4H), 1.76 (d, J = 6.9 Hz, 2H), 1.63 (s, 2H), 1.47 (s, 4H), 1.39 (s, 1H), 1.17 (s, 2H), 0.91 (s, 2H), 0.78 (s, 3H). LC-MS(ES): m/z = 489.1 [M + H]+, RT(min) = 1.70 | S10 |
| 62 | 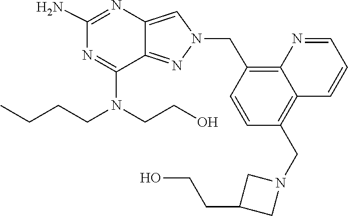 [SMILES: CCCCN(CCO)c1nc(N)nc2cn(Cc3ccc(CN4CC(CCO)C4)c4cccnc34)nc12] | δ 9.00 (s, 1H), 8.68 (d, J = 8.6 Hz, 1H), 8.09 (s, 1H), 7.65 (s, 1H), 7.52 (d, J = 7.4 Hz, 1H), 7.38 (s, 1H), 6.87 (s, 2H), 6.12 (s, 2H), 4.17 (s, 1H), 4.09 (s, 3H), 3.75 (s, 2H), 2.95 (s, 1H), 1.92 (s, 2H), 1.66 (q, J = 6.7 Hz, 3H), 1.50 (s, 2H), 1.33 (s, 1H), 1.14 (s, 2H), 0.93 (s, 2H), 0.75 (s, 3H). LC- MS(ES): m/z = 505.2 [M + H]+, RT(min) = 1.24 | S10 |
| 64 | 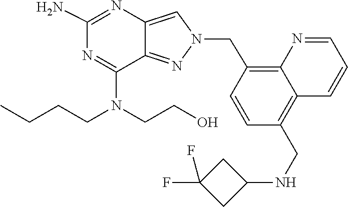 [SMILES: CCCCN(CCO)c1nc(N)nc2cn(Cc3ccc(CNC4CC(F)(F)C4)c4cccnc34)nc12] | δ 9.00 (s, 1H), 8.71-8.65 (m, 1H), 7.97 (s, 1H), 7.64 (dd, J = 8.5, 4.1 Hz, 1H), 7.53 (d, J = 7.4 Hz, 1H), 7.35 (s, 1H), 6.21 (s, 2H), 6.10 (s, 2H), 4.08 (s, 2H), 3.72 (s, 1H), 2.71 (s, 3H), 2.35 (d, J = 17.7 Hz, 3H), 1.92 (s, 1H), 1.61 (s, 1H), 1.51 (s, 2H), 1.32 (s, 1H), 1.17 (s, 2H), 0.92 (s, 2H), 0.78 (s, 3H). LC-MS(ES): m/z = 511.1 [M + H]+, RT(min) = 1.81 | S10 |
| 65 | 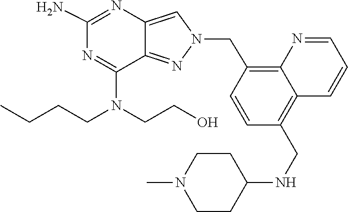 [SMILES: CCCCN(CCO)c1nc(N)nc2cn(Cc3ccc(CNC4CCN(C)CC4)c4cccnc34)nc12] | δ 9.06 (s, 1H), 8.73 (d, J = 8.6 Hz, 1H), 8.22 (s, 1H), 7.80 (s, 2H), 7.74 (s, 2H), 7.57 (d, J = 7.6 Hz, 1H), 6.20 (s, 2H), 4.69 (s, 3H), 4.20 (s, 1H), 4.11 (d, J = 8.1 Hz, 2H), 3.81 (d, J = 14.0 Hz, 2H), 2.93 (s, 1H), 2.76 (s, 4H), 2.37 (s, 3H), 1.64 (d, J = 7.8 Hz, 2H), 1.54 (s, 2H), 1.33 (q, J = 7.4 Hz, 1H), 1.17 (dd, J = 8.2, 6.0 Hz, 3H), 0.93 (t, J = 7.3 Hz, | S10 |
| | | 2H), 0.80 (q, J = 8.9, 7.4 | |
| | | Hz, 3H). LC-MS(ES): | |
| | | m/z = 518.2 [M + H]+, RT | |
| | | (min) = 1.41 | |
| 66 | 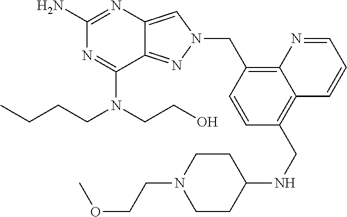 [SMILES: CCCCN(CCO)c1nc(N)nc2cn(Cc3ccc(CNC4CCN(CCOC)CC4)c4cccnc34)nc12] | δ 8.98 (s, 1H), 8.71-8.66 (m, 1H), 7.86 (s, 1H), 7.62 (dd, J = 8.5, 4.2 Hz, 1H), 7.54 (d, J = 7.3 Hz, 1H), 7.30 (s, 1H), 6.06 (s, 2H), 5.57 (s, 1H), 4.15 (s, 2H), 3.21 (s, 3H), 2.79 (d, J = 11.2 Hz, 2H), 2.42 (t, J = 6.0 Hz, 3H), 1.96 (t, J = 11.3 Hz, 2H), 1.90 (s, 7H), 1.83 (d, J = 12.7 Hz, 2H), 1.51 (s, 2H), 1.30 (d, J = 12.2 Hz, 3H), 1.17 (s, 2H), 0.91 (s, 2H), 0.78 (s, 3H). | S10 |
| | | LC-MS(ES): m/z = 562.2 | |
| | | [M + H]+, RT(min) = 1.40 | |
| 67 | 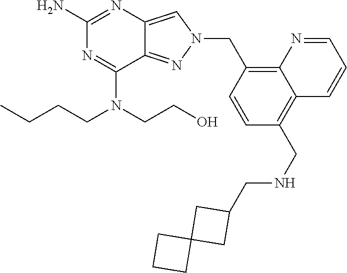 [SMILES: CCCCN(CCO)c1nc(N)nc2cn(Cc3ccc(CNCC4CC5(CCC5)C4)c4cccnc34)nc12] | δ 9.07 (dd, J = 8.2, 4.3 Hz, 1H), 8.93 (s, 1H), 8.74 (d, J = 8.6 Hz, 1H), 7.96 (s, 1H), 7.77 (dd, J = 14.7, 6.7 Hz, 2H), 7.56 (d, J = 7.5 Hz, 1H), 7.15 (s, 1H), 6.20 (s, 2H), 4.63 (s, 2H), 4.10 (d, J = 8.2 Hz, 2H), 3.79 (s, 1H), 3.70 (s, 1H), 3.08 (d, J = 7.2 Hz, 3H), 2.47 (d, J = 7.4 Hz, 1H), 2.12 (t, J = 10.0 Hz, 2H), 1.98 (t, J = 7.4 Hz, 2H), 1.85 (d, J = 7.7 Hz, 3H), 1.79-1.70 (m, 4H), 1.64 (d, J = 7.4 Hz, 1H), 1.33 (q, J = 7.6 Hz, 1H), 1.17 (q, J = 7.2, 6.8 Hz, 2H), 0.93 (t, J = 7.4 Hz, | S10 |
| | | 2H), 0.77 (t, J = 7.3 Hz, | |
| | | 3H). LC-MS(ES): | |
| | | m/z = 529.3 [M + H]+, | |
| | | RT(min) = 1.94 | |
| 68 | 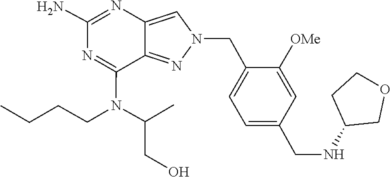 [SMILES: CCCCN(c1nc(N)nc2cn(Cc3ccc(CN[C@@H]4CCOC4)cc3OC)nc12)C(C)CO] | δ 8.06 (d, J = 8.2 Hz, 1H), 7.74 (d, J = 12.2 Hz, 3H), 7.26 (s, 1H), 7.19 (t, J = 7.7 Hz, 2H), 7.07 (d, J = 7.9 Hz, 1H), 5.51 (d, J = 7.0 Hz, 3H), 4.37 (d, J = 14.5 Hz, 1H), 4.13 (dd, J = 20.8, 8.1 Hz, 4H), 3.90 (dd, J = 9.1, 6.3 Hz, 3H), 3.77 (dd, J = 9.7, 6.2 Hz, 1H), 3.65 (p, J = 8.5, 7.9 Hz, 2H), 2.23 (q, J = 6.6 Hz, 1H), 2.03 (d, J = | S7 |
| | | 11.9 Hz, 1H), 1.63 (t, J = | |
| | | 7.5 Hz, 1H), 1.29 (dq, J = | |
| | | 28.0, 7.5 Hz, 3H), 1.17 (t, | |
| | | J = 7.3 Hz, 1H), 1.12 (d, J = | |
| | | 6.2 Hz, 2H), 1.04 (d, J = 6.1 | |
| | | Hz, 2H), 0.93 (t, J = 7.4 Hz, | |
| | | 2H), 0.87 (t, J = 7.3 Hz, | |
| | | 2H). ). LC-MS(ES): | |
| | | m/z = 484.3 [M + H]+, | |
| | | RT(min) = 1.42 | |
| 70 | 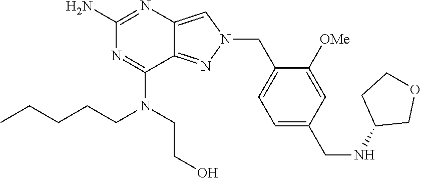 [SMILES: CCCCCN(CCO)c1nc(N)nc2cn(Cc3ccc(CN[C@@H]4CCOC4)cc3OC)nc12] | δ 8.06 (s, 1H), 8.01 (s, 0H), 7.77 (s, 2H), 7.26 (s, 1H), 7.15 (dd, J = 20.3, 7.7 Hz, 1H), 7.06 (d, J = 7.7 Hz, 1H), 5.51 (d, J = 2.5 Hz, 2H), 4.20 (d, J = 11.4 Hz, 1H), 4.18-4.06 (m, 3H), 3.99-3.87 (m, 2H), 3.83- 3.75 (m, 3H), 3.74-3.59 (m, 3H), 2.51 (d, J = 2.0 Hz, 2H), 2.23 (q, J = 6.4 Hz, 1H), 2.03 (s, 1H), 1.79- | S7 |
| | | 1.55 (m, 3H), 1.42-1.20 | |
| | | (m, 6H), 0.87 (dt, J = 16.9, | |
| | | 7.0 Hz, 4H). LC-MS(ES): | |
| | | m/z = 484.3 [M + H]+, | |
| | | RT(min) = 1.19 | |
| 71 | 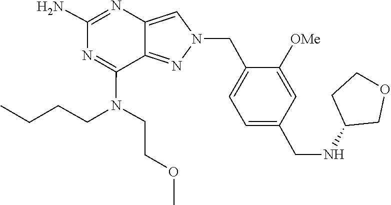 [SMILES: CCCCN(CCOC)c1nc(N)nc2cn(Cc3ccc(CN[C@@H]4CCOC4)cc3OC)nc12] | δ 8.06 (d, J = 13.5 Hz, 1H), 7.82 (d, J = 7.6 Hz, 2H), 7.26 (s, 1H), 7.16 (dd, J = 25.6, 7.7 Hz, 1H), 7.07 (d, J = 7.6 Hz, 1H), 5.51 (s, 2H), 4.32 (d, J = 6.0 Hz, 1H), 4.15 (dd, J = 18.4, 6.1 Hz, 3H), 3.91 (dd, J = 12.9, 7.4 Hz, 3H), 3.85 (d, J = 4.6 Hz, 3H), 3.79-3.73 (m, 1H), 3.73-3.54 (m, 2H), 3.28 (s, 1H), 3.21 (s, 1H), 2.93 (s, 1H), 2.54-2.38 (m, 3H), 2.23 (dd, J = 13.9, 6.8 Hz, | S7 |
| | | 1H), 2.04 (s, 1H), 1.61 (dd, | |
| | | J = 24.6, 9.8 Hz, 2H), 1.30 | |
| | | (dq, J = 31.9, 7.5 Hz, 2H), | |
| | | 1.17 (t, J = 7.3 Hz, 1H), | |
| | | 0.93 (t, J = 7.3 Hz, 1H), | |
| | | 0.86 (t, J = 7.4 Hz, 2H). LC- | |
| | | MS(ES): m/z = 484.2 | |
| | | [M + H]+, RT(min) = 1.28 | |
| 74 | 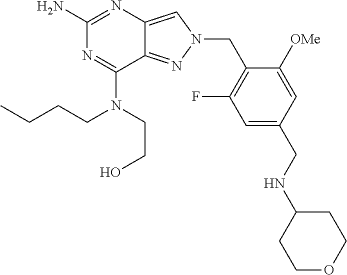 [SMILES: CCCCN(CCO)c1nc(N)nc2cn(Cc3c(F)cc(CNC4CCOCC4)cc3OC)nc12] | δ 8.30 (s, 0H), 7.68 (s, 1H), 6.92 (s, 1H), 6.86 (d, J = 9.9 Hz, 1H), 5.75 (s, 1H), 5.40 (s, 2H), 4.04 (s, 1H), 3.84 (s, 3H), 3.80 (s, 1H), 3.74 (s, 1H), 3.65 (d, J = 23.8 Hz, 1H), 3.24 (t, J = 11.3 Hz, 2H), 2.53 (d, J = 19.5 Hz, 6H), 1.91 (s, 4H), 1.76 (d, J = 12.9 Hz, 2H), 1.51 (s, 2H), 1.34-1.15 (m, 4H), 0.86 (s, 3H). LC-MS(ES): m/z = 502.2 [M + H]+, RT(min) = 1.12 | S1 |
| 80 | 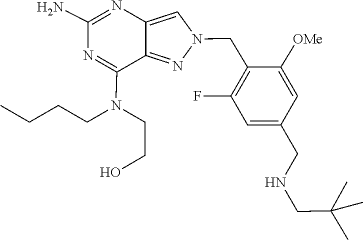 [SMILES: CCCCN(CCO)c1nc(N)nc2cn(Cc3c(F)cc(CNCC(C)(C)C)cc3OC)nc12] | δ 8.07 (s, 1H), 7.77 (s, 2H), 7.12 (s, 1H), 7.04 (d, J = 9.7 Hz, 1H), 5.52 (d, J = 7.6 Hz, 2H), 4.16 (s, 2H), 4.08 (t, J = 7.9 Hz, 1H), 3.87 (d, J = 7.6 Hz, 3H), 3.79 (q, J = 7.9, 5.8 Hz, 2H), 3.74-3.56 (m, 2H), 2.89 (t, J = 8.1 Hz, 2H), 2.55 (s, 1H), 1.58 (dt, J = 37.5, 8.0 Hz, 5H), 1.43- 1.10 (m, 7H), 1.03-0.66 (m, 6H). LC-MS(ES): m/z = 488.3 [M + H]+, RT(min) = 1.3 | S1 |
| 81 | 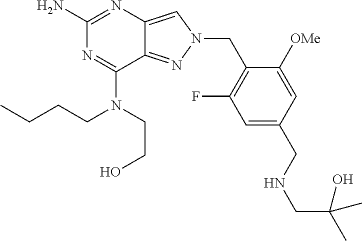 [SMILES: CCCCN(CCO)c1nc(N)nc2cn(Cc3c(F)cc(CNCC(C)(C)O)cc3OC)nc12] | δ 7.70 (s, 1H), 6.92 (s, 1H), 6.84 (d, J = 9.9 Hz, 1H), 5.81 (s, 1H), 5.40 (s, 2H), 4.06 (d, J = 27.2 Hz, 1H), 3.83 (s, 4H), 3.75 (s, 2H), 3.65 (d, J = 23.9 Hz, 2H), 2.54-2.43 (m, 2H), 2.36 (s, 2H), 1.91 (s, 3H), 1.54 (d, J = 42.3 Hz, 3H), 1.25 (d, J = 22.1 Hz, 1H), 1.10 (s, 7H), 0.85 (s, 3H). LC-MS(ES): m/z = 490.0 [M + H]+, RT(min) = 1.32 | S1 |
| 83 | 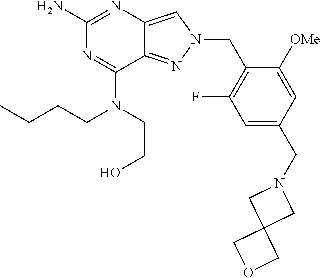 [SMILES: CCCCN(CCO)c1nc(N)nc2cn(Cc3c(F)cc(CN4CC5(COC5)C4)cc3OC)nc12] | δ 7.78 (s, 1H), 6.98-6.48 (m, 2H), 6.20 (s, 1H), 5.41 (s, 2H), 4.60 (s, 4H), 4.04 (s, 1H), 3.86 (d, J = 38.0 Hz, 4H), 3.67 (d, J = 27.7 Hz, 1H), 3.32 (s, 2H), 2.51 (s, 5H), 1.91 (s, 3H), 1.54 (d, J = 48.8 Hz, 2H), 1.41- 1.04 (m, 2H), 0.87 (d, J = 35.1 Hz, 4H). LC-MS(ES): m/z = 500.3 [M + H]+, RT(min) = 1.11 | S1 |
| 85 | 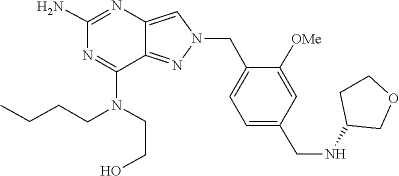 | δ 7.03 (s, 1H), 6.95 (s, 2H), 6.86 (d, J = 7.2 Hz, 1H), 5.62 (s, 1H), 5.37 (s, 2H), 3.82 (s, 3H), 3.77-3.59 (m, 5H), 2.55 (s, 3H), 1.97- 1.87 (m, 1H), 1.91 (s, 4H), 1.67 (dt, J = 11.9, 6.2 Hz, 1H), 1.56 (s, 3H), 1.27 (s, 3H), 0.87 (s, 5H). LC-MS (ES): m/z = 470.2 [M + H]+, RT (min) = 1.09 | S1 |
| 87 | 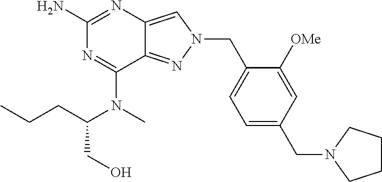 [SMILES: CCC[C@@H](CO)N(C)c1nc(N)nc2cn(Cc3ccc(CN4CCCC4)cc3OC)nc12] | δ 7.67 (br s, 1H), 7.03-6.79 (m, 3H), 5.86-5.51 (m, 2H), 5.51-5.11 (m, 3H), 3.81 (br s, 2H), 3.68-3.60 (m, 3H), 3.50 (br s, 3H), 2.98 (br d, J = 11.9 Hz, 1H), 2.55 (s, 3H), 1.68 (br s, 4H), 1.59-1.42 (m, 2H), 1.17 (br s, 2H), 0.95-0.71 (m, 3H), 3 protons were not observed due to water suppression. LC-MS (ES, m/z): | S7 |
| | | [M + H]+ = 454.4, r.t. | |
| | | 1.14 mins | |
| 89 | 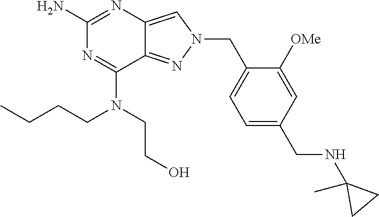 [SMILES: CCCCN(CCO)c1nc(N)nc2cn(Cc3ccc(CNC4(C)CC4)cc3OC)nc12] | 1H NMR (400 MHz, DMSO-d6) δ = 7.73 (br s, 1H), 7.01 (s, 1H), 6.96 (br s, 1H), 6.88-6.83 (m, 1H), 6.04-5.88 (m, 2H), 5.38 (s, 2H), 4.82-4.60 (m, 1H), 4.24-4.03 (m, 2H), 3.81 (s, 3H), 3.74 (s, 3H), 3.71 (br s, 1H), 3.65 (br d, J = 3.4 Hz, 3H), 1.91 (s, 1H), 1.57 (br s, 2H), 1.37-1.17 (m, 6H), 1.00-0.74 (m, 4H), 0.57- 0.49 (m, 2H), 0.37-0.27 (m, 2H); LC-MS (ES): | S1 |
| | | m/z = 454.3 [M + H]+, | |
| | | RT (min) = 1.001 | |
| 90 | 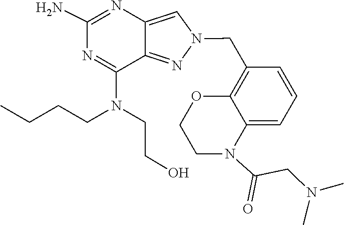 [SMILES: CCCCN(CCO)c1nc(N)nc2cn(Cc3cccc4c3OCCN4C(=O)CN(C)C)nc12] | δ 7.75 (br s, 2H), 7.00-6.60 (m, 2H), 5.64 (br s, 1H), 5.40 (br s, 2H), 4.35 (br s, 2H), 4.12 (br s, 1H), 3.93 (br s, 2H), 3.82-3.20 (m, 6H), 2.23 (br s, 5H), 1.56 (br s, 2H), 1.25 (br s, 2H), 0.85 (br s, 3H), 4 protons were not observed due to water suppression. LC-MS (ES, m/z): [M + H]+ = 483.2, r.t. 1.31 mins | S12 |
| 91 | 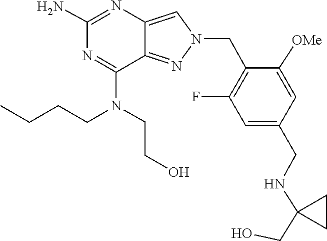 [SMILES: CCCCN(CCO)c1nc(N)nc2cn(Cc3c(F)cc(CNC4(CO)CC4)cc3OC)nc12] | δ 7.78 (s, 1H), 6.89 (s, 1H), 6.82 (d, J = 10.0 Hz, 1H), 6.30 (s, 2H), 5.42 (s, 2H), 4.09 (d, J = 32.7 Hz, 2H), 3.81 (d, J = 24.1 Hz, 5H), 3.68 (d, J = 30.1 Hz, 3H), 2.55 (s, 3H), 1.92 (s, 2H), 1.55 (d, J = 46.9 Hz, 2H), 1.26 (d, J = 44.0 Hz, 3H), 0.89 (d, J = 31.4 Hz, 4H), 0.49 (s, 2H), 0.43 (s, 1H). LC-MS (ES, m/z): [M + H]+ = 488.2, r.t. 1.36 mins | S1 |
| 92 | 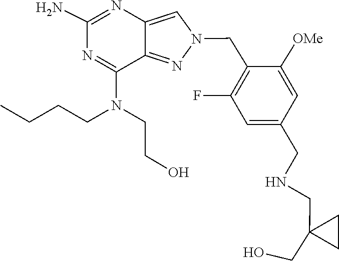 [SMILES: CCCCN(CCO)c1nc(N)nc2cn(Cc3c(F)cc(CNCC4(CO)CC4)cc3OC)nc12] | 1H NMR (500 MHz, DMSO-d6) δ 7.80-7.54 (m, 1H), 6.93 (br s, 1H), 6.84 (br d, J = 10.1 Hz, 1H), 5.70 (br s, 2H), 5.40 (s, 2H), 4.18-3.91 (m, 2H), 3.84 (br s, 3H), 3.74 (s, 2H), 3.73- 3.58 (m, 2H), 1.72-1.41 (m, 2H), 1.37-1.14 (m, 2H), 1.01-0.74 (m, 3H), 0.33 (br d, J = 15.6 Hz, 4H) | S1 |
| 93 | 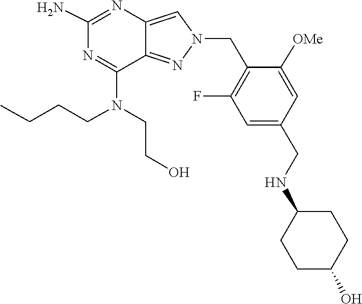 [SMILES: CCCCN(CCO)c1nc(N)nc2cn(Cc3c(F)cc(CN[C@H]4CC[C@H](O)CC4)cc3OC)nc12] | δ 7.77 (s, 1H), 7.09-6.57 (m, 2H), 6.11 (s, 1H), 5.44 (s, 2H), 4.05 (s, 1H), 3.85 (s, 2H), 3.67 (d, J = 29.0 Hz, 3H), 3.01 (s, 1H), 2.53 (d, J = 19.7 Hz, 13H), 1.92 (s, 1H), 1.82 (d, J = 10.6 Hz, 2H), 1.52 (s, 1H), 1.47- 1.02 (m, 5H), 0.86 (s, 3H). LC-MS (ES): m/z = 516.3 [M + H]+, RT (min) = 1.16 | S1 |
| 94 | 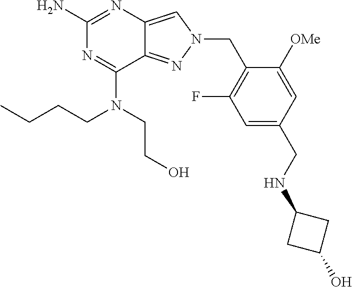 [SMILES: CCCCN(CCO)c1nc(N)nc2cn(Cc3c(F)cc(CN[C@H]4C[C@H](O)C4)cc3OC)nc12] | δ 8.18-7.92 (m, 1H), 7.73 (br s, 2H), 7.10 (s, 1H), 7.03 (br d, J = 10.1 Hz, 1H), 5.71- 5.39 (m, 2H), 4.35 (br d, J = 3.1 Hz, 1H), 4.20-4.02 (m, 3H), 3.93-3.84 (m, 2H), 3.83-3.72 (m, 2H), 3.72-3.59 (m, 1H), 2.41 (dt, J = 12.2, 6.1 Hz, 2H), 2.20-2.04 (m, 2H), 1.69- 1.41 (m, 2H), 1.38-1.11 (m, 2H), 1.01-0.71 (m, 3H). Seven protons were not visible due to water supression. LC-MS (ES): m/z = 488.2 [M + H]+, RT (min) = 1.32 | S1 |
| 95 | 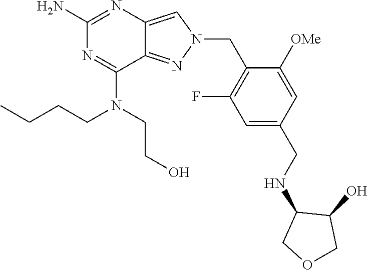 [SMILES: CCCCN(CCO)c1nc(N)nc2cn(Cc3c(F)cc(CN[C@@H]4COC[C@@H]4O)cc3OC)nc12] | δ 8.07 (s, 1H), 7.78 (s, 2H), 7.15 (s, 1H), 7.07 (d, J = 9.7 Hz, 1H), 5.53 (d, J = 7.3 Hz, 3H), 4.43 (s, 1H), 4.17 (d, J = 12.7 Hz, 3H), 4.09 (t, J = 8.1 Hz, 2H), 3.88 (d, J = 6.9 Hz, 4H), 3.86-3.76 (m, 2H), 3.72 (d, J = 12.5 Hz, 4H), 2.52 (s, 2H), 1.82- 1.46 (m, 1H), 1.33 (q, J = 7.4 Hz, 1H), 1.23 (q, J = 7.5 Hz, 2H), 0.93 (t, J = 7.3 Hz, 1H), 0.87 (t, J = 7.3 Hz, 3H). LC-MS (ES): m/z = 504.3 [M + H]+, RT (min) = 1.07 | S1 |
| 96 | 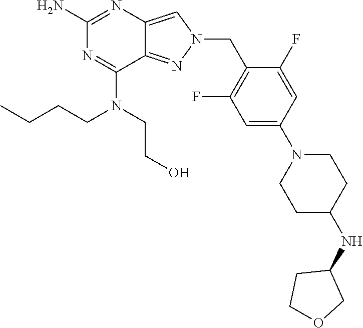 [SMILES: CCCCN(CCO)c1nc(N)nc2cn(Cc3c(F)cc(N4CCC(N[C@@H]5CCOC5)CC4)cc3F)nc12] | δ 7.88-7.61 (m, 1H), 6.65 (br d, J = 11.2 Hz, 2H), 6.20- 5.99 (m, 1H), 5.38 (s, 2H), 4.04 (br d, J = 3.4 Hz, 1H), 3.87-3.54 (m, 5H), 2.92- 2.68 (m, 3H), 2.13-1.97 (m, 1H), 1.90-1.78 (m, 2H), 1.67 (td, J = 12.5, 6.4 Hz, 1H), 1.63-1.42 (m, 2H), 1.38-1.08 (m, 4H), 0.85 (br d, J = 6.5 Hz, 3H). Eight protons were not visible due to water supression and the overlap with the DMSO-d6 peak. Two exchangeble protons were not visible either. LC- MS (ES): m/z = 545.2 [M + H]+, RT (min) = 1.23 | S9 |
| 97 | 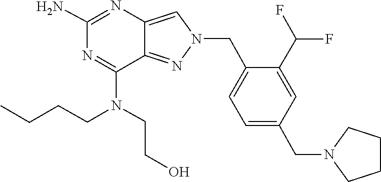 [SMILES: CCCCN(CCO)c1nc(N)nc2cn(Cc3ccc(CN4CCCC4)cc3C(F)F)nc12] | δ 7.92 (br s, 1H), 7.58 (s, 1H), 7.45 (br d, J = 7.9 Hz, 2H), 7.18 (d, J = 8.0 Hz, 1H), 6.09 (br s, 2H), 5.65 (s, 2H), 4.08 (br s, 1H), 3.87-3.52 (m, 4H), 1.70 (br s, 4H), 1.53 (br s, 2H), 1.20 (br s, 2H), 1.02-0.69 (m, 3H), 8 protons were not observed due to water suppression. LC-MS (ES, m/z): [M + H]+ = 474.1, 1.39 mins | S1 |
| 98 | 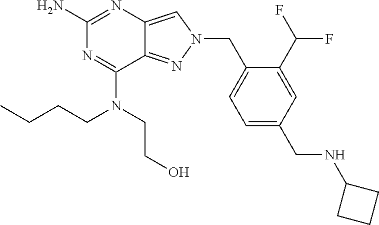 [SMILES: CCCCN(CCO)c1nc(N)nc2cn(Cc3ccc(CNC4CCC4)cc3C(F)F)nc12] | δ 7.84 (br s, 1H), 7.62 (s, 1H), 7.55-7.25 (m, 2H), 7.18 (br d, J = 7.8 Hz, 1H), 5.71 (br s, 1H), 5.63 (s, 2H), 4.07 (br s, 1H), 3.81-3.54 (m, 4H), 3.17 (br t, J = 7.6 Hz, 1H), 2.04 (br d, J = 8.3 Hz, 2H), 1.78-1.67 (m, 2H), 1.66-1.46 (m, 4H), 1.23 (br s, 2H), 1.04-0.73 (m, 3H), 6 protons were not observed due to water suppression. LC-MS (ES, m/z): [M + H]+ = 474.2, r.t. 1.47 mins | S1 |
| 99 | 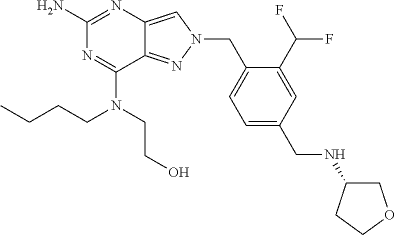 [SMILES: CCCCN(CCO)c1nc(N)nc2cn(Cc3ccc(CN[C@H]4CCOC4)cc3C(F)F)nc12] | δ 7.89 (br s, 1H), 7.63 (s, 1H), 7.47 (br d, J = 7.7 Hz, 1H), 7.38 (br s, 1H), 7.20 (br d, J = 7.4 Hz, 1H), 6.01 (br s, 1H), 5.64 (s, 2H), 4.08 (br s, 2H), 3.83-3.56 (m, 7H), 3.26 (br s, 1H), 1.99- 1.88 (m, 1H), 1.68 (br d, J = 6.3 Hz, 1H), 1.55 (br s, 2H), 1.41-1.15 (m, 2H), 0.99-0.75 (m, 3H), 6 protons were not observed due to water suppression. LC-MS (ES, m/z): [M + H]+ = 490.3, r.t. 1.39 mins | S1 |
| 100 | 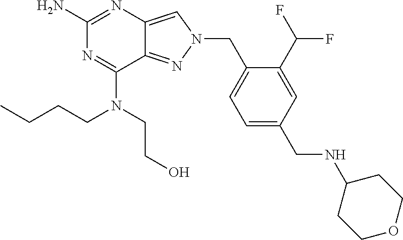 [SMILES: CCCCN(CCO)c1nc(N)nc2cn(Cc3ccc(CNC4CCOCC4)cc3C(F)F)nc12] | δ 7.85 (br s, 1H), 7.64 (s, 1H), 7.48 (br d, J = 7.5 Hz, 1H), 7.37 (br s, 1H), 7.20 (br d, J = 7.9 Hz, 1H), 5.73 (br s, 2H), 5.62 (br s, 2H), 4.07 (br s, 1H), 3.86-3.78 (m, 3H), 3.75-3.58 (m, 2H), 3.57-3.48 (m, 1H), 3.32-3.15 (m, 2H), 2.63 (br s, 1H), 1.91 (br s, 2H), 1.77 (br d, J = 12.9 Hz, 2H), 1.54 (br s, 2H), 1.38-1.15 (m, 4H), 0.99-0.75 (m, 3H), 3 protons were not observed due to water suppression. LC-MS (ES, m/z): | S1 |
| | | [M + H]+ = 504.3, r.t. | |
| | | 1.36 mins | |
| 101 | 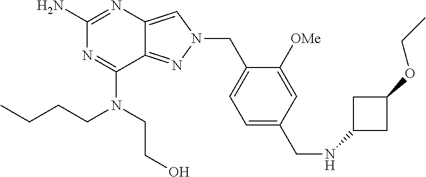 | δ = 12.03 (br d, J = 5.8 Hz, 1H), 9.33-9.07 (m, 1H), 8.14-7.93 (m, 1H), 7.75 (br s, 2H), 7.22 (s, 1H), 7.17 (d, J = 7.5 Hz, 1H), 7.11 (d, J = 8.0 Hz, 1H), 7.03 (br d, J = 7.3 Hz, 1H), 5.51 (s, 2H), 4.92-4.77 (m, 1H), 4.21 (br t, J = 5.4 Hz, 1H), 4.18- 4.10 (m, 2H), 4.06 (s, 2H), 3.89-3.84 (m, 3H), 3.84-3.77 (m, 2H), 3.74-3.64 (m, | S1 |
| | | 2H), 2.45-2.37 (m, 2H), | |
| | | 2.22 (dt, J = 4.1, 8.9 Hz, | |
| | | 2H), 1.73-1.53 (m, 2H), | |
| | | 1.42-1.21 (m, 2H), 1.17- | |
| | | 1.07 (m, 3H), 0.97-0.79 | |
| | | (m, 3H) LC-MS (ES, m/z): | |
| | | [M + H]+ = 498.3, r.t. 1.44 | |
| | | mins | |
| 102 | 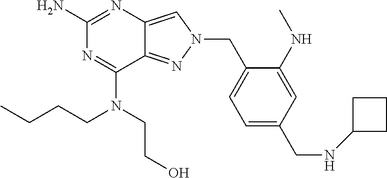 [SMILES: CCCCN(CCO)c1nc(N)nc2cn(Cc3ccc(CNC4CCC4)cc3NC)nc12] | 1H NMR (500 MHz, DMSO-d6) δ 9.09 (br s, 1H), 8.20-8.01 (m, 1H), 7.78 (br s, 1H), 7.05-6.95 (m, 1H), 6.82-6.60 (m, 2H), 5.58-5.34 (m, 2H), 4.26-4.09 (m, 2H), 3.92 (br s, 2H), 3.81 (br s, 1H), 3.74- 3.57 (m, 2H), 3.56-3.44 (m, 5H), 2.81-2.73 (m, 2H), 2.14 (br d, J = 7.6 Hz, 3H), 1.99-1.53 (m, 4H), | S1 |
| | | 1.31 (br dd, J = 15.0, 6.7 Hz, | |
| | | 2H), 1.01-0.80 (m, 3H), 4 | |
| | | protons were not observed | |
| | | due to water suppression. | |
| | | LC-MS (ES, m/z): | |
| | | [M + H]+ = 453.2, | |
| | | 1.25 mins | |
| 104 | 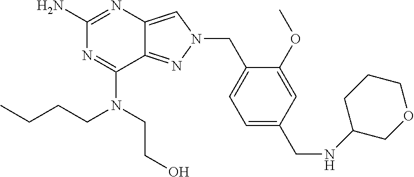 [SMILES: CCCCN(CCO)c1nc(N)nc2cn(Cc3ccc(CNC4CCCOC4)cc3OC)nc12] | 1H NMR (400 MHz, DMSO-d6) δ = 7.73-7.62 (m, 1H), 7.07-7.00 (m, 1H), 6.96-6.75 (m, 2H), 5.44-5.31 (m, 4H), 4.16- 4.02 (m, 1H), 3.85-3.75 (m, 5H), 3.72-3.61 (m, 9H), 3.21-3.19 (m, 2H), 2.69-2.64 (m, 1H), 1.95- 1.84 (m, 2H), 1.62-1.51 (m, 3H), 1.46-1.35 (m, 3H), 0.93-0.84 (m, 3H); | S1 |
| | | LC-MS (ES): m/z = 484.2 | |
| | | [M + H]+, RT (min) = 1.388 | |
| 105 | 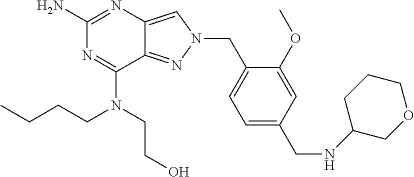 [SMILES: CCCCN(CCO)c1nc(N)nc2cn(Cc3ccc(CNC4CCCOC4)cc3OC)nc12] | 1H NMR (400 MHz, DMSO-d6) δ = 7.72-7.59 (m, 1H), 7.04-7.01 (m, 1H), 6.96-6.89 (m, 1H), 6.87-6.83 (m, 1H), 5.44- 5.35 (m, 4H), 4.19-4.01 (m, 1H), 3.86-3.75 (m, 5H), 3.74-3.72 (m, 1H), 3.72-3.67 (m, 7H), 3.66- 3.60 (m, 3H), 3.03-2.96 (m, 1H), 1.94-1.85 (m, 2H), 1.63 1.49 (m, 2H), | S1 |
| | | 1.46-1.35 (m, 2H), 1.34- | |
| | | 1.26 (m, 2H), 0.94-0.82 | |
| | | (m, 3H); LC-MS (ES): | |
| | | m/z = 484.3 [M + H]+, | |
| | | RT (min) = 1.386 | |
| 106 | 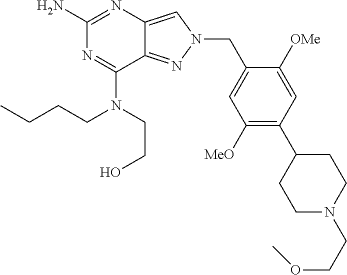 [SMILES: CCCCN(CCO)c1nc(N)nc2cn(Cc3cc(OC)c(C4CCN(CCOC)CC4)cc3OC)nc12] | δ 7.79 (s, 1H), 6.84 (d, J = 17.6 Hz, 3H), 6.10 (s, 1H), 5.38 (s, 2H), 4.13 (s, 2H), 3.78 (s, 4H), 3.67 (d, J = 8.2 Hz, 4H), 3.25 (s, 2H), 3.07- 2.96 (m, 2H), 2.86 (t, J = 13.7 Hz, 1H), 2.53-2.26 (m, 5H), 2.14 (t, J = 11.4 Hz, 2H), 1.91 (s, 2H), 1.81- 1.40 (m, 7H), 1.27 (s, 2H), 0.88 (d, J = 33.0 Hz, 3H). LC-MS (ES, m/z): [M + H]+ = 542.4, 1.4 mins | S9 |
| 107 | 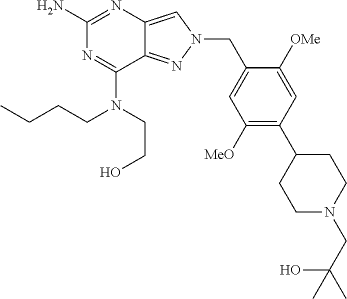 [SMILES: CCCCN(CCO)c1nc(N)nc2cn(Cc3cc(OC)c(C4CCN(CC(C)(C)O)CC4)cc3OC)nc12] | δ 7.80-7.62 (m, 1H), 6.84 (s, 1H), 6.79 (s, 1H), 5.66 (br d, J = 1.1 Hz, 2H), 5.35 (s, 2H), 4.22-4.01 (m, 1H), 3.78 (br s, 3H), 3.73-3.53 (m, 5H), 3.09-2.93 (m, 2H), 2.87-2.73 (m, 1H), 2.31-2.12 (m, 4H), 1.78- 1.62 (m, 2H), 1.58 (br d, J = 11.4 Hz, 4H), 1.37-1.18 (m, 2H), 1.09 (s, 6H), 0.99- 0.67 (m, 3H). Four protons were not visible due to water supression, one exchangeble protons were not visible either. LC-MS (ES, m/z): [M + H]+ = 556.2, 1.26 mins | S9 |
| 108 | 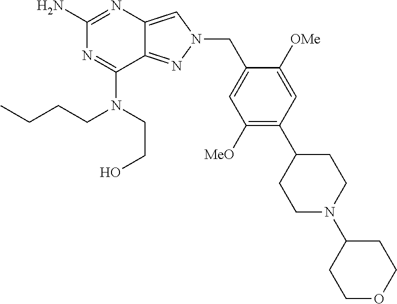 [SMILES: CCCCN(CCO)c1nc(N)nc2cn(Cc3cc(OC)c(C4CCN(C5CCOCC5)CC4)cc3OC)nc12] | δ 7.72 (br s, 1H), 6.86 (s, 1H), 6.80 (s, 1H), 5.67 (br s, 2H), 5.36 (s, 2H), 4.22- 4.02 (m, 1H), 3.89 (br dd, J = 10.5, 2.3 Hz, 3H), 3.84- 3.74 (m, 3H), 3.73-3.58 (m, 5H), 3.28 (br t, J = 11.3 Hz, 1H), 2.99 (br d, J = 8.9 Hz, 2H), 2.91-2.78 (m, 1H), 2.49-2.41 (m, 1H), 2.29-2.13 (m, 2H), 1.74- 1.54 (m, 8H), 1.45 (qd, J = 11.9, 3.4 Hz, 2H), 1.35- 1.18 (m, 2H), 0.99-0.75 (m, 3H). Four protons were not visible due to water supression. LC-MS (ES, m/z): [M + H]+ = 568.4, 1.18 mins | S9 |
| 109 | 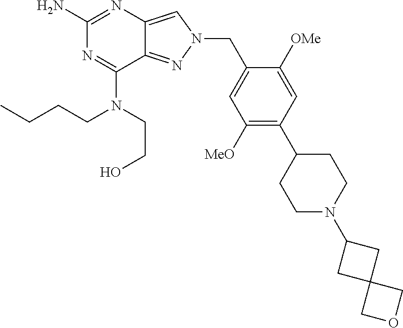 [SMILES: CCCCN(CCO)c1nc(N)nc2cn(Cc3cc(OC)c(C4CCN(C5CC6(COC6)C5)CC4)cc3OC)nc12] | δ 7.81-7.67 (m, 1H), 6.85 (s, 1H), 6.81 (s, 1H), 6.07- 5.77 (m, 2H), 5.37 (s, 2H), 4.58 (s, 2H), 4.45 (s, 2H), 4.20-4.06 (m, 1H), 3.84- 3.74 (m, 2H), 3.75-3.58 (m, 3H), 2.87 (br d, J = 11.0 Hz, 2H), 2.37-2.24 (m, 2H), 1.99-1.93 (m, 1H), 1.83-1.67 (m, 2H), 1.68- 1.48 (m, 4H), 1.38-1.10 (m, 2H), 1.03-0.67 (m, 3H). Twelev protons were not visible due to water supression and the overlap with DMSO-d6 peak. LC- MS (ES, m/z): [M + H]+ = 580.3, 1.53 mins | S9 |
| 113 | 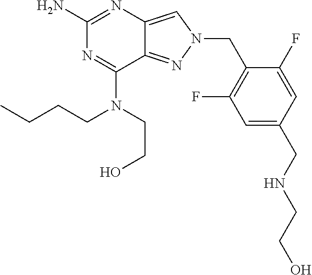 [SMILES: CCCCN(CCO)c1nc(N)nc2cn(Cc3c(F)cc(CNCCO)cc3F)nc12] | δ 8.28-8.09 (m, 1H), 7.79 (br s, 2H), 7.35 (br d, J = 8.2 Hz, 2H), 5.73-5.53 (m, 2H), 4.21 (s, 2H), 4.14- 4.00 (m, 2H), 3.78 (br t, J = 5.7 Hz, 2H), 3.73-3.59 (m, 3H), 3.59-3.37 (m, 2H), 2.97 (br t, J = 5.0 Hz, 2H), 1.68-1.46 (m, 2H), 1.39-1.15 (m, 2H), 0.96- 0.75 (m, 3H). Two protons were not visible due to water supression. LC-MS (ES, m/z): [M + H]+ = 450.3, 1.35 mins | S1 |
| 114 | 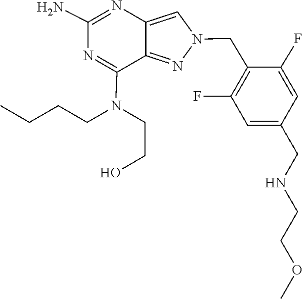 [SMILES: CCCCN(CCO)c1nc(N)nc2cn(Cc3c(F)cc(CNCCOC)cc3F)nc12] | δ 7.94-7.67 (m, 1H), 7.12 (br d, J = 8.8 Hz, 2H), 6.07- 5.74 (m, 2H), 5.51 (s, 2H), 4.26-3.90 (m, 2H), 3.80- 3.47 (m, 6H), 3.23 (s, 3H), 2.61 (br t, J = 5.5 Hz, 2H), 1.71-1.40 (m, 2H), 1.36- 1.07 (m, 2H), 1.00-0.67 (m, 3H). Four protons were not visible due to water supression. LC-MS (ES, m/z): [M + H]+ = 464.2, 1.03 mins | S1 |
| 115 | 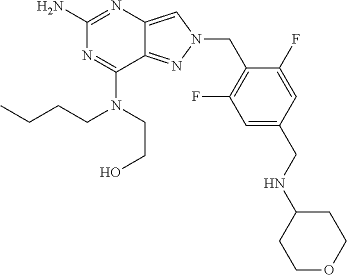 [SMILES: CCCCN(CCO)c1nc(N)nc2cn(Cc3c(F)cc(CNC4CCOCC4)cc3F)nc12] | δ 7.79 (br s, 1H), 7.13 (br d, J = 8.2 Hz, 2H), 5.57-5.47 (m, 2H), 4.09-3.91 (m, 1H), 3.84-3.76 (m, 2H), 3.74 (s, 2H), 3.70-3.56 (m, 1H), 3.30-3.16 (m, 1H), 1.73 (br d, J = 11.9 Hz, 2H), 1.65-1.39 (m, 2H), 1.36- 1.03 (m, 4H), 0.98-0.62 (m, 3H). Ten protons were not visible due to water supression. LCMS [M + H]+ = 490.2, 1.11 mins | S1 |
| 116 | 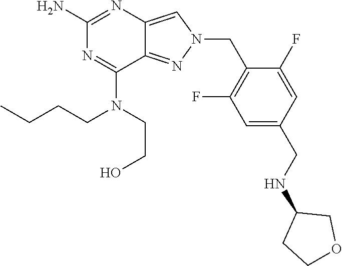 [SMILES: CCCCN(CCO)c1nc(N)nc2cn(Cc3c(F)cc(CN[C@@H]4CCOC4)cc3F)nc12] | δ 7.88-7.63 (m, 1H), 7.12 (br d, J = 8.2 Hz, 2H), 5.73 (br s, 2H), 5.50 (s, 2H), 3.98 (br s, 1H), 3.82-3.46 (m, 3H), 3.40 (dd, J = 8.4, 4.1 Hz, 1H), 3.25-3.12 (m, 1H), 1.90-1.84 (m, 1H), 1.71-1.61 (m, 1H), 1.61- 1.38 (m, 2H), 1.36-1.10 (m, 2H), 0.97-0.68 (m, 3H). Nine protons were not visible due to water supression and the overlap with DMSO-d6 peak. LC- MS (ES, m/z): [M + H]+ = 476.2, 1.1 mins | S1 |
| 117 | 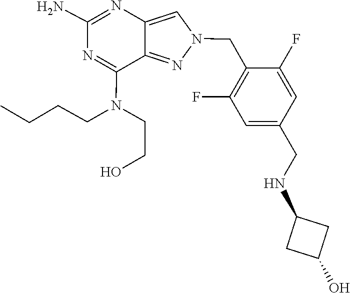 [SMILES: CCCCN(CCO)c1nc(N)nc2cn(Cc3c(F)cc(CN[C@H]4C[C@H](O)C4)cc3F)nc12] | δ 8.30-8.08 (m, 1H), 7.78 (s, 2H), 7.34 (d, J = 8.1 Hz, 3H), 7.17 (s, 0H), 5.76- 5.49 (m, 2H), 4.35 (s, 2H), 4.08 (d, J = 28.5 Hz, 5H), 3.91-3.63 (m, 3H), 2.51 (s, 3H), 2.40 (d, J = 7.2 Hz, 2H), 2.29-2.03 (m, 2H), 1.71-1.46 (m, 3H), 1.24 (ddq, J = 55.9, 14.2, 7.4 Hz, 2H), 0.92 (t, J = 7.3 Hz, 1H). LC-MS (ES, m/z): [M + H]+ = 476.3, 1.14 mins | S1 |
| 118 | 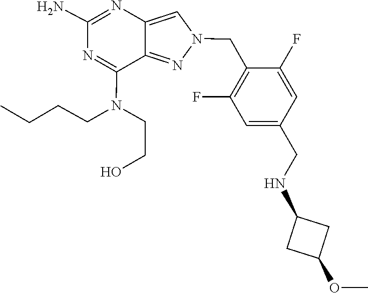 [SMILES: CCCCN(CCO)c1nc(N)nc2cn(Cc3c(F)cc(CN[C@H]4C[C@@H](OC)C4)cc3F)nc12] | δ 8.20 (s, 1H), 8.14 (s, 0H), 7.79 (s, 2H), 7.34 (d, J = 8.2 Hz, 3H), 5.65 (d, J = 8.0 Hz, 2H), 4.11 (d, J = 3.5 Hz, 3H), 4.05 (t, J = 8.0 Hz, 1H), 3.79 (q, J = 7.2, 5.7 Hz, 2H), 3.68 (dt, J = 14.7, 6.4 Hz, 2H), 3.14 (s, 3H), 2.51 (d, J = 1.7 Hz, 4H), 2.02 (d, J = 9.3 Hz, 3H), 1.74-1.49 (m, 1H), 1.31 (q, J = 7.4 Hz, 1H), 1.20 (q, J = 7.5 Hz, 1H), 0.92 (t, J = 7.3 Hz, 1H), 0.84 (t, J = 7.3 Hz, 3H). LC-MS (ES, m/z): [M + H]+ = 490.3, 1.15 mins | S1 |
| 119 | 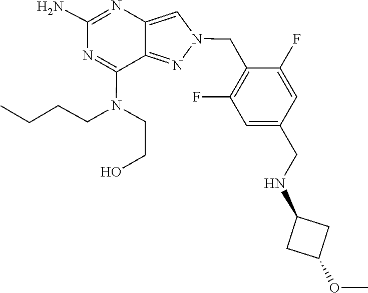 [SMILES: CCCCN(CCO)c1nc(N)nc2cn(Cc3c(F)cc(CN[C@H]4C[C@H](OC)C4)cc3F)nc12] | δ 8.04-7.78 (m, 1H), 7.12 (br d, J = 8.2 Hz, 2H), 6.70- 6.35 (m, 2H), 5.54 (s, 2H), 4.15-3.96 (m, 2H), 3.94 (br d, J = 5.1 Hz, 1H), 3.81- 3.62 (m, 4H), 3.60-3.42 (m, 1H), 3.24 (br dd, J = 5.5, 4.6 Hz, 1H), 3.09 (s, 3H), 2.09-1.91 (m, 4H), 1.70- 1.38 (m, 2H), 1.37-1.08 (m, 2H), 0.98-0.63 (m, 3H). Three protons were not visible due to water supression. LC-MS (ES, m/z): [M + H]+ = 490.1, 1.5 mins | S1 |
| 120 | 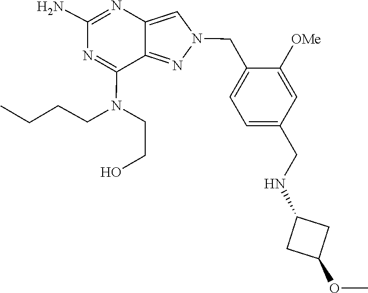 [SMILES: CCCCN(CCO)c1nc(N)nc2cn(Cc3ccc(CN[C@H]4C[C@H](OC)C4)cc3OC)nc12] | δ 7.68 (s, 1H), 7.01 (s, 1H), 6.93 (s, 1H), 6.84 (d, J = 7.7 Hz, 1H), 5.58 (s, 1H), 5.37 (s, 2H), 4.03-3.88 (m, 1H), 3.82 (s, 2H), 3.35-3.20 (m, 1H), 3.10 (d, J = 1.3 Hz, 3H), 2.55-2.36 (m, 6H), 1.97 (d, J = 5.4 Hz, 5H), 1.91 (d, J = 1.3 Hz, 4H), 1.57 (s, 2H), 1.28 (s, 3H), 0.88 (s, 3H). LC-MS (ES, m/z): [M + H]+ = 484.3, 1.16 mins | S1 |
| 121 | 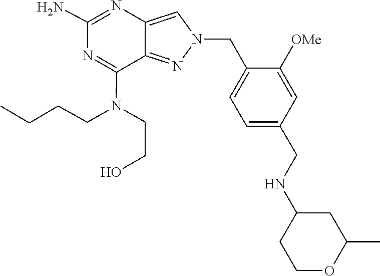 [SMILES: CCCCN(CCO)c1nc(N)nc2cn(Cc3ccc(CNC4CCOC(C)C4)cc3OC)nc12] | 1H NMR (400 MHz, DMSO-d6) δ = 7.74 (br s, 1H), 7.10 (s, 1H), 6.95 (s, 2H), 5.87 (s, 1H), 5.40 (s, 2H), 4.72 (s, 1H), 4.09 (br s, 2H), 3.84 (s, 3H), 3.76- 3.63 (m, 8H), 3.47 (td, J = 3.8, 7.7 Hz, 2H), 1.66-1.44 (m, 6H), 1.19 (br s, 6H), 0.92 (br s, 3H).; LC-MS (ES): m/z = 498.2 [M + H]+, RT (min) = 1.386 | S1 |
| 123 | 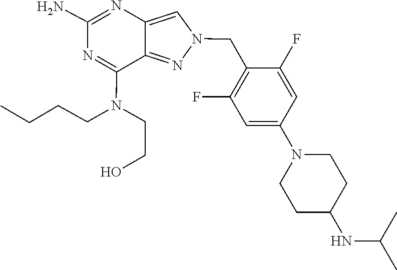 | δ 7.88-7.61 (m, 1H), 6.65 (br d, J = 11.2 Hz, 2H), 6.20- 5.99 (m, 1H), 5.38 (s, 2H), 4.04 (br d, J = 3.4 Hz, 1H), 3.87-3.54 (m, 5H), 2.92- 2.68 (m, 3H), 2.13-1.97 (m, 1H), 1.90-1.78 (m, 2H), 1.67 (td, J = 12.5, 6.4 Hz, 1H), 1.63-1.42 (m, 2H), 1.38-1.08 (m, 4H), 0.85 (br d, J = 6.5 Hz, 3H). Eight protons were not visible due to water supression and the overlap with the DMSO-d6 peak. Two exchangeble protons were not visible either. [M + H]+ = 545.2, 1.23 mins | S9 |
| 124 | 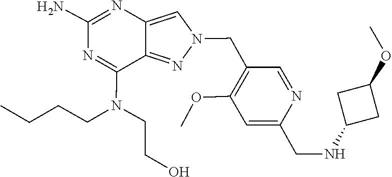 [SMILES: CCCCN(CCO)c1nc(N)nc2cn(Cc3cnc(CN[C@H]4C[C@H](OC)C4)cc3OC)nc12] | 1H NMR (400 MHz, DMSO-d6) δ = 8.17 (br s, 1H), 7.80 (br s, 1H), 7.13 (s, 1H), 5.88 (br s, 2H), 5.41 (s, 2H), 4.75 (br s, 1H), 4.16- 4.01 (m, 2H), 3.98-3.92 (m, 1H), 3.87 (br s, 3H), 3.74-3.62 (m, 7H), 3.10 (s, 3H), 2.00 (t, J = 5.9 Hz, 4H), 1.91 (s, 1H), 1.52 (br s, 2H), 1.24 (br s, 2H), 0.85 (br s, 3H); LC-MS (ES): | S1 |
| | | m/z = 485.3 [M + H]+, RT | |
| | | (min) = 1.117 | |
| 125 | 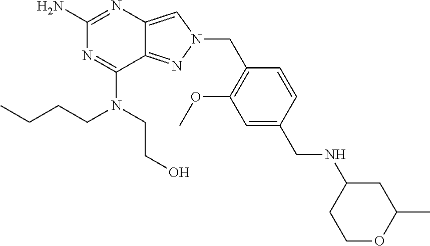 [SMILES: CCCCN(CCO)c1nc(N)nc2cn(Cc3ccc(CNC4CCOC(C)C4)cc3OC)nc12] | 1H NMR (400 MHz, DMSO-d6) δ = 7.71 (br d, J = 2.4 Hz, 1H), 7.06 (s, 1H), 6.95 (br s, 1H), 6.90-6.83 (m, 1H), 5.76-5.54 (m, 2H), 5.38 (s, 2H), 4.69 (br s, 2H), 4.17-3.94 (m, 3H), 3.86-3.75 (m, 5H), 3.64 (br s, 4H), 3.23 (br s, 2H), 2.63 (br d, J = 3.3 Hz, 1H), 1.97- 1.71 (m, 3H), 1.67-1.47 (m, 2H), 1.34-1.17 (m, 3H), 1.07 (d, J = 6.1 Hz, 3H), 0.98-0.78 (m, 3H); LC-MS (ES): m/z = 498.3 [M + H]+, RT (min) = 1.361 | S1 |
| 126 | 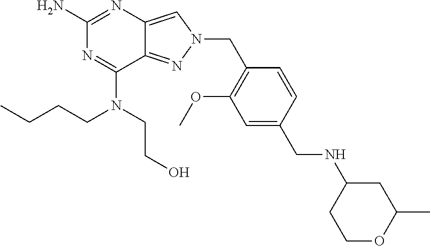 [SMILES: CCCCN(CCO)c1nc(N)nc2cn(Cc3ccc(CNC4CCOC(C)C4)cc3OC)nc12] | 1H NMR (400 MHz, DMSO-d6) δ = 7.69 (br s, 1H), 7.05 (s, 1H), 6.93 (br s, 1H), 6.90-6.81 (m, 1H), 5.53 (br s, 2H), 5.37 (s, 2H), 4.20-3.92 (m, 3H), 3.85- 3.79 (m, 4H), 3.74-3.61 (m, 5H), 3.20 (br d, J = 17.9 Hz, 3H), 2.61-2.54 (m, 2H), 2.00-1.80 (m, 2H), 1.80-1.72 (m, 1H), 1.57 (br s, 3H), 1.34-1.12 (m, 2H), 1.07 (d, J = 6.3 Hz, 3H), 0.97-0.75 (m, 3H); LC- MS (ES): m/z = 498.3 [M + H]+, RT (min) = 1.360 | S1 |
| 127 | 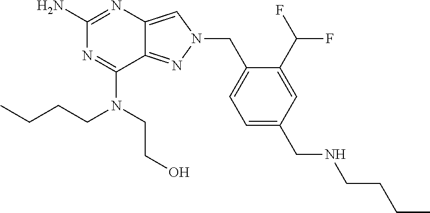 [SMILES: CCCCNCc1ccc(Cn2cc3nc(N)nc(N(CCO)CCCC)c3n2)c(C(F)F)c1] | δ 7.90-7.77 (m, 1H), 7.68- 7.60 (m, 1H), 7.52-7.45 (m, 1H), 7.45-7.32 (m, 1H), 7.25-7.17 (m, 1H), 5.66-5.60 (m, 2H), 4.15- 3.98 (m, 1H), 3.86-3.77 (m, 1H), 3.74-3.41 (m, 2H), 1.68-1.48 (m, 2H), 1.48-1.38 (m, 2H), 1.37- 1.11 (m, 4H), 0.98-0.74 (m, 5H), 11 protons were not visible due to water suppression. LC-MS (ES, m/z): [M + H]+ = 476.3, r.t. 1.45 mins | S1 |
| 128 | 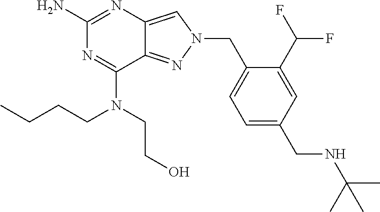 [SMILES: CCCCN(CCO)c1nc(N)nc2cn(Cc3ccc(CNC(C)(C)C)cc3C(F)F)nc12] | δ 8.14 (d, J = 15.6 Hz, 1H), 7.84 (s, 1H), 7.71 (br s, 1H), 7.67 (br d, J = 7.3 Hz, 1H), 7.60-7.26 (m, 2H), 5.76 (br d, J = 8.9 Hz, 2H), 4.18 (s, 2H), 4.16-4.10 (m, 1H), 3.84-3.49 (m, 3H), 1.61 (br dd, J = 15.0, 6.7 Hz, 2H), 1.38-1.29 (m, 9H), 1.29- 1.23 (m, 1H), 0.97-0.82 (m, 3H), 8 protons were not observed due to water suppression. LC-MS (ES, m/z): [M + H]+ = 476.3, 1.35 mins | S1 |
| 129 | 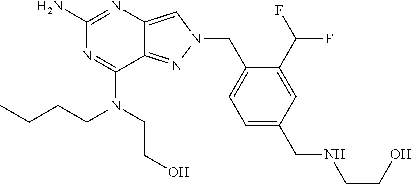 [SMILES: CCCCN(CCO)c1nc(N)nc2cn(Cc3ccc(CNCCO)cc3C(F)F)nc12] | δ 7.85 (br s, 1H), 7.64 (s, 1H), 7.53-7.25 (m, 2H), 7.20 (br d, J = 7.6 Hz, 1H), 5.71 (br s, 1H), 5.63 (s, 2H), 4.07 (br s, 1H), 3.93-3.55 (m, 3H), 2.60 (br s, 1H), 1.55 (br s, 2H), 1.38-1.17 (m, 2H), 1.01-0.70 (m, 3H), 11 protons were not observed due to water suppression. LC-MS (ES, m/z): [M + H]+ = 464.1, r.t. 1.24 mins | S1 |
| 130 | 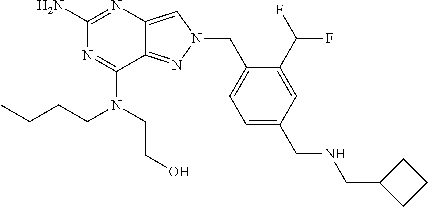 [SMILES: CCCCN(CCO)c1nc(N)nc2cn(Cc3ccc(CNCC4CCC4)cc3C(F)F)nc12] | δ 7.84 (br s, 1H), 7.61 (s, 1H), 7.45 (br d, J = 7.6 Hz, 2H), 7.17 (br d, J = 7.9 Hz, 1H), 5.62 (s, 2H), 5.55 (br s, 1H), 4.07 (br s, 1H), 3.76- 3.60 (m, 3H), 2.39 (dt, J = 14.8, 7.2 Hz, 1H), 1.97 (br d, J = 7.9 Hz, 2H), 1.88- 1.72 (m, 2H), 1.70-1.47 (m, 4H), 1.23 (br s, 2H), 1.03-0.75 (m, 3H), 11 protons were not observed due to water suppression. LC-MS (ES, m/z): | S1 |
| | | [M + H]+ = 488.3, r.t. | |
| | | 1.55 min | |
| 131 | 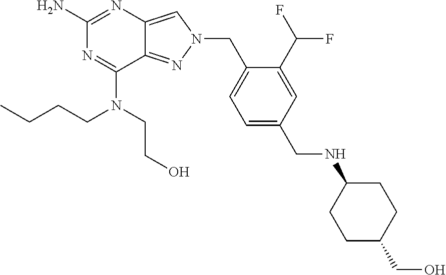 [SMILES: CCCCN(CCO)c1nc(N)nc2cn(Cc3ccc(CN[C@H]4CC[C@H](CO)CC4)cc3C(F)F)nc12] | δ 7.83 (br s, 1H), 7.62 (s, 1H), 7.54-7.23 (m, 2H), 7.17 (br d, J = 7.9 Hz, 1H), 5.62 (s, 2H), 5.52 (s, 1H), 4.07 (br s, 1H), 3.87-3.56 (m, 4H), 3.18 (br d, J = 6.1 Hz, 2H), 2.30 (br s, 1H), 1.71 (br d, J = 11.3 Hz, 2H), 1.55 (br s, 2H), 1.28 (br s, 3H), 1.09-0.74 (m, 8H), 8 protons were not observed due to water suppression. LC-MS (ES, m/z): [M + H]+ = 532.4, r.t. 1.27 mins | S1 |
| 132 | 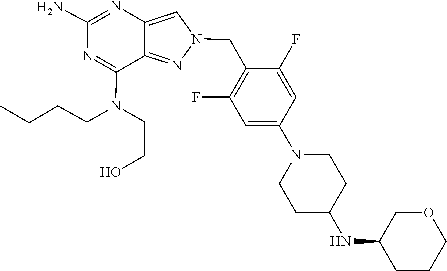 [SMILES: CCCCN(CCO)c1nc(N)nc2cn(Cc3c(F)cc(N4CCC(N[C@@H]5CCCOC5)CC4)cc3F)nc12] | δ 7.80-7.57 (m, 1H), 6.63 (br d, J = 11.3 Hz, 2H), 5.61 (br s, 2H), 5.36 (s, 2H), 4.16- 3.97 (m, 1H), 3.84-3.56 (m, 4H), 3.30-3.18 (m, 1H), 2.97 (br t, J = 9.8 Hz, 1H), 2.90-2.69 (m, 3H), 2.65 (td, J = 8.8, 3.8 Hz, 1H), 1.89 (br s, 1H), 1.86-1.73 (m, 2H), 1.69-1.40 (m, 4H), 1.40-1.10 (m, 5H), 0.99-0.74 (m, 3H). Seven protons were not visible due to water supression and the overlap with DMSO-d6 peak. LC-MS (ES, m/z): [M + H]+ = 559.4, r.t. 1.51 mins | S9 |
| 133 | 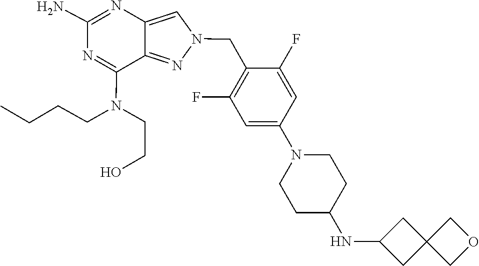 [SMILES: CCCCN(CCO)c1nc(N)nc2cn(Cc3c(F)cc(N4CCC(NC5CC6(COC6)C5)CC4)cc3F)nc12] | δ 7.79-7.55 (m, 1H), 6.61 (br d, J = 11.9 Hz, 2H), 5.56 (br s, 2H), 5.35 (s, 2H), 4.55 (s, 2H), 4.43 (s, 2H), 4.14- 3.94 (m, 1H), 3.75-3.56 (m, 2H), 3.09 (quin, J = 7.6 Hz, 1H), 2.87-2.72 (m, 2H), 2.46-2.36 (m, 2H), 1.87-1.81 (m, 2H), 1.74 (br d, J = 11.3 Hz, 2H), 1.65- 1.42 (m, 2H), 1.35-1.10 (m, 4H), 0.97-0.74 (m, 3H). Eight protons were not visible due to water supression and the overlap with DMSO-d6-peak. LC- MS (ES, m/z): [M + H]+ = 571.4, r.t. 1.2 mins | S9 |
| 134 | 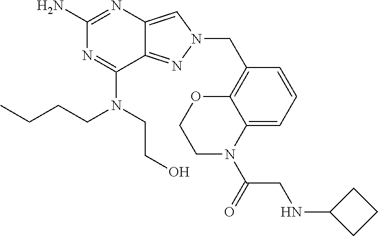 [SMILES: CCCCN(CCO)c1nc(N)nc2cn(Cc3cccc4c3OCCN4C(=O)CNC3CCC3)nc12] | δ 8.05-7.84 (m, 2H), 7.26 (br s, 1H), 6.92 (br d, J = 7.9 Hz, 2H), 5.49 (s, 2H), 4.38 (br s, 2H), 4.28-4.08 (m, 2H), 3.98 (br s, 2H), 3.92- 3.63 (m, 4H), 2.22-1.96 (m, 4H), 1.79-1.54 (m, 4H), 1.45-1.17 (m, 3H), 1.02-0.81 (m, 3H), 5 protons were not observed due to water suppression. LC-MS (ES, m/z): [M + H]+ = 509.2, r.t. 1.38 mins | S12 |
| 135 | 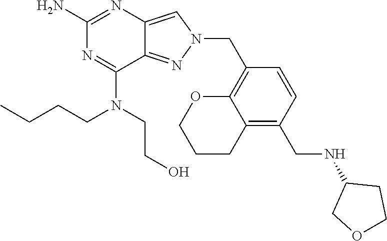 | 1H NMR (400 MHz, DMSO-d6) δ = 7.70 (br s, 1H), 6.86-6.65 (m, 2H), 5.79 (br s, 2H), 5.34 (s, 2H), 4.72 (br s, 1H), 4.22-4.01 (m, 3H), 3.77-3.54 (m, 7H), 3.44 (br s, 1H), 2.76 (br t, J = 6.6 Hz, 2H), 2.00-1.87 (m, 3H), 1.68 (br d, J = 5.1 Hz, 1H), 1.59 (br s, 2H), 1.30 (br s, 2H), 0.90 (br s, 3H); LC-MS (ES): m/z = 496.3 [M + H]+, RT (min) = 0.910 | S12 |
| 137 | 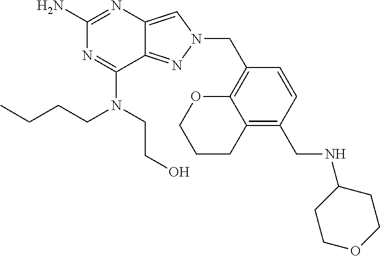 [SMILES: CCCCN(CCO)c1nc(N)nc2cn(Cc3ccc(CNC4CCOCC4)c4c3OCCC4)nc12] | 1H NMR (400 MHz, DMSO-d6) δ = 7.71 (br s, 1H), 6.89-6.83 (m, 1H), 6.79 (br s, 1H), 5.84 (br s, 1H), 5.35 (s, 1H), 4.22- 4.02 (m, 2H), 3.87-3.81 (m, 1H), 3.77-3.58 (m, 3H), 3.25 (br s, 2H), 2.76 (br t, J = 6.4 Hz, 1H), 1.99-1.92 (m, 1H), 1.82 (br dd, J = 2.1, 13.1 Hz, 1H), 1.59 (br s, 1H), 1.37-1.25 (m, 2H), 0.90 (br s, 2H); LC-MS (ES): m/z = 510.3 [M + H]+, RT (min) = 0.929 | S12 |
| 138 | 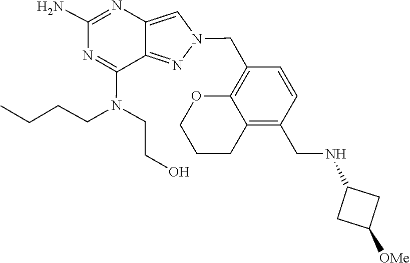 | 1H NMR (400 MHz, DMSO-d6) δ = 7.84-7.74 (m, 1H), 6.83 (br s, 2H), 6.45 (br s, 1H), 5.37 (s, 2H), 4.78 (br s, 1H), 4.16 (br s, 4H), 3.95 (br t, J = 5.6 Hz, 1H), 3.79-3.63 (m, 5H), 3.58 (br s, 2H), 3.11 (s, 4H), 2.75 (br t, J = 6.2 Hz, 2H), 2.04 (br t, J = 5.7 Hz, 4H), 1.97-1.92 (m, 2H), 1.60 (br s, 2H), 1.36-1.24 (m, 2H), 0.95-0.83 (m, 3H); LC-MS (ES): m/z = 510.3 [M + H]+, RT (min) = 0.972 | S12 |
| 139 | 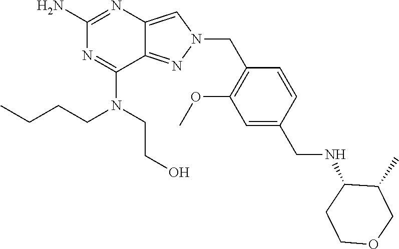 [SMILES: CCCCN(CCO)c1nc(N)nc2cn(Cc3ccc(CN[C@H]4CCOC[C@H]4C)cc3OC)nc12] | δ = 7.76 (br d, J = 1.4 Hz, 1H), 7.07 (s, 1H), 6.97 (br s, 1H), 6.93-6.84 (m, 1H), 5.98 (br s, 2H), 5.40 (s, 2H), 4.76 (br s, 1H), 4.12 (br s, 2H), 3.86-3.63 (m, 10H), 3.56 (br d, J = 11.8 Hz, 1H), 2.73 (br s, 1H), 1.89-1.80 (m, 1H), 1.68-1.45 (m, 4H), 1.33-1.24 (m, 2H), 0.95-0.78 (m, 6H). LC-MS (ES): m/z = 498.3 [M + H]+, RT (min) = 0.90 | S1 |
| 142 | 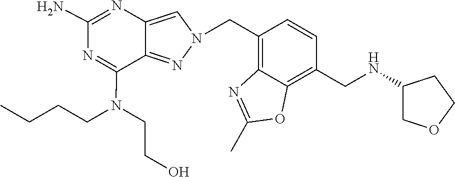 | 1H NMR (400 MHz, DMSO-d6) δ = 8.22-7.97 (m, 1H), 7.36 (br d, J = 7.6 Hz, 3H), 7.21 (br d, J = 7.4 Hz, 1H), 5.80 (s, 2H), 4.79 (br s, 1H), 4.24-4.07 (m, 2H), 4.00 (br s, 2H), 3.78 (q, J = 7.3 Hz, 3H), 3.72- 3.60 (m, 5H), 3.53-3.49 (m, 1H), 2.65 (s, 3H), 2.04- 1.94 (m, 1H), 1.79-1.70 (m,1H), 1.67-1.45 (m, 2H), | S20 |
| | | 1.36-1.15 (m, 2H), 0.98- | |
| | | 0.71 (m, 3H). LC-MS (ES): | |
| | | m/z = 495.3 [M + H]+, RT | |
| | | (min) = 1.18 | |
| 143 | 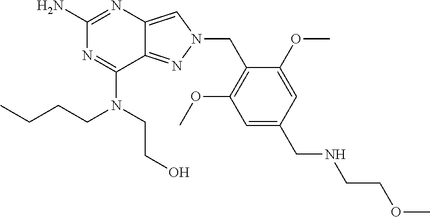 [SMILES: CCCCN(CCO)c1nc(N)nc2cn(Cc3c(OC)cc(CNCCOC)cc3OC)nc12] | δ 7.67-7.34 (m, 1H), 6.75 (s, 2H), 6.05 (br s, 2H), 5.38 (s, 2H), 4.20-4.02 (m, 1H), 3.89 (s, 1H), 3.80 (br s, 6H), 3.75-3.61 (m, 2H), 3.62- 3.51 (m, 1H), 3.44 (br t, J = 5.5 Hz, 1H), 3.25 (s, 3H), 2.75 (br t, J = 5.0 Hz, 2H), 1.71-1.46 (m, 2H), 1.37- 1.14 (m, 2H), 0.89 (br d, J = 7.6 Hz, 3H). Six protons were not visible due to water supression and the overlap with DMSO-d6 LC- MS (ES, m/z): [M + H]+ = | S1 |
| | | 474.3, r.t. 1.17 mins | |
| | | peak. LC-MS (ES, m/z): | |
| | | [M + H]+ = 488.3, r.t. 1.45 | |
| | | mins | |
| 144 | 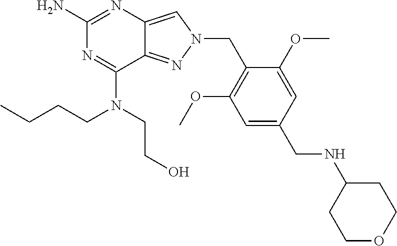 [SMILES: CCCCN(CCO)c1nc(N)nc2cn(Cc3c(OC)cc(CNC4CCOCC4)cc3OC)nc12] | δ 7.67-7.34 (m, 1H), 6.75 (s, 2H), 6.05 (br s, 2H), 5.38 (s, 2H), 4.20-4.02 (m, 1H), 3.89 (s, 1H), 3.80 (br s, 6H), 3.75-3.61 (m, 2H), 3.62- 3.51 (m, 1H), 3.44 (br t, J = 5.5 Hz, 1H), 3.25 (s, 3H), 2.75 (br t, J = 5.0 Hz, 2H), 1.71-1.46 (m, 2H), 1.37- 1.14 (m, 2H), 0.89 (br d, J = 7.6 Hz, 3H). Three protons were not visible due to water supression. LC-MS (ES, m/z): [M + H]+ = 514.4, r.t. 1.06 mins | S1 |
| 145 | 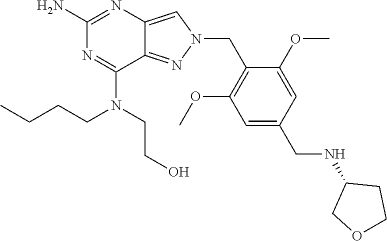 [SMILES: CCCCN(CCO)c1nc(N)nc2cn(Cc3c(OC)cc(CN[C@@H]4CCOC4)cc3OC)nc12] | δ 7.54-7.33 (m, 1H), 6.72 (s, 2H), 5.58 (br s, 2H), 5.36 (s, 2H), 4.28-4.02 (m, 1H), 3.87-3.58 (m, 13H), 3.54- 3.35 (m, 1H), 3.32-3.22 (m, 1H), 3.00 (s, 2H), 1.99- 1.91 (m, 1H), 1.75-1.64 (m, 1H), 1.63-1.48 (m, 2H), 1.37-1.15 (m, 2H), 0.90 (br s, 3H). Three protons were not visible due to water supression and overlap with DMSO-d6 peak. LC-MS (ES, m/z): [M + H]+ = 500.4, r.t. 1.18 mins | S1 |
| 146 | 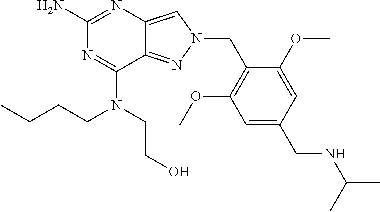 [SMILES: CCCCN(CCO)c1nc(N)nc2cn(Cc3c(OC)cc(CNC(C)C)cc3OC)nc12] | δ 7.55-7.33 (m, 1H), 6.75 (s, 2H), 5.46 (br s, 2H), 5.36 (s, 2H), 4.20-4.00 (m, 1H), 3.87-3.68 (m, 6H), 3.69- 3.49 (m, 2H), 2.91-2.76 (m, 1H), 1.63-1.49 (m, 2H), 1.36-1.20 (m, 2H), 1.07 (br d, J = 6.1 Hz, 6H), 0.89 (br s, 3H). Seven protons were not visible due to water supression. LC-MS (ES, m/z): [M + H]+ = 472.4, r.t. 1.21 mins | S1 |
| 147 | 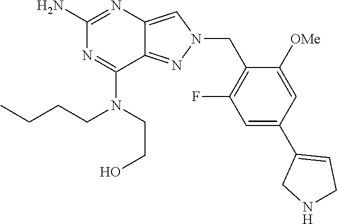 [SMILES: CCCCN(CCO)c1nc(N)nc2cn(Cc3c(F)cc(C4=CCNC4)cc3OC)nc12] | 1H NMR (400 MHz, DMSO-d6) δ = 7.60-7.80 (m, 1H), 6.90-7.10 (m, 2H), 6.55-7.55 (m, 1H), 3.80- 4.10 (m, 7H), 3.50-3.70 (m, 6H), 1.40-1.60 (m, 2H), 1.10-1.30 (m, 2H), 0.70- 0.90 (m, 3H); LC-MS (ES): m/z = 456.3 [M + H]+, RT (min) = 0.844 | S9 |
| 148 | 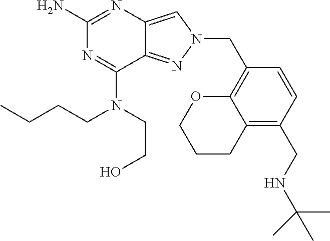 [SMILES: CCCCN(CCO)c1nc(N)nc2cn(Cc3ccc(CNC(C)(C)C)c4c3OCCC4)nc12] | 1H NMR (400 MHz, DMSO-d6) δ = 7.69 (s, 1H), 6.92-6.86 (m, 1H), 6.81 (br s, 1H), 5.57 (br s, 1H), 5.36 (s, 2H), 4.73 (s, 1H), 4.19 (br d, J = 4.5 Hz, 3H), 3.83- 3.69 (m, 2H), 3.65 (br d, J = 4.0 Hz, 2H), 2.79 (br t, J = 6.6 Hz, 2H), 2.01-1.94 (m, 2H), 1.63-1.54 (m, 2H), 1.30 (br d, J = 8.4 Hz, 2H), 1.22 (br s, 9H), 0.90 (br s, 3H); LC-MS (ES): m/z = 482.3 [M + H]+, RT (min) = 1.313 | S12 |
| 149 | 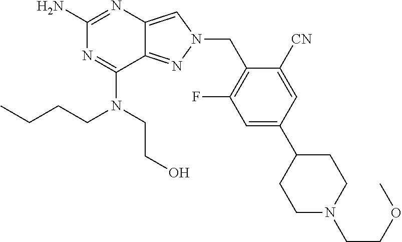 [SMILES: CCCCN(CCO)c1nc(N)nc2cn(Cc3c(F)cc(C4CCN(CCOC)CC4)cc3C#N)nc12] | δ 8.31-8.16 (m, 1H), 7.81 (br s, 2H), 7.71 (br d, J = 0.6 Hz, 1H), 7.65-7.50 (m, 1H), 5.80-5.65 (m, 2H), 4.15-3.99 (m, 2H), 3.86- 3.63 (m, 4H), 3.58-3.41 (m, 1H), 3.13-3.02 (m, 1H), 3.01-2.91 (m, 1H), 2.10-1.87 (m, 3H), 1.67- 1.41 (m, 2H), 1.36-1.02 (m, 3H), 0.95-0.64 (m, 3H), Ten protons were not visible due to water supression and the overlap with DMSO-d6 peak. LC- MS (ES, m/z): [M + H]+ = 525.3, r.t. 1.13 mins | S9 |
| 150 | 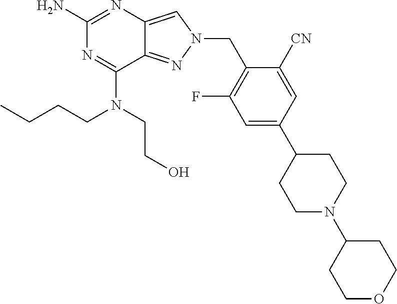 [SMILES: CCCCN(CCO)c1nc(N)nc2cn(Cc3c(F)cc(C4CCN(C5CCOCC5)CC4)cc3C#N)nc12] | δ 8.37-8.16 (m, 1H), 7.86- 7.55 (m, 2H), 5.86-5.60 (m, 2H), 4.15-3.96 (m, 2H), 3.86-3.74 (m, 1H), 3.73-3.49 (m, 3H), 3.40- 3.27 (m, 1H), 3.13-2.92 (m, 2H), 2.07 (br dd, J = 9.6, 4.4 Hz, 1H), 1.93-1.80 (m, 1H), 1.75-1.56 (m, 2H), 1.45 (br d, J = 7.0 Hz, 1H), 1.36-1.01 (m, 3H), 0.96- 0.55 (m, 3H) fourteen protons were not visible due to water supression and the overlap with DMSO-d6 peak. LC-MS (ES, m/z): [M + H]+ = 551.4, r.t. 1.12 mins | S9 |
| 151 | 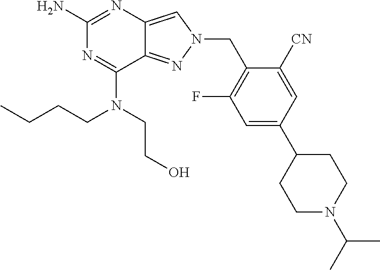 [SMILES: CCCCN(CCO)c1nc(N)nc2cn(Cc3c(F)cc(C4CCN(C(C)C)CC4)cc3C#N)nc12] | δ 8.33-8.12 (m, 1H), 7.80 (br s, 2H), 7.70 (br s, 1H), 7.58 (br d, J = 10.7 Hz, 1H), 5.80-5.58 (m, 2H), 4.17- 3.98 (m, 2H), 3.86-3.64 (m, 2H), 3.61-3.46 (m, 1H), 3.17-2.94 (m, 3H), 2.17-1.78 (m, 4H), 1.69- 1.39 (m, 2H), 1.28 (d, J = 6.7 Hz, 6H), 1.09 (br d, J = 7.6 Hz, 1H), 0.96-0.51 (m, 3H), six protons were not visible due to water supression and the overlap with DMSO-d6 peak. LC- MS (ES, m/z): [M + H]+ = 509.2, r.t. 1.07 mins | S9 |
| 152 | 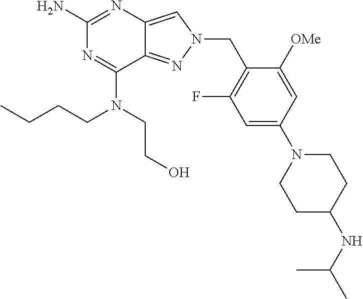 [SMILES: CCCCN(CCO)c1nc(N)nc2cn(Cc3c(F)cc(N4CCC(NC(C)C)CC4)cc3OC)nc12] | δ 7.70-7.37 (m, 2H), 6.35 (br s, 2H), 5.46 (br s, 2H), 5.28 (s, 2H), 4.23-3.96 (m, 1H), 3.87-3.72 (m, 3H), 3.71-3.51 (m, 4H), 3.09- 2.97 (m, 1H), 2.90-2.69 (m, 3H), 1.86 (br d, J = 11.0 Hz, 2H), 1.66-1.41 (m, 2H), 1.39-1.15 (m, 4H), 1.03 (br d, J = 6.1 Hz, 6H), 0.87 (br s, 3H.).Four protons were not visible due to water supression. LC-MS (ES, m/z): [M + H]+ = 529.3, r.t. 1.26 mins | S9 |
| 154 | 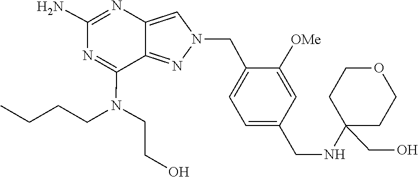 [SMILES: CCCCN(CCO)c1nc(N)nc2cn(Cc3ccc(CNC4(CO)CCOCC4)cc3OC)nc12] | 1H NMR (400 MHz, DMSO-d6) δ = 7.75 (br d, J = 0.9 Hz, 1H), 7.09 (s, 1H), 7.04-6.86 (m, 2H), 5.97 (br s, 1H), 5.40 (s, 2H), 4.73 (br s, 1H), 4.20-4.00 (m, 2H), 3.83 (s, 3H), 3.75-3.56 (m, 9H), 3.53-3.47 (m, 3H), 1.48 (br s, 6H), 1.35-1.24 (m, 2H), 0.89 (br s, 3H); LC-MS (ES); m/z = 514.3 [M + H]+, RT (min) = 0.845 | S1 |
| 155 | 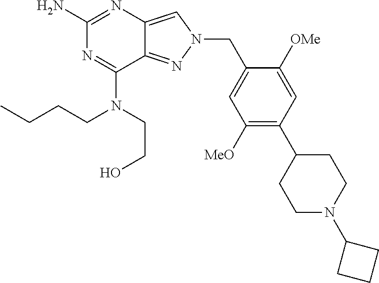 | δ 7.81-7.67 (m, 1H), 6.85 (s, 1H), 6.81 (s, 1H), 6.07- 5.77 (m, 2H), 5.37 (s, 2H), 4.58 (s, 2H), 4.45 (s, 2H), 4.20-4.06 (m, 1H), 3.84- 3.74 (m, 2H), 3.75-3.58 (m, 3H), 2.87 (br d, J = 11.0 Hz, 2H), 2.37-2.24 (m, 2H), 1.99-1.93 (m, 1H), 1.83-1.67 (m, 2H), 1.68- 1.48 (m, 4H), 1.38-1.10 (m, 2H), 1.03-0.67 (m, 3H). Twelev protons were not visible due to water supression and the overlap with DMSO-d6 peak. LC- MS (ES, m/z): [M + H]+ = 580.3, r.t. 1.46 mins | S9 |
| 156 | 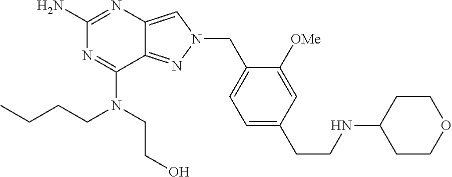 [SMILES: CCCCN(CCO)c1nc(N)nc2cn(Cc3ccc(CCNC4CCOCC4)cc3OC)nc12] | 1H NMR (400 MHz, DMSO-d6) δ = 8.82 (br s, 2H), 7.67 (br s, 1H), 6.90 (s, 2H), 6.75 (dd, J = 1.1, 7.7 Hz, 1H), 5.37 (d, J = 14.1 Hz, 4H), 4.73 (br s, 1H), 4.11 (br s, 2H), 3.86-3.76 (m, 5H), 3.72-3.57 (m, 5H), 2.81-2.73 (m, 2H), 2.71-2.58 (m, 3H), 1.77- 1.65 (m, 2H), 1.58 (br s, 2H), 1.35-1.13 (m, 4H), | S1 |
| | | 0.87 (br d, J = 2.6 Hz, 3H). | |
| | | LC-MS (ES): m/z = 498.3 | |
| | | [M + H]+, RT (min) = 1.28 | |
| 157 | 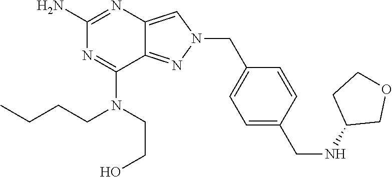 [SMILES: CCCCN(CCO)c1nc(N)nc2cn(Cc3ccc(CN[C@@H]4CCOC4)cc3)nc12] | δ 7.93 (s, 1H), 7.50-7.01 (m, 5H), 6.26 (s, 2H), 5.46 (s, 3H), 4.14 (s, 2H), 3.98- 3.35 (m, 8H), 2.55 (s, 3H), 2.16 (s, 1H), 2.08-1.84 (m, 1H), 1.71 (d, J = 6.5 Hz, 1H), 1.30 (s, 3H), 0.89 (s, 3H). LC-MS (ES, m/z): [M + H]+ = 440.1, r.t. 1.02 mins | S1 |
| 158 | 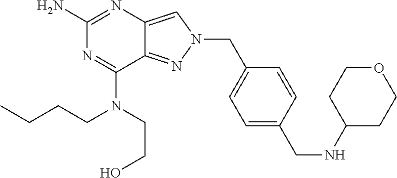 [SMILES: CCCCN(CCO)c1nc(N)nc2cn(Cc3ccc(CNC4CCOCC4)cc3)nc12] | δ 8.18 (d, J = 23.2 Hz, 1H), 7.76 (s, 2H), 7.51 (d, J = 7.8 Hz, 4H), 7.42 (d, J = 7.8 Hz, 3H), 5.58 (s, 2H), 4.21 (d, J = 37.7 Hz, 4H), 3.93 (dd, J = 11.5, 4.5 Hz, 2H), 3.82 (t, J = 6.3 Hz, 2H), 3.72 (s, 1H), 3.30 (t, J = 11.6 Hz, 2H), 2.10-1.88 (m, 2H), 1.80-1.49 (m, 5H), 1.33 (p, J = 7.4 Hz, 2H), 0.92 (dt, J = 20.2, 7.4 Hz, 3H). LC- | S1 |
| | | MS (ES, m/z): [M + H]+ = | |
| | | 454.2, r.t. 1.01 mins | |
| 159 | 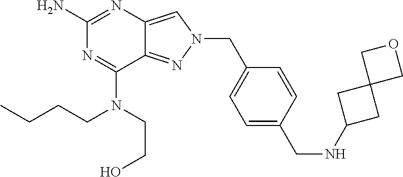 [SMILES: CCCCN(CCO)c1nc(N)nc2cn(Cc3ccc(CNC4CC5(COC5)C4)cc3)nc12] | δ 7.88 (s, 1H), 7.29 (q, J = 7.8 Hz, 4H), 5.97 (s, 1H), 5.44 (s, 2H), 4.48 (d, J = 35.6 Hz, 4H), 4.13 (s, 1H), 3.93-3.49 (m, 4H), 3.05 (s, 1H), 2.51 (s, 3H), 2.39 (d, J = 10.6 Hz, 2H), 1.94 (dd, J = 17.4, 6.9 Hz, 3H), 1.59 (s, 3H), 1.30 (s, 3H), 0.89 (s, 3H). LC-MS (ES, m/z): [M + H]+ = 466.2, r.t. 1.1 mins | S1 |
| 160 | 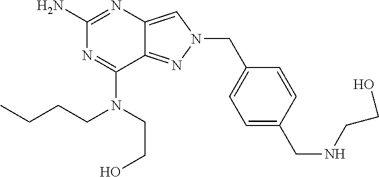 [SMILES: CCCCN(CCO)c1nc(N)nc2cn(Cc3ccc(CNCCO)cc3)nc12] | δ 7.82 (s, 1H), 7.45-6.95 (m, 5H), 5.62 (s, 1H), 5.43 (s, 2H), 4.13 (s, 2H), 3.82- 3.41 (m, 3H), 2.57 (dd, J = 11.2, 5.6 Hz, 3H), 1.91 (s, 5H), 1.58 (s, 3H), 1.30 (s, 3H), 0.89 (s, 3H). LC-MS (ES, m/z): [M + H]+ = 414.2, r.t. 1.04 mins | S1 |
| 162 | 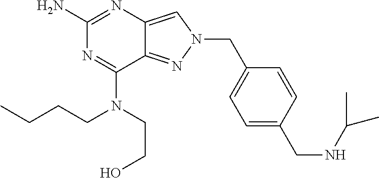 [SMILES: CCCCN(CCO)c1nc(N)nc2cn(Cc3ccc(CNC(C)C)cc3)nc12] | δ 7.81 (s, 1H), 7.53-7.03 (m, 4H), 5.52 (s, 1H), 4.12 (s, 1H), 3.70 (d, J = 47.8 Hz, 2H), 2.78 (d, J = 7.5 Hz, 1H), 2.53 (d, J = 19.3 Hz, 6H), 1.91 (s, 2H), 1.58 (s, 2H), 1.30 (s, 2H), 1.03 (d, J = 6.3 Hz, 5H), 0.89 (s, 3H). 3 protons were not visible due to water supression and the overlap with DMSO-d6 peak. LC-MS (ES, m/z): | S1 |
| | | [M + H]+ = 412.2, r.t. 1.12 | |
| | | mins | |
| 163 | 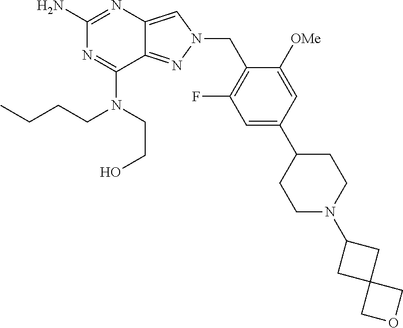 [SMILES: CCCCN(CCO)c1nc(N)nc2cn(Cc3c(F)cc(C4CCN(C5CC6(COC6)C5)CC4)cc3OC)nc12] | δ 7.72-7.49 (m, 1H), 6.78 (br s, 1H), 6.71 (br d, J = 10.1 Hz, 1H), 5.37 (s, 2H), 4.58 (s, 2H), 4.44 (s, 2H), 4.03 (br d, J = 7.6 Hz, 1H), 3.83 (br s, 3H), 3.73- 3.48 (m, 2H), 2.85 (br d, J = 11.0 Hz, 2H), 2.50-2.44 (m, 2H), 2.36-2.24 (m, 2H), 1.95-1.91 (m, 2H), 1.82-1.65 (m, 4H), 1.65- 1.43 (m, 4H), 1.38-1.15 (m, 2H), 0.98-0.73 (m, 3H). Six protons were not visible due to water supression and the overlap with DMSO-d6 peak. LC- MS (ES, m/z): [M + H]+ = 568.4, r.t. 1.45 mins | S9 |
| 165 | 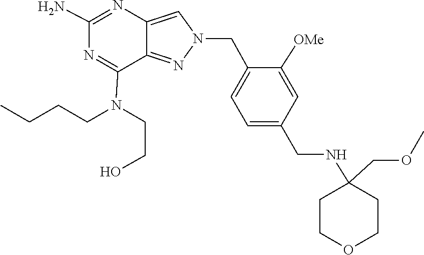 [SMILES: CCCCN(CCO)c1nc(N)nc2cn(Cc3ccc(CNC4(COC)CCOCC4)cc3OC)nc12] | 1H NMR (400 MHz, DMSO-d6) δ = 7.69 (br s, 1H), 7.05 (s, 1H), 7.01- 6.93 (m, 1H), 6.92-6.83 (m, 1H), 5.62 (br s, 2H), 5.37 (s, 2H), 4.74-4.60 (m, 1H), 4.09 (br s, 3H), 3.82 (s, 3H), 3.73-3.61 (m, 7H), 3.52-3.48 (m, 2H), 3.28 (s, 3H), 3.25 (s, 2H), 1.60- 1.38 (m, 5H), 1.23 (br s, 2H), 0.85 (br s, 3H); LC- MS (ES): m/z = 528.3 [M + H]+, RT (min) = 0.994 | S1 |
| 167 | 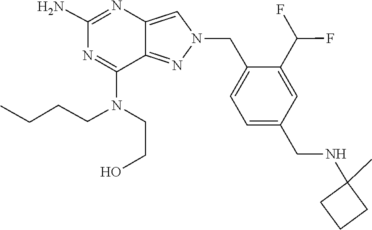 [SMILES: CCCCN(CCO)c1nc(N)nc2cn(Cc3ccc(CNC4(C)CCC4)cc3C(F)F)nc12] | δ 8.08 (br s, 1H), 7.83 (br s, 1H), 7.65 (br d, J = 7.9 Hz, 1H), 7.60-7.43 (m, 1H), 7.35 (br d, J = 7.0 Hz, 1H), 5.75 (br s, 2H), 4.29-3.99 (m, 4H), 3.83-3.60 (m, 3H), 2.33 (br d, J = 15.9 Hz, 2H), 1.95-1.73 (m, 4H), 1.60 (br s, 2H), 1.48 (br s, 2H), 1.40-1.18 (m, 3H), 0.99-0.82 (m, 3H), 5 protons were not observed due to water suppression. LC-MS (ES, m/z): [M + H]+ = 488.2, r.t. 1.48 mins | S1 |
| 168 | 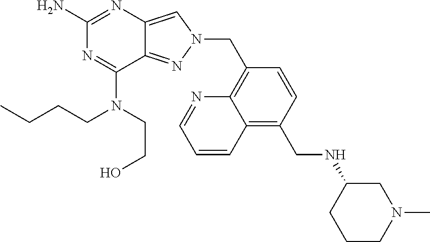 [SMILES: CCCCN(CCO)c1nc(N)nc2cn(Cc3ccc(CN[C@H]4CCCN(C)C4)c4cccnc34)nc12] | δ = 8.99 (dd, J = 1.6, 4.1 Hz, 1H), 8.69 (dd, J = 1.8, 8.8 Hz, 1H), 7.89 (br s, 1H), 7.62 (dd, J = 4.0, 8.5 Hz, 1H), 7.55 (d, J = 7.3 Hz, 1H), 7.31 (br s, 1H), 6.07 (s, 2H), 5.64 (br dd, J = 0.8, 4.3 Hz, 1H), 4.18 (s, 4H), 3.63 (br s, 4H), 2.85 ddd, J = 1.9, 3.5, 7.6 Hz, 1H), 2.65-2.55 (m, 2H), 2.18 (s, 2H), 2.01- 1.92 (m, 1H), 1.84 (br s, 2H), 1.67-1.38 (m, 4H), 1.24 (s, 3H), 0.79 (br s, 3H). LC-MS (ES, m/z): [M + H]+ = 518.3, r.t. 0.67 mins | S10 |
| 169 | 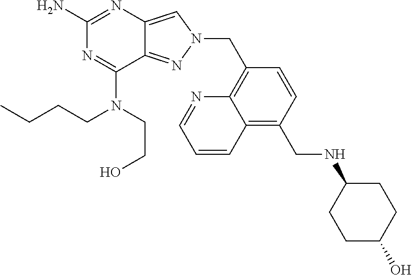 [SMILES: CCCCN(CCO)c1nc(N)nc2cn(Cc3ccc(CN[C@H]4CC[C@H](O)CC4)c4cccnc34)nc12] | δ = 8.98 (br dd, J = 1.5, 4.0 Hz, 1H), 8.67 (dd, J = 1.6 Hz, 1H), 7.86 (br s, 1H), 7.64-7.60 (m, 1H), 7.53 (d, J = 7.0 Hz, 1H), 7.29 (br s, 1H), 6.06 (s, 2H), 5.55 (br s, 2H), 4.45 (br s, 4H), 4.15 (s, 4H), 3.75-3.59 (m, 4H), 2.44 (br d, J = 2.3 Hz, 1H), 1.96-1.88 (m, 3H), 1.83- 1.73 (m, 2H), 1.52 (br s, 2H), 1.30-1.02 (mm 5H), 0.78 (br s, 3H). ). LC-MS (ES, m/z): [M + H]+ = 519.3, r.t. 0.73 mins | S10 |
| 170 | 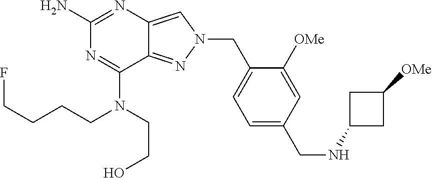 [SMILES: COc1cc(CN[C@H]2C[C@H](OC)C2)ccc1Cn1cc2nc(N)nc(N(CCO)CCCCF)c2n1] | 1H NMR (400 MHz, DMSO-d6) δ = 7.81 (br d, J = 2.9 Hz, 1H), 7.07 (s, 2H), 6.90 (d, J = 7.4 Hz, 1H), 6.31 (br s, 2H), 5.42 (s, 2H), 4.77 (br s, 1H), 4.57-4.30 (m, 2H), 4.24-4.06 (m, 2H), 3.96 (br t, J = 5.1 Hz, 1H), 3.83 (s, 3H), 3.77- 3.59 (m, 6H), 3.11 (s, 3H), 2.11-1.96 (m, 4H), 1.79- 1.49 (m, 3H). LC-MS (ES): | S1 |
| | | m/z = 502.3 [M + H]+, RT | |
| | | (min) = 0.803 | |
| 171 | 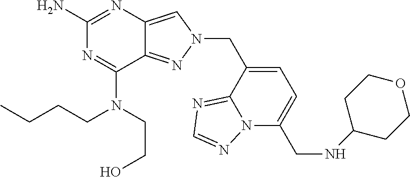 [SMILES: CCCCN(CCO)c1nc(N)nc2cn(Cc3ccc(CNC4CCOCC4)n4ncnc34)nc12] | 1H NMR (400 MHz, DMSO-d6) δ = 8.55 (s, 1H), 7.95 (br d, J = 0.9 Hz, 1H), 7.45 (br d, J = 6.4 Hz, 1H), 7.26 (d, J = 7.4 Hz, 1H), 5.82 (s, 2H), 5.70 (br s, 2H), 4.67 (br s, 1H), 4.22 (s, 2H), 4.01 (br s, 2H), 3.81 (td, J = 3.5, 11.2 Hz, 2H), 3.63 (br d, J = 2.3 Hz, 5H), 3.24 (br d, J = 2.0 Hz, 3H), 1.83- 1.72 (m, 2H), 1.66-1.38 | S18 |
| | | (m, 2H), 1.35-1.21 (m, | |
| | | 3H), 1.12 (br s, 1H), 0.73 | |
| | | (br s, 3H). LC-MS (ES): | |
| | | m/z = 495.2 [M + H]+, RT | |
| | | (min) = 1.07 | |
| 172 | 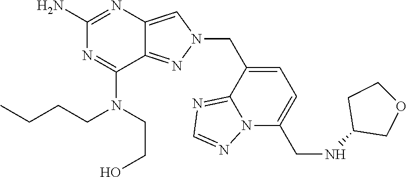 [SMILES: CCCCN(CCO)c1nc(N)nc2cn(Cc3ccc(CN[C@@H]4CCOC4)n4ncnc34)nc12] | 1H NMR (400 MHz, DMSO-d6) δ = 8.56 (s, 1H), 7.98 (br s, 1H), 7.46 (br s, 1H), 7.23-7.23 (m, 1H), 7.24 (d, J = 7.4 Hz, 1H), 6.02-5.72 (m, 4H), 4.71 (br s, 1H), 4.23-3.89 (m, 4H), 3.80-3.53 (m, 7H), 3.48- 3.44 (m, 2H), 2.01-1.87 (m, 1H), 1.75-1.39 (m, 3H), 1.36-1.02 (m, 2H), 0.72 (brs, 3H). LC-MS | S18 |
| | | (ES): m/z = 481.3 [M + H]+, | |
| | | RT (min) = 1.03 | |
| 173 | 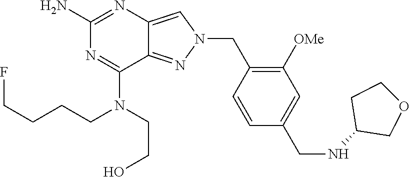 [SMILES: COc1cc(CN[C@@H]2CCOC2)ccc1Cn1cc2nc(N)nc(N(CCO)CCCCF)c2n1] | 1H NMR (400 MHz, DMSO-d6) δ = 7.80 (br d, J = 2.9 Hz, 1H), 7.07 (s, 1H), 7.03-6.94 (m, 1H), 6.90 (br d, J = 7.3 Hz, 1H), 6.25 (br s, 1H), 5.41 (s, 2H), 4.76- 4.74 (m, 1H), 4.81-4.60 (m, 1H), 4.55-4.30 (m, 2H), 4.16 (br s, 2H), 3.83 (s, 3H), 3.79-3.60 (m, 10H), 3.50-3.46 (m, 1H), 2.00- 1.92 (m, 1H), 1.77-1.49 | S1 |
| | | (m, 6H). LC-MS (ES): | |
| | | m/z = 488.3 [M + H]+, RT | |
| | | (min) = 0.728 | |
| 174 | 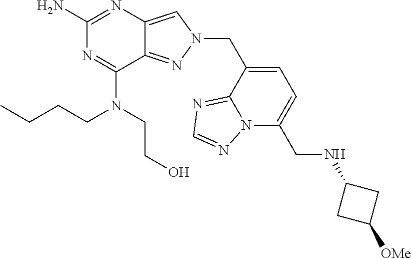 [SMILES: CCCCN(CCO)c1nc(N)nc2cn(Cc3ccc(CN[C@H]4C[C@H](OC)C4)n4ncnc34)nc12] | 1H NMR (400 MHz, DMSO-d6) δ = 8.55 (s, 1H), 7.94 (br s, 1H), 7.43 (br d, J = 2.3 Hz, 1H), 7.20 (d, J = 7.4 Hz, 1H), 5.82 (s, 2H), 5.70 (br s, 2H),4.74 (br s, 1H), 4.21-3.99 (m, 4H), 3.96-3.89 (m, 1H), 3.63 (br s, 5H), 3.09 (s, 3H), 2.02- 1.89 (m, 5H), 1.67-1.22 (m, 3H), 1.19-0.99 (m, 1H), 0.73 (br d, J = 2.5 Hz, 3H). LC-MS (ES): m/z = 495.2[M + H]+, RT (min) = 1.14 | S18 |
| 176 | 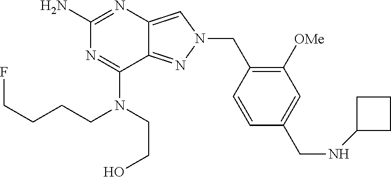 [SMILES: COc1cc(CNC2CCC2)ccc1Cn1cc2nc(N)nc(N(CCO)CCCCF)c2n1] | ¹H NMR (400 MHz, DMSO-d₆) δ = 7.73 (br s, 1H), 7.06 (s, 1H), 6.97 (br s, 1H), 6.92-6.80 (m, 1H), 5.77 (br s, 2H), 5.40 (s, 2H), 4.84-4.29 (m, 3H), 4.22- 3.97 (m, 2H), 3.83 (s, 3H), 3.73-3.64 (m, 5H), 2.13- 2.00 (m, 2H), 1.86-1.48 (m, 9H). LC-MS (ES): m/z = 472.3 [M + H]⁺, RT (min) = 0.819 | S1 |
| 177 | 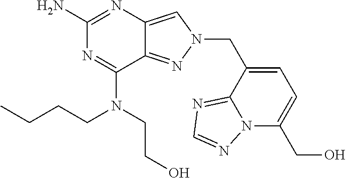 [SMILES: CCCCN(CCO)c1nc(N)nc2cn(Cc3ccc(CO)n4ncnc34)nc12] | 1H NMR (400 MHz, DMSO-d6) δ = 8.55 (s, 1H), 8.04 (br s, 1H), 7.52 (br d, J = 5.5 Hz, 1H), 7.23 (d, J = 7.3 Hz, 1H), 6.30 (br s, 1H), 5.91-5.80 (m, 3H), 4.94 (d, J = 5.5 Hz, 2H), 4.73 (br s, 1H), 4.16-3.96 (m, 2H), 3.80-3.53 (m, 4H), 1.68- 1.26 (m, 2H), 1.19-1.04 (m, 1H), 0.91 (br d, J = 4.8 Hz, 1H), 0.71 (br s, 3H). | S18 |
| | | LC-MS (ES): m/z = | |
| | | 412.1[M + H]+, RT (min) = | |
| | | 0.951 | |
| 178 | 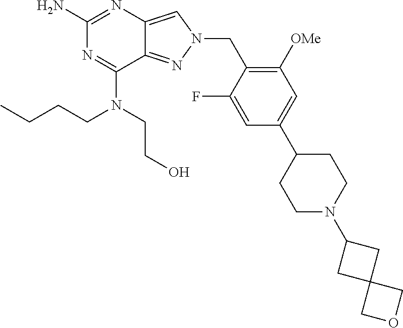 [SMILES: CCCCN(CCO)c1nc(N)nc2cn(Cc3c(F)cc(C4CCN(C5CC6(COC6)C5)CC4)cc3OC)nc12] | δ 7.72-7.49 (m, 1H), 6.78 (br s, 1H), 6.71 (br d, J = 10.1 Hz, 1H), 5.37 (s, 2H), 4.58 (s, 2H), 4.44 (s, 2H), 4.03 (br d, J = 7.6 Hz, 1H), 3.83 (br s, 3H), 3.73- 3.48 (m, 2H), 2.85 (br d, J = 11.0 Hz, 2H), 2.50-2.44 (m, 2H), 2.36-2.24 (m, 2H), 1.95-1.91 (m, 2H), 1.82-1.65 (m, 4H), 1.65- 1.43 (m, 4H), 1.38-1.15 (m, 2H), 0.98-0.73 (m, 3H). Six protons were not visible due to water supression and the overlap with DMSO-d6 peak. LC- MS (ES, m/z): [M + H]+ = 568.4, r.t. 1.45 mins | S9 |
| 180 | 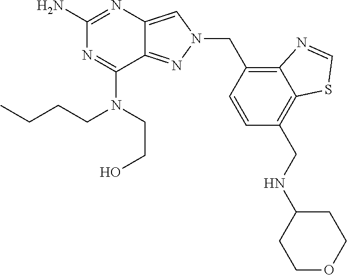 [SMILES: CCCCN(CCO)c1nc(N)nc2cn(Cc3ccc(CNC4CCOCC4)c4scnc34)nc12] | ¹H NMR (400 MHz, DMSO-d₆) δ 9.37-9.26 (m, 1H), 8.42-8.28 (m, 1H), 7.90-7.74 (m, 1H), 7.42- 7.16 (m, 2H), 5.94-5.81 (m, 2H), 3.79-3.46 (m, 5H), 3.27-3.10 (m, 2H), 1.84-1.68 (m, 2H), 1.61- 1.33 (m, 2H), 1.32-1.14 (m, 3H), 1.14-0.96 (m, 2H), 0.90-0.78 (m, 1H), 0.74-0.53 (m, 3H). LC-MS (ES): m/z = 511.2 [M + H]+, RT (min) = 0.77 | S11 |
| 182 | 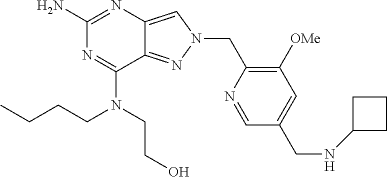 [SMILES: CCCCN(CCO)c1nc(N)nc2cn(Cc3ncc(CNC4CCC4)cc3OC)nc12] | 1H NMR (400 MHz, DMSO-d6) δ = 8.02 (d, J = 1.1 Hz, 1H), 7.82 (br s, 1H), 7.48 (s, 1H), 6.26 (br s, 2H), 5.54 (s, 2H), 4.76 (br s, 1H), 4.07 (br s, 2H), 3.86 (br s, 3H), 3.69 (s, 7H), 3.21 (br d, J = 7.0 Hz, 3H), 2.13- 1.99 (m, 2H), 1.82-1.70 (m, 2H), 1.66-1.43 (m, 4H), 1.24 (s, 2H), 0.82 (br s, 3H). LC-MS (ES): m/z = | S1 |
| | | 455.3 [M + H]+, RT (min) = | |
| | | 0.797 | |
| 186 | 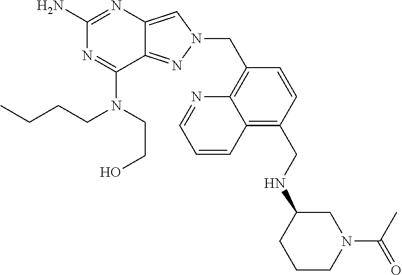 [SMILES: CCCCN(CCO)c1nc(N)nc2cn(Cc3ccc(CN[C@@H]4CCCN(C(C)=O)C4)c4cccnc34)nc12] | 1H NMR (400 MHz, DMSO-d6) δ = 9.05-8.94 (m, 1H), 8.77-8.66 (m, 1H), 7.96-7.81 (m, 1H), 7.66-7.46 (m, 2H), 7.39- 7.18 (m, 1H), 6.56-6.44 (m, 1H), 6.17-6.01 (m, 2H), 5.99-5.75 (m, 1H), 4.80-4.59 (m, 1H), 4.27- 4.01 (m, 4H), 3.98-3.85 (m, 1H), 3.80-3.50 (m, 6H), 3.21-3.15 (m, 1H), 3.12-3.01 (m, 2H), 2.37- 2.29 (m, 1H), 1.95-1.85 (m, 3H), 1.74-1.45 (m, 3H), 1.41-1.07 (m, 5H), 0.96-0.66 (m, 3H); LC-MS (ES): m/z = 546.3 [M + H]+, RT (min) = 1.649 | S10 |
| 188 | 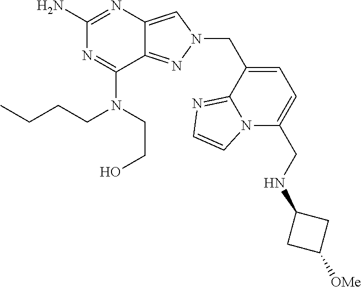 [SMILES: CCCCN(CCO)c1nc(N)nc2cn(Cc3ccc(CN[C@H]4C[C@H](OC)C4)n4ccnc34)nc12] | 1H NMR (400 MHz, DMSO-d6) δ = 11.83- 11.66 (m, 1H), 9.44-9.28 (m, 1H), 8.30-8.16 (m, 1H), 7.81-7.57 (m, 2H), 7.23-7.12 (m, 1H), 7.11- 7.04 (m, 1H), 6.98-6.94 (m, 1H), 5.94-5.79 (m, 1H), 4.86-4.76 (m, 1H), 4.61-4.39 (m, 2H), 4.24- 4.18 (m, 1H), 4.15-4.09 (m, 1H), 4.05-3.90 (m, 2H), 3.84-3.76 (m, 2H), 3.72-3.58 (m, 2H), 3.18- 3.11 (m, 3H), 2.30-2.16 (m, 2H), 1.69-1.44 (m, 2H), 1.41-1.29 (m, 1H), 1.27-1.07 (m, 3H), 0.87- 0.76 (m, 3H).; LC-MS (ES): | S17 |
| | | m/z = 494.2 [M + H]+, RT | |
| | | (min) = 1.477 | |
| 190 | 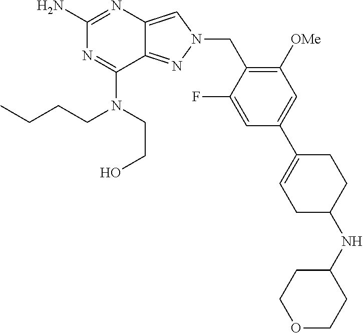 [SMILES: CCCCN(CCO)c1nc(N)nc2cn(Cc3c(F)cc(C4=CCC(NC5CCOCC5)CC4)cc3OC)nc12] | δ 7.74-7.52 (m, 1H), 7.01- 6.75 (m, 2H), 6.22 (br d, J = 1.2 Hz, 1H), 5.54 (br s, 2H), 5.40 (s, 2H), 4.02 (br d, J = 2.4 Hz, 1H), 3.94- 3.76 (m, 4H), 3.75-3.47 (m, 2H), 3.31 (br t, J = 11.4 Hz, 1H), 3.05-2.93 (m, 1H), 2.90 (br s, 1H), 2.49- 2.34 (m, 3H), 2.06-1.94 (m, 2H), 1.79 (br d, J = 11.9 Hz, 2H), 1.54-1.37 (m, 2H), 1.36-1.07 (m, 4H), 0.98-0.74 (m, 3H). Eight protons were not visible due to water supression and the overlap with DMSO-d6 peak. LC-MS (ES, m/z): [M + H]+ = 568.3, r.t. 1.34 mins | S9 |
| 191 | 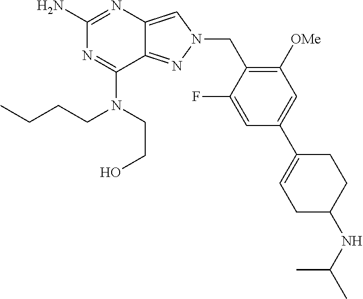 | δ 7.74-7.59 (m, 1H), 7.59- 7.45 (m, 1H), 6.78 (br s, 1H), 6.72 (br d, J = 9.9 Hz, 1H), 5.40 (br s, 2H), 5.37 (s, 2H), 4.14-3.94 (m, 2H), 3.84 (br s, 3H), 3.72-3.60 (m, 2H), 2.86 (br d, J = 11.3 Hz, 2H), 2.77-2.61 (m, 1H), 2.49-2.41 (m, 1H), 2.18 (br t, J = 11.4 Hz, 2H), 1.78-1.67 (m, 2H), 1.67- 1.35 (m, 4H), 1.36-1.14 (m, 2H), 0.98 (br d, J = 6.6 Hz, 6H), 0.84 (br dd, J = 8.5, 3.7 Hz, 3H). Two protons were not visible due to water supression. LC-MS (ES, m/z): [M + H]+ = 514.4, r.t. 1.26 mins | S9 |
| 192 | 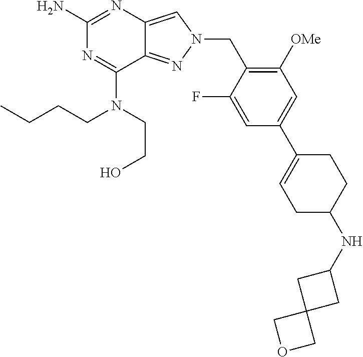 [SMILES: CCCCN(CCO)c1nc(N)nc2cn(Cc3c(F)cc(C4=CCC(NC5CC6(COC6)C5)CC4)cc3OC)nc12] | δ 7.75-7.57 (m, 1H), 6.96- 6.83 (m, 2H), 6.29-6.15 (m, 1H), 5.51 (br s, 2H), 5.43-5.36 (m, 2H), 4.57 (s, 2H), 4.45 (s, 2H), 4.08- 3.95 (m, 1H), 3.85 (br s, 3H), 3.74-3.44 (m, 2H), 3.18 (br t, J = 7.5 Hz, 1H), 2.76-2.62 (m, 1H), 2.49- 2.33 (m, 4H), 1.67-1.35 (m, 4H), 1.35-1.12 (m, 3H), 0.97-0.70 (m, 3H). Eight protons were not visible due to water supression and the overlap with DMSO-d6 peak. LC- MS (ES, m/z): [M + H]+ = 580.6, r.t. 1.34 mins | S9 |
| 193 | 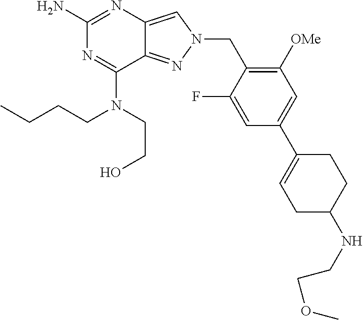 [SMILES: CCCCN(CCO)c1nc(N)nc2cn(Cc3c(F)cc(C4=CCC(NCCOC)CC4)cc3OC)nc12] | δ 7.75-7.51 (m, 1H), 6.93- 6.79 (m, 2H), 6.22 (br s, 1H), 5.49 (br s, 2H), 5.39 (s, 2H), 4.14-3.95 (m, 1H), 3.86 (br s, 3H), 3.77-3.49 (m, 2H), 3.26 (s, 3H), 2.84- 2.65 (m, 3H), 2.49-2.29 (m, 3H), 2.02-1.92 (m, 2H), 1.64-1.38 (m, 3H), 1.35-1.12 (m, 2H), 0.98- 0.74 (m, 3H). Seven protons were not visible due to water supression and the overlap with DMSO-d6 peak. LC-MS (ES, m/z): [M + H]+ = 542.3, r.t. 1.3 mins | S9 |
| 194 | 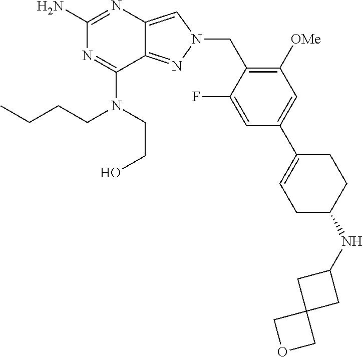 [SMILES: CCCCN(CCO)c1nc(N)nc2cn(Cc3c(F)cc(C4=CC[C@@H](NC5CC6(COC6)C5)CC4)cc3OC)nc12] | δ 7.75-7.57 (m, 1H), 6.96- 6.83 (m, 2H), 6.29-6.15 (m, 1H), 5.51 (br s, 2H), 5.43-5.36 (m, 2H), 4.57 (s, 2H), 4.45 (s, 2H), 4.08- 3.95 (m, 1H), 3.85 (br s, 3H), 3.74-3.44 (m, 2H), 3.18 (br t, J = 7.5 Hz, 1H), 2.76-2.62 (m, 1H), 2.49- 2.33 (m, 4H), 1.67-1.35 (m, 4H), 1.35-1.12 (m, 3H), 0.97-0.70 (m, 3H). Eight protons were not visible due to water supression and the overlap with DMSO-d6 peak. LC- MS (ES, m/z): [M + H]+ = 580.6, r.t. 1.34 mins | S9 |
| 195 | 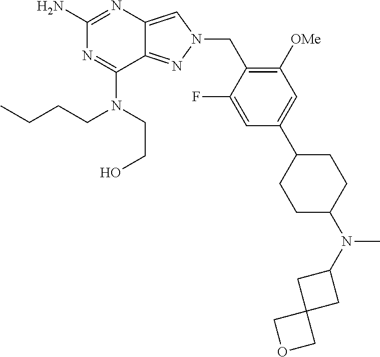 [SMILES: CCCCN(CCO)c1nc(N)nc2cn(Cc3c(F)cc(C4CCC(N(C)C5CC6(COC6)C5)CC4)cc3OC)nc12] | δ 7.65 (s, 1H), 6.94-6.43 (m, 2H), 5.59-5.21 (m, 5H), 4.58 (d, J = 6.7 Hz, 2H), 4.43 (d, J = 5.0 Hz, 2H), 4.03 (s, 2H), 3.84 (s, 3H), 3.62 (s, 2H), 3.00 (s, 1H), 2.91 (dt, J = 23.0, 8.2 Hz, 1H), 2.77 (s, 1H), 2.55 (s, 3H), 2.29 (d, J = 19.6 Hz, 3H), 2.00 (s, 2H), 1.93 (d, J = 21.5 Hz, 4H), 1.81 (d, J = 12.4 Hz, 1H), 1.64 (dd, J = 29.3, 13.5 Hz, 3H), 1.54-1.40 (m, 3H), 1.22 (d, J = 17.2 Hz, 2H), 0.84 (s, 3H). LC-MS (ES, m/z): [M + H]+ = 596.4, r.t. 1.29 mins | S9 |
| 196 | 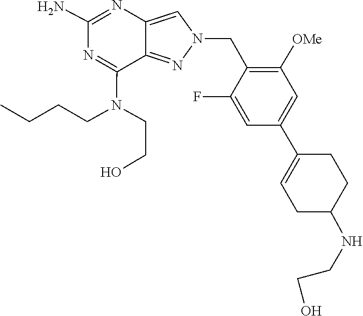 | δ 7.61 (d, J = 50.8 Hz, 1H), 6.87 (d, J = 19.2 Hz, 3H), 6.22 (s, 1H), 5.49 (s, 1H), 4.02 (s, 1H), 3.86 (s, 4H), 3.62 (s, 3H), 3.26 (s, 2H), 2.76 (dq, J = 12.5, 6.7, 6.2 Hz, 4H), 2.55 (s, 3H), 2.42 (d, J = 24.3 Hz, 4H), 1.96 (s, 3H), 1.47 (s, 3H), 1.20 (s, 2H), 0.87 (d, J = 38.1 Hz, 3H). 2 protons were not visible due to water supression and the overlap with DMSO-d6 peak. LC- MS (ES, m/z): [M + H]+ = 542.3, r.t. 1.3 mins | S9 |
| 197 | 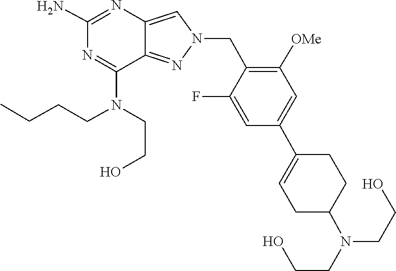 [SMILES: CCCCN(CCO)c1nc(N)nc2cn(Cc3c(F)cc(C4=CCC(N(CCO)CCO)CC4)cc3OC)nc12] | δ 8.28 (s, 1H), 8.17 (s, 0H), 7.24-6.77 (m, 2H), 6.23 (s, 1H), 5.47 (d, J = 6.4 Hz, 2H), 4.16 (s, 1H), 4.07 (d, J = 6.4 Hz, 4H), 3.90 (s, 1H), 3.87 (s, 1H), 3.84 (s, 2H), 3.77 (d, J = 6.2 Hz, 1H), 3.75-3.67 (m, 2H), 2.78- 2.61 (m, 3H), 2.55 (s, 4H), 2.46 (d, J = 18.4 Hz, 1H), 1.96 (s, 2H), 1.76 (s, 5H), 1.62 (d, J = 7.8 Hz, 1H), 1.49 (d, J = 8.1 Hz, 1H), 1.32 (q, J = 7.4 Hz, 1H), 1.26-1.07 (m, 2H), 0.92 (t, J = 7.4 Hz, 1H), 0.81 (t, J = 7.3 Hz, 3H). LC-MS (ES, | S9 |
| | | m/z): [M + H]+ = 572.1, r.t. | |
| | | 1.18 mins | |
| 198 | 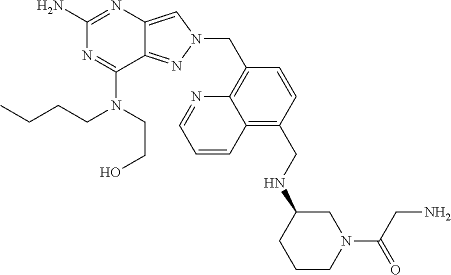 [SMILES: CCCCN(CCO)c1nc(N)nc2cn(Cc3ccc(CN[C@@H]4CCCN(C(=O)CN)C4)c4cccnc34)nc12] | 1H NMR (400 MHz, DMSO-d6) δ = 9.04-8.89 (m, 1H), 8.70 (dd, J = 1.4, 8.4 Hz, 1H), 7.83 (br s, 1H), 7.61 (dd, J = 4.3, 8.6 Hz, 1H), 7.54 (d, J = 7.5 Hz, 1H), 7.27 (br s, 1H), 6.06 (s, 2H), 5.41 (s, 2H), 4.22- 3.95 (m, 4H), 3.62 (br s, 5H), 2.96-2.74 (m, 2H), 2.67-2.57 (m, 1H), 2.08 (s, 1H), 1.74 (s, 5H), 1.58 (br s, 3H), 1.24 (s, 3H), 0.80 (br s, 3H); LC-MS (ES): m/z = 561.4 [M + H]+, RT (min) = 0.728 | S10 |
| 199 | 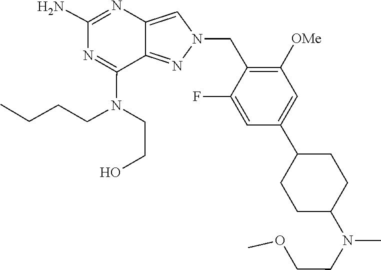 [SMILES: CCCCN(CCO)c1nc(N)nc2cn(Cc3c(F)cc(C4CCC(N(C)CCOC)CC4)cc3OC)nc12] | δ 8.21 (d, J = 54.5 Hz, 1H), 7.13-6.48 (m, 2H), 5.45 (d, J = 6.7 Hz, 2H), 4.52- 4.00 (m, 4H), 3.93-3.55 (m, 9H), 3.22 (d, J = 10.1 Hz, 2H), 2.66 (s, 1H), 2.28 (d, J = 11.8 Hz, 2H), 2.18 (s, 1H), 1.75 (d, J = 20.5 Hz, 8H), 1.63 (s, 1H), 1.48 (dd, J = 25.8, 12.5 Hz, 5H), 1.40-1.27 (m, 1H), 1.27- 1.13 (m, 1H), 0.92 (t, J = 7.3 Hz, 1H), 0.83 (t, J = 7.4 Hz, 3H). LC-MS (ES, m/z): [M + H]+ = 558.4, r.t. 1.45 mins | S9 |
| 201 | 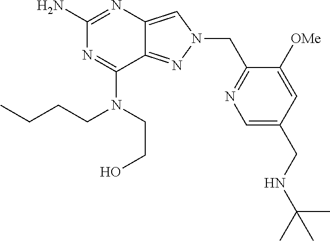 [SMILES: CCCCN(CCO)c1nc(N)nc2cn(Cc3ncc(CNC(C)(C)C)cc3OC)nc12] | 1H NMR (400 MHz, DMSO-d6) δ = 8.88 (br s, 1H), 8.53 (br s, 1H), 8.09 (s, 1H), 7.73 (br s, 1H), 7.55 (s, 1H), 5.79 (br s, 2H), 5.53 (s, 2H), 4.71 (s, 1H), 4.06 (br s, 2H), 3.86 (br s, 6H), 3.62 (br dd, J = 2.3, 3.5 Hz, 4H), 2.10 (s, 2H), 1.66-1.42 (m, 3H), 1.18 (s, 13H), 0.84 (br s, 3H). LC-MS (ES): m/z = 457.3 [M + H]+, RT (min) = 0.803 | S1 |
| 203 | 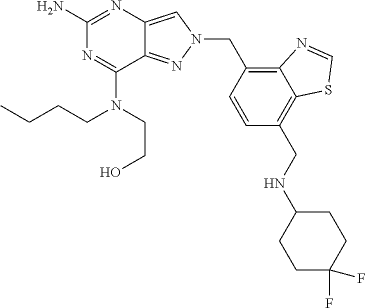 [SMILES: CCCCN(CCO)c1nc(N)nc2cn(Cc3ccc(CNC4CCC(F)(F)CC4)c4scnc34)nc12] | 1H NMR (400 MHz, DMSO-d6) δ = 9.42 (s, 1H), 7.83 (br s, 1H), 7.37 (d, J = 7.5 Hz, 1H), 7.22 (br s, 1H), 5.95 (s, 2H), 5.42 (br s, 3H), 4.00 (s, 5H), 3.63 (br d, J = 3.5 Hz, 6H), 2.63-2.56 (m, 1H), 2.11-1.93 (m, 6H), 1.89-1.68 (m, 10H), 1.64- 1.41 (m, 4H), 1.24 (s, 3H), 0.95-0.64 (m, 4H); LC-MS (ES): m/z = 545.2 [M + H]+, RT (min) = 1.32 | S11 |
| 204 | 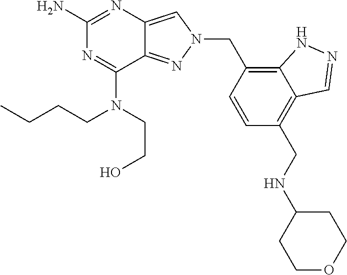 [SMILES: CCCCN(CCO)c1nc(N)nc2cn(Cc3ccc(CNC4CCOCC4)c4cn[nH]c34)nc12] | 1H NMR (400 MHz, DMSO-d6) δ = 13.25 (br s, 1H), 8.28 (s, 1H), 7.92 (br s, 1H), 7.12-6.97 (m, 2H), 5.72 (s, 2H), 5.45 (br s, 2H), 4.69 (s, 1H), 4.04 (s, 4H), 3.85-3.77 (m, 2H), 3.60 (br s, 4H), 3.22 (br d, J = 2.0 Hz, 2H), 2.59 (br d, J = 4.3 Hz, 1H), 1.78 (br d, J = 12.0 Hz, 2H), 1.49 (br s, 2H), 1.33-1.08 (m, 4H), 0.79 (br s, 3H); LC-MS (ES): m/z = 494.2 [M + H]+, RT (min) = 1.023 | S11 |
| 205 | 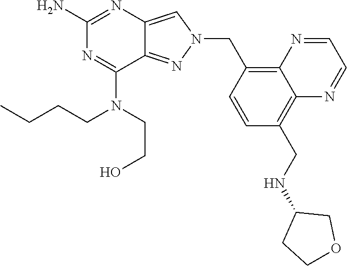 [SMILES: CCCCN(CCO)c1nc(N)nc2cn(Cc3ccc(CN[C@H]4CCOC4)c4nccnc34)nc12] | δ 9.03 (br d, J = 6.4 Hz, 2H), 7.92-7.78 (m, 2H), 7.64- 7.42 (m, 1H), 6.07 (s, 2H), 5.50 (s, 1H), 4.34-4.21 (m, 2H), 4.18-3.94 (m, 1H), 3.90 (s, 1H), 3.80-3.59 (m, 3H), 3.50-3.42 (m, 1H), 3.31 (br s, 1H), 1.93 (br dd, J = 12.4, 7.4 Hz, 1H), 1.70 (br d, J = 5.0 Hz, 1H), 1.63- 1.21 (m, 4H), 1.04 (br d, J = 6.2 Hz, 2H), 0.98-0.59 (m, 4H), 4 protons were not observed due to water suppression. LC-MS (ES, m/z): [M + H]+ = 492.2, 1.28 mins | S10 |
| 206 | 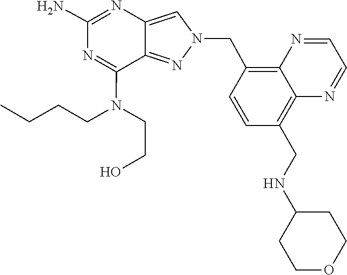 [SMILES: CCCCN(CCO)c1nc(N)nc2cn(Cc3ccc(CNC4CCOCC4)c4nccnc34)nc12] | δ 9.02 (br d, J = 7.7 Hz, 2H), 7.87 (br d, J = 7.7 Hz, 2H), 7.52 (br s, 1H), 6.07 (s, 2H), 5.52 (s, 1H), 4.33 (s, 2H), 4.02 (br s, 2H), 3.81 (br d, J = 11.5 Hz, 2H), 3.62 (br s, 2H), 3.23 (br t, J = 10.7 Hz, 2H), 2.63 (br d, J = 10.2 Hz, 1H), 1.78 (br d, J = 13.7 Hz, 2H), 1.43 (br s, 1H), 1.38- 1.21 (m, 3H), 1.09 (br s, 1H), 0.99-0.86 (m, 1H), 0.72 (br s, 2H), six protons were not observed due to water suppression. LC-MS (ES, m/z): [M + H]+ = 506.5, 1.24 mins | S10 |
| 207 | 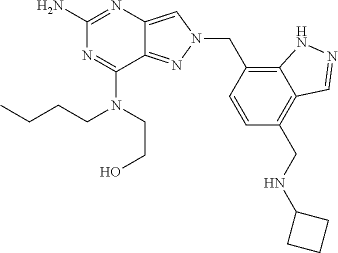 [SMILES: CCCCN(CCO)c1nc(N)nc2cn(Cc3ccc(CNC4CCC4)c4cn[nH]c34)nc12] | 1H NMR (400 MHz, DMSO-d6) δ = 13.25 (br s, 1H), 8.24 (s, 1H), 7.92 (br d, J = 1.5 Hz, 1H), 7.10- 7.05 (m, 1H), 7.03-6.96 (m, 1H), 5.72 (s, 2H), 5.43 (s, 2H), 4.67 (s, 1H), 4.08 (br s, 2H), 3.91 (s, 2H), 3.61 (br s, 4H), 3.17 (br d, J = 2.3 Hz, 1H), 2.07-1.99 (m, 2H), 1.72-1.44 (m, 6H), 1.24 (s, 2H), 0.99-0.76 (m, 3H); LC-MS (ES): m/z = 464.3 [M + H]+, RT (min) = 0.89 | S11 |
| 208 | 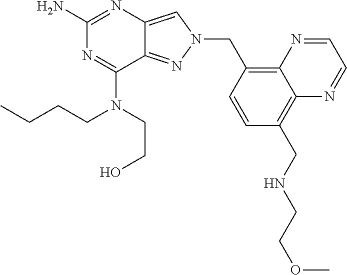 [SMILES: CCCCN(CCO)c1nc(N)nc2cn(Cc3ccc(CNCCOC)c4nccnc34)nc12] | 1H NMR (500 MHz, DMSO-d6) δ 9.18-9.09 (m, 2H), 8.33-8.15 (m, 1H), 8.09-7.99 (m, 1H), 7.89- 7.76 (m, 1H), 7.75-7.62 (m, 1H), 6.23-6.16 (m, 2H), 4.77 (s, 2H), 4.20- 4.12 (m, 1H), 4.11-4.01 (m, 1H), 3.86-3.74 (m, 2H), 3.72-3.66 (m, 1H), 3.65-3.54 (m, 2H), 3.30 (s, 1H), 3.25-3.15 (m, 1H), 1.71-1.57 (m, 1H), 1.54- 1.43 (m, 1H), 1.39-1.27 (m, 1H), 1.14-1.01 (m, 1H), 0.96-0.87 (m, 1H), 0.75-0.66 (m, 2H), 7 protons were not observed | S10 |
| | | due to water suppression. | |
| | | LC-MS (ES, m/z): | |
| | | [M + H]+ = 480.2, r.t. | |
| | | 1.3 mins | |
| 209 | 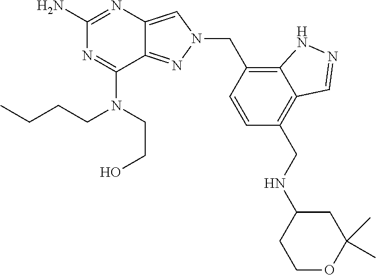 [SMILES: CCCCN(CCO)c1nc(N)nc2cn(Cc3ccc(CNC4CCOC(C)(C)C4)c4cn[nH]c34)nc12] | 1H NMR (400 MHz, DMSO-d6) δ = 13.29 (br s, 1H), 8.29 (s, 1H), 7.96 (br s, 1H), 7.16-6.93 (m, 2H), 5.74 (s, 4H), 4.68 (s, 1H), 4.09 (s, 4H), 3.68-3.60 (m, 4H), 3.50 (br d, J = 1.8 Hz, 2H), 2.88 (br s, 1H), 1.87- 1.75 (m, 2H), 1.62-1.35 (m, 2H), 1.30-1.09 (m, 7H), 1.07 (s, 3H), 0.99- 0.77 (m, 3H); LC-MS (ES): m/z = 522.3 [M + H]+, RT (min) = 1.41 | S11 |
| 210 | 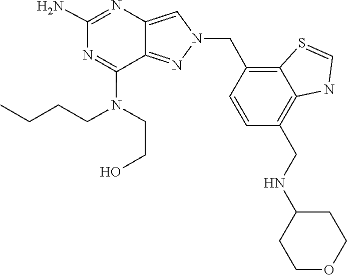 | 1H NMR (400 MHz, DMSO-d6) δ = 9.37 (s, 1H), 7.87 (br s, 1H), 7.60 (d, J = 7.5 Hz, 1H), 7.51-7.40 (m, 1H), 5.74 (s, 2H), 5.55 (br s, 2H), 4.26 (s, 2H), 4.04 (br s, 2H), 3.85-3.78 (m, 2H), 3.67-3.60 (m, 4H), 3.25- 3.22 (m, 4H), 2.65-2.58 (m, 1H), 1.84-1.74 (m, 2H), 1.65-1.39 (m, 2H), 1.36-1.23 (m, 3H), 1.17-0.61 (m, 3H). LC-MS [M + H] 511.2, RT (min) = 1.103 min | S11 |
| 211 | 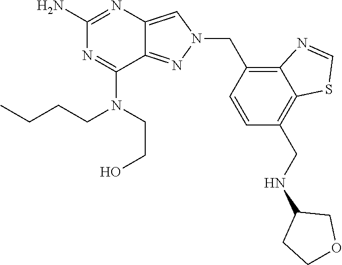 | 1H NMR (400 MHz, DMSO-d6) δ = 9.40-9.26 (m, 2H), 8.27-8.20 (m, 1H), 7.77-7.70 (m, 1H), 7.68-7.61 (m, 1H), 7.52- 7.40 (m, 1H), 6.10-6.04 (m, 2H), 4.86-4.74 (m, 2H), 4.56-4.41 (m, 2H), 4.22-4.16 (m, 1H), 4.14- 4.05 (m, 2H), 4.01-3.85 (m, 3H), 3.84-3.76 (m, 2H), 3.72-3.55 (m, 3H), 2.36-2.20 (m, 2H), 2.12- 1.97 (m, 1H), 1.70-1.46 (m, 1H), 1.36-1.09 (m, 2H), 0.98-0.88 (m, 2H), 0.86-0.73 (m, 3H); LC-MS | S11 |
| | | (ES): m/z = 497.2 [M + H]+, | |
| | | RT (min) = 1.29 | |

### BIOLOGICAL EXAMPLES

The biological activity of compounds disclosed herein as TLR7 agonists can be assayed by the procedures following.

### Example B1. Human TLR7 Agonist Activity Assay

This procedure describes a method for assaying human TLR7 (hTLR7) and human TLR8 (hTLR8) agonist activity of the compounds disclosed herein.

Engineered human embryonic kidney blue cells (HEK-Blue™ TLR cells; Invivogen) possessing a human TLR7-secreted embryonic alkaline phosphatase (SEAP) reporter transgene were suspended in a non-selective, culture medium (DMEM high-glucose (Invitrogen), supplemented with 1000 fetal bovine serum (Sigma)). HEK-Blue™ TLR7 cells were added to each well of a 384-well tissue-culture plate (15,000 cells per well) and incubated 16-18 h at 37° C., 5% CO₂. Compounds (100 nL) were dispensed into wells containing the HEK-Blue™ TLR cells and the treated cells were incubated at 37° C., 5% CO₂. After 18 h, ten microliters 5 of freshly prepared Quanti-Blue™ reagent (Invivogen) was added to each well and incubated for 30 min (37° C., 50% CO₂). SLAP levels were measured using an Envision plate reader (OD=620 nm). The half maximal effective concentration values (EC_{SO}; compound concentration which induced a response halfway between the assay baseline and maximum) were calculated. Some of the reported activities were the average of multiple measurements.

TLR8 activity was measured in a similar manner.

TABLE 2

| Compound No. | TLR7 Agonist EC50 (nM) | TLR8 Agonist EC50 (nM) |
|---|---|---|
| 1 | 1,291 | 1,143 |
| 2 | 1,337 | 1,007 |
| 3 | 970 | 596 |
| 4 | 694 | 633 |
| 5 | 643 | 700 |
| 6 | 680 | 892 |
| 7 | 969 | 602 |
| 8 | 969 | 602 |
| 9 | 1,615 | 1,251 |
| 10 | 1,329 | 1,446 |
| 11 | 732 | 727 |
| 12 | 661 | 477 |
| 13 | 733 | 1,125 |
| 15 | 688 | 2,513 |
| 16 | 749 | 2,500 |
| 17 | 1,148 | 745 |
| 18 | 380 | 447 |
| 19 | 696 | 1,466 |
| 20 | 1,183 | 705 |
| 21 | 587 | 834 |
| 22 | 614 | 547 |
| 23 | 1,910 | 1,897 |
| 24 | 433 | 509 |
| 25 | 430 | 360 |
| 26 | 828 | 1,145 |
| 27 | 1,966 | 1,953 |
| 28 | 284 | 266 |
| 29 | 2,500 | 2,498 |
| 30 | 1,415 | 1,620 |
| 31 | 471 | 499 |
| 32 | 2,500 | 1,199 |
| 33 | 548 | 524 |
| 34 | 66 | 357 |
| 35 | 551 | 1,846 |
| 36 | 144 | 525 |
| 37 | 176 | 516 |
| 38 | 91 | 287 |
| 39 | 396 | 437 |
| 40 | 392 | 801 |
| 41 | 251 | 858 |
| 42 | 101 | 496 |
| 43 | 5,000 | 1,742 |
| 44 | 825 | 705 |
| 45 | 280 | 205 |
| 46 | 326 | 360 |
| 47 | 186 | 538 |
| 48 | 182 | 227 |
| 49 | 385 | 682 |
| 49-1 | 374 | 949 |
| 49-2 | 385 | 682 |
| 50 | 374 | 949 |
| 52 | 1,191 | 1,074 |
| 53 | 731 | 486 |
| 54 | 1,331 | 1,503 |
| 55 | 349 | 2,032 |
| 56 | 1,699 | 2,313 |
| 57 | 1,998 | 1,389 |
| 58 | 757 | 488 |
| 59 | 1,550 | 1,142 |
| 60 | 326 | 469 |
| 61 | 1,502 | 952 |
| 62 | 1,311 | 675 |
| 63 | 915 | 736 |
| 64 | 2,115 | 1,555 |
| 65 | 2,092 | 2,380 |
| 66 | 1,592 | 2,132 |
| 67 | 5,000 | 2,161 |
| 68 | 349 | 1,324 |
| 70 | 1,046 | 1,065 |
| 71 | 621 | 1,876 |
| 72 | 738 | 835 |
| 73 | 320 | 593 |
| 74 | 741 | 1,283 |
| 75 | 370 | 670 |
| 80 | 1,413 | 1,621 |
| 81 | 1,000 | 1,427 |
| 82 | 323 | 567 |
| 83 | 511 | 2,265 |
| 84 | 199 | 497 |
| 85 | 148 | 1,971 |
| 86 | 229 | 311 |
| 87 | 5,000 | 1,819 |
| 88 | 805 | 535 |
| 89 | 1,549 | 961 |
| 90 | 2,145 | 2,411 |
| 91 | 1,222 | 2,083 |
| 92 | 1,327 | 2,106 |
| 93 | 2,585 | 1,913 |
| 94 | 1,500 | 2,661 |
| 95 | 1,239 | 2,500 |
| 96 | 540 | 1,986 |
| 97 | 1,047 | 1,046 |
| 98 | 1,560 | 1,153 |
| 99 | 1,912 | 1,580 |
| 100 | 1,760 | 1,421 |
| 101 | 1,602 | 1,574 |
| 102 | 2,195 | 1,799 |
| 103 | 322 | 202 |
| 104 | 1,749 | 2,164 |
| 105 | 1,823 | 2,776 |
| 106 | 1,142 | 2,500 |
| 107 | 1,279 | 3,035 |
| 108 | 1,156 | 1,877 |
| 109 | 1,458 | 1,986 |
| 110 | 39 | 702 |
| 111 | 654 | 453 |
| 112 | 585 | 562 |
| 113 | 937 | 2,960 |
| 114 | 1,056 | 1,830 |
| 115 | 108 | 1,501 |
| 116 | 31 | 1,023 |
| 117 | 1,187 | 1,973 |
| 118 | 1,577 | 2,500 |
| 119 | 136 | 1,965 |
| 120 | 1,776 | 1,811 |
| 121 | 1,365 | 564 |
| 122 | 561 | 189 |
| 123 | 778 | 2,003 |
| 124 | 5,000 | 1,870 |
| 125 | 2,252 | 2,034 |
| 126 | 5,000 | 689 |
| 127 | 1,801 | 2,417 |
| 128 | 1,190 | 628 |
| 129 | 5,000 | 2,709 |
| 130 | 1,904 | 1,879 |
| 131 | 1,281 | 3,328 |
| 132 | 389 | 1,860 |
| 133 | 513 | 2,104 |
| 134 | 2,238 | 1,855 |
| 135 | 1,144 | 491 |
| 136 | 861 | 360 |
| 137 | 1,052 | 400 |
| 138 | 1,408 | 608 |
| 139 | 1,218 | 542 |
| 140 | 993 | 674 |
| 141 | 439 | 421 |
| 142 | 1,629 | 915 |
| 143 | 1,696 | 1,865 |
| 144 | 1,867 | 2,063 |
| 145 | 1,438 | 2,017 |
| 146 | 1,825 | 1,773 |
| 147 | 1,814 | 2,024 |
| 148 | 1,838 | 462 |
| 149 | 561 | 1,810 |
| 150 | 1,125 | 2,098 |
| 151 | 612 | 1,835 |
| 152 | 749 | 1,835 |
| 153 | 615 | 306 |
| 154 | 2,140 | 1,096 |
| 155 | 5,000 | 2,302 |
| 156 | 1,549 | 2,168 |
| 157 | 1,159 | 460 |
| 158 | 1,163 | 389 |
| 159 | 5,000 | 1,194 |
| 160 | 5,000 | 1,974 |
| 161 | 658 | 506 |
| 162 | 1,568 | 805 |
| 163 | 200 | 1,882 |
| 164 | 196 | 614 |
| 165 | 5,000 | 460 |
| 166 | 694 | 529 |
| 167 | 5,000 | 1,609 |
| 168 | 587 | 1,095 |
| 169 | 1,094 | 628 |
| 170 | 1,796 | 2,500 |
| 171 | 1,705 | 1,900 |
| 172 | 870 | 1,348 |
| 173 | 2,632 | 3,129 |
| 174 | 757 | 1,448 |
| 175 | 307 | 328 |
| 176 | 1,638 | 1,950 |
| 177 | 949 | 1,463 |
| 178 | 59 | 1,513 |
| 179 | 121 | 213 |
| 180 | 294 | 230 |
| 182 | 865 | 1,774 |
| 183 | 266 | 348 |
| 184 | 401 | 310 |
| 185 | 499 | 508 |
| 186 | 1,694 | 3,486 |
| 187 | 674 | 860 |
| 188 | 781 | 1,405 |
| 189 | 793 | 570 |
| 190 | 563 | 1,685 |
| 191 | 309 | 1,063 |
| 192 | 550 | 1,601 |
| 193 | 333 | 1,079 |
| 194 | 422 | 1,134 |
| 195 | 541 | 1,747 |
| 196 | 1,308 | 2,384 |
| 197 | 570 | 2,744 |
| 198 | 1,309 | 2,272 |
| 199 | 274 | 1,619 |
| 200 | 301 | 879 |
| 201 | 1,348 | 3,140 |
| 202 | 614 | 725 |
| 203 | 5,000 | 2,397 |
| 204 | 5,000 | 1,269 |
| 205 | 5,000 | 1,337 |
| 206 | 1,898 | 1,187 |
| 207 | 2,380 | 1,179 |
| 208 | 1,895 | 1,273 |
| 209 | 5,000 | 2,847 |
| 210 | 5,000 | 1,864 |
| 211 | 1,469 | 1,473 |
| 212 | 148 | 112 |
| 213 | 396 | 677 |

1. A compound of Formula (I): 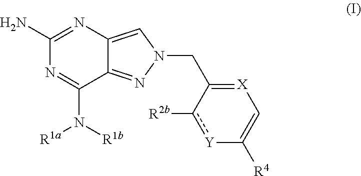
or a pharmaceutically acceptable salt thereof, wherein:
R^{1a} and R^{1b} are each independently C₁-C₆ alkyl, optionally substituted by halo, —OH or —O(C₁-C₃ alkyl);
X is N or CR^{2a};
R^{2a} and R^{2b} are each independently H, —O(C₁-C₃ alkyl), —(C₁-C₃ haloalkyl), halo, —CN, or —NH(C₁-C₃ alkyl);
Y is CR³, NR³, or N;
R³ is H or —O(C₁-C₃ alkyl);
or R^{2b} and R³ can be taken together to form 5- to 6-membered heteroaryl or 5- to 6-membered heterocyclyl, optionally substituted with C₁-C₃ alkyl, —C(O)CH₂N(C₁-C₃ alkyl)₂, or —C(O)(CH₂)NH(C₃-C₆ cycloalkyl), wherein the heteroaryl and heterocyclyl contain 1-3 heteroatoms selected from N, O, and S;
R⁴ is H, —CH₂OH, optionally substituted 3- to 6-membered heterocyclyl containing 1 heteroatom selected from N and O, optionally substituted C₃-C₆ cycloalkyl, or —CH₂NR^{5a}R^{5b}; and
R^{5a} is H and R^{5b} is optionally substituted 4- to 7-membered heterocyclyl, optionally substituted C₃-C₆ cycloalkyl, or optionally substituted C₁-C₆ alkyl, wherein the heterocyclyl contains 1 heteroatom selected from N and O, or R^{5a} and R^{5b} are taken together to form optionally substituted 4- to 8-membered heterocyclyl containing 1-2 heteroatoms selected from N and O.

2. (canceled)

3. The compound of claim 1, or a pharmaceutically acceptable salt thereof, wherein:
R^{1a} and R^{1b} are each independently C₁-C₅ alkyl, optionally substituted by F, —OH or —OCH₃.

4. (canceled)

5. The compound of claim 1, or a pharmaceutically acceptable salt thereof, wherein:
X is CR^{2a}.

6. (canceled)

7. The compound of claim 1, or a pharmaceutically acceptable salt thereof, wherein:
R^{2a} and R^{2b} are each independently H, —OCH₃, —CHF₂, F, Cl, Br, I, —CN, or —NHCH₃.

8. (canceled)

9. The compound of claim 1, or a pharmaceutically acceptable salt thereof, wherein:
Y is CR³; and R³ is H or —OCH₃.

10-12. (canceled)

13. The compound of claim 1, or a pharmaceutically acceptable salt thereof, wherein:
R^{2b} and R³ are taken together to form 5- to 6-membered heteroaryl or 5- to 6-membered heterocyclyl, optionally substituted with methyl, —C(O)CH₂N(CH₃)₂, or —C(O)(CH₂)NH(cyclobutyl), wherein the heteroaryl and heterocyclyl contain 1-3 heteroatoms selected from N, O, and S.

14-16. (canceled)

17. The compound of claim 1, or a pharmaceutically acceptable salt thereof, wherein:
R⁴ is optionally substituted 5- to 6-membered heterocyclyl containing 1 heteroatom selected from N and O.

18. (canceled)

19. The compound of claim 1, or a pharmaceutically acceptable salt thereof, wherein:
R⁴ is —CH₂NR^{5a}R^{5b}; and
R^{5a} is H and R^{5b} is optionally substituted 4- to 7-membered heterocyclyl containing 1 heteroatom selected from N and O; or
R^{5a} is H and R^{5b} is C₁-C₆ alkyl optionally substituted with —OH, —O(C₁-C₃ alkyl), or C₃-C₆ cycloalkyl; or
R^{5a} is H and R^{5b} is optionally substituted C₃-C₆ cycloalkyl.

20-23. (canceled)

24. The compound of claim 1, or a pharmaceutically acceptable salt thereof, wherein:
R⁴ is

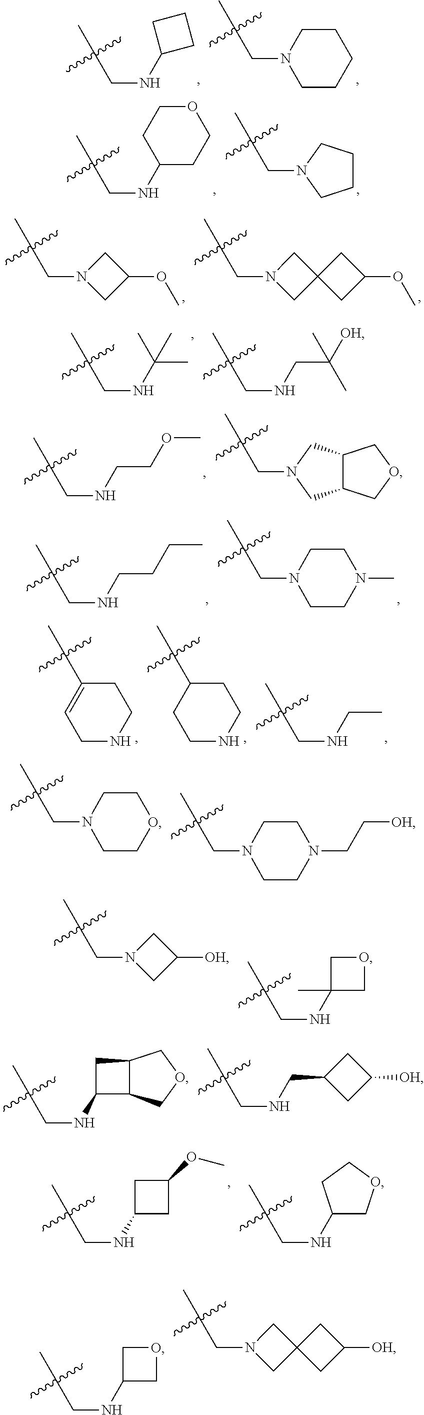

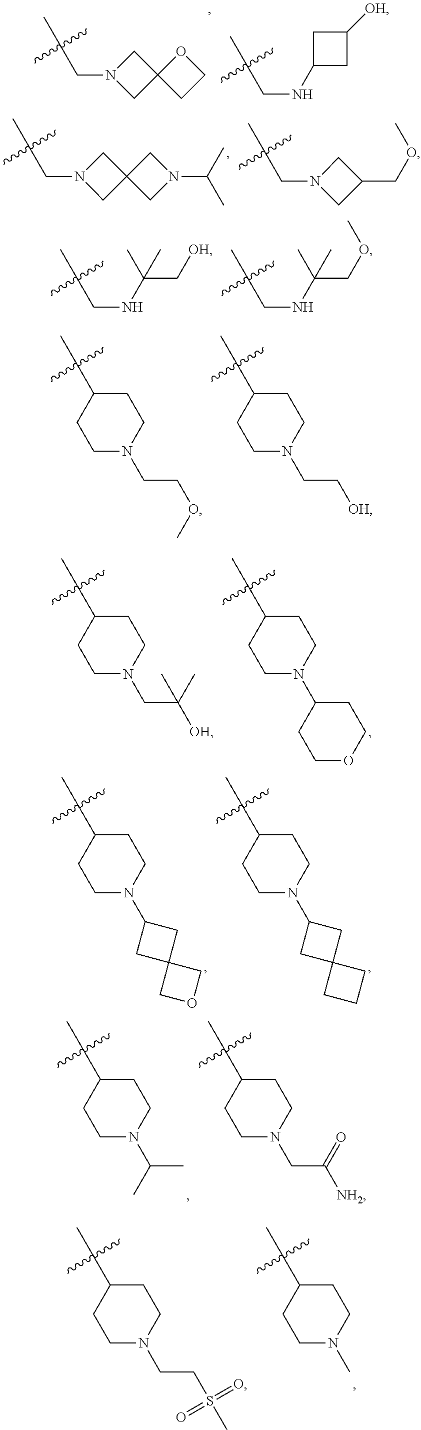

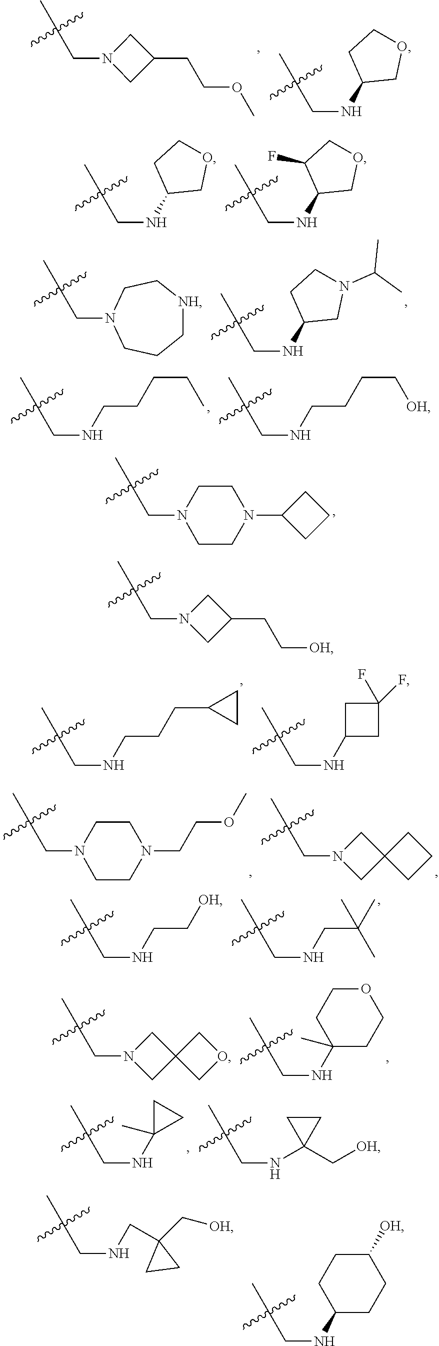

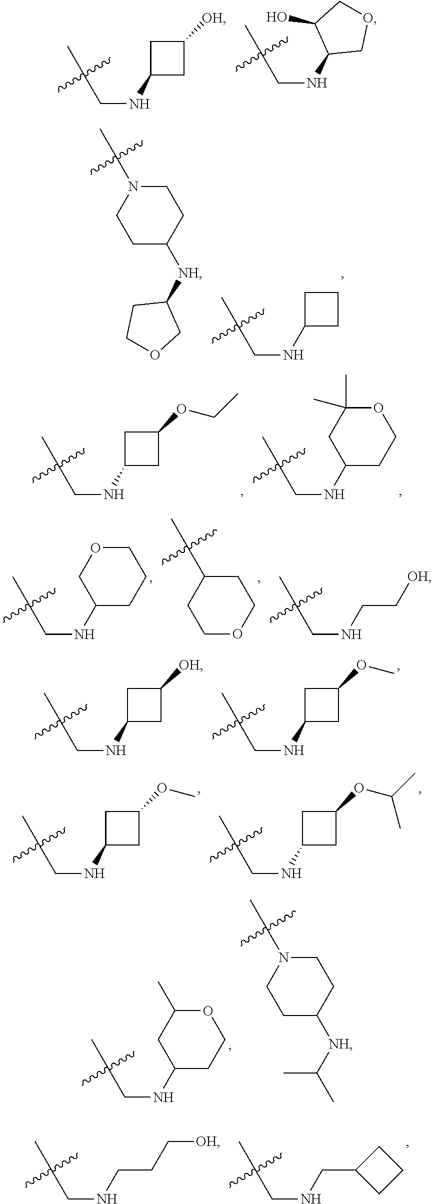

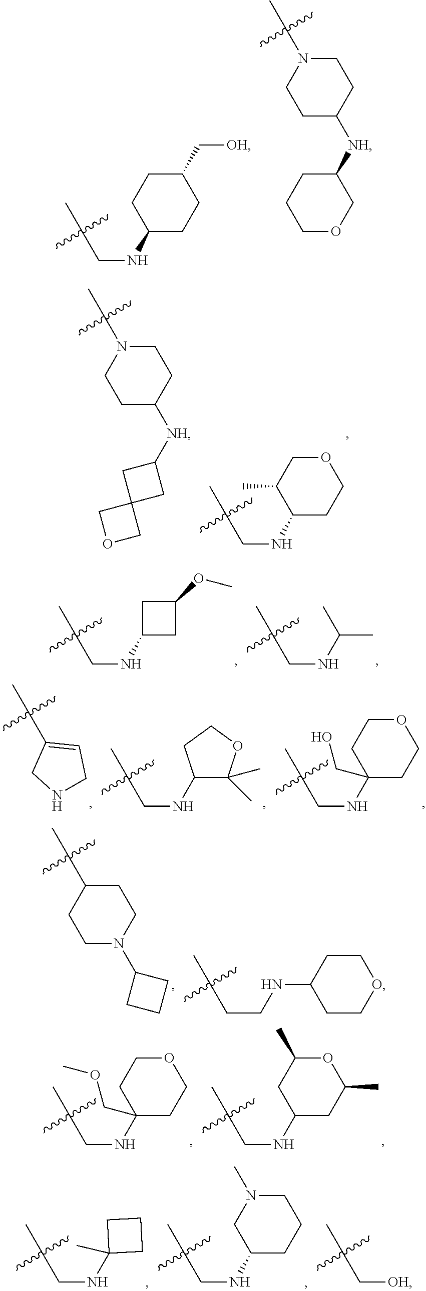

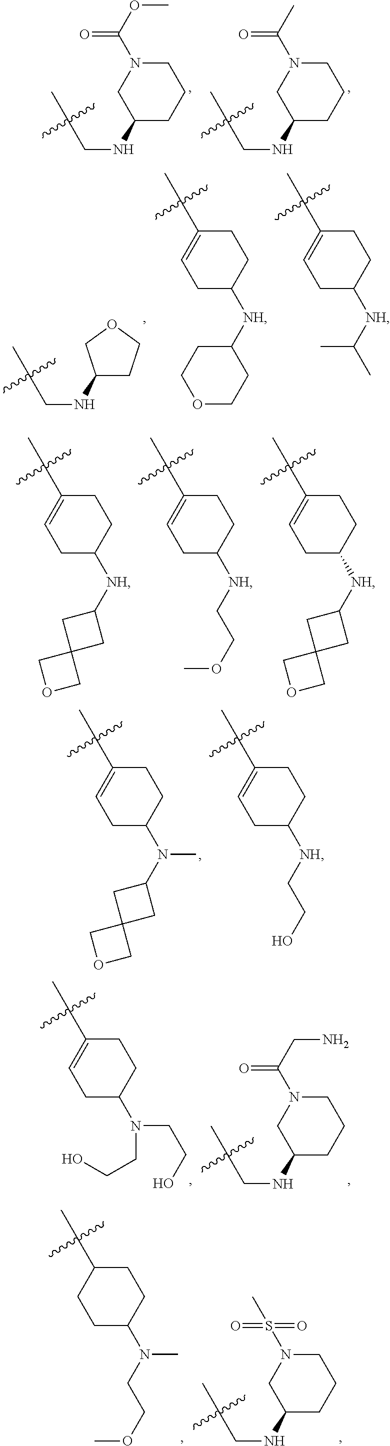

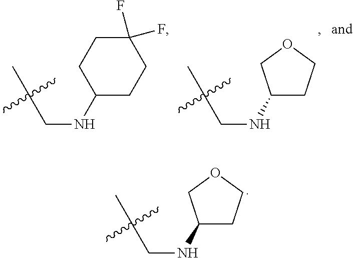 [SMILES: *CNC1CCC(F)(F)CC1 ; *CN[C@H]1CCOC1 ; *CN[C@@H]1CCOC1]

25-30. (canceled)

31. A compound selected from: 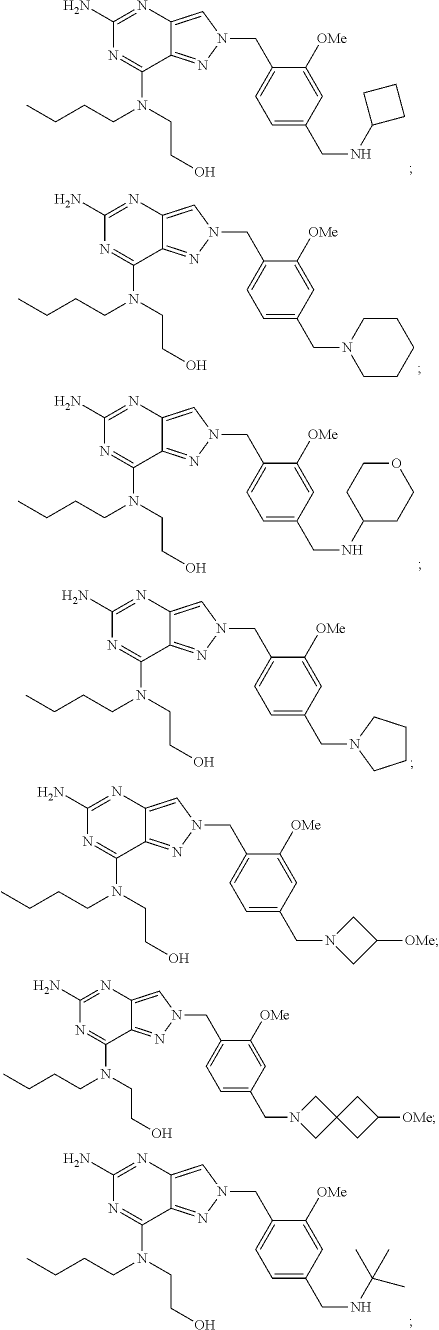 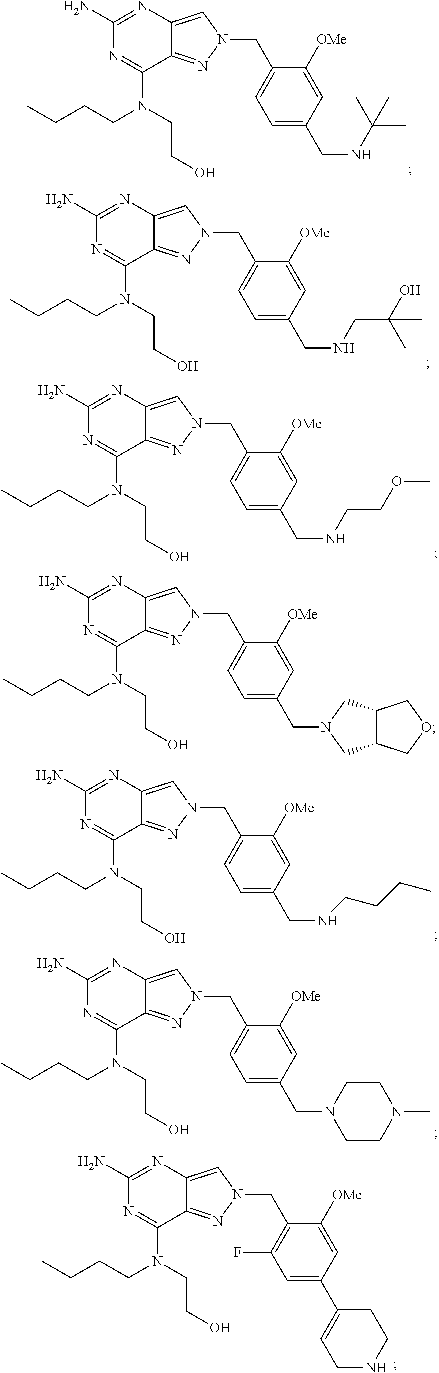 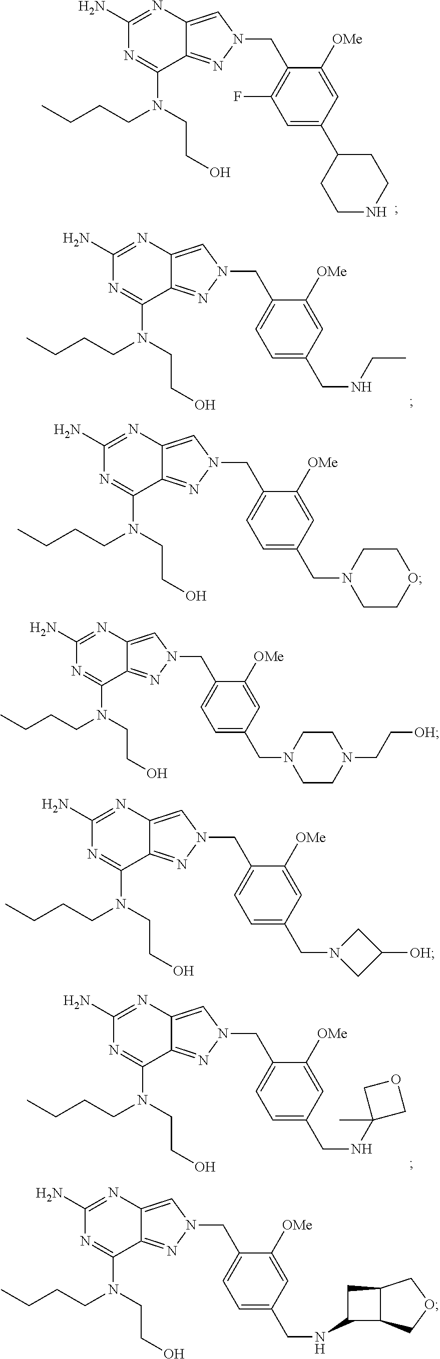 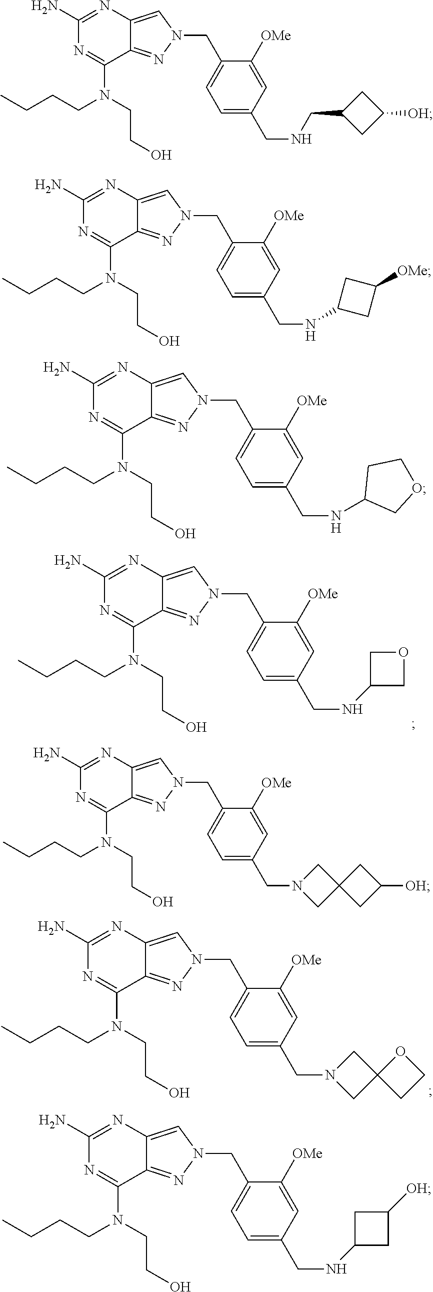 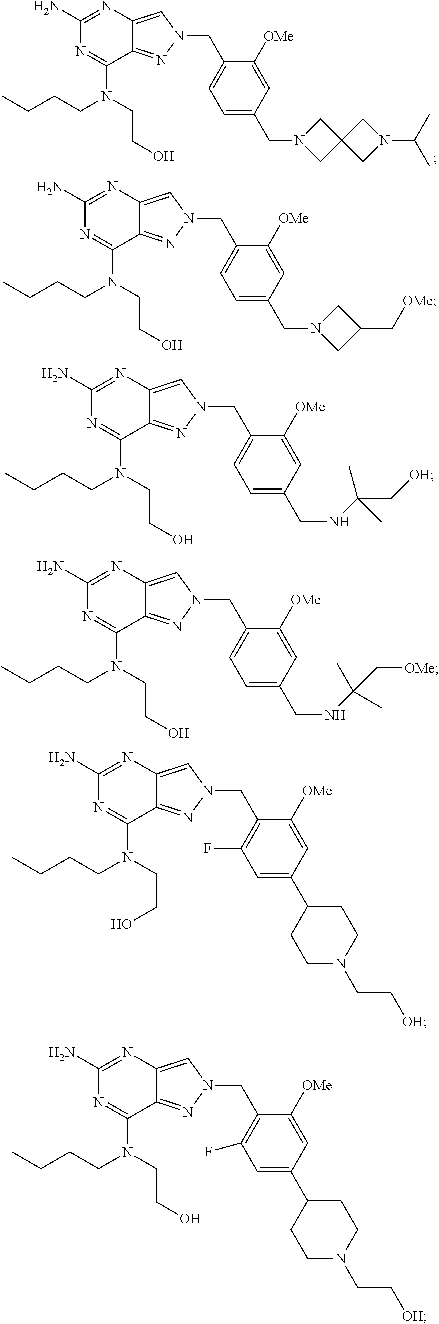 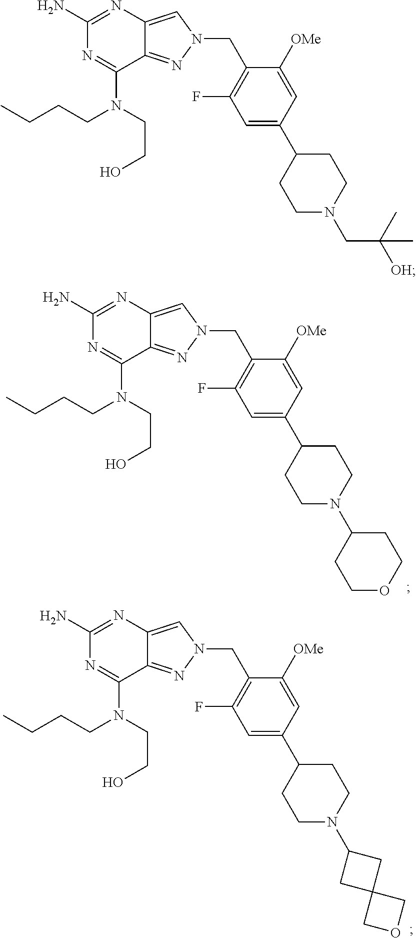 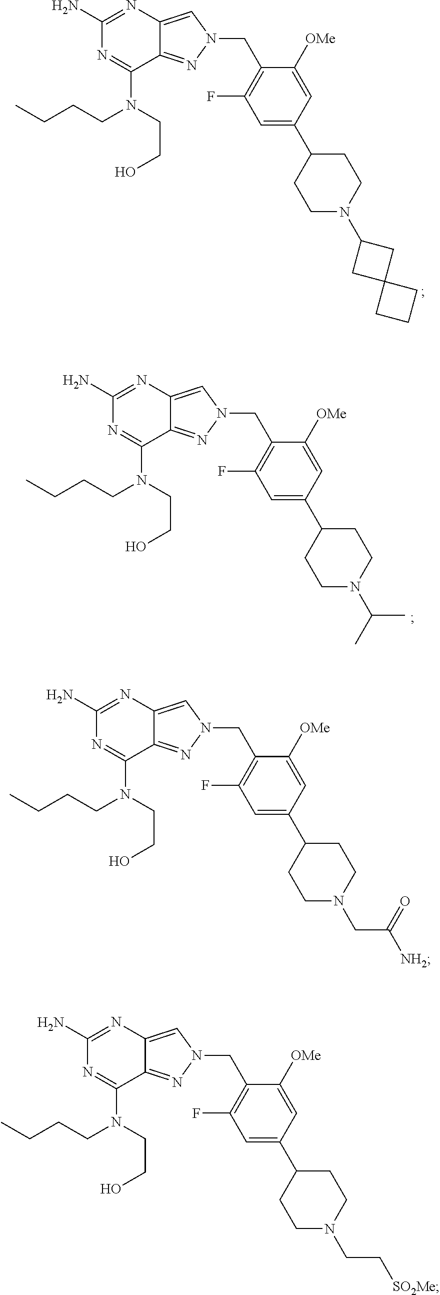 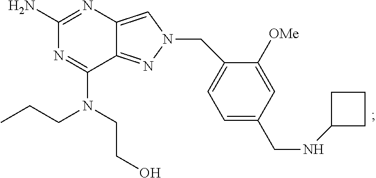 [SMILES: CCCN(CCO)c1nc(N)nc2cn(Cc3ccc(CNC4CCC4)cc3OC)nc12] 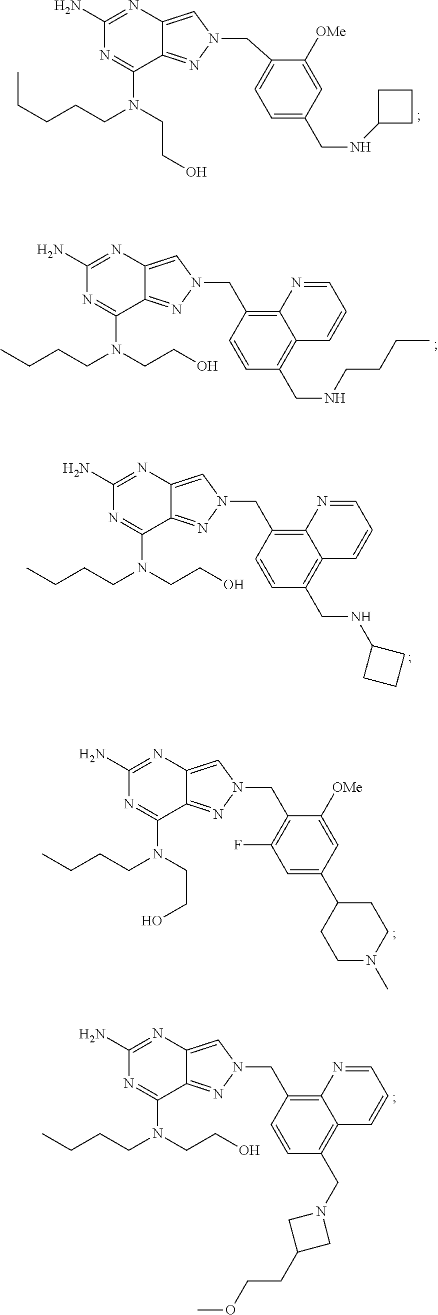 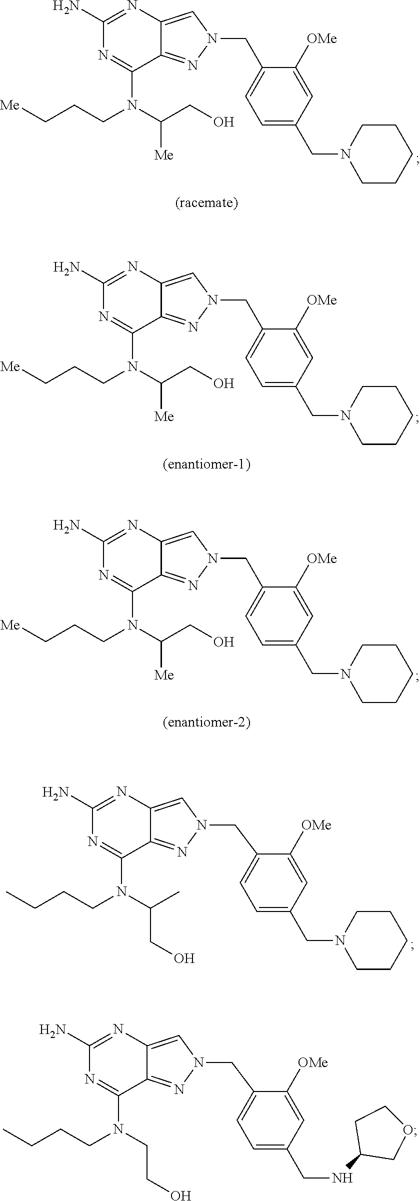 [SMILES: CCCCN(c1nc(N)nc2cn(Cc3ccc(CN4CCCCC4)cc3OC)nc12)C(C)CO ; CCCCN(c1nc(N)nc2cn(Cc3ccc(CN4CCCCC4)cc3OC)nc12)C(C)CO ; CCCCN(c1nc(N)nc2cn(Cc3ccc(CN4CCCCC4)cc3OC)nc12)C(C)CO ; CCCCN(c1nc(N)nc2cn(Cc3ccc(CN4CCCCC4)cc3OC)nc12)C(C)CO ; CCCCN(CCO)c1nc(N)nc2cn(Cc3ccc(CN[C@H]4CCOC4)cc3OC)nc12] 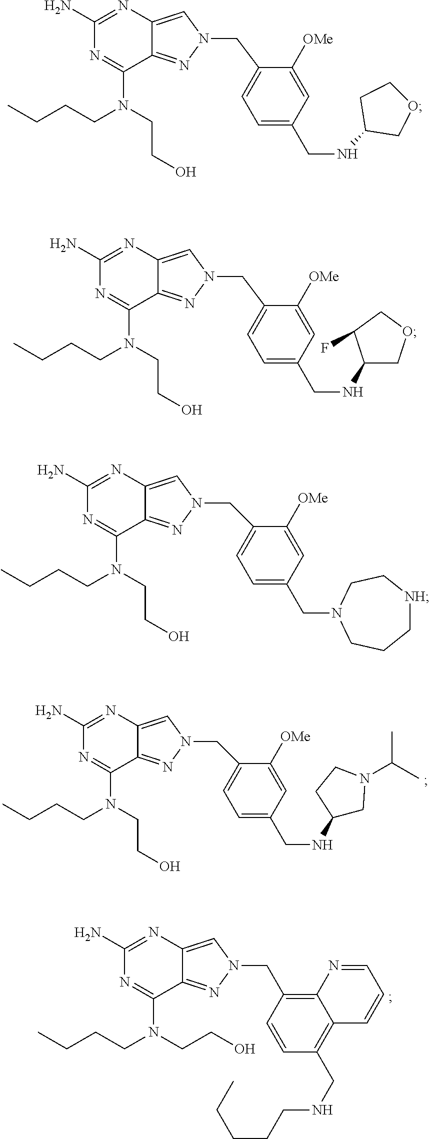 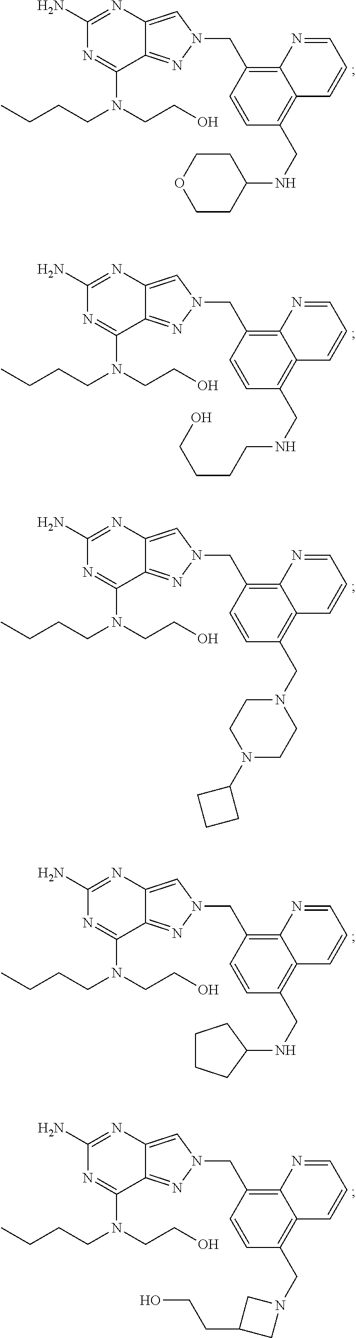 [SMILES: CCCCN(CCO)c1nc(N)nc2cn(Cc3ccc(CNC4CCOCC4)c4cccnc34)nc12 ; CCCCN(CCO)c1nc(N)nc2cn(Cc3ccc(CNCCCCO)c4cccnc34)nc12 ; CCCCN(CCO)c1nc(N)nc2cn(Cc3ccc(CN4CCN(C5CCC5)CC4)c4cccnc34)nc12 ; CCCCN(CCO)c1nc(N)nc2cn(Cc3ccc(CNC4CCCC4)c4cccnc34)nc12 ; CCCCN(CCO)c1nc(N)nc2cn(Cc3ccc(CN4CC(CCO)C4)c4cccnc34)nc12] 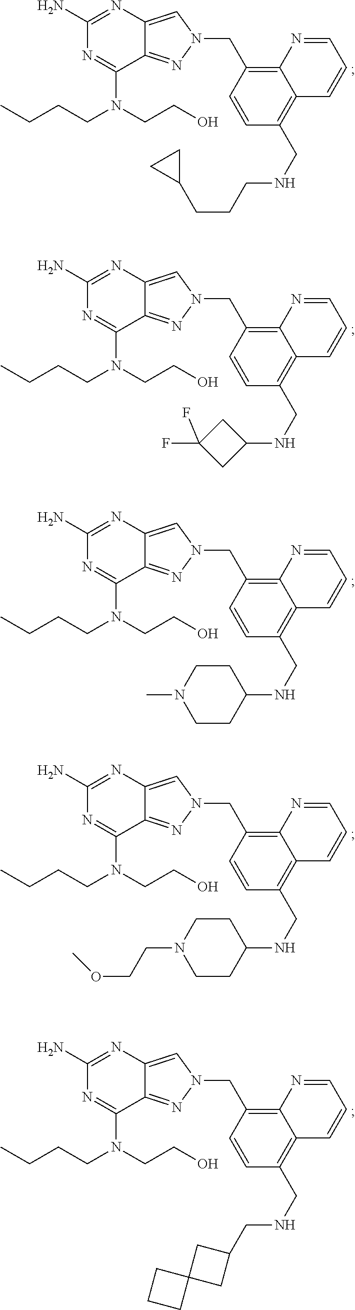 [SMILES: CCCCN(CCO)c1nc(N)nc2cn(Cc3ccc(CNCCCC4CC4)c4cccnc34)nc12 ; CCCCN(CCO)c1nc(N)nc2cn(Cc3ccc(CNC4CC(F)(F)C4)c4cccnc34)nc12 ; CCCCN(CCO)c1nc(N)nc2cn(Cc3ccc(CNC4CCN(C)CC4)c4cccnc34)nc12 ; CCCCN(CCO)c1nc(N)nc2cn(Cc3ccc(CNC4CCN(CCOC)CC4)c4cccnc34)nc12 ; CCCCN(CCO)c1nc(N)nc2cn(Cc3ccc(CNCC4CC5(CCC5)C4)c4cccnc34)nc12] 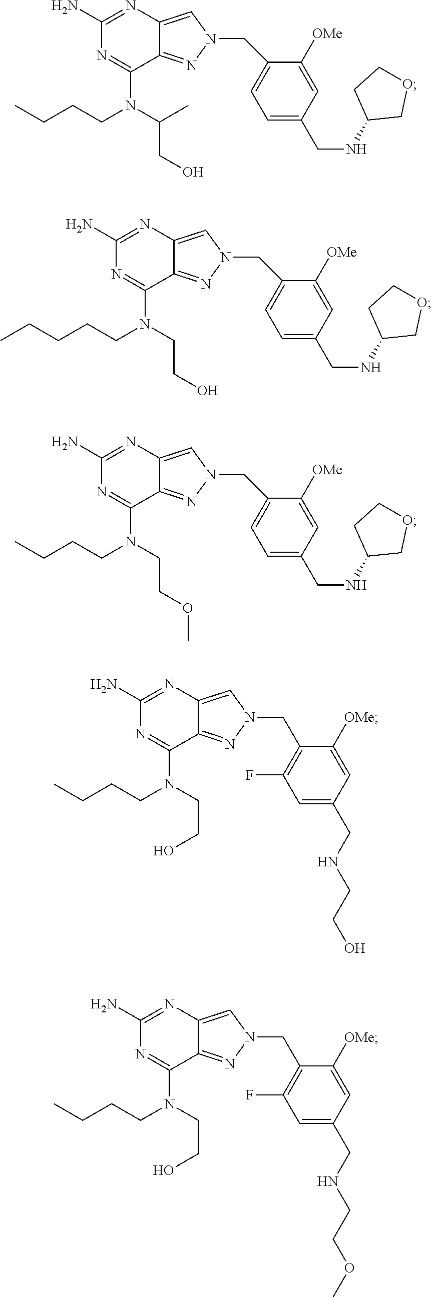 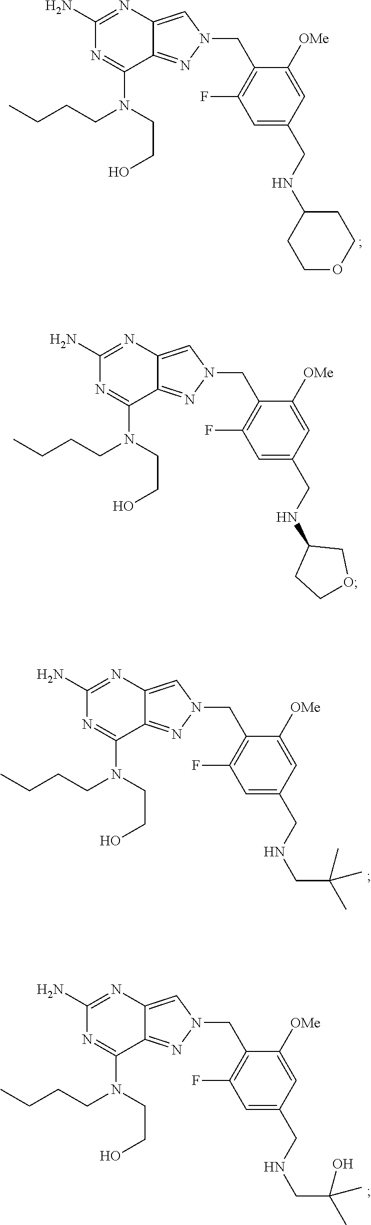 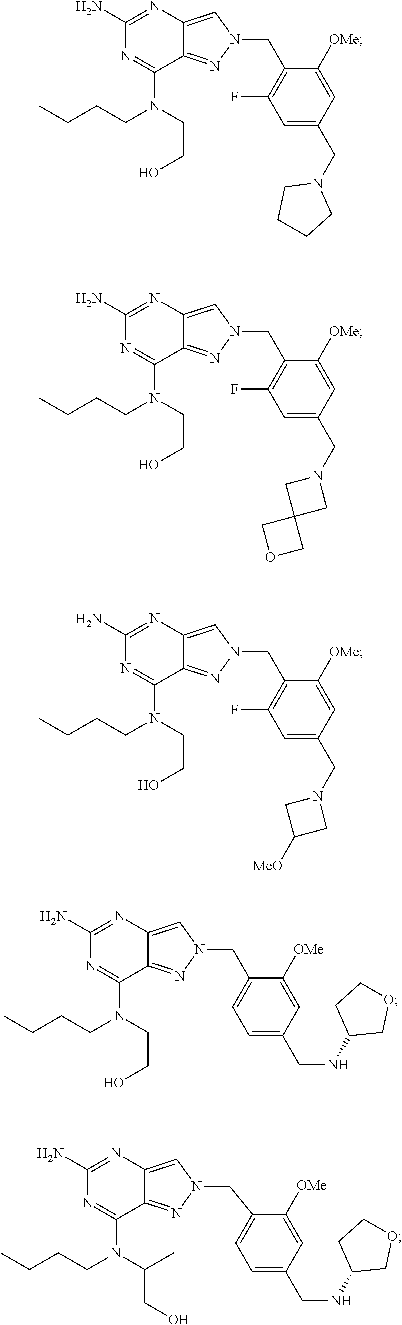 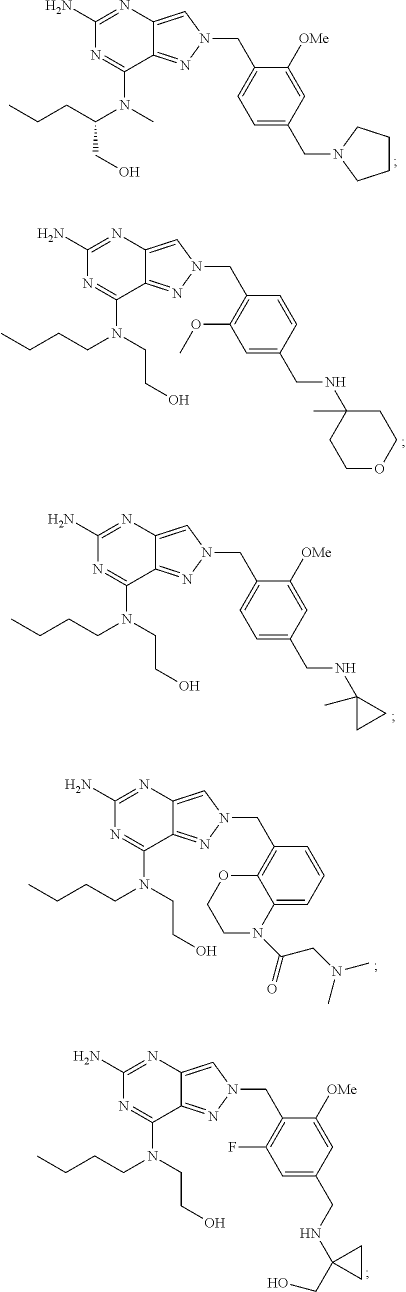 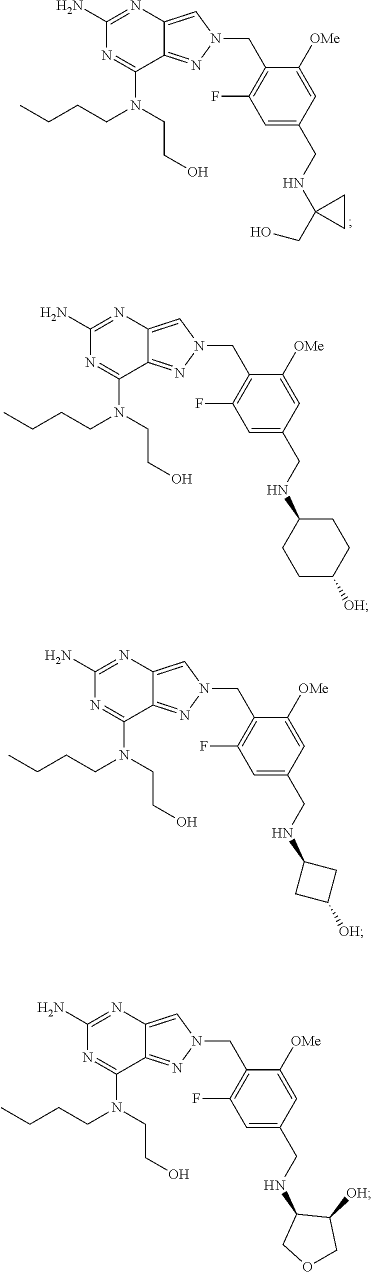 [SMILES: CCCCN(CCO)c1nc(N)nc2cn(Cc3c(F)cc(CNC4(CO)CC4)cc3OC)nc12 ; CCCCN(CCO)c1nc(N)nc2cn(Cc3c(F)cc(CN[C@H]4CC[C@H](O)CC4)cc3OC)nc12 ; CCCCN(CCO)c1nc(N)nc2cn(Cc3c(F)cc(CN[C@H]4C[C@H](O)C4)cc3OC)nc12 ; CCCCN(CCO)c1nc(N)nc2cn(Cc3c(F)cc(CN[C@@H]4COC[C@@H]4O)cc3OC)nc12] 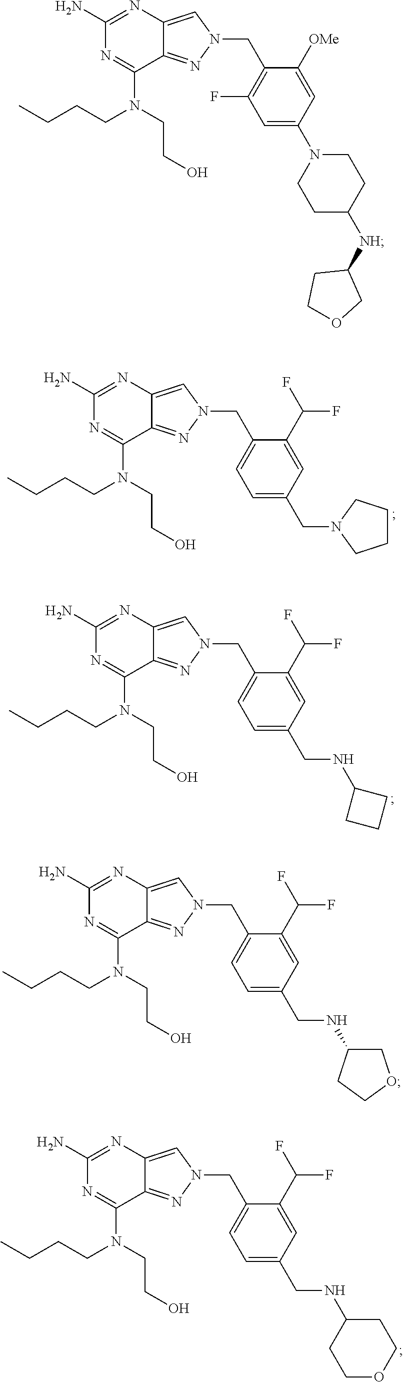 [SMILES: CCCCN(CCO)c1nc(N)nc2cn(Cc3c(F)cc(N4CCC(N[C@@H]5CCOC5)CC4)cc3OC)nc12 ; CCCCN(CCO)c1nc(N)nc2cn(Cc3ccc(CN4CCCC4)cc3C(F)F)nc12 ; CCCCN(CCO)c1nc(N)nc2cn(Cc3ccc(CNC4CCC4)cc3C(F)F)nc12 ; CCCCN(CCO)c1nc(N)nc2cn(Cc3ccc(CN[C@H]4CCOC4)cc3C(F)F)nc12 ; CCCCN(CCO)c1nc(N)nc2cn(Cc3ccc(CNC4CCOCC4)cc3C(F)F)nc12] 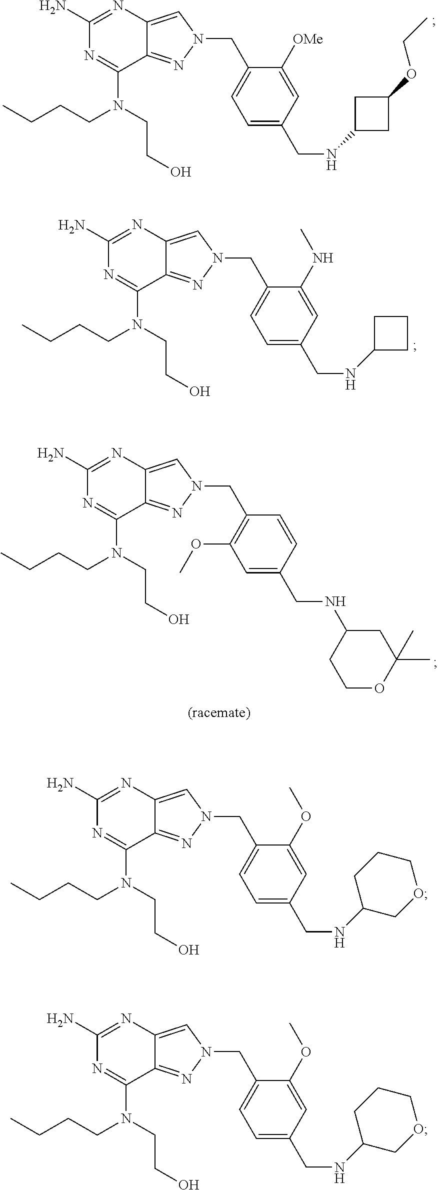 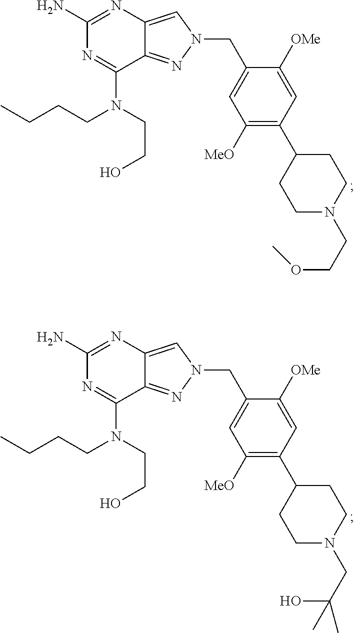 [SMILES: CCCCN(CCO)c1nc(N)nc2cn(Cc3cc(OC)c(C4CCN(CCOC)CC4)cc3OC)nc12 ; CCCCN(CCO)c1nc(N)nc2cn(Cc3cc(OC)c(C4CCN(CC(C)(C)O)CC4)cc3OC)nc12] 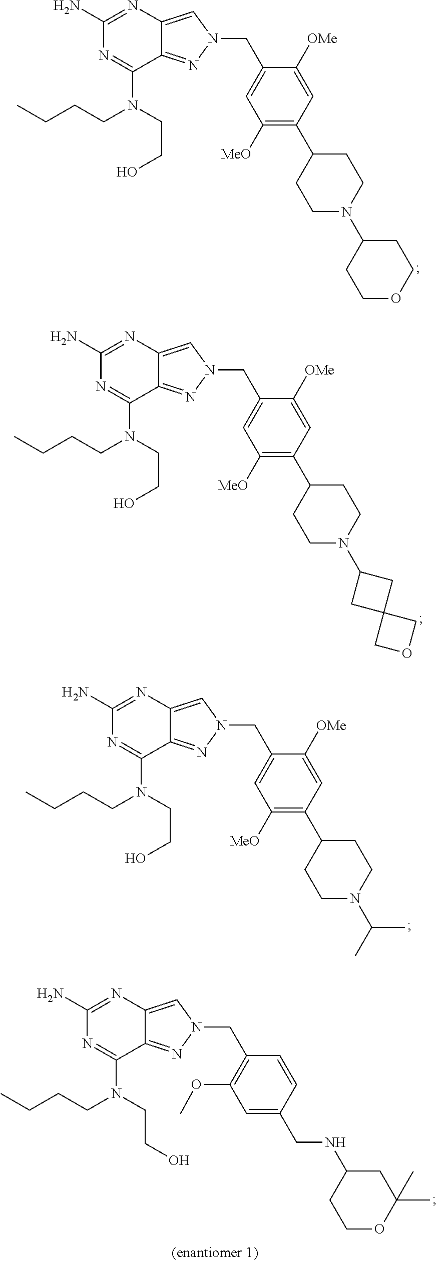 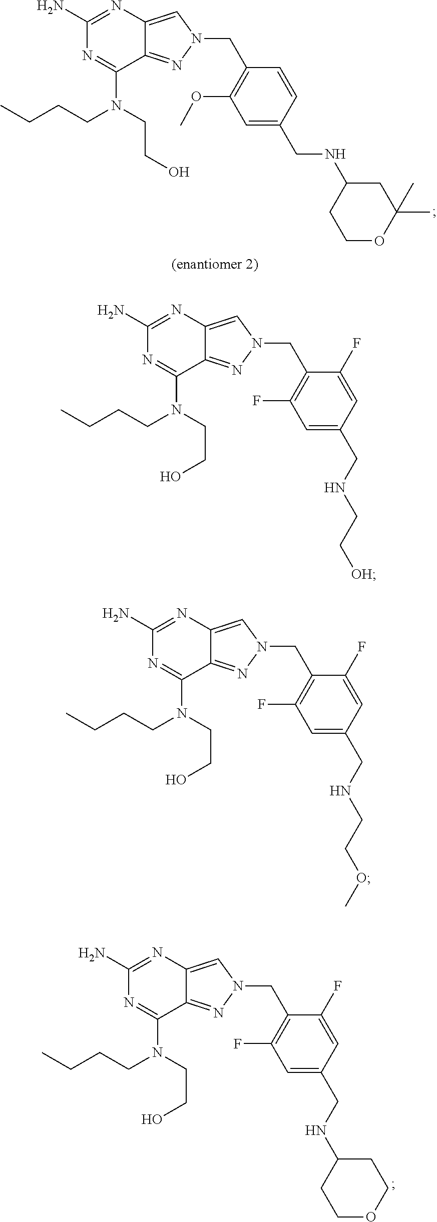 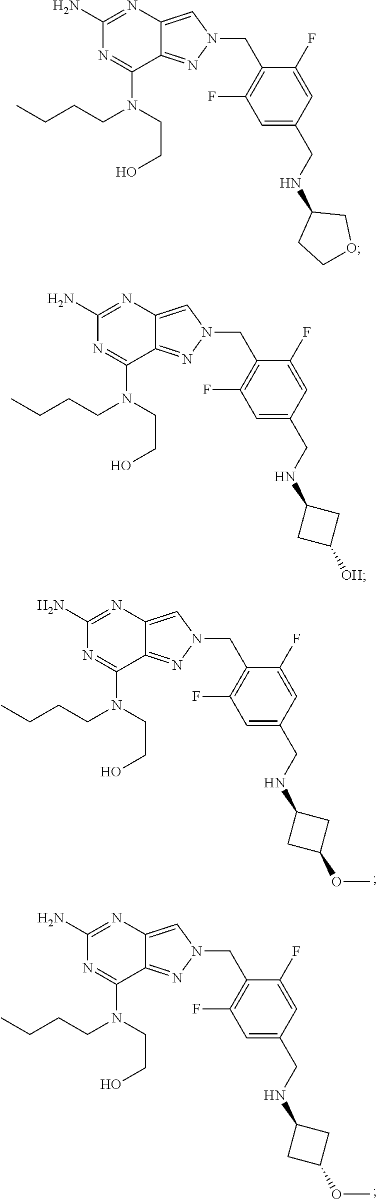 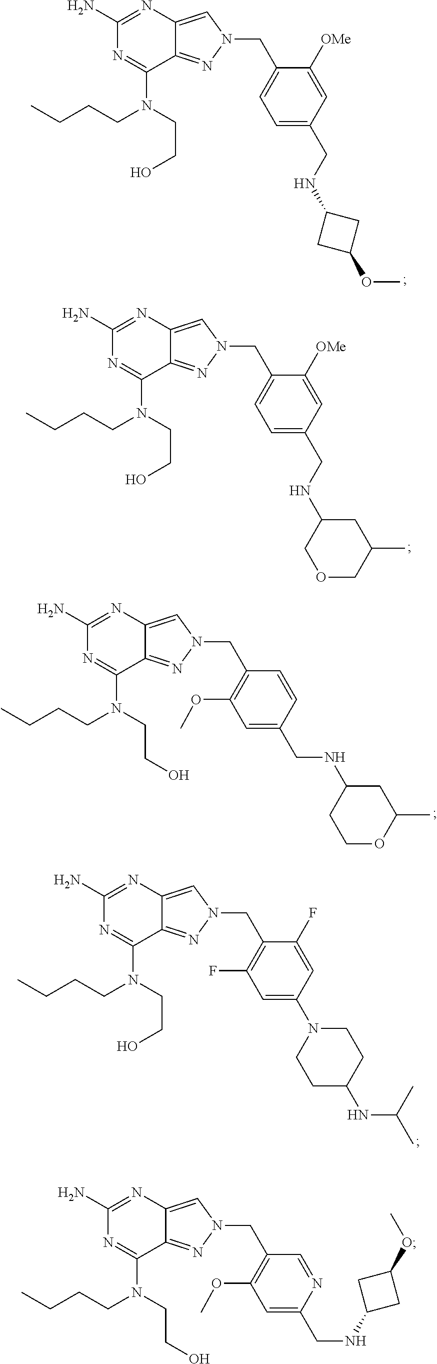 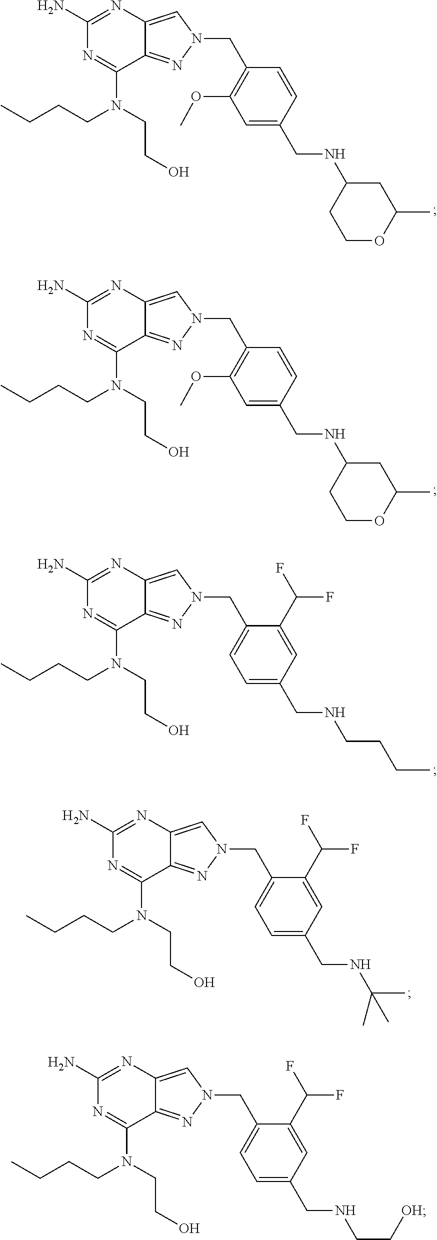 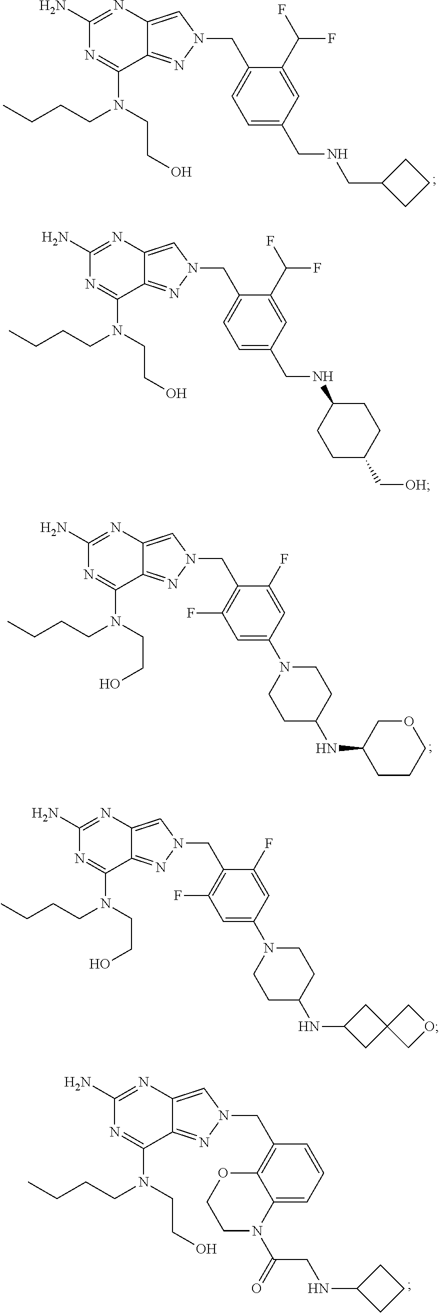 [SMILES: CCCCN(CCO)c1nc(N)nc2cn(Cc3ccc(CNCC4CCC4)cc3C(F)F)nc12 ; CCCCN(CCO)c1nc(N)nc2cn(Cc3ccc(CN[C@H]4CC[C@H](CO)CC4)cc3C(F)F)nc12 ; CCCCN(CCO)c1nc(N)nc2cn(Cc3c(F)cc(N4CCC(N[C@@H]5CCCOC5)CC4)cc3F)nc12 ; CCCCN(CCO)c1nc(N)nc2cn(Cc3c(F)cc(N4CCC(NC5CC6(COC6)C5)CC4)cc3F)nc12 ; CCCCN(CCO)c1nc(N)nc2cn(Cc3cccc4c3OCCN4C(=O)CNC3CCC3)nc12] 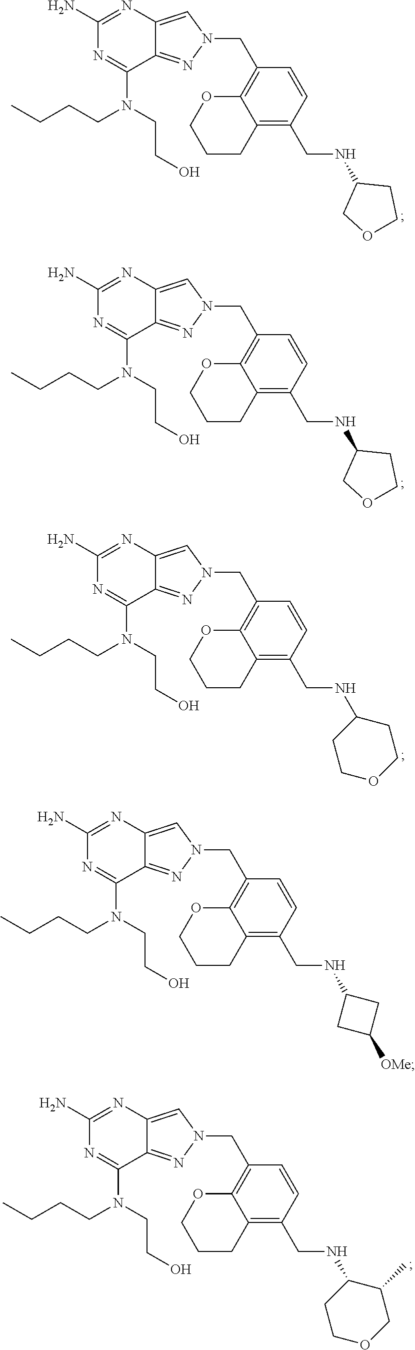 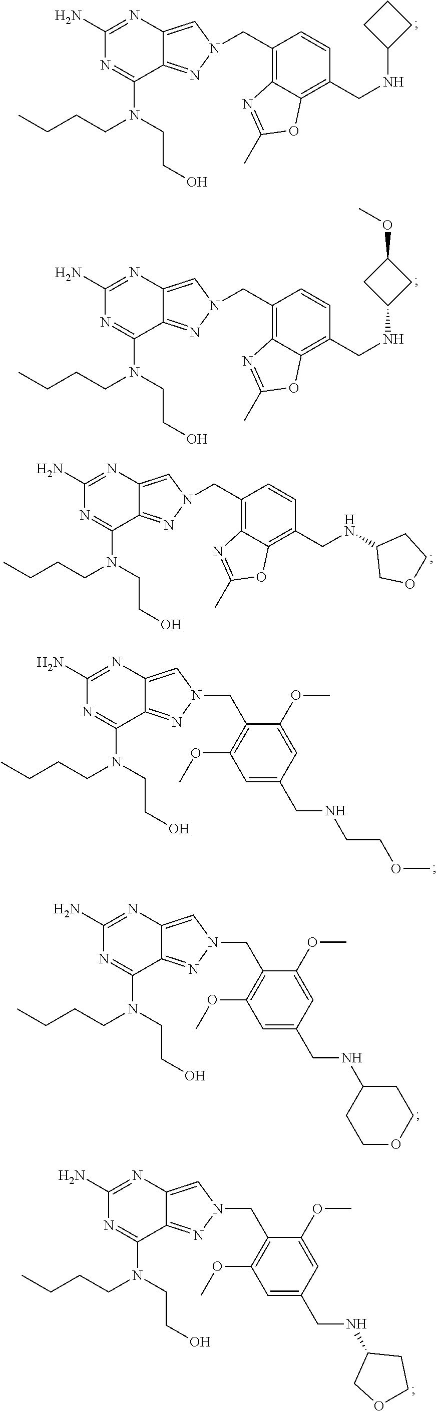 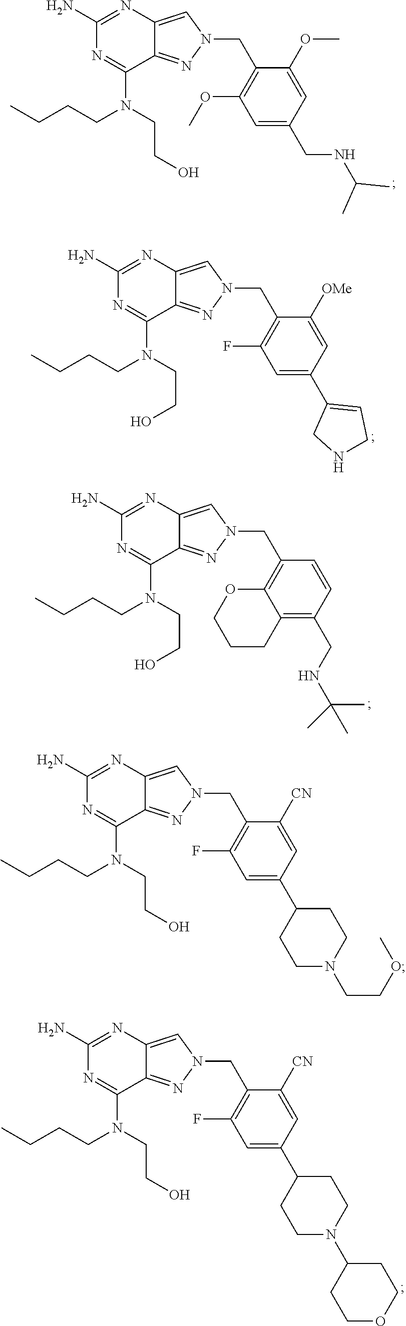 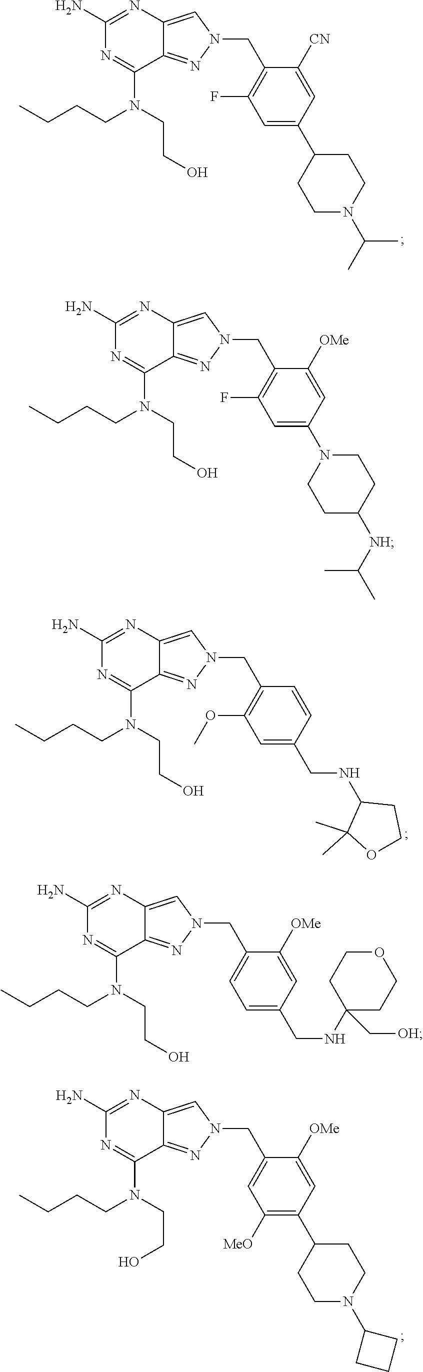 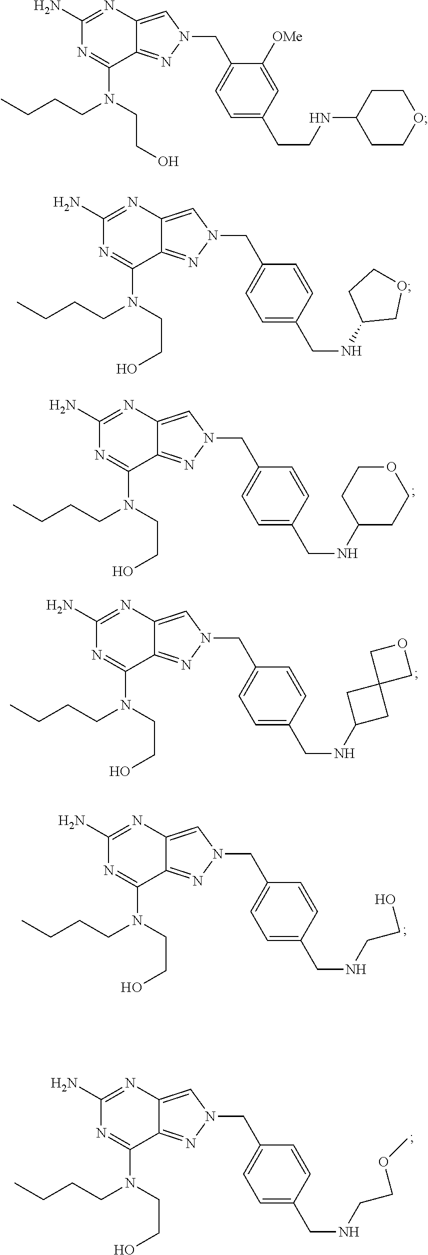 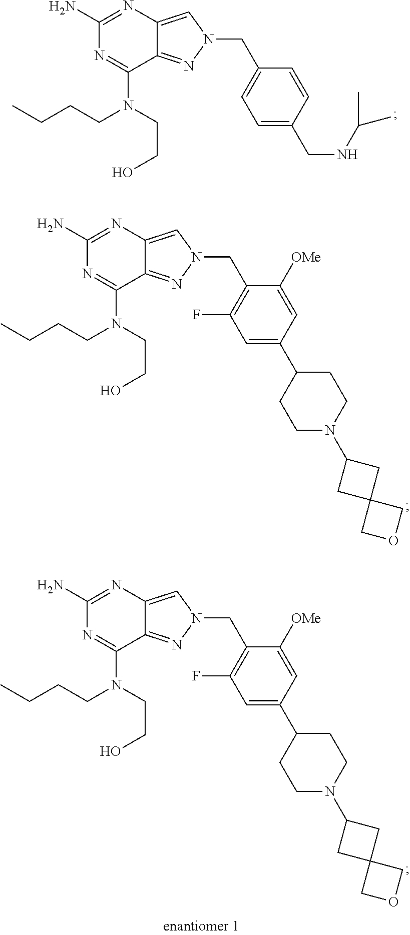 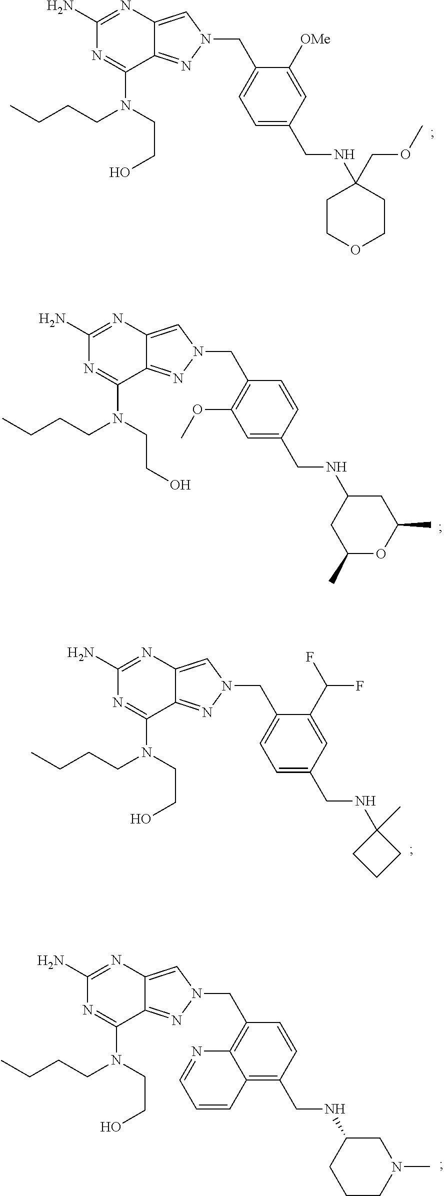 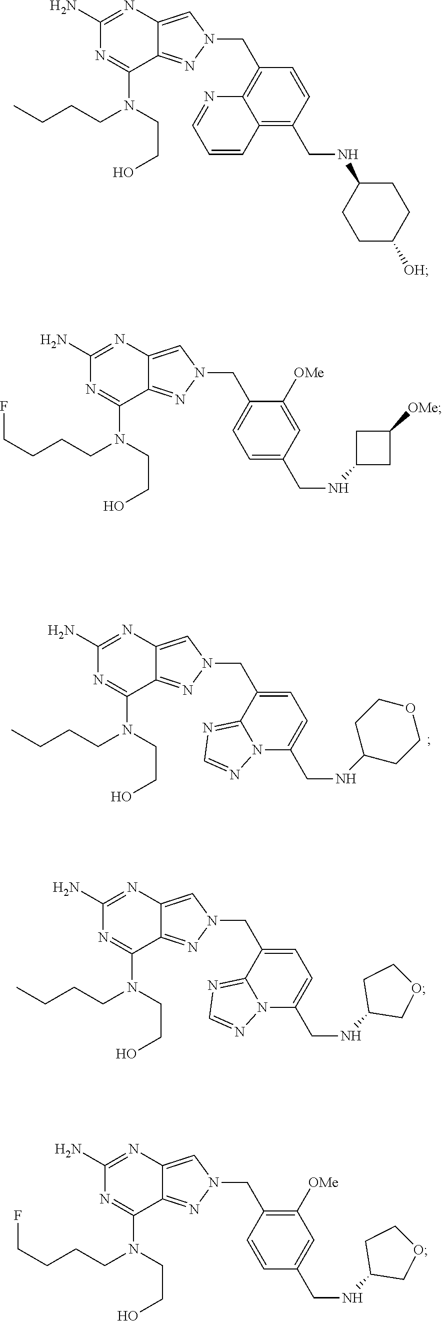 [SMILES: CCCCN(CCO)c1nc(N)nc2cn(Cc3ccc(CN[C@H]4CC[C@H](O)CC4)c4cccnc34)nc12 ; COc1cc(CN[C@H]2C[C@H](OC)C2)ccc1Cn1cc2nc(N)nc(N(CCO)CCCCF)c2n1 ; CCCCN(CCO)c1nc(N)nc2cn(Cc3ccc(CNC4CCOCC4)n4ncnc34)nc12 ; CCCCN(CCO)c1nc(N)nc2cn(Cc3ccc(CN[C@@H]4CCOC4)n4ncnc34)nc12 ; COc1cc(CN[C@@H]2CCOC2)ccc1Cn1cc2nc(N)nc(N(CCO)CCCCF)c2n1] 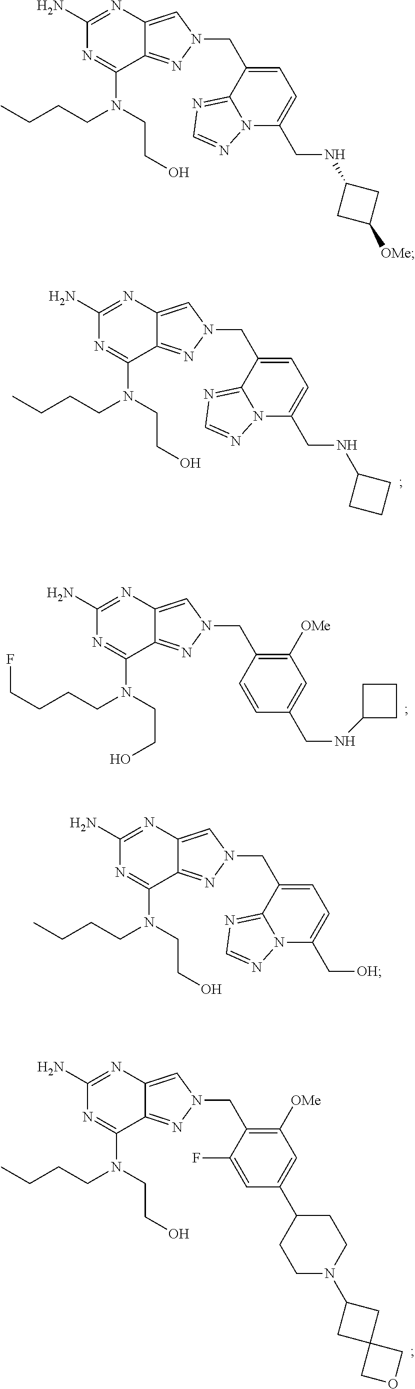 [SMILES: CCCCN(CCO)c1nc(N)nc2cn(Cc3ccc(CN[C@H]4C[C@H](OC)C4)n4ncnc34)nc12 ; CCCCN(CCO)c1nc(N)nc2cn(Cc3ccc(CNC4CCC4)n4ncnc34)nc12 ; COc1cc(CNC2CCC2)ccc1Cn1cc2nc(N)nc(N(CCO)CCCCF)c2n1 ; CCCCN(CCO)c1nc(N)nc2cn(Cc3ccc(CO)n4ncnc34)nc12 ; CCCCN(CCO)c1nc(N)nc2cn(Cc3c(F)cc(C4CCN(C5CC6(COC6)C5)CC4)cc3OC)nc12] 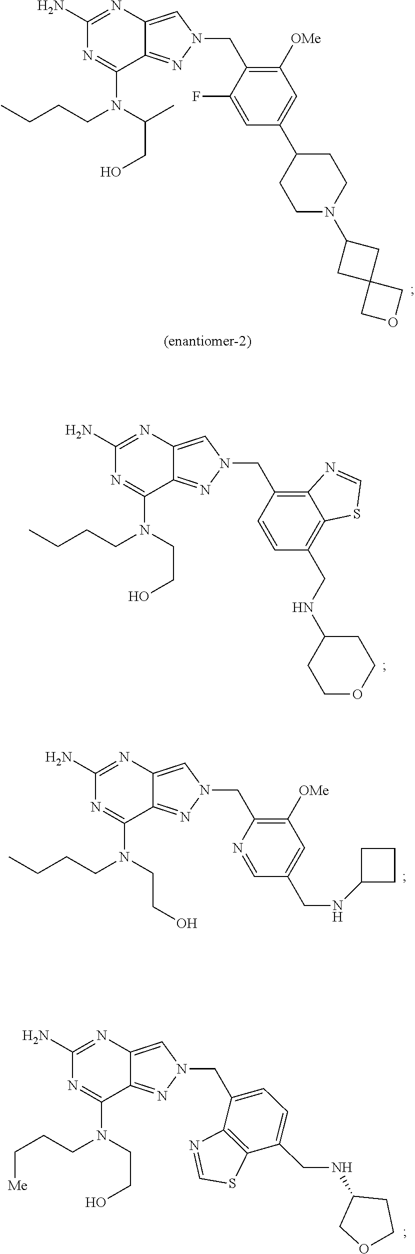 [SMILES: CCCCN(c1nc(N)nc2cn(Cc3c(F)cc(C4CCN(C5CC6(COC6)C5)CC4)cc3OC)nc12)C(C)CO ; CCCCN(CCO)c1nc(N)nc2cn(Cc3ccc(CNC4CCOCC4)c4scnc34)nc12 ; CCCCN(CCO)c1nc(N)nc2cn(Cc3ncc(CNC4CCC4)cc3OC)nc12 ; CCCCN(CCO)c1nc(N)nc2cn(Cc3ccc(CN[C@@H]4CCOC4)c4scnc34)nc12] 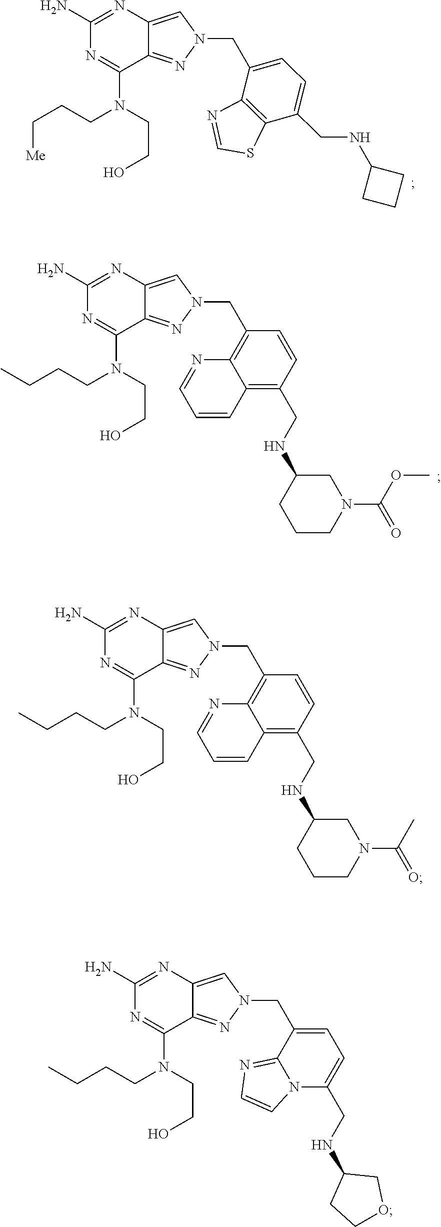 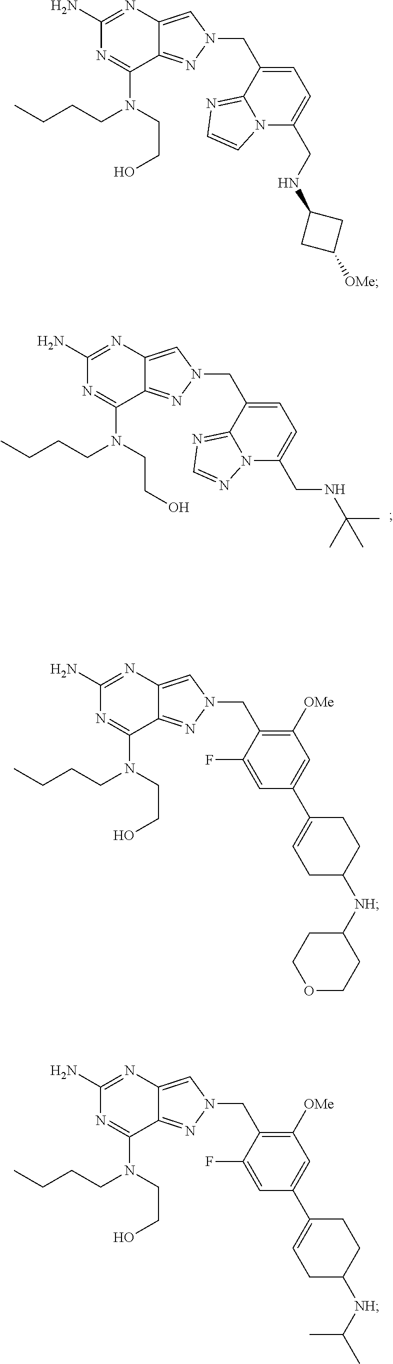 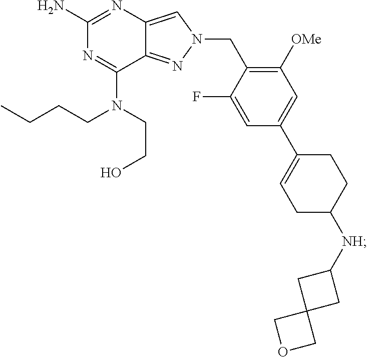 [SMILES: CCCCN(CCO)c1nc(N)nc2cn(Cc3c(F)cc(C4=CCC(NC5CC6(COC6)C5)CC4)cc3OC)nc12] 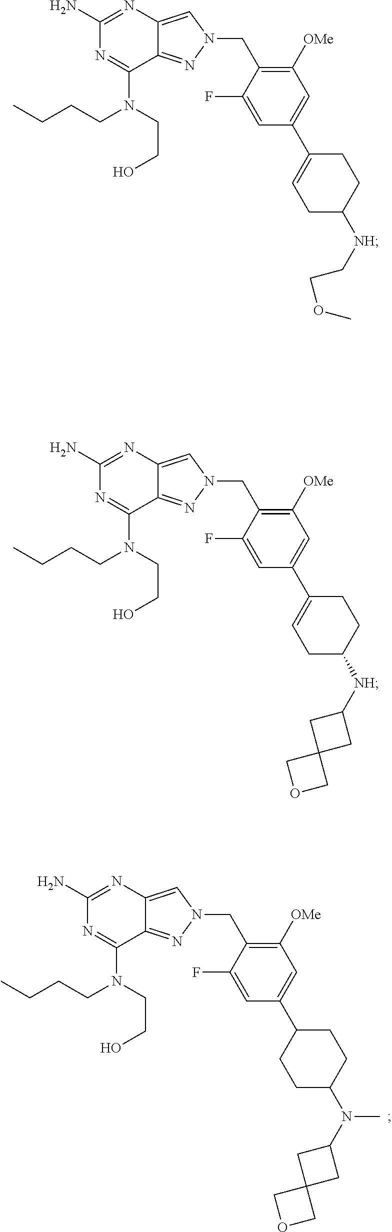 [SMILES: CCCCN(CCO)c1nc(N)nc2cn(Cc3c(F)cc(C4=CCC(NCCOC)CC4)cc3OC)nc12 ; CCCCN(CCO)c1nc(N)nc2cn(Cc3c(F)cc(C4=CC[C@@H](NC5CC6(COC6)C5)CC4)cc3OC)nc12 ; CCCCN(CCO)c1nc(N)nc2cn(Cc3c(F)cc(C4CCC(N(C)C5CC6(COC6)C5)CC4)cc3OC)nc12] 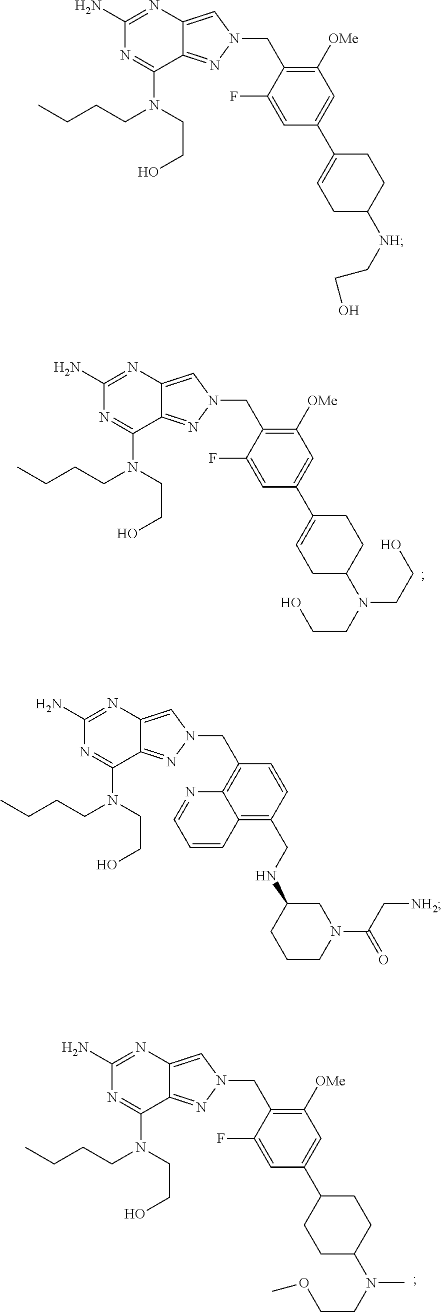 [SMILES: CCCCN(CCO)c1nc(N)nc2cn(Cc3c(F)cc(C4=CCC(NCCO)CC4)cc3OC)nc12 ; CCCCN(CCO)c1nc(N)nc2cn(Cc3c(F)cc(C4=CCC(N(CCO)CCO)CC4)cc3OC)nc12 ; CCCCN(CCO)c1nc(N)nc2cn(Cc3ccc(CN[C@@H]4CCCN(C(=O)CN)C4)c4cccnc34)nc12 ; CCCCN(CCO)c1nc(N)nc2cn(Cc3c(F)cc(C4CCC(N(C)CCOC)CC4)cc3OC)nc12] 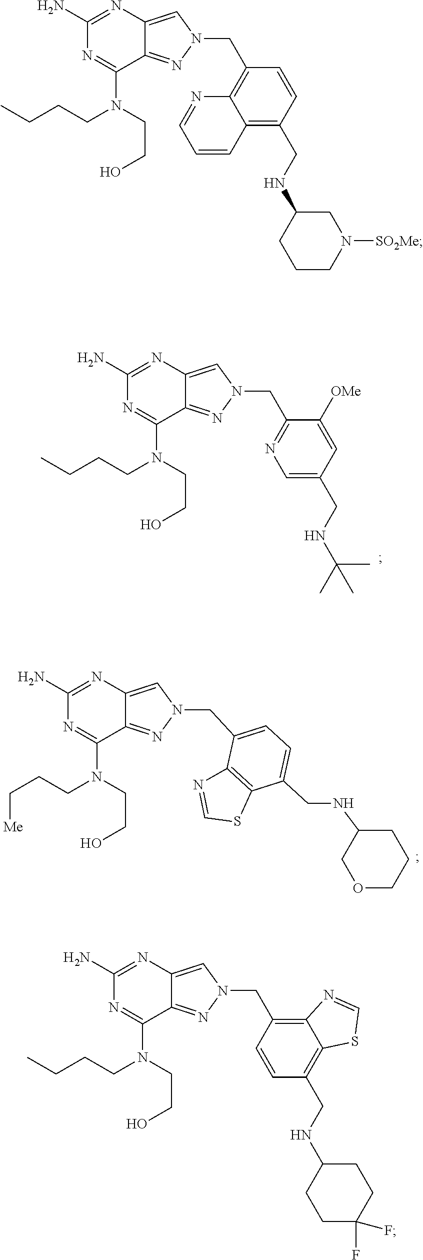 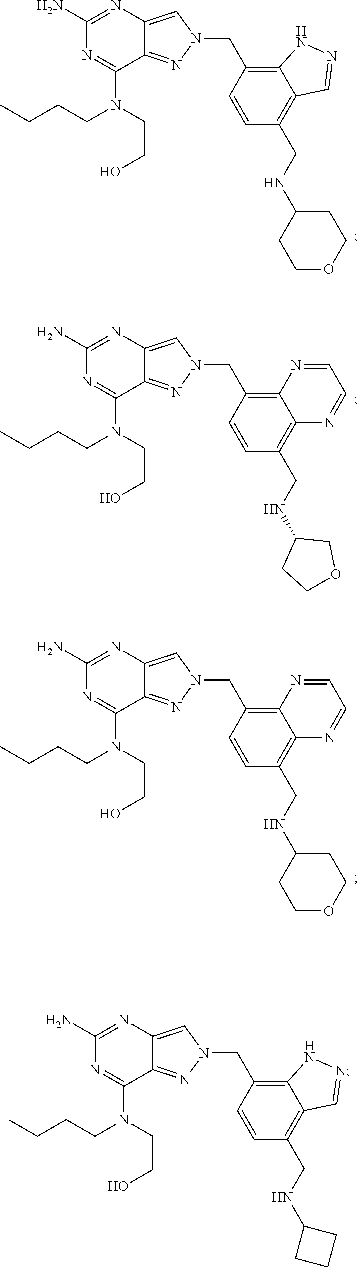 [SMILES: CCCCN(CCO)c1nc(N)nc2cn(Cc3ccc(CNC4CCOCC4)c4cn[nH]c34)nc12 ; CCCCN(CCO)c1nc(N)nc2cn(Cc3ccc(CN[C@H]4CCOC4)c4nccnc34)nc12 ; CCCCN(CCO)c1nc(N)nc2cn(Cc3ccc(CNC4CCOCC4)c4nccnc34)nc12 ; CCCCN(CCO)c1nc(N)nc2cn(Cc3ccc(CNC4CCC4)c4cn[nH]c34)nc12] 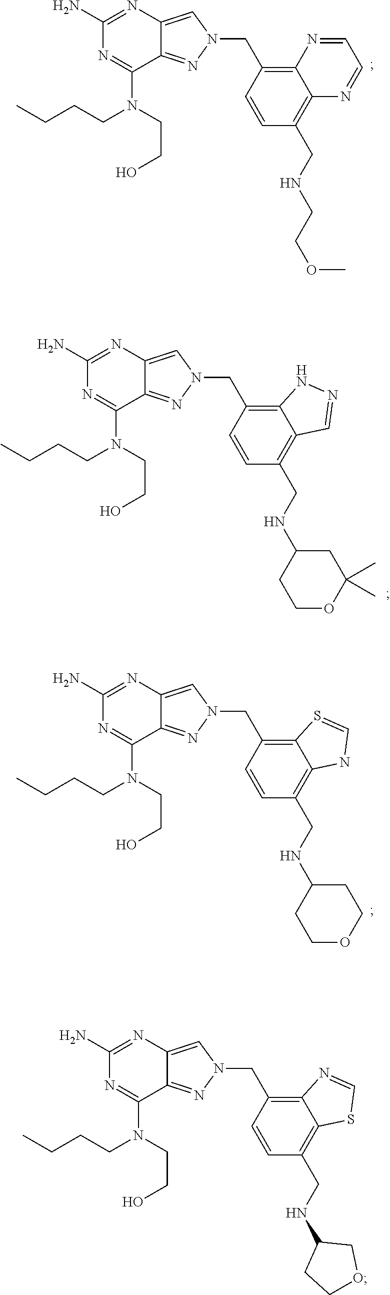 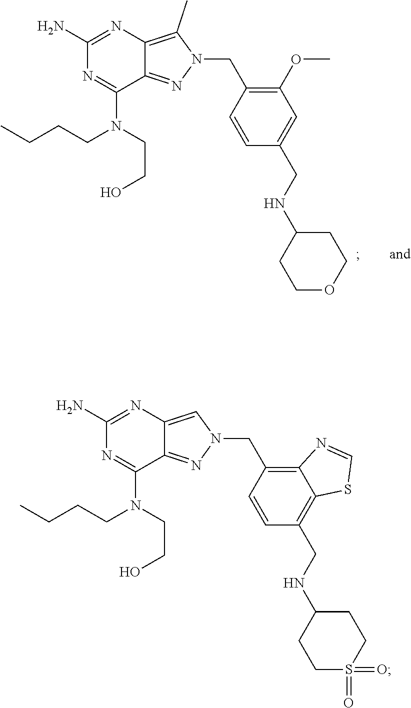 [SMILES: CCCCN(CCO)c1nc(N)nc2c(C)n(Cc3ccc(CNC4CCOCC4)cc3OC)nc12 ; CCCCN(CCO)c1nc(N)nc2cn(Cc3ccc(CNC4CCS(=O)(=O)CC4)c4scnc34)nc12]
or a pharmaceutically acceptable salt thereof.

32. A pharmaceutical composition comprising the compound of claim 1, or a pharmaceutically acceptable salt thereof, and a pharmaceutically acceptable excipient.

33. An antibody-drug conjugate comprising the compound of claim 1 and a targeting moiety, wherein the conjugate is represented by Formula (VI):
[D(X^{D})ₐ(C)_{c}(X^{Z})_{b}]ₘZ (VI)
wherein;
Z is a targeting moiety;
D is the compound; and
—(X^{D})ₐ(C)_{c}(X^{Z})_{b}— is a linker that links Z and D;
wherein:
C, if present, is a cleavable group;
X^{D} and X^{Z} are spacer moieties;
subscripts a, b, and c are each independently 0 or 1; and
subscript m is 1, 2, 3, 4, 5, 6, 7, 8, 9, or 10.

34. The antibody-drug conjugate of claim 33, wherein the targeting moiety is an antibody or antigen-binding portion thereof, a single-domain antibody or antigen-binding portion thereof, an antibody mimetic, an affibody, a nanobody, a unibody, a DARPin, an anticalin, a versabody, a duocalin, a lipocalin, or an avimer.

35. The antibody-drug conjugate of claim 33, wherein the targeting moiety binds a target selected from mesothelin, prostate specific membrane antigen (PSMA), CD19, CD22, CD30, CD70, B7H3, B7H4, protein tyrosine kinase 7 (PTK7), glypican-3, RG1, fucosyl-GM1, CTLA-4, and CD44.

36. A method of modulating Toll-Like Receptor 7 (TLR7) activity comprising contacting TLR7 with an effective amount of the compound of claim 1, or a pharmaceutically acceptable salt thereof.

37. A method of treating cancer in a subject in need thereof, comprising administering to the subject an effective amount of the compound of claim 1.

38-42. (canceled)

43. A method of modulating Toll-Like Receptor 7 (TLR7) activity comprising contacting TLR7 with an effective amount of the antibody-drug conjugate of claim 33.

44. A method of treating cancer in a subject in need thereof, comprising administering to the subject an effective amount of the antibody-drug conjugate of claim 33.

45. A compound selected from: 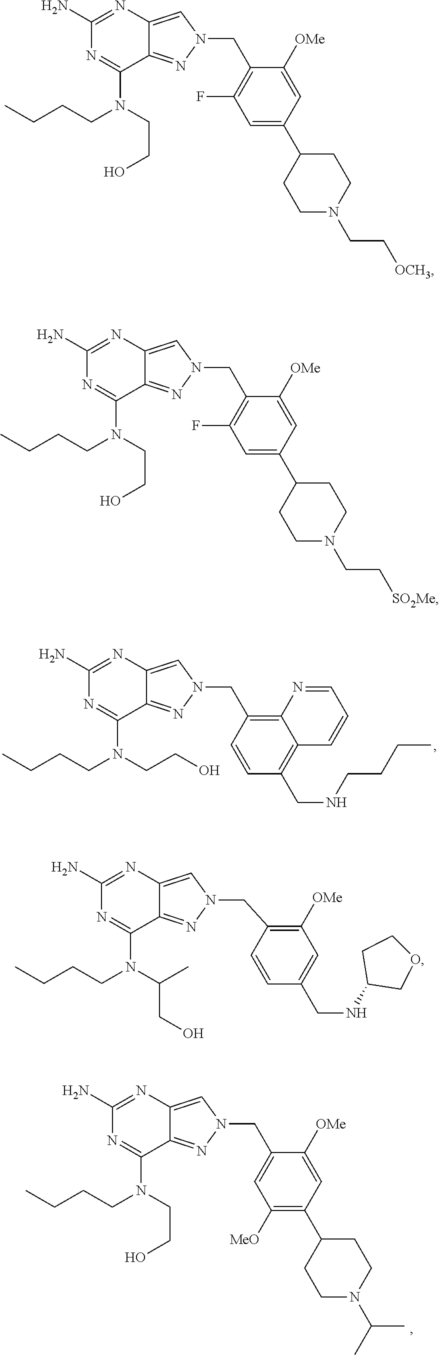 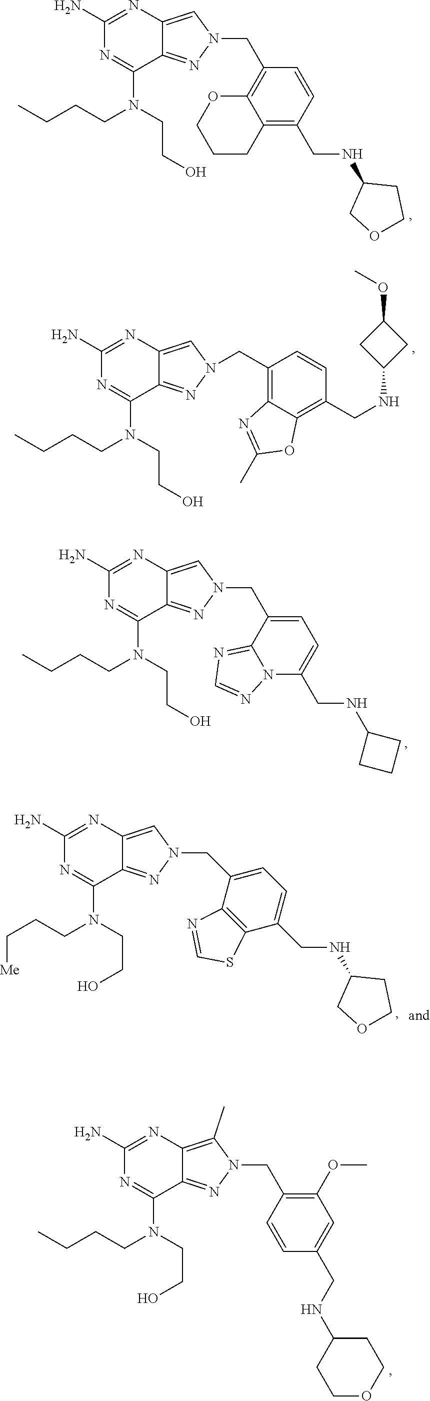
or a pharmaceutically acceptable salt thereof.
